(19) United States
(12) Patent Application Publication
(10) Pub. No.: US 2026/0166252 A1
(43) Pub. Date: Jun. 18, 2026

(54) METHOD AND SYSTEM FOR SIZING A PATIENT INTERFACE

(71) Applicant: Fisher & Paykel Healthcare Limited, Auckland (NZ)

(72) Inventors: Christopher Harding CAMPBELL, Auckland (NZ); Yun Chung JUNG, Auckland (NZ); Jonathan Ian ELLIS, Auckland (NZ); Jeremy Clive READ, Auckland (NZ); Benjamin Wilson CASSE, Auckland (NZ); Farlei Jose HEINEN, Auckland (NZ); Matthew James MCCONWAY, Auckland (NZ); Mark Jonathan BERRY, Auckland (NZ); Franco Magno Camino DANCEL, Auckland (NZ)

(21) Appl. No.: 19/114,357

(22) PCT Filed: Sep. 26, 2023
(86) PCT No.: PCT/NZ2023/050099

§ 371 Date: Mar. 21, 2025

Related U.S. Application Data
(60) Provisional application No. 63/377,158, filed on Sep. 26, 2022.
(60) Provisional application No. 63/483,955, filed on Feb. 8, 2023.
(60) Provisional application No. 63/494,451, filed on Apr. 5, 2023.
(60) Provisional application No. 63/517,818, filed on Aug. 4, 2023.

(51) Int. Cl. A61M 16/06; G06T 7/60; G06T 7/70; H04N 23/60

(52) U.S. Cl. CPC A61M 16/06; G06T 7/60; G06T 7/70; H04N 23/64; A61M 2016/0661; A61M 2205/3306; G06T 2207/30004; G06T 2207/30201; G06T 2207/30244

(57) ABSTRACT

A system for selecting a patient interface for a patient for use with a respiratory therapy device, the patient interface suitable to deliver respiratory therapy to the patient, the system comprising: receiver configured to receive data representing at least one first digital image of a face of a patient, the first digital image being a first facial image type; and configured to receive data representing at least one second digital image of the face of the patient, the second digital image being a second digital image type; image processor for determining a scaling factor from the first digital image; the image processor identifying a predefined facial feature appearing in the first or second digital image, and calculating a dimension for the facial feature using the scaling factor; and, a comparison engine for using the dimension to select a patient interface for the patient.

This application claims priority from the following patent applications: U.S. Provisional Patent Application No. 63/377,158 filed 26 Sep. 2022; U.S. Provisional Patent Application No. 63/483,955 filed 8 Feb. 2023; U.S. Provisional Patent Application No. 63/494,451 filed 5 Apr. 2023; and U.S. Provisional Patent Application No. 63/517,818 filed 4 Aug. 2023; the contents of all of which are incorporated herein by reference in their entirety.

### FIELD OF INVENTION

The present disclosure relates to a method and system for selecting a patient interface for a patient and sizing the patient interface for a patient, for use in providing respiratory therapy to a patient. The present disclosure in particular relates to selecting and sizing a mask or other patient interface that engages with an underside of the nose or engages the nostrils of a patient.

### BACKGROUND

The administration of continuous positive airway pressure (CPAP) therapy is common to treat obstructive sleep apnea. CPAP therapy is administered to a patient using a CPAP respiratory system which delivers therapy to the patient through a patient interface, generally a patient interface. Different patient interface types i.e. interface categories are available to patients including full face masks, nasal face masks and sub nasal masks i.e. under nose masks and nasal pillows. In each category of patient interfaces, there are different sizes available to fit faces of different shapes and sizes. Correct fitting of patient interfaces is important to avoid leaks in the CPAP system which can reduce the effectiveness of the therapy. Poorly fitted patient interfaces can also be uncomfortable to the patient and result in a negative or painful therapy experience. Similar considerations are also taken into account when providing other pressure therapies via a patient interface e.g. BiLevel pressure therapy.

Nasal high flow therapy is a common therapy used to treat spontaneously breathing patients with or at risk of respiratory distress. Nasal high flow therapy can be used to provide respiratory assistance to patients suffering from chronic respiratory diseases such as for example COPD. Nasal high flow therapy can be used in the home or hospital. Nasal high flow therapy is administered through an unsealed nasal cannula that comprise nasal prongs insertable into a patient's nostrils. Nasal cannula are available in different sizes to fit nostrils of different shapes and sizes. Incorrect sizing could lead to the prongs sealing with the nostrils. This can diminish the effectiveness of high flow therapy, increase the risk of barotrauma occurring due to the high flow rates and also increase discomfort. The prongs need to be unsealed for patient safety (i.e. to minimize risk of barotrauma) and for effectiveness of high flow therapy.

Patient interfaces and nasal cannula are often fitted by medical professionals during the prescription of therapy. Often, patients have to go to an equipment provider or physician or sleep lab. The fitting process may be a trial and error process and can take an extended time period. More recently patient interfaces can be selected remotely by patients, for example via online ordering stores rather than physically purchasing the patient interfaces in an environment where the patient interfaces may be professionally fitted. In some cases patients, at home, are sent all sizes of available patient interfaces to fit by trial and error. This can lead to incorrect fitting and also leads to waste as the patient interfaces of the wrong size have to be disposed of. A similar process occurs for nasal cannula sizing and fitting for patients being treated with high flow therapy at home.

CPAP therapy, BiLevel therapy and nasal high flow therapy can also be administered to patients in hospitals. In hospitals a nurse or other clinician will size the patient interface or nasal cannula by eye, and then by trial and error. This is process can be iterative and time consuming.

### SUMMARY OF THE INVENTION

In one aspect the invention provides a method for selecting a patient interface for a patient for use with a respiratory therapy device, the patient interface suitable to deliver respiratory therapy to the patient, comprising the steps of: receiving data representing at least one first digital image of a face of a patient, the first digital image being a first facial image type; determining a scaling factor from the first digital image; receiving data representing at least one second digital image of the face of the patient, the second digital image being a second digital image type; identifying a predefined facial feature appearing in the first or second digital image; calculating a dimension for the facial feature using the scaling factor; and, using the dimension to select a patient interface for the patient.

The step of using the dimension to select a patient interface for the patient may comprise the steps of comparing the calculated dimension of the facial feature with patient interface sizing data associated with patient patient interfaces and selecting a patient interface for the patient in dependence on the comparison.

Embodiments include the step of selecting the facial feature for identification in the second digital image. The facial feature may be selected from a plurality of facial features. The step of selecting the facial feature may be performed based on a designated patient interface category for the patient. The step of determining a designated patient interface category for the patient may be performed by:
presenting at least one user question to a user;
receiving at least one user response to the at least one user question; and
determining a patient interface category for the patient in dependence on the received user response.

The facial feature may be one of: depth of the nose; or nostril size.

Examples may comprise the steps wherein the first facial image type and the second facial image type include the face of the patient at different orientations.

Examples may comprise the steps of:
determining at least one attribute of the first digital image; comparing the at least one attribute with predefined attribute criteria; and determining whether the at least one attribute meets the predefined attribute criteria;
wherein the step of determining a scaling factor is performed in dependence on the at least one attribute meeting the predefined attribute criteria.

Examples may comprise the steps of:
determining at least one attribute of the second digital image; comparing the at least one attribute with predefined attribute criteria; and determining whether the at least one attribute meets the predefined attribute criteria;
wherein the steps of determining a measurement of the facial feature in the second image; and calculating a dimension of the facial feature using the scaling factor and the measurement of the facial feature are performed in dependence on the at least one attribute meeting the predefined attribute criteria.

The at least one attribute comprises may at least one of: an angle of the face of the user within the image, the angle being at least one of the pitch angle, the yaw angle or the roll angle.

The at least one attribute may further comprise at least one of: the focal length of the image; depth of the patient's face in the image; and at least one predefined landmark being identified in the image.

The first predefined attribute may be the angle, the predefined angle being between 0 to +−6 degrees with respect to the plane of the image. The second predefined attribute may be the pitch angle, the predefined angle being between 35 to 45 degrees with respect to the plane of the image.

The first digital image type may be a front facial image. The second digital image may be an underside facial image.

Examples comprise the further step of providing feedback relating to whether the at least one attribute meets the predefined attribute criteria.

In examples, the step of calculating a scaling factor comprises the steps of:
identifying a predefined reference facial feature appearing in the image, the predefined reference facial feature being an eye of the patient;
determining a measurement for the eye of the patient within the image;
allocating a predefined dimension to the measurement, and determining a scaling factor for the image, the scaling factor being a ratio between the measurement and the predefined dimension.

In one aspect the invention provides system for selecting a patient interface for a patient for use with a respiratory therapy device, the patient interface suitable to deliver respiratory therapy to the patient, the system configured to perform the steps of: receiving data representing at least one first digital image of a face of a patient, the first digital image being a first facial image type; determining a scaling factor from the first digital image; receiving data representing at least one second digital image of the face of the patient, the second digital image being a second digital image type; identifying a predefined facial feature appearing in the first or second digital image; calculating a dimension for the facial feature using the scaling factor; and, using the dimension to select a patient interface for the patient.

In a further aspect the invention provides a system for selecting a patient interface for a patient for use with a respiratory therapy device, the patient interface suitable to deliver respiratory therapy to the patient, the system comprising:
receiver configured to receive data representing at least one first digital image of a face of a patient, the first digital image being a first facial image type; and configured to receive data representing at least one second digital image of the face of the patient, the second digital image being a second digital image type;
image processor for determining a scaling factor from the first digital image;
the image processor identifying a predefined facial feature appearing in the first or second digital image, and calculating a dimension for the facial feature using the scaling factor; and, a comparison engine for using the dimension to select a patient interface for the patient.

The step of using the dimension to select a patient interface for the patient may comprise the steps of comparing the calculated dimension of the facial feature with patient interface sizing data associated with patient interfaces and selecting a patient interface for the patient in dependence on the comparison.

Examples include the step of selecting the facial feature for identification in the second digital image. In examples the facial feature is selected from a plurality of facial features. In examples, the step of selecting the facial feature is performed based on a designated patient interface category for the patient. In examples, the further step of determining a designated patient interface category for the patient.

Examples further comprise a user interface, the user interface being configured to:
present at least one user question to a user; and, receiving at least one user response to the at least one user question;
wherein the processor determines a patient interface category for the patient in dependence on the received user response, the predefined facial feature being selected in dependence on the determined patient interface category.

The facial feature may be one of: depth of the nose; or nostril size.

The first facial image type and the second facial image type may include the face of the patient at different orientations.

Examples may comprise an orientation sensor, the orientation sensor configured to measure at least one attribute of the first digital image; the processor is configured to compare the at least one attribute with predefined attribute criteria; and determine whether the at least one attribute meets the predefined attribute criteria;
wherein the image processor performs the step of determining a scaling factor in dependence on the at least one attribute meeting the predefined attribute criteria.

The orientation sensor may comprise at least one of an accelerometer or a gyroscope.

The at least one attribute may further comprise at least one of: the focal length of the image; depth of the patient's face in the image; and at least one predefined landmark being identified in the image.

The first predefined attribute may be the angle, the predefined angle being between 0 to +−6 degrees with respect to the plane of the image. The second predefined attribute may be the pitch angle, the predefined angle being between 35 to 45 degrees with respect to the plane of the image.

The first digital image type may be a front facial image. The second digital image is an underside facial image.

Examples comprise a user interface, the user interface being configured to provide feedback relating to whether the at least one attribute meets the predefined attribute criteria.

The image processor may calculate a scaling factor by performing the steps of:
identifying a predefined reference facial feature appearing in the image, the predefined reference facial feature being an eye of the patient;
determining a measurement for the eye of the patient within the image;
allocating a predefined dimension to the measurement, and determining a scaling factor for the image, the scaling factor being a ratio between the measurement and the predefined dimension.

In one aspect the invention provides a method for selecting a patient interface for a patient for use with a respiratory therapy device, the patient interface suitable to deliver respiratory therapy to the patient, comprising the steps of:
presenting at least one user question to a patient;
receiving at least one user response to the at least one user question; and
determining a designated patient interface category suitable for the patient in dependence on the at least one received user response;
identifying at least one patient facial feature dimension required to select a patient interface for the patient within the designated patient interface category; and
determining at least one facial image type required to calculate the patient facial feature dimension.

The designated patient interface category may define a suitable patient interface category for delivering respiratory therapy to the patient.

The facial image type may be defined by the orientation of the face of the patient in the image. The facial image type may include: a front facial image; and, an underside facial image.

Examples may comprise the further step of providing instructions to the patient to capture the determined at least one facial image type.

Examples may comprise the further steps of receiving at least one image of the determined at least one facial image type; calculating from the received at least one facial image type the identified at least one patient facial feature dimension; and using the calculated at least one facial feature dimension to select an interface for the patient. The step of using may comprise the steps of comparing the calculated dimension of the facial feature with interface sizing data associated with patient interface and selecting a patient interface for the patient in dependence on the comparison.

In a further aspect the invention provides a system for selecting a patient interface for a patient for use with a respiratory therapy device, the patient interface suitable to deliver respiratory therapy to the patient, the system configured to preform the steps of:
presenting at least one user question to a patient;
receiving at least one user response to the at least one user question; and
determining a designated patient interface category suitable for the patient in dependence on the at least one received user response;
identifying at least one patient facial feature dimension required to select a patient interface for the patient within the designated patient interface category; and
determining at least one facial image type required to calculate the patient facial feature dimension.

In one aspect the invention provides a method for selecting an interface for a patient for use with a respiratory therapy device, the interface suitable to deliver respiratory therapy to the patient, comprising the steps of: presenting at least one user question to a patient; receiving at least one user response to the at least one user question; and determining a designated interface category suitable for the patient in dependence on the at least one received user response; and determining at least one facial image type required to select an interface for the patient of the designated interface category.

The step of determining the at least one facial image type may be performed by identifying at least one patient facial feature dimension required to select a patient interface of the designated patient interface category; and identifying at least one facial image type from which the patient facial feature dimension can be calculated.

Examples may comprise the further steps of receiving at least one image of the determined at least one digital image type; calculating from the received at least one image the identified at least one patient facial feature dimension; and using the calculated at least one facial feature dimension to select an interface for the patient.

The step of using may comprise the steps of comparing the calculated dimension of the facial feature with interface sizing data associated with patient interface and selecting a patient interface for the patient in dependence on the comparison.

The designated patient interface category may define a preferred interface category suitable for delivering respiratory therapy to the patient. The at least one facial image type may be defined by the orientation of the head of the patient in the image. The at least one facial image types may be defined by orientation of the patient's face in the image, the facial image types include: front facial image and underside facial image.

Examples may comprise the further step of providing instructions to the patient to capture the determined at least one facial image type.

In one aspect the invention provides a system for selecting an interface for a patient for use with a respiratory therapy device, the interface suitable to deliver respiratory therapy to the patient, the system being configured to perform the steps of:
presenting at least one user question to a patient;
receiving at least one user response to the at least one user question; and
determining a designated interface category suitable for the patient in dependence on the at least one received user response; and determining at least one facial image type required to select an interface for the patient of the designated interface category.

In one aspect the invention provides a system for selecting an interface for a patient for use with a respiratory therapy device, the interface suitable to deliver respiratory therapy to the patient, the system comprising:
user interface configured to present at least one user question to a patient and to receive at least one user response to the at least one user question; and
processor configured to determine a designated interface category suitable for the patient in dependence on the at least one received user response; and
determining at least one facial image type required to select an interface for the patient of the designated interface category.

In one aspect the disclosure provides a method for selecting a patient interface for a patient for use with a respiratory therapy device, the patient interface suitable to deliver respiratory therapy to the patient, comprising the steps of:
receiving data representing at least one digital image of a face of a patient;
identifying a predefined reference facial feature appearing in the image, the predefined reference facial feature being an eye of the patient;
determining a measurement for the eye of the patient within the image;
allocating a predefined dimension to the measurement, and
determining a scaling factor for the image, the scaling factor being a ratio between the measurement and the predefined dimension;
identifying a further facial feature in the image;
determining a measurement of the further facial feature in the image; and calculating a dimension of the further facial feature using the scaling factor and the measurement of the further facial feature; and,
comparing the calculated dimension of the further facial feature with patient interface sizing data associated with patient patient interfaces; and,
selecting a patient interface for the patient in dependence on the comparison.

The measurement for the eye of the patient may be a width measurement. The measurement for the eye of the patient may be a height measurement.

The step of selecting a patient interface may comprise the step of identifying a patient interface.

The step of identifying an eye of the patient in the image may be performed by identifying at least two predefined facial landmarks in the image associated with the eye. The at least two predefined facial landmarks in the image may be the corners of the eye. The predefined facial landmarks may be the medial canthus and the lateral canthus. The measurement for the eye may be the width of the palpebral fissure.

The further facial feature may be identified by identifying at least two facial landmarks associated with the further facial feature. The further facial feature may be used to size the patient interface.

The step of determining a measurement of a facial feature may be performed by calculating a number of pixels of the image between at least two facial landmarks in the image associated with the facial feature.

The step of determining a measurement for the reference feature within the image may be performed by identifying two eyes of the patient within the image and calculating a measurement for each eye and calculating an average measurement for the two eyes.

The facial landmarks may be anthropometric features of a patient's face identified within the image.

The method may comprise the further steps of:
determining at least one attribute of the digital image;
comparing the at least one attribute with predefined attribute criteria; and determining whether the at least one attribute meets the predefined attribute criteria;
wherein the step of selecting a patient interface for the patient is performed in dependence on the at least one attribute meeting the predefined attribute criteria. The at least one attribute may comprise at least one of:
an angle of the face of the user within the image, the angle being at least one of the pitch angle, the yaw angle or the roll angle;
the focal length of the image;
depth of the patient's face in the image; and
at least one predefined landmark being identified in the image.

The at least one attribute may be the pitch angle, the predefined angle being between 0 to +−6 degrees with respect to the plane of the image.

The method may comprise the further step of providing feedback relating to whether the at least one attribute meets the predefined attribute criteria.

In embodiments, the step of calculating the dimension of the further facial feature may be performed for multiple images, to produce multiple calculated dimensions, the method comprising the further step of calculating an average dimension of the further facial feature across the multiple images; and using the average dimension to compare with the patient interface sizing data. The average dimension may be calculated across a predetermined number of images.

Embodiments may include the step of determining at least one attribute of the digital images;
comparing the at least one attribute with predefined attribute criteria; and determining whether the at least one attribute meets the predefined attribute criteria;
wherein the average dimension is calculated for images which meet the predefined attribute criteria.

Embodiments may comprise the further steps of:
presenting at least one user question to a user;
receiving at least one user response to the at least one user question; and
determining a patient interface category for the patient in dependence on the received user response.

The further facial feature may be selected from a plurality of facial features in dependence on the patient interface category.

The patient interface sizing data associated with patient patient interfaces may be associated with patient interfaces of the determined patient interface category.

Patient interface may be defined as being in a patient interface category, wherein different patient interface categories have different relationships between patient interface sizing data and dimensions of facial features.

The further facial feature may be selected from a plurality of facial features, the selection being made based on a designated patient interface category.

In a further aspect the disclosure provides a method for selecting a patient interface for a patient for use with a respiratory therapy device, the patient interface suitable to deliver respiratory therapy to the patient, comprising the steps of: presenting at least one user question to a user; receiving at least one user response to the at least one user question; determining a patient interface category associated with the user in dependence on the received user response; receiving a digital image of a face of a patient; within the image, identifying a predefined reference feature of the patient's face appearing in the image, allocating a dimension to the reference feature in the image, and determining a scaling factor for the image based on the reference feature; within the image, identifying at least one preselected feature of the patient's face appearing in the image, wherein the at least one preselected feature is selected in dependence on the determined patient interface type category, and calculating a dimension associated with the at least one preselected feature using the measurement scale; and, comparing the calculated dimension of the preselected feature with patient interface sizing data associated with patient patient interfaces and, selecting a patient interface for the patient in dependence on the comparison.

The calculated dimension of the preselected feature may be compared with patient interface sizing data associated with patient patient interfaces of the determined patient interface type category. Embodiments may determine if the preselected feature appears in the image and provide user feedback in dependence on whether it appears in the image.

In a further aspect the disclosure provides a method for selecting a patient interface for a patient for use with a respiratory therapy device, the patient interface suitable to deliver respiratory therapy to the patient, comprising the steps of:
receiving a digital image of a face of a patient;
determining attributes of the digital image;
comparing the attributes with predefined attribute criteria; and, provide user feedback relating to whether the attributes meet the predefined attribute criteria;
within the image, identifying a predefined reference feature of the patient's face appearing in the image, allocating a dimension to the reference feature in the image, and
determining a measurement scale for the image using the reference feature;
within the image, identifying at least one preselected feature of the patient's face appearing in the image, and calculating a dimension associated with the at least one preselected feature using the measurement scale; and,
comparing the calculated dimension of the preselected feature with patient interface sizing data associated with patient patient interfaces; and,
selecting a patient interface for the patient in dependence on the comparison.

In a further aspect the disclosure provides a system for selecting a patient interface for a patient for use with a respiratory therapy device, the patient interface suitable to deliver respiratory therapy to the patient, the system comprising:
a processor configured to:
   receive data representing at least one digital image of a face of a patient;
identify a predefined reference facial feature appearing in the image, the predefined reference facial feature being an eye of the patient;
   determine a measurement for the eye of the patient within the image;
   allocate a predefined dimension to the measurement, and
determine a scaling factor for the image, the scaling factor being a ratio between the measurement and the predefined dimension;
   identify a further facial feature in the image;
   determine a measurement of the further facial feature in the image; and calculate a dimension of the further facial feature using the scaling factor and the measurement of the further facial feature; and,
   a memory for storing patient interface sizing data associated with patient patient interfaces;
   the processor further configured to:
   compare the calculated dimension of the further facial feature with the stored patient interface sizing data associated with patient patient interfaces and select a patient interface for the patient in dependence on the comparison.

The system may comprise a display to display the selected patient interface to the patient. The system may comprise an image capture device for capturing digital image data representing a face of a patient.

In a further aspect the disclosure provides a software application configured to be executed on a client device, the software application configured to perform the method of any of the previous aspects.

In a further aspects the disclosure provides a mobile communication device configured to select a patient interface for a patient for use with a respiratory therapy device, the patient interface suitable to deliver respiratory therapy to the patient, the mobile communication device comprising:
an image capture device for capturing digital image data;
a processor configured to:
receive, from the image capture device, data representing at least one digital image of a face of a patient;
identify a predefined reference facial feature appearing in the image, the predefined reference facial feature being an eye of the patient;
determine a measurement for the eye of the patient within the image;
allocate a predefined dimension to the measurement, and
determine a scaling factor for the image, the scaling factor being a ratio between the measurement and the predefined dimension;
identify a further facial feature in the image;
determine a measurement of the further facial feature in the image; and calculate a dimension of the further facial feature using the scaling factor and the measurement of the further facial feature; and,
a memory for storing patient interface sizing data associated with patient patient interfaces;
   the processor further configured to:
compare the calculated dimension of the further facial feature with the stored patient interface sizing data associated with patient patient interfaces and select at least one patient interface for the patient in dependence on the comparison; and
a user interface to display data related to the at least one selected patient interface.

The method comprises the steps of receiving data representing at least one digital image of a face of a patient. The method identifies a predefined reference facial feature appearing in the image, where the predefined reference facial feature is an eye of the patient. The method determines a measurement for the eye of the patient within the image and allocates a predefined dimension to the measurement. The method determines a scaling factor for the image, where the scaling factor is a ratio between the measurement and the predefined dimension. The method identifies a further facial feature in the image, determines a measurement of the further facial feature in the image and calculates a dimension of the further facial feature using the scaling factor and the measurement of the further facial feature. The method compares the calculated dimension of the further facial feature with patient interface sizing data associated with patient patient interfaces and selects a patient interface for the patient in dependence on the comparison. Embodiments provide an accurate measurement system that allows a non-technical expert, to accurately and reliably capture the information required for the system to recommend a well-fitting patient interface. The method can be implemented, using non-professional equipment. Embodiments capture images of the patient face which allow accurate and reliable sizing to be derived using a reference scale. The described method and system provide a convenient method for patient interface sizing as a user (e.g. an OSA patient) can perform this method at home without having to visit a clinician and without the need of any professional equipment. Further the method for sizing is convenient as it can be executed on a mobile device of a user e.g. a smartphone or tablet. The described method and system for patient interface sizing are also advantageous because there is no requirement for a separate reference object that needs to be held in front of the patient's face to perform the patient interface sizing.

In a further embodiment the invention provides, a method for selecting a patient interface for a patient for use with a respiratory therapy device, the patient interface suitable to deliver respiratory therapy to the patient, comprising the steps of: determining a dimension of a facial feature required in order to select a patient interface for a patient; determining a desired orientation of the face of the patient to be captured in a digital image, in order to determine the dimension of the required facial feature; providing guidance to the patient to position their face in the desired orientation for image capture; receiving image capture data representing at least one digital image of a face of a patient, the image capture data representing the face of the patient orientated in the desired orientation; calculating a dimension for the facial feature from the image capture data; and, using the dimension to select a patient interface for the patient.

The method may comprise the step of determining an orientation of the image capture device (ICD), comparing the orientation of the image capture device to a predefined orientation, and providing guidance to position the image capture device into the predefined orientation in dependence on the comparison. The predetermined orientation being a vertical orientation.

The ICD may be held vertical, and the guidance may be to instruct the patient to move their head relative to the ICD. The ICD may be held vertical, and the guidance may be to instruct movement of the ICD.

The step of providing guidance may provide at least one of instructions to re-orientate the face of patient, and instructions to re-orientate the image capture device. The ICD and the head may both be angled, with the orientation being a relative angle between them; and the guidance is to instruct the patient to move their head and/or to instruct movement of the ICD.

The method may comprise the step of receiving selection of an orientation of the image capture device, the selected orientation being reference orientation, wherein the reference orientation is selected by the user or selected automatically, and wherein subsequent measurements are taken with respect to the reference orientation.

The reference orientation for the image capture device may be the orientation of the image capture device when the face of the patient is at a predefined orientation relative to the image capture device. The reference orientation for the image capture device may be an orientation where the camera of the image capture device is parallel to the face of the patient.

With reference to the base angle or starting angle: the base angle or starting angle may correspond to a position at which the IMD is substantially parallel to the patient's face; The base angle or starting angle may correspond to a position at which the IMD is substantially at a desired orientation relative to the patient's face.

In examples, the step of providing guidance to the patient to position their head in the desired orientation for image capture is performed using the steps of: receiving data representing at least one digital image of a face of a patient; determining, from the received data representing at least one digital image of the face of the patient, a first orientation of the head of the user, and presenting using a position indicator a first indicator associated with the first orientation; receiving further data representing at least one digital image of a face of a patient, and determining, from the received further data representing at least one digital image of the face of the patient, a second orientation of the face of the patient, said second orientation being different from the first orientation, and presenting using the position indicator a second indicator associated with the second orientation; wherein the first indicator indicates to the user a comparison between the first orientation and the desired orientation, and the second indicator indicates to the user a comparison between the second orientation and the desired orientation.

The step of providing guidance to the patient to position their head in the desired orientation for image capture may be performed using the steps of: on a display interface displaying a static indicator representing the desired orientation of the face, the static indicator being displayed at a fixed location on the display interface; receiving data representing at least one digital image of a face of a user; determining, from the received data representing at least one digital image of the face of the user, an orientation of the face of a user; on the display interface, displaying a dynamic indicator representing the current orientation of the face, wherein a difference between the location of the static indicator and the dynamic indicator on the display interface is representative of a difference between the current orientation of the user's face and the desired orientation of the user's face.

The step of providing guidance to the patient to position their head in the desired orientation for image capture may be performed using the steps of: receiving first data representing at least one digital image of a face of a patient; executing a height application to guide the user to attain a required height value of the user's face with respect to the image capture device; executing a distance application to guide the user to attain a required distance value between the user's face and the image capture device; executing an angle application to guide the user to attain a required angle value of the face of the user with respect to the image capture device; wherein when the when the user has attained at least one of the required height value, required distance value, and required angle value, capturing an image of the user's face with the image capture device.

The step of providing guidance to the patient to position their head in the desired orientation for image capture may be performed using the steps of: providing prompts to the user to assist the user to attain a required height of the user's face relative to the image-capture device; providing prompts to the user to assist the user to attain a required angle of the user's face relative to the image-capture device; and providing prompts to the user to assist the user to attain a required distance of the user's face relative to the image-capture device.

The step of providing guidance to the patient to position their head in the desired orientation for image capture may be performed using the steps of: detecting an image of the face of the user with the image capture device, using the image to calculate a three-dimensional relation between the face of the user and the image capture device; comparing the calculated three-dimensional relation with a required three-dimensional relation; determining change in position and/or orientation of the user's face required to meet the required three-dimensional relation; and, presenting guidance to the user to re-position and/or re-orientate their face to achieve or move towards the required three-dimensional relation.

In a further aspect the invention provides a method for guiding a user (aka patient) to position their face in a desired orientation for image capture, the method comprising: receiving first data representing at least one digital image of a face of a user; determining, from the received first data representing at least one digital image of the face of the patient, a first orientation of the face of a user, and presenting using a position indicator a first indicator associated with the first orientation; receiving second data representing at least one digital image of a face of a patient; determining, from the received further data representing at least one digital image of the face of the patient, a second orientation of the face of a user, said second orientation being different from the first orientation, and presenting using the position indicator a second indicator associated with the second orientation; wherein the first indicator indicates to the user a comparison between the first orientation and the desired orientation, and the second indicator indicates to the user a comparison between the second orientation and the desired orientation.

In a further aspect the invention provides a system for guiding a user (aka patient) to position their face in a desired orientation for image capture, comprising: processor for receiving first data representing at least one digital image of a face of a patient; the processor determining, from the received first data representing at least one digital image of the face of the user, a first orientation of the face of a user, and presenting using a position indicator a first indicator associated with the first orientation; the processor receiving second data representing at least one digital image of a face of a patient; and, determining, from the received further data representing at least one digital image of the face of the patient, a second orientation of the face of a user, said second orientation being different from the first orientation, and presenting using the position indicator a second indicator associated with the second orientation; the first indicator indicates to the user a comparison between the first orientation and the desired orientation, and the second indicator indicates to the user a comparison between the second orientation and the desired orientation.

In a further aspect the invention provides a position indicator comprising at least a portion di splayed in use on a display screen of a device, for guiding a user to position their face in a desired orientation for image capture, configured to perform the steps of: receiving first data representing at least one digital image of a face of a patient; determining, from the received first data representing at least one digital image of the face of the patient, a first orientation of the face of a user, and presenting a first indicator associated with the first orientation; receiving second data representing at least one digital image of a face of a patient; determining, from the received further data representing at least one digital image of the face of the patient, a second orientation of the face of a user, said second orientation being different from the first orientation, and presenting a second indicator associated with the second orientation; wherein the first indicator indicates to the user a comparison between the first orientation and the desired orientation, and the second indicator indicates to the user a comparison between the second orientation and the desired orientation.

In examples the first indicator and the second indicator are different. The received first data and the received second data may be obtained from an image capture device and the orientation of the face of the user is determined with respect to the image capture device.

The first indicator and the second indicator may be visual indicators. The visual indicators may be displayed on a display interface of an electronic device. The electronic device may comprise the display interface and the image capture device. The first indicator and the second indicator may be displayed at different locations on the display interface. The position indicator may comprise the first indicator and the second indicator.

The position indicator may present a progressive animation sequence to indicate the orientation of the face towards and away from the desired orientation in dependence on the current orientation, the progressive animation sequence comprising a series of indicators displayed at different locations on the display interface, the first and second indicators being indicators of the sequence of indicators.

For a given orientation of the user's face relative to the image-capture device, the relevant indicator associated with the given orientation may be displayed on the display interface at a location on the display screen visible to the user.

Examples may comprise the step of determining a field of view of the user and wherein, at a given orientation of the face, the relevant indicator is displayed on the display interface at a location on the display interface within the field of view of the user. Examples may comprise the step of determining, for a given orientation of the face of the user, a portion of the display interface which is visible to the user, wherein the relevant indicator being displayed at a location within the visible portion of the display interface. The portion of the display interface which is visible to the user may be determined using the orientation of the head of the user. The field of view of the user may be determined using an angular range with respect to the front of the face of the user at said determined orientation, as appearing in the data representing the face of the user. The field of view of the user may be determined using an angular range with respect to the eye of the user at said determined orientation, as appearing in the data representing the face of the user.

Examples may comprise the step of determining the position of the camera and wherein the field of view of the user is determined using the position of the camera. The display device may include the camera.

The portion of the display interface which is visible to the user may be determined dependent on a relative angle between the face of the user and the display interface. The greater the angle between the display interface and the face of the user, the smaller the portion of the display interface that will be visible to the user.

The position indicator may be located on a first portion of the display interface, said first portion at least partly defined by a first edge of the display interface. As a relative angle between the face and the display interface increases, the user may have progressively less visibility of said first portion, and more particularly of a portion of said first portion that is distal from the first edge. In use, the second indicator, corresponding to a greater relative angle, may be positioned closer to the first edge than the first indicator (corresponding to a lesser relative angle than the second indicator). On determining that the orientation of the face is the desired orientation, examples may present an image capture indicator (aka progress indicator) to indicate that the image capture device is capturing an image of the face.

The image capture indicator may be displayed on the display interface at a location within the field of view of the user. The image capture indicator may be displayed on the display interface at a location proximate to the position indicator at the location where the position indicator indicated that the orientation of the face of the user meets the desired orientation.

Examples, on determining that the orientation of the face is the desired orientation, trigger an image capture process, the image capture process comprising the steps of: displaying an image capture indicator (aka progress indicator) on the display interface at a location on the display interface; capturing an image of the face with the image capture device for a predefined time period; and during said predefined time period, monitoring the orientation of the face; wherein during the predetermined time period if the orientation of the face changes from the desired orientation, at least one of: terminating the image capture process; or suspending the image-capture process and displaying prompts to urge the user to return their face to the desired orientation. In examples, upon terminating or suspending the image capture process, presenting a different indicator, and/or an additional indicator, from the image capture indicator (aka progress indicator). The image capture indicator may be a progressive animation indicator which updates during the predefined time period. The indicator may be a visual indicator, an audio indicator, a haptic indicator, or other indicator type.

The position indicator may present a progressive animation sequence to indicate the orientation of the head towards and away from the desired orientation, the progressive animation sequence comprising a series of indicators displayed at different locations on the display interface, the first and second indicators being indicators of the sequence of indicators.

The desired orientation of the head with respect to the camera may be a non-frontal orientation.

In examples, in addition to the first and second indicators, the position indicator may further comprise non-current-position-indicating portions corresponding to other potential, non-current, orientations of the user's face relative to the image capture device. The indicator may be presented in real-time on determining the orientation of the head.

In a further aspect the invention provides a method for indicating the orientation of a body with respect to a camera compared with a desired orientation of the body with respect to the camera comprising: on determining the body is in a first orientation with respect to the camera presenting a first indicator; subsequently, on determining the body is in a second, different, orientation with respect to the camera, presenting a second indicator; wherein the position indicator is configured to indicate whether the second orientation is closer to or further from the desired orientation compared with the first orientation.

In a further aspect the invention provides a system configured to generate a position indicator for display on a display screen, the position indicator being configured to assist a user in positioning the user's face at a required non-frontal angle relative to an image-capture device to enable capture by the image-capture device of an image of the user's face at said required angle, the position indicator being configured to dynamically change position and/or appearance on the display screen in response to a detected change in angle of the user's face relative to the image-capture device, such that, for a given angle of the user's face relative to the image-capture device, at least a current-position-indicating portion of the position indicator is visible on the display screen to the user, wherein, for a first angle of the user's face, the at least a current-position-indicating portion is positioned in a first position on the display screen; and for a second, different, angle of the user's face, the at least a current-position-indicating portion is positioned in a second, different position on the display screen, said second angle of the user's face being a greater angle relative to the display screen than the first angle, wherein the second position of the at least a current-position-indicating portion of the position indicator compensates for a reduced field of vision, relative to the display screen, of the user at the second angle compared to the first angle.

For the first angle, a first portion of the display screen proximate the first edge is within the user's field of vision; and for the second angle, a second portion of the display screen proximate the first edge is within the user's field of vision, said second portion being smaller than said first portion; wherein the first position (of the indicator) is within the first portion, and the second position (of the indicator) is within the second portion. Based on the detected angle and/or position of the user's face relative to the image-capture device, the system is configured to determine a visible portion of the display screen. The system may also be configured to determine a boundary of a field of vision of the user. The boundary being a line extending at an angle from a portion of the face of the user. The portion of the face of the user may be an eye of the user. The system may be configured to determine an intersection point between the boundary line and the image-capture device, in order to determine the visible portion of the display screen.

In a further aspect the invention provides a method for guiding a user to position their face in a desired orientation for image capture, the method comprising the steps of: receiving first data representing at least one digital image of a face of a patient; determining, from the received first data representing at least one digital image of the face of the patient, a first orientation of the face of a user, and presenting using a position indicator a first indicator associated with the first orientation; receiving second data representing at least one digital image of a face of a patient; determining, from the received further data representing at least one digital image of the face of the patient, a second orientation of the face of a user, said second orientation being different from the first orientation, and presenting using the position indicator a second indicator associated with the second orientation; wherein the first indicator indicates to the user a comparison between the first orientation and the desired orientation, and the second indicator indicates to the user a comparison between the second orientation and the desired orientation.

The position indicator may be a visual indicator for display on a display interface of an electronic device. The position indicator may present a real-time animation to indicate the orientation of the face towards and away from the desired orientation in dependence on the current orientation, the real-time animation comprising a series of indicators, the first and second indicators being indicators of the series of indicators.

In a further aspect the invention provides a method configured to display: a static indicator representing the desired orientation of the face and a dynamic indicator representing the real-time orientation of the face; the static indicator being displayed at a fixed location on the display interface; the dynamic indicator being displayed at a location on the display interface representative of the current orientation of the face, the dynamic indicator comprising the first indicator and the second indicator; wherein a difference between the display location of the static and the display location of the dynamic indicator on the display interface represents a difference between current orientation of the user's head and the desired orientation of the user's head.

The location of the dynamic indicator on the display interface may be updated dynamically in dependence on the current orientation of the face.

The dynamic animation may include an image of the user's face. The dynamic indicator may be configured to be displayed on the display interface substantially around a displayed real-time image of the user's face, such that both the user's face and the dynamic indicator move dynamically on the display interface as the orientation of the user's face changes.

Alignment between the static and dynamic indicators may indicate that the user's face is at the desired orientation.

The static indicator may comprise a circle, and the dynamic indicator comprises a circle and, wherein, the circles are located concentrically when the orientation of the user's face meets the criteria for the desired orientation. The desired orientation may comprise at least one of: angle, height and distance. The desired orientation may comprise angle, height and distance.

In a further aspect the invention provides a method for guiding a user to position their face in a desired orientation for image capture, comprising the steps of: on a display interface displaying a static indicator representing the desired orientation of the face and a dynamic indicator representing the current orientation of the face; the static indicator being displayed at a fixed location on the display interface; the dynamic indicator being displayed at a location on the display interface representative of the current orientation of the face; receiving data representing at least one digital image of a face of a user; determining, from the received data representing at least one digital image of the face of the user, an orientation of the face of a user; on the display interface, displaying a dynamic indicator representing the current orientation of the face, wherein a difference between the location of the static indicator and the dynamic indicator on the display interface is representative of a difference between the current orientation of the user's face and the desired orientation of the user's face.

In a further aspect the invention provides a method for guiding a user to position and orientate their face and an image capture device in a required three-dimensional relation relative to one another for image capture, the method comprising the steps of: executing of a height application to guide the user to attain a required height value of the user's face with respect to the image capture device; when the user has attained the required height value, triggering a distance application to guide the user to attain a required distance value between the user's face and the image capture device; during execution of the distance application, monitoring the height of the user's face with respect to the image capture device, wherein if the height of the user's face is outside the required height value, interrupting (and/or supplementing with additional prompts) the distance application; when the user has attained the required distance value, triggering execution of an angle application to guide the user to attain a required angle value of the face of the user with respect to the image capture device; during execution of the angle application, monitoring the height of the user's face with respect to the image capture device, and the distance from the user's face from image capture device, wherein if the height of the user's face or the distance to the image capture device are outside the predefined height value or distance value, interrupting (and/or supplementing with additional prompts) the angle application.

Examples may include the step of determining the pitch of the image capture device and comparing the determined pitch with a predefined pitch value. The step of determining the pitch of the image capture device may be performed during execution of at least one of the height application, the distance application and the angle application, wherein if the determined pitch is outside the predefined pitch value the application is interrupted (and/or supplemented with additional prompts).

Examples comprise the steps of, when an application is interrupted, providing guidance to the user to re-attain the predefined pitch value. Examples comprise the further steps of when the value has been re-attained executing (i.e. resuming) the interrupted application. When an application is interrupted, it may be terminated. The pitch may be determined using a gyroscope.

The values of required height value, required distance value, required angle value, required pitch value may comprise at least one of: a specific numerical value, a range of numerical values, a functional value.

In a further aspect the invention provides a method for guiding a user to position and orientate their face and an image capture device in a required three-dimensional relation relative to one another for image capture, the method comprising the steps of: receiving first data representing at least one digital image of a face of a patient; executing a height application to guide the user to attain a required height value of the user's face with respect to the image capture device; executing a distance application to guide the user to attain a required distance value between the user's face and the image capture device; executing an angle application to guide the user to attain a required angle value of the face of the user with respect to the image capture device; wherein when the when the user has attained at least one of the required height value, required distance value, and required angle value, capturing an image of the user's face with the image capture device.

In a further aspect the invention provides a system for orienting an image-capture device and a user's face in a required three-dimensional relation relative to one another to enable capture of an image of the user's face at a required position by the image-capture device, the system being configured for: providing prompts to the user to assist the user to attain a required height of the user's face relative to the image-capture device; providing prompts to the user to assist the user to attain a required angle of the user's face relative to the image-capture device; and providing prompts to the user to assist the user to attain a required distance of the user's face relative to the image-capture device.

In a further aspect the invention provides a method of orienting an image-capture device and a user's face in a required three-dimensional relation relative to one another to enable capture of an image of the user's face at a required position by the image-capture device, the method comprising the steps of: providing prompts to the user to assist the user to attain a required height of the user's face relative to the image-capture device; providing prompts to the user to assist the user to attain a required angle of the user's face relative to the image-capture device; and providing prompts to the user to assist the user to attain a required distance of the user's face relative to the image-capture device.

The height application, the distance application and the angle application may be executed in a preferred sequence. When a required value has been attained, examples may monitor the attained value and interrupting (or supplementing with additional prompts) the executing application if the value is outside the predefined value. Embodiment may comprise the steps of, when an application is interrupted, providing guidance to the user to re-attain the required value which is outside the required value. When the user has attained at least one of the required height value, required distance value, and required angle value, capturing an image of the user's face with the image capture device.

In examples at least one of the prompts is a visual prompt. In examples, at least one of the prompts is displayed on the display interface.

In a further aspect the invention provides a method for guiding a user to position and orientate their face and an image capture device in a required three-dimensional relation relative to one another for image capture, the method comprising the steps of: detecting an image of the face of the user with the image capture device, using the image to calculate a three-dimensional relationship between the face of the user and the image capture device; comparing the calculated three-dimensional relation with the required three-dimensional relation; determining change in position and/or orientation of the user's face required to meet the required three-dimensional relation; and, presenting guidance to the user to re-position and/or re-orientate their face to create the required three-dimensional relation.

In examples, the three dimensional relation includes at least one of: vertical height of the face of the user compared with the image capture device; pitch of the face of the user compared with the image capture device; distance between the face of the user and the image capture device; yaw angle of the face of the user with respect to the image capture device; and roll angle of the face of the user with respect to the image capture device.

In examples, the image capture device may be located in a communications device, the display device including a display interface, the guidance is presented on the display interface. The image is, or is used to create, a three dimensional mapping of the face.

In a further aspect the invention provides a method for selecting a patient interface for a patient for use with a respiratory therapy device, the patient interface suitable to deliver respiratory therapy to the patient, comprising the steps of: receiving data representing at least one digital image of a face of a patient, the digital image being a first facial image type; determining a scaling factor from the first digital image; identifying a predefined facial feature appearing in the first digital image; calculating a dimension for the facial feature using the scaling factor; and, using the dimension to select a patient interface for the patient; wherein the digital image is a non-front facial image. The digital image may be an underside facial image.

In a further aspect the invention provides a method for selecting a patient interface for a patient for use with a respiratory therapy device, the patient interface suitable to deliver respiratory therapy to the patient, comprising the steps of: retrieving patient interface sizing information for two or more patient interface sizes, the patient interface sizing information including sizing dimensions of at least one defined patient facial feature suitable to fit the patient interface, wherein different patient interface sizes have different patient interface sizing information; for each of the defined patient facial features, retrieving a dimension of the defined facial feature of the patient; for each defined patient facial feature comparing the patient interface sizing information with the retrieved dimension of the relevant facial feature of the patient, and performing this step for each of the two or more different patient interfaces sizes; selecting a patient interface size for the patient in dependence on the comparison.

The step of comparing may comprise the step of, for each patient interface size, determining whether the retrieved dimensions of the defined facial feature of the patient matches the patient interface sizing information for the relevant patient facial feature.

The step of comparing may comprise the step of, for each patient interface size, determining whether the retrieved dimensions of the facial feature of the patient match the interface sizing information of the relevant facial feature for all defined facial features; and if the retrieved dimensions of the facial features of the patient do not match the interface sizing information of the relevant facial feature for all defined facial features for at least one patient interface size, applying a rule to determine which patient interface size to select for the patient.

The patient interface sizing information may comprise a range of dimensions suitable for the patient interface size for each defined patient facial feature. The retrieved dimensions of the relevant patient facial feature may match the interface sizing information when the retrieved dimension of the defined facial feature is within the range of dimensions.

In a further aspect the invention provides a method for assessing the suitability of a prescribed patient interface for a patient for use with a respiratory therapy device, the patient interface suitable to deliver respiratory therapy to the patient, comprising the steps of: receiving a prompt to initiate a patient interface suitability assessment for a patient; retrieving patient interface information associated with a prescribed patient interface for the patient; receiving data representing at least one digital image of a face of a patient; determining dimensions of at least one facial feature of the patient in the image; and, comparing the determined facial dimensions with the patient interface information associated with the prescribed patient interface to determine the suitability of the prescribed patient interface to the patient.

Examples may comprise the step of identifying specific facial features associated with the prescribed patient interface, wherein the step of determining dimensions of at least one facial feature of the patient is performed by determining the dimensions of the specific facial features. Examples may comprise the step of identifying at least one digital image type required to determine the specific facial dimensions of the patient, the digital image type defining a spatial arrangement between the face of the patient and an image capture device for image capture, and notifying the patient of the required spatial arrangement. The step of notifying the patient may comprises presenting dynamic spatial guidance to the patient to position the image capture device in response to detecting a spatial arrangement between the image device and the face of the user.

In a further aspect the invention provides a method for guiding a user to position their face and an image capture device in a required three-dimensional relation relative to one another for image capture, the method comprising the steps of: detecting an image of the face of the user with the image capture device, using the image to calculate a three-dimensional relationship between the face of the user and the image capture device; calculating a distance between the face of the user and the image capture device; comparing the calculated distance with a predefined distance between the face of the user and the image capture device; if the calculated distance does not match the predefined distance, presenting guidance to the user to re-position their face to create the predefined distance.

The three-dimensional relationship between the face of the user and the image capture device may be the distance between the face of the user and the image capture device.

Examples may comprise the further steps of: detecting a change in the three-dimensional relationship between the face of the user and the image capture device; comparing the changed three-dimensional relationship with the predefined distance between the face of the user and the camera; and, on detecting that the calculated three dimensional relationship meets the required three-dimensional relationship performing at least one of the following steps: presenting an indication to the user that the three dimensional relationship meets the required three dimensional relationship; or capturing an image of the user's face, and using the captured image to retrieve facial dimensions of the user for use in selecting a patient interface for a patient for use with a respiratory therapy device, the patient interface suitable to deliver respiratory therapy to the patient. The guidance may comprise an indicator displayed on a user device.

In a further aspect the invention provides a system for sizing a patient interface for a patient for use with a respiratory therapy device, the patient interface suitable to deliver respiratory therapy to the patient, comprising the steps of: Initiating a patient interface sizing application; Identifying at least one patient interface type required for sizing for a patient; determining at least one facial image type required to size the at least one patient interface type required for sizing; executing an image capture sequence to capture the at least one facial image type required for sizing and calculating a dimension of at least one facial feature required for sizing for the patient; based on the calculated dimension, determining a suitable size, for the patient, of each of a plurality of patient interfaces within said patient interface type.

The at least one patient interface type may be a subset of patient interface types. The system may further comprise selecting a camera of a plurality of cameras of an image-capture device for operation during the image capture sequence, the image capture sequence being dependent on the selected camera. The image capture sequence may comprise user instructions and/or animations.

In a further aspect the invention provides a patient interface sizing system for selecting a patient interface size for a patient for use with a respiratory therapy device, the patient interface suitable to deliver respiratory therapy to the patient, comprising: identifying dimensions for multiple facial features required to size a selected patient interface; receiving the dimensions of the multiple facial features; comparing the multiple facial dimensions with patient interface sizing data for the selected patient interface to determine a size of the selected patient interface for the patient; wherein one or more sizing rules related to the multiple facial dimensions are dependent on the selected patient interface; and displaying, on a display, an icon representative of the determined size of the patient interface, said icon being superposed on a chart comprising segments representing one or more sizes of the patient interface; said chart comprising at least a first and second axis representing at least a first and second of the identified dimensions. The one or more sizing rules may be different for different patient interfaces. The one or more rules may comprise assigning weightings to the respective facial dimensions. The one or more rules may specify that a first facial dimension takes precedence over a second facial dimension to determine interface size.

In a further aspect the invention provides a patient interface fitting system comprising the steps of: identifying multiple facial dimension measurements required to fit a patient interface; receiving the multiple facial dimension measurements; combining the multiple facial dimension measurements using a combination operation; and comparing the combined multiple facial measurements with facial interface sizing data for at least one patient interface type to identify a patient interface size for the patient associated with patient interface type; wherein combination operation is dependent on the patient interface type.

In a further aspect the invention provides a patient interface sizing system for selecting a patient interface size for a patient for use with a respiratory therapy device, the patient interface suitable to deliver respiratory therapy to the patient, comprising: identifying a dimension of a facial feature required to size a selected patient interface; receiving the dimension of the facial feature; comparing the facial dimension with patient interface sizing data for the selected patient interface to determine a size of the selected patient interface for the patient; wherein one or more sizing rules related to the facial dimension are dependent on the selected patient interface; and displaying, on a display, an icon representative of the determined size of the patient interface, said icon being superposed on a chart comprising segments representing all sizes of the patient interface; said chart comprising an axis representing the identified dimension.

In a further aspect the invention provides a system for sizing a plurality of patient interfaces for a patient for use with a respiratory therapy device, the patient interfaces suitable to deliver respiratory therapy to the patient, comprising the steps of: Initiating a patient interface sizing application; Identifying a plurality of patient interfaces required for sizing for a patient; For each of the plurality of patient interfaces, determining at least one facial feature whose dimension is required in order to size the respective interface; determining at least one facial image type required in respect of each of the at least one facial feature; executing an image capture sequence to capture the at least one facial image type; using the captured at least one facial image type, calculating the dimension of each of the at least one facial feature; based on the calculated dimension of each of the at least one facial feature, determining a suitable size, for the patient, of each of the plurality of patient interfaces.

In a further aspect the invention provides a method for guiding a user to orientate a face of a patient and an image capture device in a required three-dimensional relation relative to one another for image capture, the method comprising:
receiving image data representing a digital image of the face of a patient;
identifying within the digital image at least one predefined portion of the face of the patient;
determining a three-dimensional relation between the face of the patient and the image capture device;
displaying on a display device a framing identifier, the framing identifier representing the predefined portion of the face of the patient, the framing identifier being positioned on the display device at a position representing the three-dimensional relation between the face of the patient and the image capture device.

In a further aspect the invention provides a system for guiding a user to orientate a face of a patient and an image capture device in a required three-dimensional relation relative to one another for image capture, comprising:
receiver for receiving image data representing a digital image of the face of a patient;
processor for identifying within the digital image at least one predefined portion of the face of the patient, determining a three-dimensional relation between the face of the patient and the image capture device; and
display device for displaying a framing identifier, the framing identifier representing the predefined portion of the face of the patient, the framing identifier being positioned on the display device at a position representing the three-dimensional relation between the face of the patient and the image capture device.

The image data representing a digital image of the patient may be displayed simultaneously with the framing indicator. The framing identifier may be a feature framing indicator. The feature framing indicator may surround the predefined portion of the face within the displayed image data.

The framing identifier may represent a predefined portion of the face of which dimensions are required.

Further examples may include a static indicator displayed on the display device to illustrate or indicate a required position of the feature framing indicator on the display screen. The static indicator providing a target position for, or relative to, the feature framing indicator on the display of the display device. When the face of the patient and the image capture device are in the required three-dimensional relation relative to one another for image capture the feature framing indicator and the static indicator may be co-located on the display screen. Alternatively, when the face of the patient and the image capture device are in the required three-dimensional relation relative to one another for image capture the feature framing indicator and the static indicator may be in a predefined relationship relative to one another on the display screen; for instance, the feature framing indicator may be centrally disposed within the static indicator.

The user may be the patient. The portion of the face of the patient may be a feature of the face of the patient.

### BRIEF DESCRIPTION OF THE FIGURES

The ensuing description is given by way of non-limitative example only and is with reference to the accompanying drawings, wherein:

FIG. 1 is a schematic diagram of a respiratory therapy device including a blower for generating a flow of breathable gas, a conduit, and a patient interface for delivering the flow of breathable gas to the patient.

FIGS. 2A(i) and 2A(ii) are illustrations of a full face mask showing the mask positioned on the face and the contact area of the mask on the face.

FIGS. 2B(i) and 2b(ii) are illustrations of a nasal mask showing the mask positioned on the face and the contact area of the mask on the face.

FIGS. 3(i) and 3(ii) are illustrations of an under nose nasal mask showing the mask positioned on the face and the contact points of the mask on the face.

FIG. 4 is a schematic illustration of a mobile communications device.

FIG. 5 represents a basic architecture showing an interaction of a server with a mobile communications device.

FIG. 6 is a diagram showing facial features relating to an eye.

FIG. 7 is a flow chart showing steps performed in an embodiment.

FIG. 8 shows the alignment of a camera with the face of a patient when capturing an image for the mask sizing application.

FIG. 9 is an illustration of an image of a patient's face being displayed on the screen of the mobile communications device during image capture.

FIG. 10 is an illustration of an image of a patient's face identifying anthropometric features of the face.

FIGS. 11A and 11B are illustrations of an image of a patient's face identifying the eye width.

FIGS. 12A and 12B are illustrations of an image of a patient's face identifying various facial landmarks.

FIG. 13 is an example display of a mask recommendation to a patient.

FIG. 14 shows axes of rotation of the head, including pitch, yaw and roll.

FIG. 15 is a flow diagram showing the steps taken to analyse an image to determine if it meets various predefined criteria.

FIGS. 16, 16A, and 16B show image capture of a face of a patient and visual feedback provided to the patient.

FIGS. 17, 17A, and 17B show image capture of a face of a patient and visual feedback provided to the patient.

FIGS. 18, 18A, and 18B show image capture of a face of a patient and visual feedback provided to the patient.

FIG. 19 is a flow diagram showing steps performed by an embodiment.

FIG. 20 is an illustration of an example question displayed on a mobile communications device.

FIG. 21 is an illustration of a recommended mask displayed to a patient.

FIG. 22 shows example mask data scores for various questionnaire questions.

FIG. 23 shows the scores of a patient after completing a questionnaire.

FIG. 24 shows example relevant feature dimensions associated with fitting a full face mask.

FIG. 25 shows example relevant feature dimensions associated with fitting a nasal mask.

FIG. 26 shows example relevant feature dimensions associated with fitting an under nose nasal mask.

FIG. 27 shows example relevant feature dimensions associated with fitting a nasal pillow interface.

FIG. 28 is a flow diagram showing steps performed by an embodiment.

FIG. 29 is a flow diagram showing steps performed by an embodiment.

FIGS. 30, 30A, 30B show image capture of a face of a patient and visual feedback provided to the patient.

FIGS. 31, 31A, and 31B show image capture of a face of a patient and visual feedback provided to the patient.

FIGS. 32, 32A, and 32B show image capture of a face of a patient and visual feedback provided to the patient.

FIG. 33 illustrates animations presented during scanning.

FIG. 34 is a flow diagram showing steps performed by an embodiment.

FIG. 35 is a flow diagram showing steps performed by an embodiment.

FIG. 36 shows an image capture of a face of a patient.

FIG. 37 illustrates image processing steps.

FIG. 38 illustrates image processing steps.

FIG. 39 illustrates image processing steps.

FIG. 40 illustrates image processing steps.

FIG. 41 illustrates image processing steps.

FIG. 42 illustrates image processing steps.

FIG. 43 illustrates image processing steps.

FIG. 44 is a flow diagram showing steps performed in an embodiment.

FIGS. 45 46 and 47 show the field of view of a patient at various head tilting positions.

FIGS. 48 to 51 illustrate a visual indicator of a user interface.

FIGS. 52 to 54 illustrate a position indicator for a user interface including a bar displayed on the user interface.

FIG. 55 shows a further user interface including a position indicator.

FIGS. 56 and 57 show a user interface including a static indicator and a dynamic indicator.

FIG. 58 and FIG. 58A show examples of user interface.

FIGS. 59 to 62 show a further user interface including a static indicator and a dynamic interface.

FIGS. 63 and 64 show further examples of visual indicators to assist with face positioning.

FIGS. 65 to 69 show various exemplary animation indicators presented to assist with face positioning.

FIGS. 70 and 71 show examples of animated instructions.

FIGS. 72 and 73 show an animated face mesh.

FIG. 74 shows an example of a full face mask.

FIG. 75 shows the steps taken to select a patient interface for a patient.

FIG. 76 and FIG. 77 show examples of sizing information for a patient interface.

FIG. 78 is a flow diagram showing the steps to select a patient interface for a patient.

FIGS. 79 to 86 show examples of sizing information for patient interfaces.

FIGS. 87 to 95 show user interface animations to assist the user positioning the camera relative to the face of the patient.

FIG. 96 shows an example of the steps performed in a resizing process.

FIGS. 97 and 98 show examples of facial features for headgear sizing.

FIG. 99 is a flow diagram showing example steps performed in a clinician mode.

FIG. 100 shows examples of user interfaces for clinician mode.

FIG. 101 shows examples of user interfaces.

FIGS. 102 to 109 show examples of sizing charts for patient interfaces.

FIG. 110 shows an example of a user interface displaying a QR code.

FIG. 111 and FIG. 112 show examples of user interfaces presenting indicators to assist with camera positioning for image capture.

FIG. 113 shows example user interfaces displaying instructions for positioning for image capture.

FIG. 114 shows steps performed to assist in positioning the patient correctly with respect to the camera for image capture.

FIG. 115 to FIG. 124 show examples of user interfaces providing guidance for correctly position the face of the patient with respect to the camera.

FIG. 125 to FIG. 130 show steps taken to position the face of the patient correctly with respect to the camera using a base angle.

FIGS. 131 to 135 show user interfaces including indicators for assisting a user to correctly position the face of the patient with respect to the camera.

FIG. 136 is a flow diagram showing the steps of a course/fine adjustment sequence.

FIG. 137 is a flow diagram showing steps to select a patient interface including providing guidance to a user for positioning the image capture device relative to a face.

FIGS. 138 and 139 are flow diagrams showing steps taken to guide a user to position the image capture device relative to the face.

FIG. 140 is a flow diagram showing the steps to providing an indication on a sizing chart.

FIG. 141 shows the steps taken during patient interface selection including combining facial feature dimensions.

FIG. 142 shows the steps taken during patient interface selection.

### DETAILED DESCRIPTION

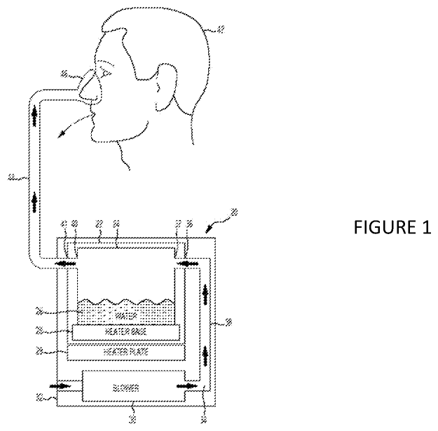

A method and system for selecting a patient interface for a patient for use with a respiratory therapy device are now described with reference to the accompanying FIGS. 1 to 44. The selected patient interface is used to provide respiratory therapy to a patient. The system for selecting the patient interface is configured to select a patient interface for a patient to use with a respiratory therapy device. The patient interface is automatically selected by capturing at least one image of a patient's face and determining dimensions of various features of the patient's face using a reference scale. The features may be landmarks on a patient's face. The dimensions are compared with patient interface sizing data associated with different patient interfaces and system is configured to automatically identify a suitable patient interface for the patient. The suitable patient interface may be an appropriate sized patient interface. The system may further be configured to determine an appropriate patient interface category i.e. Patient interface type.

An exemplary embodiment will now be described in the following text which includes reference numerals that correspond to features illustrated in the accompanying figures.

FIG. 1 is a schematic illustration of a respiratory therapy device 20. The respiratory therapy device 20 can be used to provide CPAP (continuous positive airway pressure) therapy or BiLevel pressure therapy. The respiratory therapy device 20 comprises a humidification compartment 22 and a removable humidification chamber 24 that is inserted into and received by the compartment 22.

The humidification chamber 24 is inserted in a vertical direction when the compartment 22 is in an upright state. The compartment 22 has a top opening, through which the chamber 24 is introduced into the compartment 22. The top opening may have a lid so the humidification chamber 24 within the humidification compartment 22 may be accessed for removal for cleaning or re-filling. But this is optional, and other arrangements can be envisaged. For example, in other embodiments it is possible that the chamber 24 is inserted horizontally into the humidification compartment 22. Additionally/alternatively the respiratory therapy device may comprise a receptacle that includes a heater plate. The chamber is slidable into and out of the receptacle so that a conductive base of the chamber is brought into contact with the heater plate.

The humidification chamber 24 is fillable with a volume of water 26 and the humidification chamber 24 has, or is coupled to, a heater base 28. The heater plate 29 is powered to generate heat which is transferred to the heater base 28 of the chamber 24 (via the heat transfer plate 29) to heat the water 26 in the humidification chamber 24 during use.

The respiratory therapy device 20 has a blower 30 which draws atmospheric air and/or other therapeutic gases through an inlet and generates a gas flow 34 at an outlet of the blower 30. FIG. 1 illustrates an arrangement in which the outlet of the blower 30 is fluidly connected directly to a chamber inlet 37 via connecting conduit 38 and a compartment outlet 36. The chamber inlet 37 and the compartment outlet 36 may have a sealed connection when the humidification chamber 24 is in the operating position.

The gas flow 34 passes through the humidification chamber 24, where the humidity of the gas flow 34 is increased and exits via gases outlet 40 of the humidification chamber. The gas flow is delivered via a conduit 44 and a mask, nasal pillows or similar patient interface 46 to a patient.

In the arrangement shown in FIG. 1, a chamber outlet 40 is sealingly connected to, or sealingly engaged with, a compartment inlet 41 by a sealed connection. In this embodiment, a lid to the compartment may or may not be provided.

In the arrangement of FIG. 1, the gas flow 34 passes through the humidification chamber 24, where the humidity of the gas flow 34 is increased and exits via chamber outlet 40. The chamber outlet 40 is sealingly connected to, or sealingly engaged with, a compartment inlet 41. It will be appreciated that in alternative embodiments, the chamber outlet 40 and the compartment inlet 41 need not be sealingly connected by a connector or otherwise sealingly engaged. The gas flow is delivered via a conduit 44 to a patient interface 46. The patient interface may be a mask. The patient interface may comprise one of: a nasal mask, an oral-nasal mask, an oral mask, a full face mask, an under nose mask, nasal pillows, or any other suitable patient interface that is a sealing patient interface used for providing CPAP therapy or BiLevel therapy.

One or more sensors (not shown in FIG. 1) may be positioned within respiratory therapy device 20. Sensors are used to monitor various internal parameters of the respiratory therapy device 20.

Sensors (not shown) are connected to a control system comprising a control unit. The sensors communicate with the control system. The control unit is typically located on a PCB. In one form the control unit may be a processor or microprocessor. The control system is able to receive signals from the sensors and convert these signals into measurement data, such as pressure data and flow rate data. In some forms, the control unit may be configured to control and vary the operation of various components of the respiratory therapy device to help ensure that particular parameters (such as, for example, air pressure, humidity, power output, blower speed) fall within desired ranges or meet desired ranges, thresholds or values. Typically, the desired ranges, thresholds or values are predetermined and are programmed into the control unit of the control system. Additional sensors, for example O2 concentration sensors or humidity sensors may be included into the respiratory therapy device. Further sensors may also comprise a pulse oximeter to sense blood oxygen concentration of a patient. A pulse oximeter is preferably mounted on patient and could be connected to the controller by a wired or wireless connection.

Blower 30 may control air and/or other gases flow in the respiratory therapy device. The control system and the control unit may be configured to control the state of blower 30 through transmission of control signals to blower 30. Control signals control the speed and duration of operation of blower 30.

Control system is programmed with multiple operating states for the respiratory therapy device. The control software for each operating state is stored within a memory within the control system. Control system executes the control software by transmitting control signals to the blower 30 and various other components of the respiratory therapy device to control the operation of the respiratory therapy device to create the required operating state.

Operating states for the respiratory therapy device may include respiratory therapy states and non-respiratory therapy states. Examples of respiratory therapy states include: CPAP (continuous positive airway pressure) commonly used to treat obstructive sleep apnea in which a patient is provided with pressurized air flow typically pressurized to 4-20 cmH20; NIV (non-invasive ventilation), for example biLevel pressure therapy, used for treatment of obstructive respiration diseases such as chronic obstructive pulmonary disease (COPD-which includes emphysema, refractory asthma and chronic bronchitis. Examples of non-respiratory therapy states include: an off state, in which the blower is off and provides no airflow through the respiratory therapy device; idle state, in which the blower is on and providing airflow through the respiratory therapy device but not providing therapy; and drying mode in which the blower may be on and cycle through a predefined speed pattern but not provide therapy. In drying mode a heater wire in the tube may be activated to a predetermined level e.g. 100% power and the blower may be activated to a preset flow rate or motor speed and driven for a predetermined time e.g. 30-90 mins. Drying mode dries out the conduit of any liquid or liquid condensate.

Different airflow conditions in the respiratory therapy device are required for different operating states. The control system provides control signals to the blower 30 to control blower operating parameters, including activation and speed, to provide the required airflow conditions in the respiratory therapy device.

Software programs defining the operating conditions required for the various operating states of the respiratory therapy device are stored within memory 64 of control system 60. During operation of a particular operating condition, the control system receives signals from various sensors and components of the respiratory therapy device at a communication module 62 defining the conditions within the respiratory therapy device, for example pressure data and flow rate data. The control system 60, and in particular processor 66, is configured to compare the conditions within the respiratory therapy device with predefined operating conditions for the operating state and to control and vary the operation of various components of the respiratory therapy device to help ensure that particular conditions (such as, for example, air pressure, humidity, power output, blower speed) fall within desired ranges or meet desired thresholds or values associated with the required operating state. The desired ranges, thresholds or values are predetermined and programmed into the software program.

In some embodiments, the respiratory therapy device includes a transceiver to transmit and receive radio signals or other communication signals. The transceiver may be a Bluetooth module or WiFi module or other wireless communications module. The transceiver may be a cellular communication module for communications over a cellular network e.g 4G, 5G. In one example the transceiver may be a modem that is integrated into the device. The transceiver allows the device to communicate with one or more remote computing devices (e.g. servers). The device is configured for two way communication (i.e. to receive and transmit data) to the one or more remote computing devices (e.g. servers). For example device usage data can be transmitted from the device to the remote computing devices. In another example therapy settings for the device may be received from the one or more remote computing devices. In a further example the respiratory therapy device may comprise multiple transceivers e.g. a Wifi module, a Bluetooth module, and a modem for cellular communications or other forms of communication.

In some embodiments the transceiver may communicate with a mobile communications device.

The respiratory therapy device 20 may be a high flow therapy device and used to provide high flow therapy to a patient. The respiratory therapy device 20 may be controlled to provide high flow therapy. The blower 30 may be controlled to a set flow rate during high flow therapy. The heater plate 29 is controlled to heat water in the humidification chamber 24 to humidify gases flow. During high flow therapy, the heater plate is controlled to humidify gases to 37 degrees dew point.

Pressure therapies like CPAP and BiLevel therapies are provided through sealed interfaces as indicated earlier. High flow therapy is provided via an unsealed patient interface such as for example a nasal cannula. A nasal cannula comprises a pair of nasal prongs that engage the nostrils when in use. In use the prongs are inserted into the nostrils of a patient, but do not seal with the nostrils. There is normally a space between the prongs and the nostril to avoid a seal and allow exhaled gases to exit the nostrils around the prongs. This is in contrast to sealed interfaces such as nasal masks, full face masks, oro-nasal masks that seal with the patient's nose or nose and mouth. Further under nose masks i.e. sub nasal masks also seal with at least the nose of the patient but seal on the underside of the nose. In some patients nasal pillows are used to provide CPAP or BiLevel therapy. Nasal pillows comprise a pair of pillows i.e. Prong like structures that are inserted into the nostrils of the patient. However, pillows seal with the nostrils of the patient either on an inner surface or around an outer surface or along an edge of the nostrils.

The patient interface 46 is typically a mask or could be a nasal pillows type interface or a hybrid interface that has nasal pillows and an oral cushion (i.e, a portion for the mouth to fit into). The patient interface could also be a nasal cannula, or any other type of respiratory therapy interface. The patient interface 46 is configured for connection to the patient's face. The patient interface 46 may be held in place on the face of the patient using a headband which extends around the head of the patient. Other suitable means for holding the patient interface in place may also be used, for example adhesives or suction. The patient interface is an important part of the respiratory system and preferably provides comfortable delivery of gas to the patient without leakage. Different patient interface types are available to patients including full face masks, nasal face masks and under nose masks i.e. sub nasal masks. Sub nasal masks i.e. under nose masks may be provided as under nose nasal masks or under nose full face masks. An under nose full face mask forms a seal along a portion of an underside of a patient's face and around the mouth of the patient. An under nose nasal mask does not seal with or around the mouth and only seals the nose.

The patient interfaces are typically available in different sizes to fit faces of different shapes and sizes. Correct fitting of patient interfaces is important to avoid leaks in a CPAP system. Leaks can reduce the effectiveness of the therapy or respiratory support delivered via the patient interface. Large leaks can reduce the pressure delivered to the patient while provided CPAP or BiLevel therapy. Poorly fitted patient interfaces can also be uncomfortable to the patient and result in a negative or painful therapy experience, for example by causing pressure sores on sensitive parts of the face. Such discomfort can reduce compliance to therapy. Selecting the correct patient interface for a patient is critical to providing reliable and ongoing therapy.

A number of factors are relevant when selecting a patient interface for a patient:

A first consideration is selecting the correct patient interface category i.e. correct patient interface type, for a patient. Patients breathe in different ways, some patients breathe through their nose, some patients breathe through their mouth, and, some patients breathe in a combination through their nose and mouth. Optimal respiratory therapy or respiratory support can be provided to a patient by prescribing a patient interface type suitable to the way a patient breathes. (The type of respiratory therapy required by a patient may also constrain the types/categories of masks that are suitable for them). The main patient interface categories are: full face mask, nasal mask, under nose nasal mask or nasal pillows. Other types of patient interfaces include oral masks (seal around the mouth only), hybrid masks (seals around the mouth and has nasal pillows to seal with nostrils), under nose full face mask variation (seals around mouth and an under nose seal). Each patient interface functions to create a seal with either the mouth, nose, or both to maintain effective delivery of pressure-based therapy e.g. CPAP or BiLevel. The consideration of which patient interface a patient should use is influenced by which airway(s) they predominantly breathe from—that airway is where pressure-based therapy should be delivered to keep the tissue of the main airway open and prevent collapse. The chosen patient interface seals against the airway and essentially extends the airway fluidically to the therapy device which supports breathing E.g. if the patient predominantly breathes from their nose then they will receive the most effectively respiratory aid if a nasal mask, under nose mask or nasal pillows are used to seal with that airway and provide pressure.

A nasal cannula is used when a patient is prescribed nasal high flow therapy.

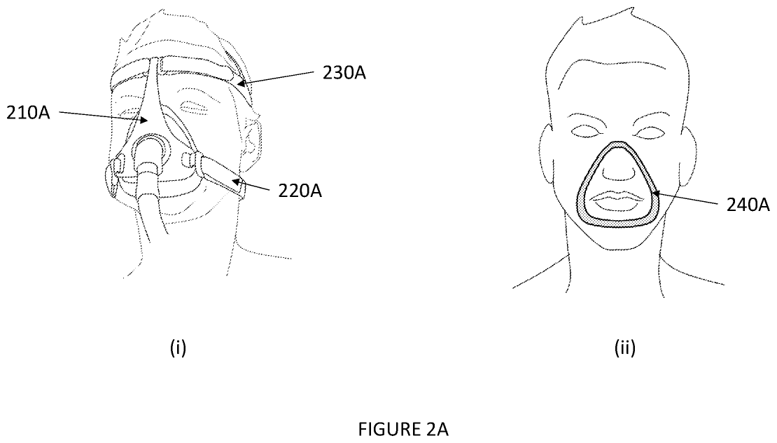

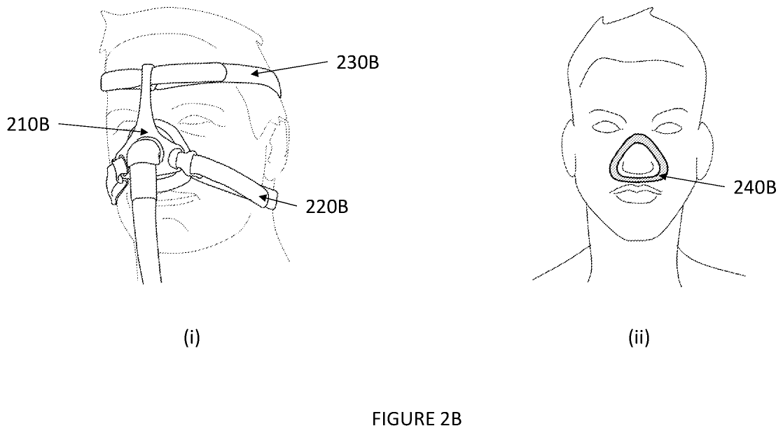

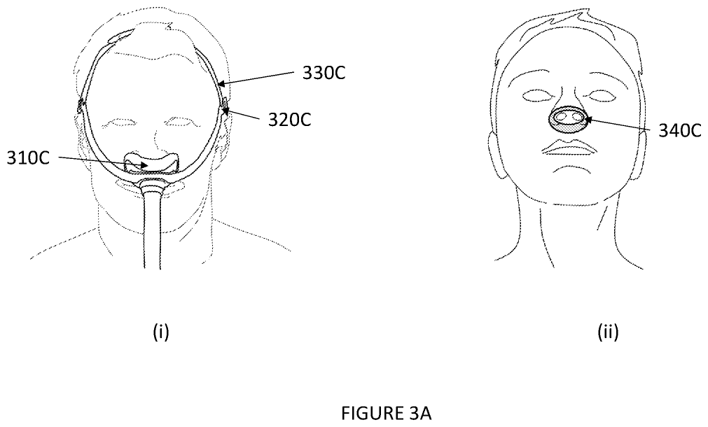

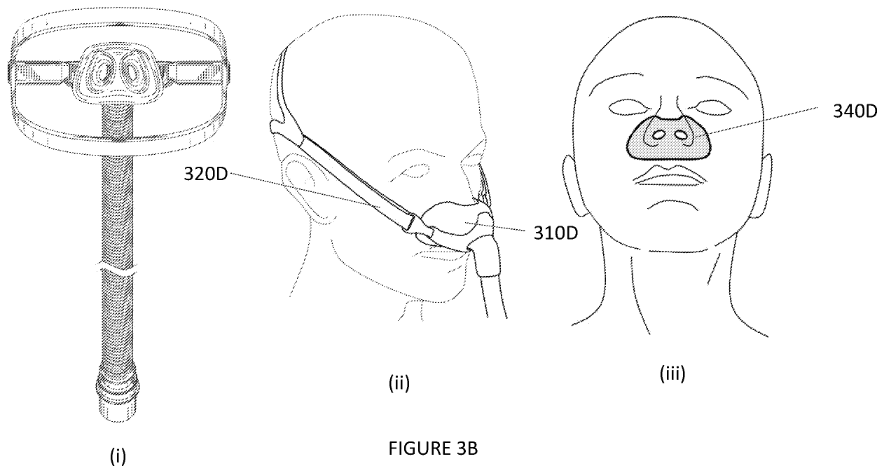

Examples of different patient interface categories are shown in FIGS. 2 and 3. FIGS. 2 and 3 illustrate each patient interface category on the face of a patient and, separately, illustrates the contact area for each patient interface category on the face of the patient.

FIG. 2A shows a full face mask 210A which covers the nose and mouth of the patient. Full face mask 210A is held to the face of the patient using headgear. Headgear includes a strap 220A extending around the jaw and/or cheek and neck of the patient and a second strap 230A extending around the top of the head of the patient. Full face masks seal around the whole mouth and nose region and over the nose bridge. As illustrated in FIG. 2A(ii), seal 240A extends under the mouth of the patient, around the sides of the nose and over the nose bridge. The flexible seal of a full face mask can conform/mould to varying surfaces around the nose and mouth to create an effective seal to maintain pressure when therapy is delivered.

FIG. 2B shows a nasal face mask. The nasal face mask covers the nose only and does not cover the mouth. Nasal face mask 210B is held to the face of the patient using a strap 220B extending around the jaw and/or cheek and neck of the patient and a second strap 230B extending around the top of the head of the patient. Nasal face masks seal around the nose region and over the nose bridge. As illustrated in FIG. 2B (ii), seal 240B extends around the nose of the patient. It seals under the nose of the patient, under the nostrils and above the mouth, around the sides of the nose and over the nose bridge. The flexible seal of a nasal face mask can conform/mould to varying surfaces around the nose to create an effective seal to maintain pressure when therapy is delivered.

FIG. 3A shows an under nose nasal mask. Under nose nasal masks only seal with the nostrils. This is a less intrusive way to create a nasal seal than using a nasal mask. The under nose nasal mask 310C is held to the face of the patient using a strap 320C extending around the back of the head of the patient and a second strap 330C extending over the top of the head of the patient. Under nose nasal masks seal around or under the nose region only. As illustrated in FIG. 3(ii), seal 340C extends around the nostrils of the patient. The seal is created on a portion of the underside of the nose of the patient. The seal 340C may also seal up around the sides of the nose or may seal around the side of the nose e.g. within a region of the alar crease or about the alar of the patient. The flexible seal of an under nose nasal mask can conform/mould to varying surfaces around the nose to create an effective seal to maintain pressure when therapy is delivered.

FIG. 3B shows a nasal pillow. Nasal pillows are sealing prongs that seal against either the outer edge or inner edge of the nostril. These prongs need to be large enough to seal against the nostril and hence have to completely cover the nostrils. Pillows need to be sized to cover the entire nostril for a seal. Some nasal pillow seal within the nostrils, for example like a sealing prong, around the nose (like the seal shown in 3A), or a combination of the two: within nostrils and around nose (all the grey area). The pillow 310D is held to the face of the patient using a strap 320D extending around the back of the head of the patient. As illustrated in FIG. 3 (iii), seal 340D extends around the nostrils of the patient. Some pillow seal within the nostrils, for example like a sealing prong, around the nose (like the seal shown in 3A), or a combination of the two: within nostrils and around nose (all the grey area).

Other patient interface types include non-sealing prongs. These patient interface types include prongs which fit within the nostrils. Nasal prongs as used on nasal cannula are unsealed and therefore do not need to seal or cover the nostrils. Prongs need to have a space between the outside of the prong and the nostril to allow leak and exhaled gases to pass around the prongs. These patient interfaces are not sealed to the face or to the nostrils. The therapy promotes expired air to be cleared from the airways through flow based therapy. These patient interfaces are used for Nasal High Flow (NHF) therapy.

Within each patient interface category, patient interfaces may be provided in different sizes, for example XS, S, M, L. The size of the patient interface is generally defined by the seal size, i.e. the size of the patient interface seal that contacts the face. Generally, patients with larger heads require a larger seal size in order to provide an optimal or working seal. The size of the headgear is also a consideration for effectiveness and comfort and the headgear may also be provided in different sizes depending on the size of the head of the patient. Some patient interface categories may also include an XL patient interface size.

When selecting a patient interface for a patient, depending on the therapy type, further considerations may be taken into account relating to the sleeping habits of the patient. Typical prescriptions for Continuous Positive Airway Pressure (CPAP) respiratory therapy require the patient to wear the patient interface throughout the night while sleeping. Factors including patient movement during the therapy session, for example whether the patient is a restless sleeper, and also whether the patient wears glasses in bed, are also factors to be considered when selecting a patient interface for a patient, in order to optimize the effects of the therapy and a patient's ongoing adherence to a therapy program. Other considerations include, safety: poorly fit patient interfaces may lead to a patient tampering with the fit and settings etc resulting in leaks or reduced compliance to therapy i.e. reduced use of therapy. Leaks may also be noisy and disrupt sleep (of the patient and partner).

When selecting a patient interface for a patient, for pressure sealing interfaces, objectives include minimize leakage (also referred to as “unintentional leak”) between the patient interface and the face in order to optimize therapy but also to avoiding patient discomfort by avoiding excessive pressure around the contact area of the patient interface with the face. Poorly fitting patient interfaces or patient interfaces which do not match the patient's breathing type can affect the effectiveness of therapy, patient comfort and patient therapy adherence.

Typically, patient interfaces are fitted by clinicians during patient diagnosis. Patient interface fitting is typically performed in person with the patient able to try on different patient interface types and sizes in order to select the most appropriate patient interface type and patient interface size for the patient under the guidance of a professional. Clinicians are technical experts and experienced with patient interface fitting for patients.

Patient interfaces are consumable products with a limited lifetime of optimal usage and typically a patient needs to replace a patient interface every few months. There has been a desire for remote ordering of patient interfaces by patients. Additionally, some patients prefer to select a patient interface without visiting a clinician.

Recently, patient interface suppliers have begun to offer remote patient interface selection and remote patient interface ordering options to patients. These options may allow a patient to view a catalogue of patient interfaces, select a patient interface from the catalogue and order the patient interface remotely, for example over the internet. One challenge with allowing patients to select a patient interface is that the fitting procedure is not undertaken by technical experts and so the patient interface selected by the patient may not be optimal in terms of patient interface category or patient interface fit. As discussed above, poorly fitting patient interfaces or patient interface which do not match the patient's breathing style and/or other sleeping factors, for example the position in which a patient tends to sleep e.g. side sleeper, can result in sub-optimal therapy and discomfort to the patient. These factors may reduce therapy results and can result in poor patient therapy adherence. Another practice is to send a multi mask pack to the patient that includes all sizes. The patient fits the mask size by trial and error. The sizes that are unused are thrown away and cannot be re-used or re-sold. This can be a waste of masks. Further sizing is reliant on trial and error by a patient, which can result in errors in correct fitting.

Nasal cannula are often sized by eye or by trial and error. This can be time consuming for an expert or result in errors when done by the patient on themselves. Another approach for sizing is a patient is sent all available sizes of nasal cannula and the patient self sizes by trial and error. The unused nasal cannula are thrown away which increases waste.

Automatic patient interface sizing software applications which collect patient data and recommend patient interfaces to patients have been developed. These can provide improved results compared to independent patient selection of patient interfaces. However, one of the challenges of automatic patient interface selection is the capture of accurate patient facial measurement data to allow the software application to identify a patient interface which fits the patient. Software applications for recommending patient interfaces to patients often provide unreliable measurement data or rely on patient expertise or input to retrieve measurements. These factors can result in the recommendation of sub-optimal patient interfaces to the patient.

Another challenge is to make the process simple to use and fast in addition to providing accurate measurements and sizing. Patients may be unfamiliar with technology or have limited mobility, and hence there is a need for a simple, intuitive sizing process.

In an embodiment, a method and system for selecting a patient interface for a patient for use with a respiratory therapy device or system is provided. The patient interface is suitable to deliver respiratory therapy or respiratory support to the patient.

Embodiments of the invention provide a method and system for selecting a patient interface for a patient for use with a respiratory therapy device. The patient interface is suitable to deliver respiratory therapy to the patient. The system receives facial images of the patient and uses the facial images to select a patient interface for the patient. The system extracts dimensions of relevant features of the patient's face from the images and selects an interface for the patient that will fit the various dimensions of the patient's face.

Facial images are digital images that include the face of the patient. Facial images may include the face of the patient at different orientations.

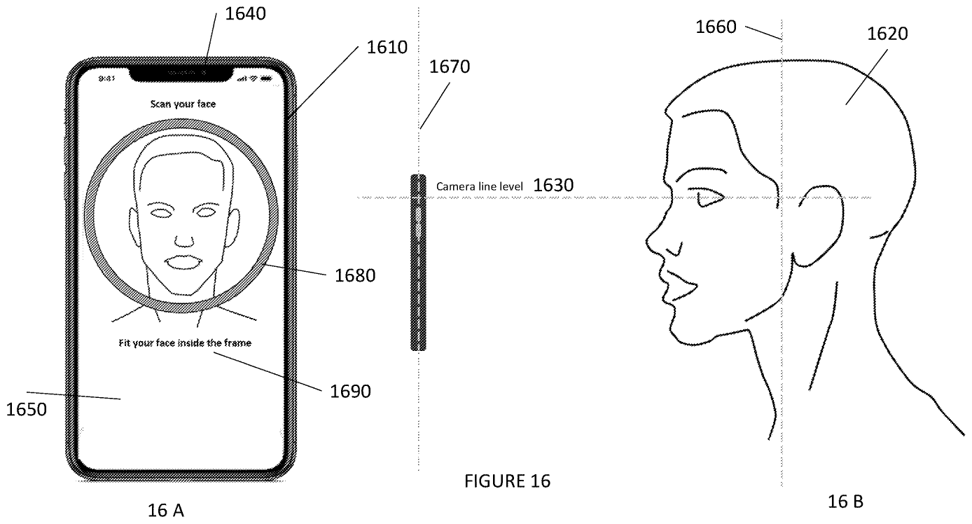

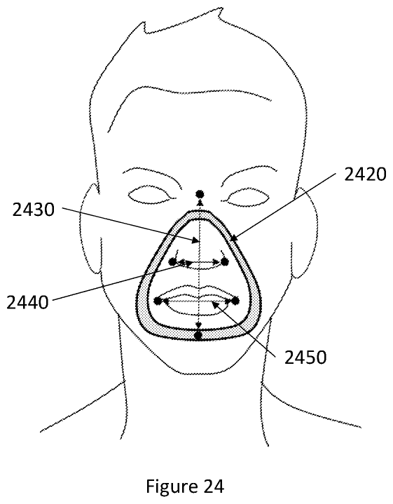

A first type of facial image may include a front view of the face of the patient. This may be referred to as a front facial image. An example of a camera/face configuration for producing a front facial image is shown in FIG. 16. In FIG. 16 the face of the patient is directly facing the camera 1640 and the angle of the face of the patient relative to the plane of the camera 1670 is approximately zero. FIG. 24 is an example of a front facial image.

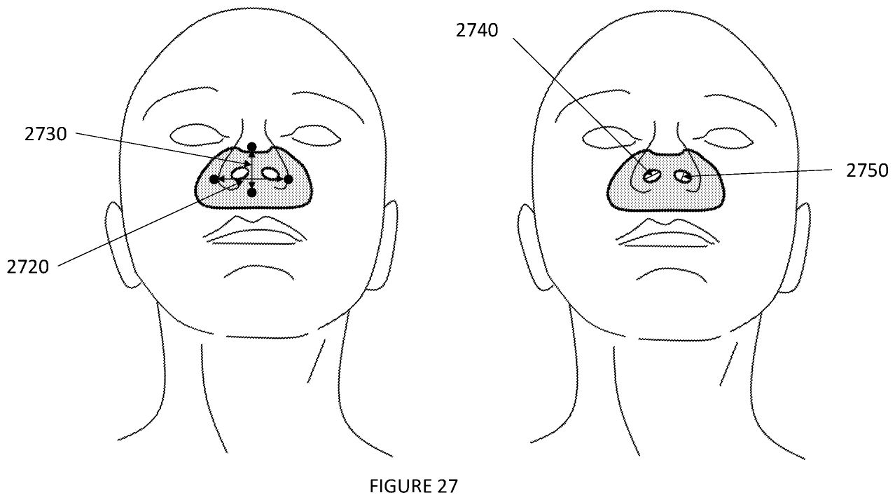

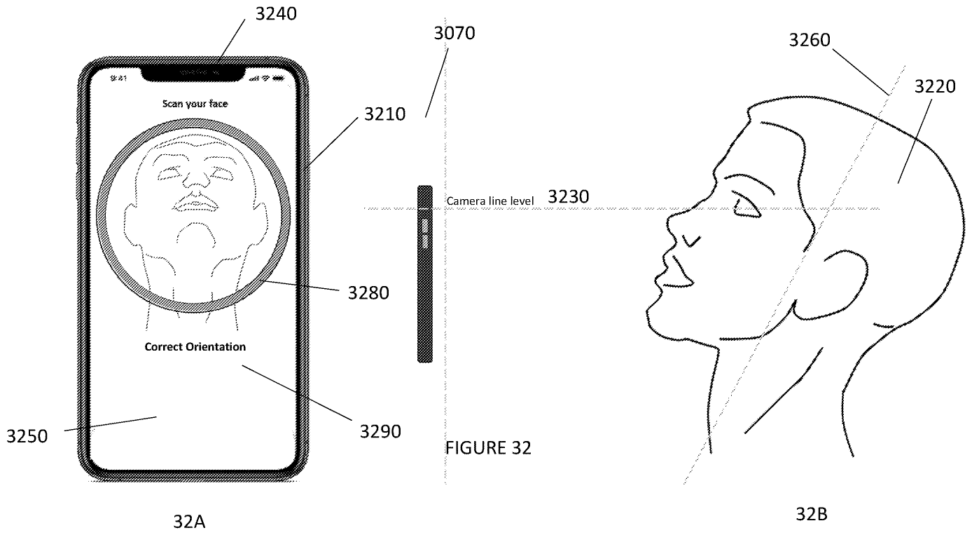

A second type of facial image may include an underside view of the face of the patient. This may be referred to as an underside facial image. An example of a camera/face configuration for producing an underside facial image is shown in FIGS. 32A and 32B. In FIGS. 32A and 32B, the head of the patient is tilted backwards with respect to the camera plane 3230. The face of the patient is not directly facing the camera and an underside view of the face of the patient appears in the image. FIG. 27 is an example of an underside facial image.

Front facial images and underside facial images are two examples of facial image types. Further facial image types include views of the face of the patient at other orientations.

The methods may be implemented on a user device. A software application may be loaded onto a user device, for example a mobile phone, tablet, desktop or other computing device. The software may operate solely on the user device or may be connected to a server across a communications network.

As described above, different categories of patient interfaces are available for patient therapy. Patients have individual needs based on therapy type, breathing habits, sleeping habits and other personal factors and so some patient interface categories are more suitable for the needs of individual patients than others. Some patients may require to a full face patient interface covering the nose and mouth of the patient, others may require a nasal mask which covers the nose only, other patients may require an under nose nasal mask which seals around the nostrils or a nasal pillow which seals inside the nostrils or around the outside of the nostrils. Different patient interfaces contact and seal to different parts of the face and so each patient interface category requires specific facial dimensions to be known in order to be fitted accurately. The facial dimensions may be the dimensions of a particular facial feature, for example the width of the nose, the height of the nose or the size of the nostrils.

Different patient interface categories contact the face at different points of the face, as described above with respect to FIGS. 2 and 3. In order to fit the patient interface accurately, different facial dimensions are relevant when fitting patient interfaces of different categories. This is because the patient interfaces seal against different parts of the face.

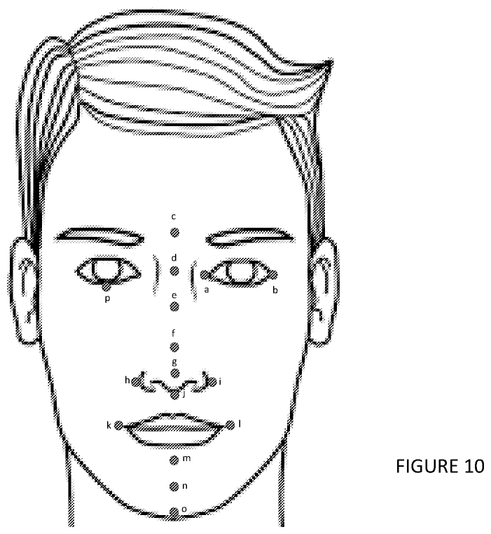

FIG. 10 is an illustration of a patient's face identifying various facial landmarks. Facial landmarks are points of the face. These landmarks may be used for measurements and are referred to below. These facial landmarks are anthropometric landmarks of the face, including for example but not limited to:
a) Medial canthus
b) Lateral canthus (i.e. ectro canthus).
c) Glabella
d) Nasion
e) Rhinion
f) Supratip lobule
g) Pronasale
h) Left alare (alar lobule)
i) Right alare (alar lobule)
j) Subnasale
k) Left labial commissure (i.e. left corner of mouth)
l) Right labial commissure (i.e. right corner of mouth)
m) Sublabial
n) Pogonion
o) Menton
p) Orbitale

Facial features are any feature related to the face. Facial features include the eye, nose, mouth. Facial features also include parameters and measurements related to the face. For example the width of the nose, the height of the nose, the depth of the nose, the width of the eye, are all facial features.

Facial features may be located between certain facial landmarks. In some cases the facial feature may be defined as the distance between certain facial landmarks. For example, the facial feature of nose width is defined between the left and right alar lobule (landmarks h and i of FIG. 10). Nose width may be calculated as the distance on the face between the left and right alar lobule. Nose width may be calculated when the coordinates of the left and right alar lobule are known.

FIG. 24 illustrates the seal 2420 between the patient interface and the face for a full face patient interface. For a full face mask, example relevant feature dimensions for sizing are shown in FIG. 24. A first relevant dimension is the dimension 2430 from the nasal bridge to the lower lip. Referring to FIG. 10, this is the dimension from landmark (d) nasion to landmark (m) sublabial. A second relevant dimension is the width of the mouth 2450. Referring to FIG. 10, this is the dimension between landmark (k) left labial commissure and landmark (l) right labial commissure. A third relevant dimension is the width of the nose 2440. Referring to FIG. 10, this is the dimension between landmark (h) left alare and landmark (i) right alare. When a patient requires a full face mask, these three dimensions should be obtained and compared to patient interface fitting data for full face masks to select a full face mask which fits the patient.

For a nasal mask, the relevant facial features are nose height 2530 and nose width 2540. The facial feature of nose height is defined between facial landmark (d) nasion and landmark (j) subnasale. The facial feature of nose width is defined between the left and right alar lobule (landmarks h and i of FIG. 10). When a patient requires a nasal face mask, these two dimensions should be obtained and compared to mask fitting data for nasal face masks to select a nasal face mask which fits the patient.

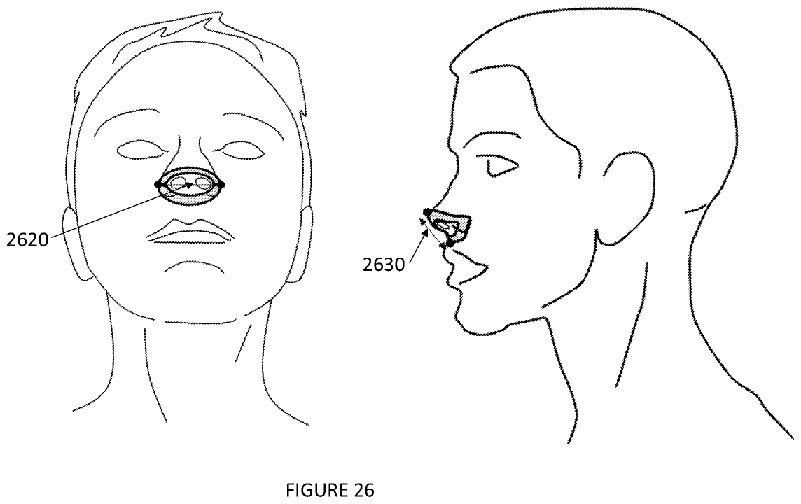

For under nose nasal masks shown in FIG. 26, the relevant facial features are nose width 2620 and the nasal length 2630 (i.e. nasal depth). This is because the seal sits under the nose and wraps around under the nose. Nose width is defined as the dimension between the left alar lobule (feature h in FIG. 10) and the right alar lobule (feature i in FIG. 10). Nasal length is determined for example based on the distance of the pronasal tip (feature g in FIG. 10) to the subnasale (feature j in FIG. 10). When a patient requires a under nose nasal mask, these two dimensions should be obtained and compared to patient interface fitting data for under nose nasal mask to select an under nose nasal mask which fits the patient.

For nasal pillow interfaces shown in FIG. 27, the relevant facial features and dimensions are nose width 2620 and nasal length 2630. Nose width is defined as the dimension between the left alar lobule (feature h in FIG. 10) and the right alar lobule (feature i in FIG. 10). Nasal length is determined for example based on the distance of the pronasal tip (feature g in FIG. 10) to the subnasale (feature j in FIG. 10). Additionally, the size of the nostrils is required. For sizing nostrils, the nostril is approximated to an elliptical shape having a major axis 2740 and a minor axis 2750. When a patient requires a nasal pillow, these four dimensions may be obtained and compared to patient interface fitting data for nasal pillow to select a nasal pillow which fits the patient. In some systems, nasal pillow interfaces may be fitted using nostril size only.

Table 1 below provides a summary of the dimensions required in order to fit different patient interface categories accurately:

TABLE 1
Facial dimensions required to Fit Different
Patient interface Categories

| Patient interface Category | Facial Feature Dimensions Required |
|---|---|
| Full Face Mask | Nasal bridge to the lower lip |
| | Mouth width |
| | Optionally: Nose width |
| Nasal Face Mask | Nose height |
| | Nose width |
| Under Nose Nasal Mask | Nose Width |
| | Nasal depth |
| Nasal Pillow | Nostril size: major axis, minor axis |
| | Optionally: Nose width, Nasal depth |

The dimensions described above and summarized in Table 1 are examples for the purposes of demonstrating different dimensions that may be required to fit different patient interface types. In some cases, some dimensions may be more dominant than others in fitting, or some dimensions may not be required.

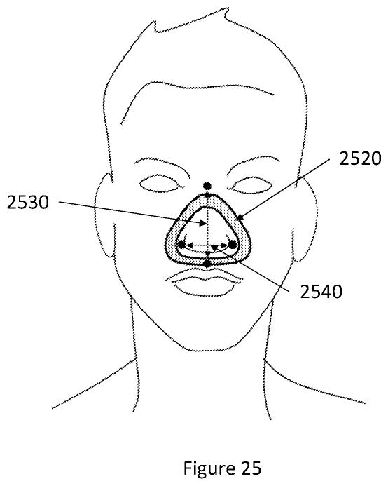

In order to obtain an accurate dimension for the required facial features, images showing particular orientations of the face or head may be required. Certain features, for example nose width are most accurately captured using a front facial image like that shown in FIGS. 24 and 25. Other features, for example the size of the nostrils or nasal depth may be most accurately captured in an underside facial image in which the head is tilted backwards, like that shown in FIG. 26 or FIG. 27. These dimensions may be skewed if the patient is face on with the screen or may not be visible.

Table 2 below shows which facial image type provides the most accurate dimensions for different facial features.

TABLE 2
Preferred facial image type for calculating
different facial feature dimensions.

| Facial Feature | Preferred Facial Image Type |
|---|---|
| Nasal bridge to lower lip | Front image of face |
| Width of mouth | Front image of face |
| Nose height | Front image of face |
| Nose width | Front image of face |
| Nasal depth | Underside image of face |
| Nostril size | Underside image of face |

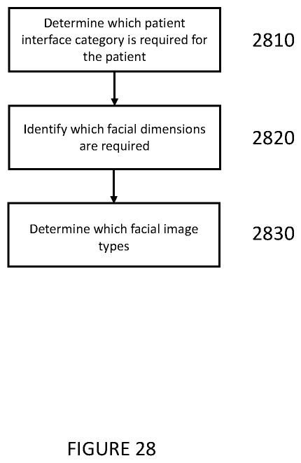

FIG. 28 is a flow diagram showing steps performed. At 2810 the system determines which patient interface category is required for the patient. Once the required patient interface category has been determined at 2810, the method identifies which dimensions are required in order to accurately fit a patient interface from the determined patient interface category in 2820. Typically the system retrieves this information from a look up table or other file type within a memory. At 2830, the system determines which facial image types are required in order to calculate the required dimensions and accurately fit a patient interface of the determined patient interface category. For some patient interface categories, multiple facial image types are required. Information defining which facial image types are required may be retrieved from a look up table or other file type within a memory.

Step 2810 of determining which patient interface category is suitable for a patient, may be performed by collecting data from the patient, in the form of subjective or objective data. Preferred embodiments present questions to the patient in the form of a questionnaire. In an example implementation in which the system is implemented on a software application on a patient electronic device, the questions are predefined and are presented to the patient on the display of the mobile communications device. The patient is prompted to respond to the questions by providing a response. In an example embodiment, the response is received through user input device on the patient electronic device. The question may be a YES/NO question or a question having predefined response options which are presented to the patient.

The patient responses in the form of subjective or objective data are used in the selection of a patient interface category for a patient. The responses from the patient are used to help the application to identify which patient interface categories are most suitable for the patient. The patient response may be used in combination with the dimension data calculated from the facial images of the patient to recommend a patient interface to the patient.

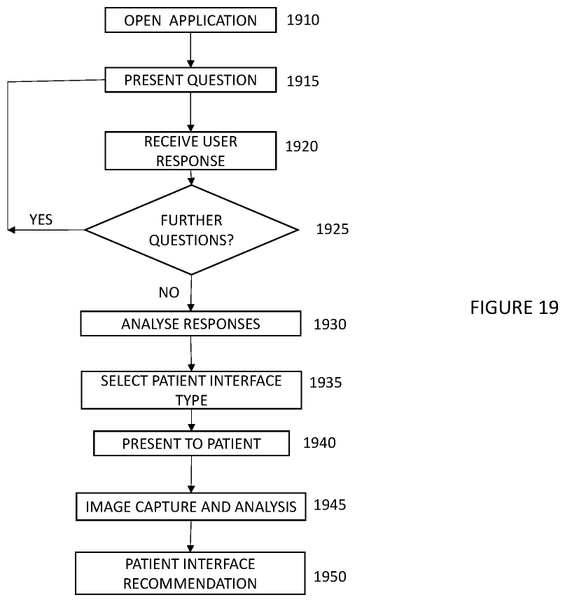

The steps performed by an example software application are now described with respect to FIG. 19. In the following embodiment, questions are presented to a patient on activation of a patient interface sizing application. The questions are presented and responses received before the application initiates a camera for the image capture process.

The questions are provided to support the patient interface selection software application in recommending an appropriate patient interface or an appropriate group of patient interfaces or a patient interface category for the patient. In the following example, the questions are presented to a patient to select a patient interface type or patient interface category suitable for the patient. Patient interface categories include full face mask, nasal mask, sub-nasal masks, under nose masks. As discussed above, each patient interface category fits differently onto the face of the patient and may engage with different features of the patient's face.

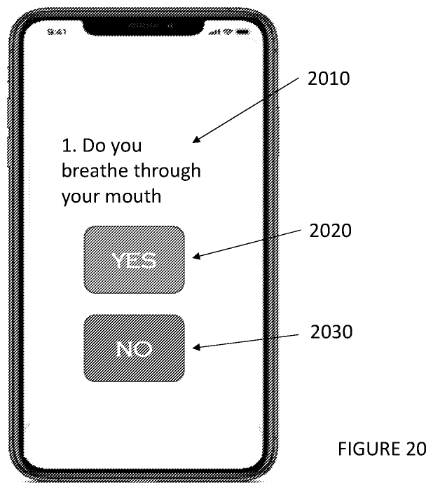

At 1910 the patient interface selection software application is accessed by a patient on a mobile communications device. At 1915, a question is presented to the patient. In an example embodiment the questions are presented on the screen of the mobile communications device. The questions may be presented individually or collectively. FIG. 20 is an illustration of a question being presented on the screen of a mobile communications device. The question is presented as text 2010 and asks the patient “Do you breathe through your mouth?”. The user is presented with response options YES 2020 or NO 2030. Preferably the display is a touchscreen display and the patient can provide a response by touching the appropriate response text on the display. The response is received by the application at 1920.

In other embodiments, audible questions are presented to the patient. Voice recognition software of the phone may be used to receive a vocal response from the patient. One example of suitable software is Apple's Siri application or Android's Voice Access application. The application may be used to present the question to the patient. Patient responses may be provided via the touchscreen via a virtual button or via an audible manner by the user in which the patient can speak their response.

Multiple questions may be presented sequentially. In an example embodiment, all questions are YES/NO questions, but in some embodiments additional predefined responses may be presented, or the patient may be able to provide an independent open text response.

Different question sets may be provided to different patients. In one example the application presents an initial question at 1915 to determine whether the patient has previously used a Positive Airway Pressure (PAP) device. Different question sets or question sequences are presented to the patient depending on whether the patient has previously used a PAP device or not.

At 1915 a question is presented to the patient:
HAVE YOU USED A PAP DEVICE OR MASK BEFORE?

The user is presented with response options YES and NO. User response is received at 1920.

At 1925, the application identifies the patient response and determines which question to ask next. The following sequences of questions are examples of sequences of questions which may be presented to the patient depending on whether they answer YES or NO to the question HAVE YOU USED A PAP DAVICE OR MASK BEFORE? The questions may be presented sequentially, displaying a single question at a time and waiting for the patient response before displaying the next question to the patient. Alternatively, the questions may be displayed concurrently or in groups.

In the exemplary embodiment, if the patient answers NO to the question HAVE YOU USED A PAP DEVICE BEFORE?, the application presents the following questions to the patient:

| QUESTION | ANSWER OPTIONS |
|---|---|
| ARE YOU A RESTLESS SLEEPER? | YES/NO |
| DO YOU WEAR GLASSES IN BED BEFORE | YES/NO |
| SLEEP? | |
| DO YOU HAVE SENSITIVE NOSTRILS? | YES/NO |
| DO YOU STRUGGLE TO HANDLE THINGS/ | YES/NO |
| HAVE ANY DEXTERITY ISSUES? | |
| WHAT IS YOUR PREFERRED SLEEPING | BACK/SIDE/ |
| POSITION? | STOMACH |
| DO YOU GET CLAUSTROPHOBIC OR | YES/NO |
| ANXIOUS? | |

In the exemplary embodiment if the patient answers YES to the question HAVE YOU USED A PAP DEVICE BEFORE?, the application presents a different set of questions to the patient:

| QUESTION | ANSWER OPTIONS |
|---|---|
| WHAT CATEGORY OF MASK HAVE | ORONASAL (FULL |
| YOU USED BEFORE/ARE | FACE)/NASAL/SUB |
| CURRENTLY USING? | NASAL/NASAL PILLOWS |
| WHAT DO YOU LIKE/DID YOU | SMALL AND COMPACT/ |
| LIKE ABOUT YOUR MASK? | COMFORTABLE/SIMPLE |
| | TO USE/COLOUR SHAPE/ |
| | NOTHING |
| WHAT DO YOU DISLIKE/DID YOU | BULKY/UNCOMORTABLE/ |
| DISLIKE ABOUT YOUR MASK? | LEAK ISSUES/HARD TO |
| | USE AND FIT/NOTHING |
| ARE YOU A RESTLESS SLEEPER | YES/NO |
| DO YOU WEAR GLASES IN BED | YES/NO |
| BEFORE SLEEP? | |
| DO YOU HAVE SENSITIVE | YES/NO |
| NOSTRILS? | |
| DO YOU STRUGGLE TO HANDLE | YES/NO |
| THINGS/HAVE ANY DEXTERITY | |
| ISSUES? | |
| WHAT IS YOUR PREFERRED | BACK/SIDE/STOMACH |
| SLEEPING POSITION? | |
| DO YOU GET CLAUSTROPHOBIC OR | YES/NO |
| ANXIOUS? | |

The questions listed above are a combination of YES/NO questions and multiple choice questions. Questions may also include an option to answer “I don't know”. This allows a more suitable score to be calculated for patients who do not know an answer to a question and prevents the patient guessing a YES or NO answer. Further embodiments may include different questions. Further embodiments include options for a patient to provide a free text response. Further examples do not have an initial question that determines the presentation of subsequent questions. Further examples have questions update as the user progresses through the questionnaire in the from of questions being skipped or changing the content of questions or further questions being added.

The sequence of questions may be predefined and fixed. In further embodiments the sequence of questions may be dependent on the responses provided by patients and the application determines which question to present next based on previous responses.

On receipt of the response by the application at 1920, the application determines whether any further questions are required at 1925. If yes, a further question is presented to the patient at 1915. If not, the patient responses are analysed at 1930. Optionally, the application may not present a single question if the user (e.g. Patient) answers YES to the question HAVE YOU USED A PAP DEVICE BEFORE? If the user answer's YES, then the application may present a question such as PLEASE SELECT THE MASK CATEGORY THAT YOU USE/HAVE USED BEFORE. The application may then present the available patient interface categories e.g. Full Face, Nasal, Under Nose etc.

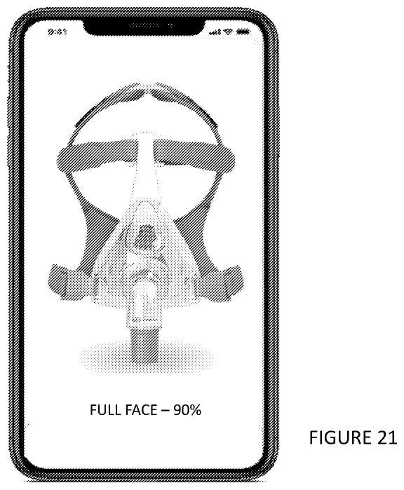

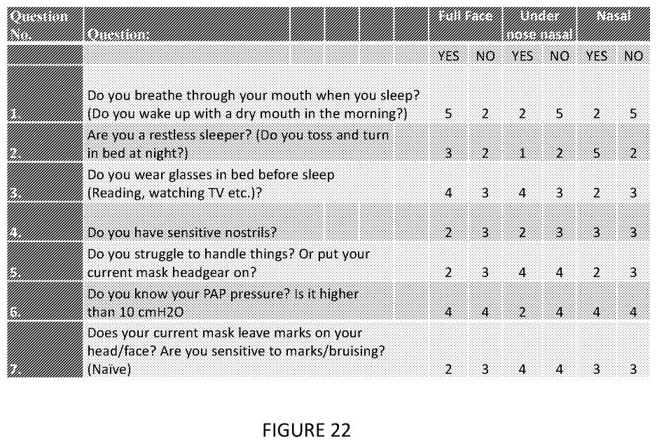

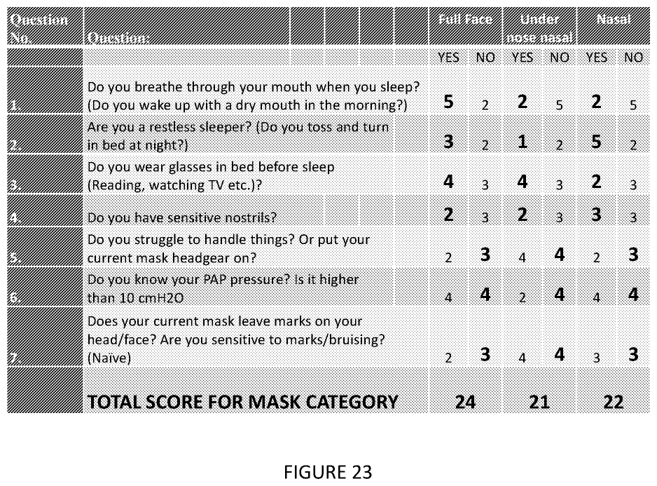

In one example, described in more detail below with reference to FIGS. 22 and 23 each of the responses received by the application is provided a score and weighted. The overall score for the patient is calculated. Patient interface categories are provided with specific scores and a patient interface category recommendation is generated at 1935. In other embodiments a list of two or more patient interface categories may be recommended, for example in order of suitability. The patient interface category recommendation may be displayed on the mobile communications device at 1940. Further information may be displayed with the patient interface recommendation. Examples of further information include an image of the patient interface, information about the patient interface, for example patient interface category, or relevance of the patient interface. FIG. 21 provides an example of a display identifying that a full face mask is recommended to the patient. The display identifies that the full face mask provides a 90% match based on the answers provided by the patient.

FIG. 22 illustrates an example of a scoring table associated with a series of questions presented to a patient. The questionnaire includes seven questions presented to the patient. In the example of FIG. 22, each question has a YES/NO answer. The patient responses are collected and mapped against three different patient interface categories, namely FULL FACE, UNDER NOSE NASAL, NASAL. Additional categories and associated mapping of answers may also be included.

The table shown in FIG. 22 is used to calculate suitability scores for each patient interface for a specific patient, based on the answers to the questions of that specific patient. This step is performed at Step 1930 of FIG. 19. As each patient interface has different characteristics, each question may have a different relevance/weighting for different patient interfaces. The weighting Is represented by different scores allocated to the YES/NO responses for the different patient interfaces, as shown in FIG. 22. For example, nasal mask provides a high score of 5 for a ‘no’ answer to the question asking if patient breathes through their mouth, since these patient interfaces are suitable for patients who breathe through their nose. The specific scores are generated based on various clinical studies and other research and can be tweaked and recalibrated in the future.

Some questions might be neutral for a specific patient interface, in which case the score given for that question is the same regardless of the answer the patient gives indicating that that question has little importance/relevance for that specific patient interface. An example question is Question 5, “Do you struggle to handle things? Or put your current patient interface headgear on?”. The patient scores a “4” regardless of whether the input answer is YES or NO for the under the nose category because this question has little relevance for that specific category.

An example of the patient responses to the questions of FIG. 22 are now described with reference to FIG. 23 to illustrate how the patient interface selection software application uses the patient responses to select a patient interface category for the patient. The patient responses are shown in the following table:

| QUESTION | ANSWER |
|---|---|
| DO YOU BREATHE THROUGH YOUR MOUTH WHEN | YES |
| YOU SLEEP (DO YOU WAKE UP WITH A DRY | |
| MOUTH IN THE MORNING)? | |
| ARE YOU A RESTLESS SLEEPER (DO YOU TOSS | YES |
| AND TURN AT NIGHT)? | |
| DO YOU WEAR GLASSES IN BED BEFORE SLEEP | YES |
| (READING, WATCHING TV, ETC)? | |
| DO YOU HAVE SENSITIVE NOSTRILS? | YES |
| DO YOU STRUGGLE TO HANDLE THINGS? OR PUT | NO |
| YOUR HEADGEAR ON? | |
| DO YOU KNOW YOUR PAP PRESSURE? IS IT | NO |
| HIGHER THAN 10 cmH20? | |
| DOES YOUR CURRENT MASK LEAVE MARKS ON | NO |
| YOUR HEAD/FACE? ARE YOU SENSITIVE TO | |
| MARKS/BRUISING? | |

The answer to each question generates a score for each patient interface category which depends on the suitability of that patient interface to the response provided by the patient. For example, question 1: Do you breathe through your mouth when you sleep? (Do you wake up with a dry mouth in the morning?). The patient input answer YES. The answer YES scores 5 in the Full Mask category. This is a high score indicating that the full face mask category is suitable for patients who breathe through their mouths. The answer YES only scores 2 in the under nose nasal and nasal mask categories, indicating that these masks are less suitable for patients who breathe through their mouths.

In the example, question 6: Do you know your PAP pressure? Is it higher than 10 cmH2O?, the patient has answered “NO”. This answer scores 4 in each of the patient interface categories. This indicates that none of the patient interfaces are more suitable than the others for a patient who does not know their PAP pressure. This is an example of a neutral response.

The patient interface scores for the patient based on the responses provided are calculated for each category of patient interface. In the example shown in FIG. 23, the highest scoring patient interface category for the patient is Full Face. The lowest scoring patient interface category is Under nose nasal. These scores indicate that the most suitable patient interface category for the patient is a full face patient interface. As discussed above, after a patient interface category is determined for a patient, the patient interface category may be displayed to the patient at 1940. FIG. 21 shows an example of a display screen presenting a patient interface category to the patient.

In an example the questionnaire is presented to the patient in a first stage of the patient interface selection process. After the responses have been received by the application and the system has analysed the responses and determined which patient interface category is suitable for the patient, the application enters a second stage of the patient interface selection process at 1945 to fit a patient interface for the patient.

The process of fitting the patient interface includes the steps of calculating the dimensions of relevant facial features of the patient and comparing the dimensions with patient interface data to determine which patient interface provides the best fit for the patient. The system performs the process of fitting the patient interface by capturing images of the patient's face and calculating dimensions of the patient's face. Typically, the first stage of the patient interface selection process of presenting the questions to the patient, is concerned with selecting the most suitable patient interface category. The second stage of the patient interface selection process is concerned with calculating the size of facial features of the patient and selecting the most appropriate size patient interface in the suitable patient interface category. This second stage of the patient interface selection process is now described in more detail below.

As described above with reference to FIG. 28, once the patient interface category has been selected, the system identifies which facial feature dimensions are required in order to fit a patient interface from the selected patient interface category, and so which facial image types are required in order to obtain those dimensions. In order to calculate the dimensions of the facial features in the images, the scale of the images must be known.

The width of the eye is a useful feature to use as a reference feature of the face because its dimension is found to have minimal variance amongst adults, typically aged 16 and above. Embodiments take a measurement of the eye within the image (i.e. the number of pixels of the image corresponding to the width of the eye) and allocate a predefined dimension to the eye width. That dimension allows a scaling factor to be created for the image. This allows dimensions of other features in the image to be calculated.

An accurate measurement for the eye may be captured in a front facial image and used to provide a scaling factor for the image. That scaling factor may be used in other facial image types, i.e. in images of the face from other orientations, to obtain dimensions of other facial features, for example nose depth or nostril dimensions.

In some situations multiple facial image types may be required to retrieve dimension information. A first image type may be used to determine a scaling factor for measurements within the image or to determine a dimension of a feature which appears in a further image. A further image type may then be used to obtain a measurement for a particular facial feature and that measurement may be converted to a dimension using the scaling factor.

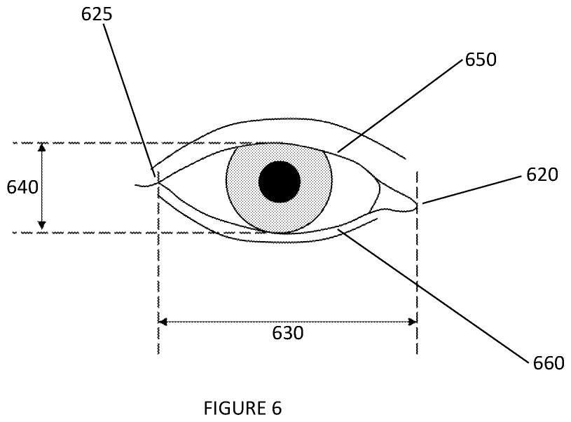

The following description describes an embodiment in which the eye of a patient in a front facing image is used as a reference facial feature to scale an image of the patient's face. FIG. 6 shows a human eye and surrounding parts of the face. The eye includes two corners: a first corner 620 positioned on the face at an innermost point of the eye, closest to the centre of the face; and a second corner 625 positioned on the face at an outermost point of the eye, furthest from the centre of the face. The distance between the corners of the eye is the eye width.

These corners may be defined by the two canthi of the eye. The facial landmark relating to the innermost point of the eye is the medial canthus 620. The facial landmark relating to the outermost point of the eye is the lateral canthus 625.

The width of the eye is a useful feature to use as a reference feature of the face because its dimension is found to have minimal variance amongst adults, typically aged 16 and above.

In one example, the width of the eye is the distance between the corners of the eye.

In other embodiments, the width of the eye is the distance between the medial canthus 620 and the lateral canthus 625.

In other examples, the width of the eye may be defined as the distance of the white region of the eye, where the corners 620 625 are defined as the point of contrast between the white of the eye and the face.

In other examples, the width of the eye is the horizontal distance between the medial canthus 620 and the lateral canthus 625. This distance is the horizontal palpebral fissure 630. The horizontal palpebral fissure is a useful feature of the face to use as a reference feature. This feature is found to have minimal variance amongst individuals aged 16 and above. In other examples the height of the eye may be used as a reference feature. The height of the eye may be defined as the distance between the upper eyelid 650 and the lower eyelid 660 when the eye is open. The height of the eye may be the maximum distance between the upper eyelid and the lower eyelid when the eye is open. This height may be defined as the vertical palpebral fissure 640.

The eye width can be detected in images or videos of a patient's face. Since the canthi are landmarks of the face, rather than parts of the eyeball, like the iris or the pupil, these landmarks are not obscured by the eye lid of the patient. Since the canthi are landmarks of the face, the eye width can be captured in an image even when the eye is closed, partly closed or during blinking. The width of the eye is a greater length than other parts that may be used as reference features, for example the iris or the pupil, so any percentage measurement error will likely be lower than for a smaller reference feature. Similarly, the eye height can be detected in images or videos of a patient's face.

A further benefit of using the width of the eye, or height of the eye, as a reference feature is that measurements can be obtained for both eyes of a patient within an image, allowing an average measurement to be calculated. This averaging can also reduce the error in the measurement value.

The scaling factor described above is obtained by measuring the width of the eye of the patient in a front facial image. The front facial image is preferred for scaling using the width of the eye.

When facial images are required at orientations other than front facing in order to obtain measurements of facial features, for example nose depth or nostril size, images at a second orientation, for example an underside orientation, may be required. Relevant facial features are identified in the image at the second orientation and measurements for those features are taken within the image, i.e. the number of pixels for a particular feature is calculated. The scaling factor calculated from the front on image of the face is used to calculate dimensions of the second image. The scaling factor is applied to the measurements of the image at a second orientation in order to calculate the dimension of the relevant facial feature. That dimension can then be used to select a patient interface for the patient from the required patient interface category. Underside facial images can provide dimensions suitable for fitting under nasal and pillows type patient interface and also nasal cannula.

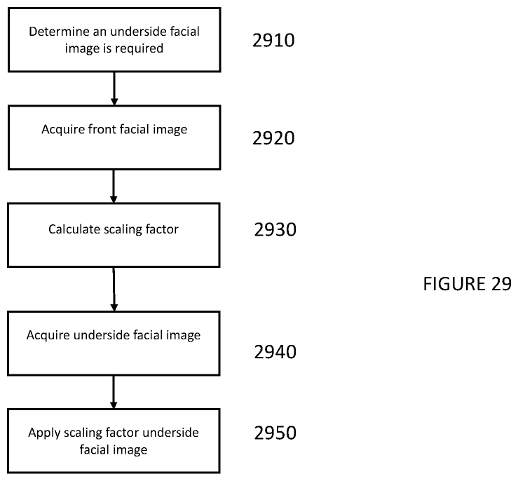

The process for scaling an image of a second facial image type, i.e. at a second orientation, is described with reference to FIG. 29. At 2910 it is determined that an image at a second orientation is required in order to obtain accurate dimensions of relevant facial features. The second orientation relates to an underside facial image. At 2920, a scaling image is acquired. The scaling image is required to calculate a scaling factor for the images. In preferred embodiments the scaling factor is calculated by acquiring a front facial image and using the width of the eye to determine a scaling factor for the front facial image at 2930. At 2940, an image of a second facial image type is acquired, for example an underside facial image. This different orientation allows a more accurate measurement of relevant facial features to be made. At 2950, the scaling factor is applied to the underside facial image. The measurement of the relevant facial feature in the underside facial image, i.e. a measurement for the facial feature in terms of number of pixels, is converted to a dimension for the facial feature using the scaling factor. The relevant facial feature is identified in the second image, a measurement for the relevant facial feature in the image is made and the scaling factor is applied to the measurement to calculate a dimension of the facial feature. The dimension can then be compared with patient interface fitting data to determine a patient interface size for the patient.

A feature may appear in both images, for example the width of the nose may appear in both images and be used as an “image scale” to scale the second image. In these embodiments, the scaling factor for the front facial image is calculated using the width of the eye of the patient and the dimension of the width of the nose is calculated using the scaling factor and a measurement of the width of the nose in front facial image. In the underside facial image, another measurement for the width of the nose is made. The calculated dimension of the width of the nose, or otherwise the width of the nose, in the front facial image is compared to the width of the nose in the underside facial image. This works as an “image scale”, in that the absolute value of the width of the nose is known to be the same across the two images; thus any discrepancy in apparent size gives an indication of the relative distance (or position), from the image capture device, of the nose in the two images, in order to scale the underside facial image to compensate for any misalignment in distance or position between the two images.

When software application executes the method on an electronic device, the device may be configured to instruct the user to capture a first image with the face in a first orientation with respect to the camera. If further images are required at different orientations, the application may provide instructions prompting the patient to re-orientate his face with respect to the camera. This would be a different orientation, i.e. not front on to the camera. Thus, multiple facial image types can be required and a sequence of instructions can be presented to a user in order to accurately obtain multiple facial image types to aid patient interface fitting.

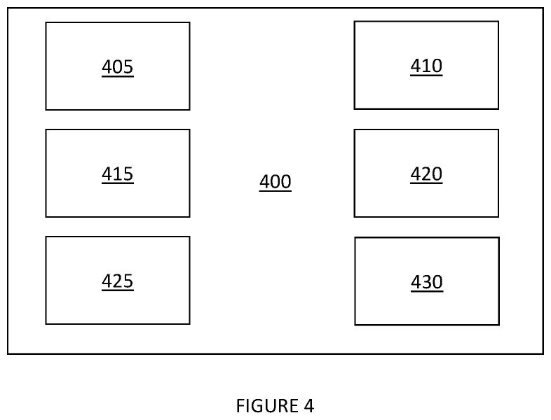

In the following description, the method is implemented by a software application executed on a mobile communications device. The terms mobile communication device, mobile communications device, user device and mobile device are used interchangeably. A schematic representation of the mobile communications device is shown in FIG. 4. Mobile communications device 400 includes an image capture device 405. In the example of FIG. 4 the image capture device is a digital camera. Mobile communications device 400 includes memory 420. Memory 420 is a local memory within communication device 400. Memory 420 is suitable for storing software applications for execution on the mobile communications device, algorithms and data. Data types include patient interface data including patient interface category data and patient interface sizing data, reference scales and dimension information for facial features and landmarks, image recognition software applications suitable for identifying facial features and landmarks within images, questions for presentation to the user, etc. Memory also stores data identifying which facial feature dimensions are required to fit different patient interface categories and which image orientations are required to obtain each facial feature orientation.

Mobile communications device 400 includes processor 410 for executing software applications stored in memory 420. The mobile communications device includes display 430. The display is suitable for presenting information to a user, for example in the form of text or images, and also for displaying images captured by camera 405. User input device 425 receives input from a user. User input device may be a touch screen or keypad suitable for receiving user input. In some embodiments user input device 425 may be combined with display 430 as a touch screen. Transceiver 415 provides communication connections across a communications network. Transceiver 415 may be a wireless transceiver. Transceiver 415 may support short range radio communications, for example Bluetooth and/or WiFi. Transceiver 415 also supports cellular communications. Alternatively multiple transceivers may be implemented, each transceiver configured to support a specific communication method (i.e. communication protocol), such as for example WiFi, Bluetooth, cellular communications etc.

In the following example mobile communications device 400 is a mobile phone but device 400 could be a tablet, laptop or other mobile communications device having the components and capabilities described with respect to FIG. 4. In some illustrated examples the mobile communications device is a smartphone.

The components of the mobile communications device 400 shown in FIG. 4 may be located at or within mobile communications device 400 or may be external to the mobile communications device. The components may be connected to each other or to the mobile communications device via a wired connection or via a wireless connection, for example communications network. For example memory 420 may be located on the mobile communications device or located externally and access by the mobile communications device via a communication channel, for example a communications network or short range connection.

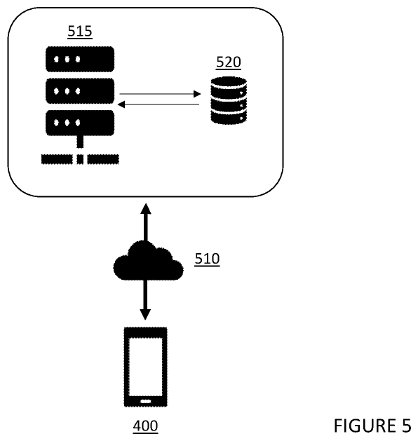

The communication path between mobile communications device 400 and various servers is shown in FIG. 5. In FIG. 5 mobile communications device 400 communicates with server 515 across a communications network 510. Server 515 accesses and/or communicates with database 520. The mobile communications device 400 exchanges data with sever 515 and database 520. Communications device 400 may request data from server 515 and/or database 520. Communications device 400 may provide data to server 515 and/or database 520. Server 515 and/or database 520 may provide data to mobile communications device 400 in response to a request from mobile communications device and/or may selectively push data to mobile communications device 400.

Data relating to the patient interface selection software application may include: questions to be presented to a patient during a patient interface selection process within a patient questionnaire; database data associating responses to questionnaire questions to various patient interface categories; data relating to sizing information associating facial feature dimensions with patient interface sizes; data identifying which facial feature dimensions are required to fit different patient interface categories and which image orientations are required to obtain each facial feature orientation; and, general information about devices or patient interfaces, for example patient interface instructions, cleaning instructions, FAQs and safety information. Details of some specific databases used in various embodiments are provided below. The diagram of FIG. 5 is for illustrative purposes only, further implementations may include communication connections between multiple servers and databases.

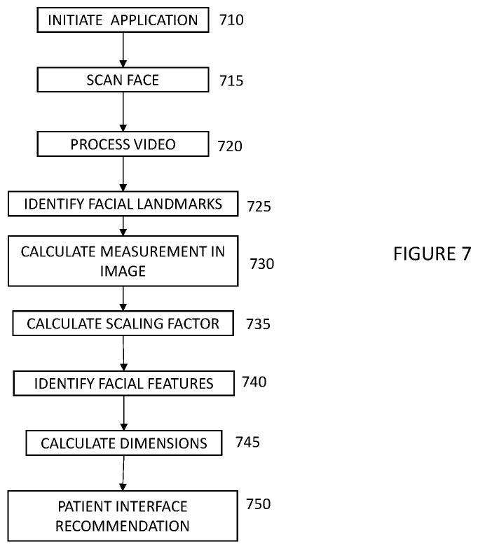

The steps performed by a patient interface selection software application operating on a mobile communications device are now described with reference to FIG. 7. In the description the terms: patient interface selection software application; patient interface sizing application; software application; and, application, are used interchangeably.

The patient interface selection software application is a software programme that may be stored in memory 420 and executed by processor 410. The software programme is a computer executable programme for execution using the processor 410 of mobile communications device 400. The computer programme may include a series of instructions to be executed by processor 410 and may be or may include algorithms. The programme is executed locally using data that is acquired at the mobile communications device 400. In the following description, the various modules, for example facial detection module, face detection module and face mesh module, the applications, and the algorithms, may specifically form part of the patient interface selection software application or may reside as separate computer programmes stored in memory 420 which are called by the patient interface selection software application during execution when required. The software programme may reside in an internal memory of the mobile communications device or in an external memory and accessed via a wired connection or wireless connection, for example across a communications network.

At 710 a patient interface selection software application is opened on mobile communications device 400. The patient interface selection software application is opened for the purpose of recommending a respiratory therapy patient interface to a patient. The patient interface selection software application is a software programme that may be stored in memory 420 and executed by processor 410.

On selection of the patient interface selection software application by the patient, the patient interface selection software application is initiated at 710. The patient interface selection software application accesses camera 405 in order to capture a digital image of the patient's face. Preferably the forward facing camera (i.e. a selfie camera) on the same side of the device as the display screen is accessed by the patient interface selection software application. This orientation is commonly recognized as capturing an image in ‘selfie’ mode, so the patient can view the image on the display screen during image capture. The patient interface selection software application may provide guidance to the patient, for example in the form of text instructions or example images on the display screen 430, to help the patient capture a suitable image.

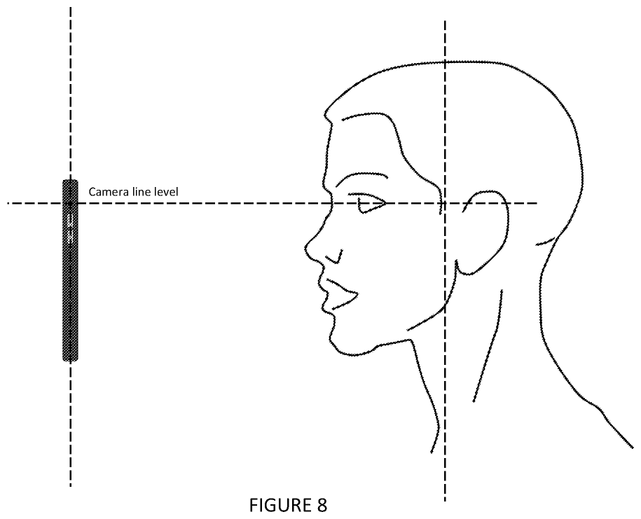

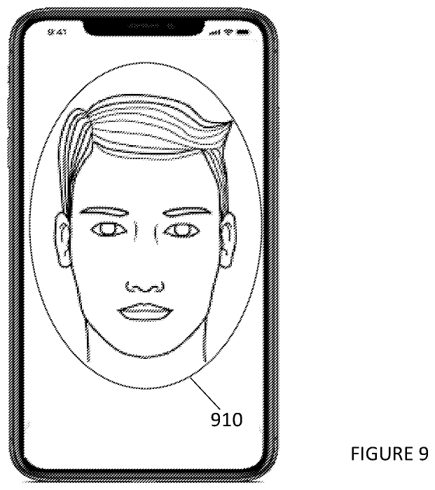

The patient interface selection software application is configured to be operated independently by a patient and so an image of the patient's face may be obtained by holding the mobile communications device away from the patient with the camera directed at the patient's face, as shown in FIG. 8. Preferably the image captured by the camera is displayed to the user on display screen 430 as shown in FIG. 9. Visual guidance to aid the patient in capturing the image may be provided, for example in the form of frame 910. Further guidance which may include text may be presented on the screen instructing the user to position their face within the frame.

During image capture, the application captures or may capture a stream of digital image frames. The rate at which frames are captured may vary between applications or devices. The rate at which frames are captured may be related to the clock in the mobile device and may be dependent on the type of mobile device. In some embodiments only a single image frame is captured. In such systems the application may prompt the patient to capture the image, for example by providing a button on the screen for taking the image. In other embodiments multiple frames are captured as part of a video in a frame sequence. Individual or multiple frames may be extracted from the multiple frames for analysis. In exemplary systems, multiple frames are automatically captured. The video image frames or image frame is captured at 720 and processed to produce a digital image file of the face of the patient. The file may be any suitable file type, for example JPEG.

The patient interface selection software application includes a facial detection module. The facial detection module is a software programme configured to analyse an image file and detect predefined facial landmarks in the image. At 725 the patient interface selection software application runs a facial detection module on the image to identify facial landmarks.

In exemplary embodiments the facial detection module is a machine learning module for face detection and facial landmark detection. The facial detection module is configured to identify and track landmarks of the face. Preferably the facial detection module operates in real time and analyses images generated by the camera of the mobile device as they are captured.

Exemplary facial detection modules may comprise a face detection module and a face mesh module. The face detection module allows for real time facial detection and tracking of the face. The face mesh module provides a machine learning approach (or another suitable approach) to detect the facial features and landmarks of the user's face and/or to superpose a mesh onto the face, so as to provide a set of (preferably three-dimensional) coordinates for the points on the mesh and thus many points on the face (including the facial landmarks and facial features). In some examples, the machine learning approach continually updates its libraries, and uses stored data on a plurality of sampled faces to correct for irregularities in a captured image. The face mesh module provides locations of face landmarks and provides a coordinate position of each landmark. The landmark positions are provided as a coordinate system. For example the coordinate system may be a cartesian coordinate system or a polar coordinate system. The zero point i.e. reference point for the coordinate system is preferably located on the patient's face e.g. at the center of the nose. Alternatively, the reference point may be located off the face i.e. a point in space that is used by the module when determining the locations of the facial landmarks and providing location information e.g. coordinates. The face detection module and the face mesh module together allow for tracking of landmarks and features. For instance, the face detection module may detect movement of the facial features, and the face mesh module may cause the face mesh, which is superposed onto the face, to “follow” the detected movement of the facial features. These may be two separate programmes or may be incorporated into a single programme or algorithm.

Alternatively, the face detection module and face mesh module may be separate computer programs i.e. that may be stored in the memory of the mobile communication device. The processor 410 is configured to execute the programs in this alternative configuration.

Exemplary embodiments may be configured to select a predefined subset of the total facial landmarks detected by the facial detection module and to calculate dimensions for features defined by these landmarks only. The particular subset of the total facial landmarks may be selected based on a current operation of the patient interface selection software application, patient input, patient interface category or other selection criteria.

At 725 the application identifies predefined facial landmarks in the image captured by the patient device. The application applies a coordinate system onto the digital image of the patient's face. In an exemplary embodiment, the coordinate system is a 3-dimensional coordinate system (x, y, z). In one implementation the centre of the nose is set as coordinate (0,0,0) and the coordinates of all landmarks are determined in relation to the (0,0,0) point.

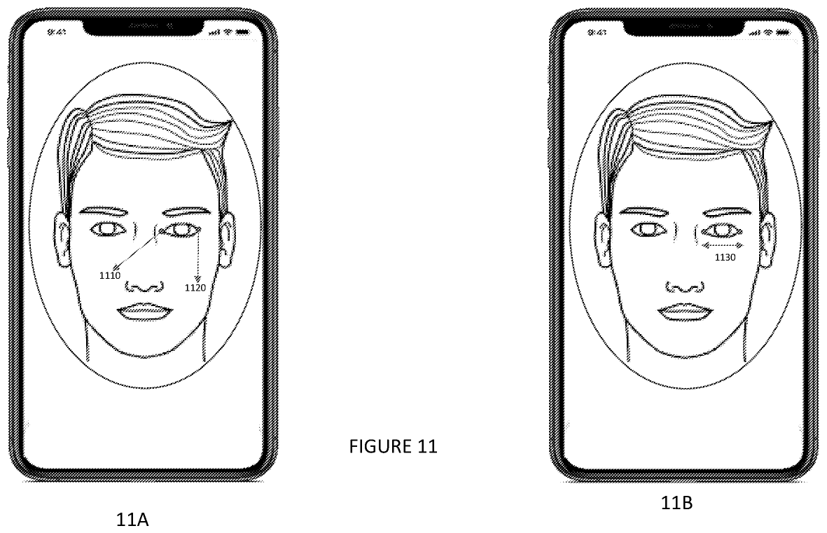

As shown in FIG. 11A, the application identifies the medial canthus 1110 and the lateral canthus 1120 within the image of the patient's face, i.e. the two corners of the eye of the patient. The x, y, z coordinates for the medial canthus and the lateral canthus are identified, lateral canthus (x1, y1, z1) and medial canthus (x2, y2, z2).

Now shown in FIG. 11B, a measurement for the reference feature of the eye width 1130 is calculated within the image. In this exemplary embodiment, the measurement for the eye width is calculated using the x and y coordinates only, z coordinates are ignored. In other embodiments, the z coordinates may also be used in calculating the measurements.

In the exemplary embodiment, the measurement for the eye width is calculated between the canthi using the formula:

$$Eye width measurement = \sqrt{{( x 1 - x 2 )}^{2} + {( y 1 - y 2 )}^{2}}$$

The measurement is the length of the feature in the image. The units of the measurement may be pixels of the image. Other units for the measurement, for example image vectors may be used. Calculations based on two dimensions (x and y coordinates) only can be useful as it saves on computation.

Further embodiments calculate the eye width measurement using the x coordinates of the canthi only. In these exemplary embodiments the eye width measurement is calculated using the formula, |x1−x2| or |x2−x1|. In some embodiments it may be useful to use more than one of the x, y, and z coordinates to account for any non-standard positioning of facial features.

The application may calculate the width of one eye in the image at step 730 as described above. In further embodiments, the application identifies the corners of both eyes of the patient's face appearing in the image. A width measurement is calculated for each eye and averaged in order to obtain an average eye width for the patient in the image. Use of an average width across both eyes can reduce errors.

At 735, a scaling factor for the image is calculated. Memory 420 stores a reference dimension associated with the eye. As discussed above the eye width is a useful reference feature as it shows minimal variance across adults. The dimension is the size of the feature on the patient's face. Exemplary embodiments use the reference dimension of the eye width to be 28 mm. The reference dimension may relate to the average eye width (i.e. horizontal palpebral fissure) of a human eye. A different reference dimension may be used for the height of the eye, for example 10 mm. This corresponds to the average eye height (i.e. vertical palprebal fissure). In the illustrated and described sizing method eye width is used.

The application calculates a scaling factor for the image using the eye width measurement in the image and the eye width dimension of 28 mm. The scaling factor is the ratio between the width measurement in the image and the width dimension. As discussed above, the width measurement may be taken in pixels or in some other suitable units.

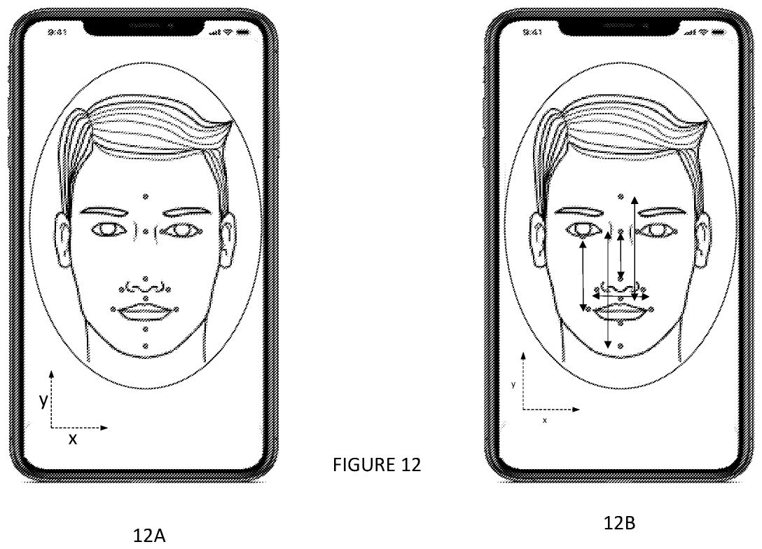

Referring to FIG. 12A, at 740 facial landmarks are identified in the image by the facial detection module and the coordinates of each facial landmark (x, y, z) in the image are determined. The processor of the mobile device is configured to receive image coordinates for each of the identified facial landmarks. The anthropometric landmarks of interest may be a preselected subset of the total anthropometric landmarks identified in the image.

Referring to FIG. 12B, the measurements of preselected facial features, for example the width of the nose, of the height of the nose may be calculated by identifying the two anthropometric landmarks associated with each preselected facial feature and determining the length between the landmarks in the image. This measurement may be the difference between the absolute value of x coordinates (e.g x1−x2) only or the absolute value of y coordinates (y1−y2) only. The horizontal dimension i.e. x dimension may be obtained by determining the difference between the x coordinates and the vertical dimension i.e. y dimension may be obtained by determining the difference between the y coordinates (as described earlier). Alternatively, the measurement between the landmarks may be calculated using the equation \sqrt{(x1−x2)²+(y1−y2)²}. Exemplary embodiments may calculate the measurements using two dimensions or three dimensions. Again, the measurements may be calculated in pixels or any other suitable unit of measurement.

In FIG. 12B, the arrows illustrate measurements of various facial features that may be calculated. The z dimension may be used for example to calculate nasal depth e.g. the z distance between the subnasale and pronasale. The measurements of the nasal features are calculated in pixels or some other measure (e.g. image vectors). The z dimension may only be relevant for particular patient interface categories, for example the under nose patient interface shown in FIG. 3. The z depth measurement |z2−z1| or |z1−z2| is calculated in the image and may be converted to a facial dimension for the patient using the same scaling factor derived from the eye width as previously described.

At 745, the facial measurements in the image, i.e. the number of pixels, is converted to a facial dimension using the scaling factor for the image calculated with respect to the eye width dimension. For example, using 28 mm as the dimension of the eye width:

$$facial feature dimension = \frac{facial feature measurement ( pixels ) \times 28 mm}{reference feature measurement ( eye width measure in pixels )}$$

Optionally each of the measurements may be multiplied by a further scaling factor, this may be referred to as a rectification factor or compensation factor. The further scaling factor is a suitable scalar that is predetermined. In some embodiments the further scaling factor may compensate for a fish eye effect of camera lenses and/or other distorting factors. This further scaling factor (for example rectification factor or compensation factor) is applied in addition to the scaling factor. For example the compensation factor is applied to account for any distortion in the image, for example due to the lens, and the scaling factor is applied in addition to the compensation factor to convert the measurement in the image to the dimension on the face.

The feature identification and dimension calculations may be calculated from a single image. In another embodiment, multiple images may be captured by the camera, each image being a separate image frame, and processed. In each image, the dimensions may be calculated for each feature of interest and the final calculated dimension for a feature on the face of the patient is an average dimension across the multiple images, to reduce errors.

In embodiments in which dimensions are required from images which are not front facial images, the facial feature measurements are calculated from the images in terms of pixels. The scaling factor from the front on image is applied to the measurement to calculate the dimension.

The facial detection module may be preprogramed to capture a minimum number of frames to calculate an average dimension across. In an exemplary embodiment at least 30 frames are captured and/or processed. In another example, at least 100 frames are captured and/or processed. The facial detection module may be preprogramed to require data to be captured over a minimum length of time, for example 10 seconds of video, to be captured and processed i.e. 10 seconds of x, y, z data of facial landmarks. Measurements are then averaged over the captured frames.

In order to manage memory storage space, frames or patient images may not be stored in the memory, i.e. nothing persists. The frames are stored for the time to process and then deleted. Temporary memory could be ROM, RAM and optionally some temporary cache memory.

The processing may be performed in real time on the mobile communications device. In an exemplary embodiment, the processor processes frame by frame on the mobile communications device in real time. In alternative embodiments, multiple frames are stored and then processed in batches, for example frames from a time period of video recording or from a predetermined number of frames are stored and processed on the phone. Additionally/alternatively, captured video/images are transmitted and processed on the cloud server. A further alternative is that each frame is captured and transmitted to the cloud for processing.

As described above, the facial detection module is a software module and may include a machine learning (ML) module. The machine learning module is configured to apply (and/or has been trained on) one or more deep neural network models. In one example two ML models are used. A first face detection module operates on the image (or frames of a video) for real time facial detection and tracking of the face. A second face mesh module detects the facial features and landmarks of the face and provides locations for face landmarks. The face mesh model may operate on the identified locations to predict and/or approximate surface geometry via regression.

The facial detection module uses the two ML models to identify facial features and landmarks. The identified facial features may be displayed on the screen. These facial features may be used as part of processing the recorded images (or processing each frame of a video recording). The landmarks may be identified and tracked in real time even as the patient may move. ML models use known facial geometries and facial landmarks to predict locations of landmarks in an image.

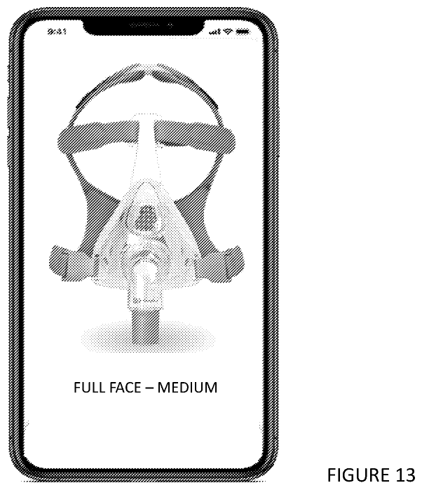

After the dimensions have been calculated at 745, the dimensions are compared to patient interface data stored in the database to identify a patient interface suitable for the patient. A patient interface size that corresponds to the dimensions of the facial features is recommended to the patient at 750. An example of a recommended patient interface displayed to a patient is shown in FIG. 13. In the example of FIG. 13 the recommended patient interface is a full face patient interface, medium size. The application may provide links to purchase options for the patient. For example the application may provide a link that allows purchase of the selected patient interface and size from a patient interface retailer or dealer that provides such patient interfaces.

Some methods are configured to check that the camera is correctly positioned to capture an image of the patient's face. The angle between the camera and the face of the patient is calculated. For example, when the method is implemented on a mobile communications device, for example a phone, the angle may be calculated using sensors within the phone that also comprises the camera. In one example the sensors may comprise one or more accelerometers and one or more gyroscopes. The one or more accelerometers and one or more gyroscopes may determine an angle of the camera (relative to the vertical). Additionally, the facial detection module and/or face mesh module may determine or be used to determine the angle of the face relative to the camera or the phone.

The angles between the camera and the face of the patient may also be calculated by processing the image to determine the angle of the face of the patient in the image. The system may define various parameters, for example angles, and if the image is captured outside of those parameters then the image may be rejected and/or feedback may be provided to the patient to re-orientate the camera with respect to the face.

Images are analysed to determine whether attributes of the image meet certain predefined criteria. If the attributes of an image do not meet the predefined criteria, measurements from those images are not used to calculate dimensions of the patient's face. The image may be discarded. This is a filtering step to ignore images in which measurements may be inaccurate, leading to the calculation of incorrect dimensions of the face of the patient. The predefined criteria are predefined filtering criteria. The steps of analysing the image to determine whether the image meets predefined criteria may be performed after the image is processed.

One example of an attribute of an image is the angle of the patient's head with respect to the camera in the image. Further examples of attributes of an image include distance between the camera and the head of the patient, lighting levels, the position of the head within the display and whether all required features are included in the image.

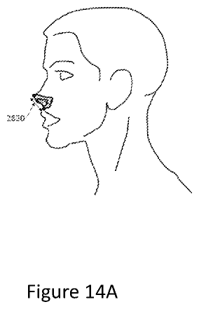

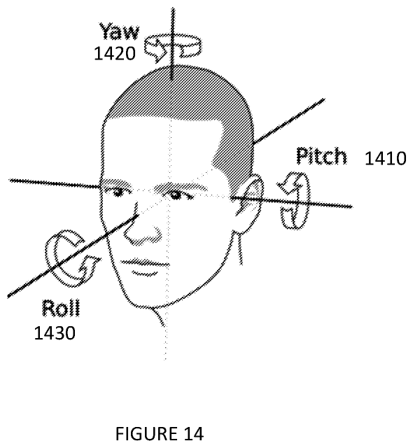

FIG. 14 shows three axes of rotation of the head of a patient. Pitch 1410 is the angle of tilt of the head up and down. Yaw 1420 is the angle of rotation left and right. Roll 1430 is the angle of rotation side to side. The angles of pitch, yaw and roll are measured with respect to the angle of the camera. The accuracy of calculations of dimensions of features within the image may be affected by variations in the angles of pitch, yaw and roll of the image. Images having different angles of pitch, yaw or roll could generate different measurements for certain features and the distance between landmarks of those features may change and landmarks may appear closer together or further apart than they actually are.

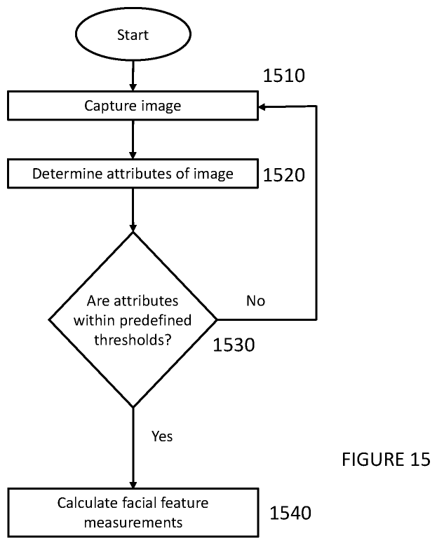

FIG. 15 shows steps that may be implemented by the application to determine whether the attributes of an image meet the predefined criteria. If the attributes of the image meet the predefined criteria, then that image may be used to calculate facial dimensions of the patient. Generally, the steps of FIG. 15 are performed in real time when the image frame is captured at step 720 of FIG. 7.

At 1510, an image is captured by the camera and processed (step 1510 is equivalent to step 720 of FIG. 7). At 1520, the application determines the pitch, yaw and roll angles of the head of the patient within the image and any other required attributes. In exemplary embodiments these attributes are determined in real time.

Various methods may be used to determine the angles of pitch, yaw and roll. In one exemplary method, the application generates a matrix of face geometry. The matrix defines x, y and z values for points on the face in a Euclidean space. The patient interface sizing application determines pitch, yaw, and roll from relative changes in the x, y, and z Euclidean values as the user's face moves and changes angles. As a user's face moves and changes angles the coordinates of a certain landmark or point can be compared with that landmark's coordinates when the face measures a pitch, yaw, and roll of (0, 0, 0), or a previous angle, or a calibration reference point, to derive the new values of pitch, yaw, and roll at the changed angle. Pitch, yaw, and roll can be measured in +ve and −ve values about various axes that intersect at a common origin point. The x, y, and z points used to measure pitch, yaw, and roll are all measured in relation to the common origin point (0,0,0) that may be located at the Nasion or Pronasale for example.

At 1530, the angles of pitch, yaw and roll are compared against predefined threshold values stored within the memory. These threshold values define tolerance levels for acceptable images. The predefined threshold values may be different for pitch, yaw and roll. In one embodiment the predefined threshold value for pitch angle is 10 degrees in either the +ve or −ve direction. If the pitch angle is greater 10 degrees in either the +ve or −ve direction, then measurements from the image are not used to calculate dimensions of the patient's face.

Predefined threshold values are also applied to yaw and roll. In one example, the predefined thresholds for roll and yaw are greater than 2 degrees in +ve or −ve directions.

Predefined threshold values may vary between embodiments. In one embodiment, the threshold values for pitch is between 10 degrees in the +ve or −ve directions. In exemplary embodiments the threshold value for pitch is 6 degrees in the +ve or −ve directions. Other threshold values may be used in other embodiments. In some embodiments, threshold values may be applied to pitch, yaw and roll. In other embodiments, threshold values may be applied to one or more of pitch, yaw and roll.

If the image meets the predefined threshold criteria at 1530 then the measurements or dimensions of the face of the patient calculated from the image may be used during patient interface selection at 1540. If the image does not meet the predefined threshold criteria at 1530 then the image is not used in the patient interface selection process towards a recommendation at Step 750 of FIG. 7. If the image does not meet the predefined threshold criteria the application may revert to 1510 to capture a further image.

The filtering steps of determining whether an image meets the predefined criteria may be performed at different stages. The timing of calculating the predefined criteria may be selected based on the processing capabilities of the device, the frame rate, or other factors.

In one embodiment, the dimensions of facial features are calculated regardless of whether the attributes of the image meet the predefined threshold criteria. In such embodiments steps 725 to 745 of FIG. 7 are performed regardless of whether the attributes of the image meet the predefined criteria. The application discards the dimensions calculated from images not meeting the predetermined criteria and these dimensions are not used when selecting a patient interface for the patient. In other applications, the attributes of the image are calculated and compared against the threshold criteria during image processing immediately after image capture. Images for which the attributes do not meet the required criteria are discarded after Step 720 of FIG. 7 and dimensions are not calculated using these images.

By discarding images in real time, immediately after image capture at Step 720, memory storage and processing load is reduced. Each frame is assessed as it is extracted from a video stream or an image frame buffer. Alternatively, the system may store all or a predetermined number of frames and then assess filtering criteria such as the image attributes described above. By discarding images having attributes which do not meet the predefined criteria, frames that could give the wrong eye width dimension or an inaccurate eye width dimension or give distorted facial features are not considered in the calculation of dimensions.

In some embodiments the application provides the patient with feedback to confirm whether or not the attributes of the image or images being captured by the patient meet the predefined criteria. The feedback may be visual feedback. The feedback may be a visual indicator. The feedback may be text. By providing feedback to the patient, the patient is able to respond to the feedback in real time in order to capture an image which meets the requirements. This can help improve user experience.

The feedback may be haptic feedback. Haptic feedback may include vibrations or a specific vibration pattern to indicate instructions to the user. For example, two short vibrations may mean tilt up and a single short vibration may mean tilt down. Similar haptic feedback can be provided for distance of face to phone, for example three vibrations could be mean move the camera closer to the head and four vibrations could mean move the camera further away from the head.

The feedback may be audio feedback. The audio feedback may provide vocal instructions or sounds to provide instructions to the patient to change the relative orientation or position of the camera with respect to the head. Audio feedback commands are particularly useful to assist patients who are hard of sight.

Some embodiments include a combination of feedback, for example a combination of haptic, visual and audio feedback. Some embodiments may include a combination of haptic and visual feedback, haptic and audio feedback, audio and visual feedback or haptic, visual and audio feedback.

In some embodiments, the application detects the orientation of the camera. The camera, or a device containing the camera, may include orientation sensors, for example a gyroscope and/or accelerometer and/or an Inertial Measurement Unit (IMU). The orientation sensors detect the orientation of the camera. In some examples the application has a predefined preferred orientation, for example vertical, of the camera. The application receives sensor data monitoring the orientation of the camera. The application compares the orientation of the camera to the predetermined preferred orientation. Indicators may be presented to the user on the screen of the device to assist the user in orientating the device and camera correctly. Various versions of the system may include caricatures or various animations on the screen, illustrating how the user should move their device. These may be used instead of, or in addition to, the text prompts. This step of orientating the camera into a preferred orientation may be performed before the image capture process. For example, the image capture process may not be initiated until the camera is positioned at a preferred angle, for example in a vertical orientation. In embodiments including this camera orientating requirement, after initiating the sizing application the application, the application determines whether a specific orientation for the camera is required for image capture. If so, the application receives orientation data from orientation sensors for example gyroscope and/or accelerometer and/or IMU to determine the current orientation of the camera. The current orientation of the camera is compared with required orientation for image capture. If the camera is not at the required orientation, the application may provide guidance to the user to change the orientation of the camera into the required orientation. The guidance may be provided using text or animation on the display of the device. Or guidance may be provided using an alternative feedback type. In order to assist the user, general guidance may be provided, for example “Position your camera vertically”. Other starting orientations may be used. During the camera orientation process, the camera may not be active and so the display screen may not display an image.

In some embodiments, when the required orientation of the camera is detected, the application initiates the image capture process, detects the orientation of the face or head of the user, and provides guidance to the user to orientate their face or head in the required orientation with respect to the camera for image capture.

In some embodiments the application receives data from the orientation sensor during the image capture process. If the orientation of the camera is changed out of the desired orientation, then indicators may be provided to the user to re-orientate the device into the preferred orientation. In some embodiments the image capture process may be paused or interrupted until the camera is re-orientated correctly.

By detecting the orientation of camera, the application is able to provide guidance to the user to orientate the camera in a predefined orientation for the image capture process. By knowing the orientation of the camera (in space) the application can also calculate the orientation of the face or head of the user in space in an image or during the image capture process by calculating the relative angle between the camera and the face of the patient, for example using a matrix of face geometry as described above. For example, if the camera is positioned in a vertical orientation, the application can guide the user to orientate their face or head into a vertical orientation by calculating the relative angle of the face in the image and by providing guidance to the user to position their face or head into the plane parallel to the plane of the camera. An advantage of having the camera in the vertical orientation in space and having the face or head of the user in a vertical orientation in space is that distortion of the face is reduced when the face or head is orientated vertically. This allows a reliable measurement of the width of the eye and so enables a reliable scaling factor to be obtained which can be used to obtain measurements of other facial features in the image, or other images.

FIG. 16 shows an example of the orientation of a patient's head 1620 with respect to the mobile communications device 1610 during image capture. In the example of FIG. 16 the system requires a front facial image and the pitch requirements are met in the image. FIG. 16B is a side view to illustrate the pitch angle of a patient's head with respect to the camera. Similar images could be provided to illustrate yaw and roll angles. The camera 1640 of the mobile communication device is on the front face 1650 of the mobile communications device which includes the display for displaying the image captured by the camera. As discussed above, this arrangement allows the patient to view the image of their face during the image capture process. Camera line level is represented as 1630. The plane of the camera, and so the plane of the image, is represented in FIG. 16B as 1670. The relevant angle of the head of the patient is shown as 1660. In the example of FIG. 16, the head of the patient is directly facing the camera and the angle of the head of the patient relative to the plane 1670 of the camera is approximately zero. This produces a pitch angle of or close to zero. FIG. 16A shows the display of the mobile communication device. In the example of FIG. 16, the image captured by camera meets the predefined threshold criteria since the pitch angle is within the threshold values. The application provides feedback to the patient confirming that the captured image meets the criteria. The feedback may be visual feedback displayed on the screen of the mobile communications device. This feedback is provided to the patient by presenting a green outline indicator 1680 on the display of mobile communication device 1610, shown in FIG. 16A. The coloured indicator provides an indication to the user that the user is correctly using the device and that the face is straight. Text feedback 1690 “Fit your face inside the frame” may also be provided on the screen of the mobile communications device.

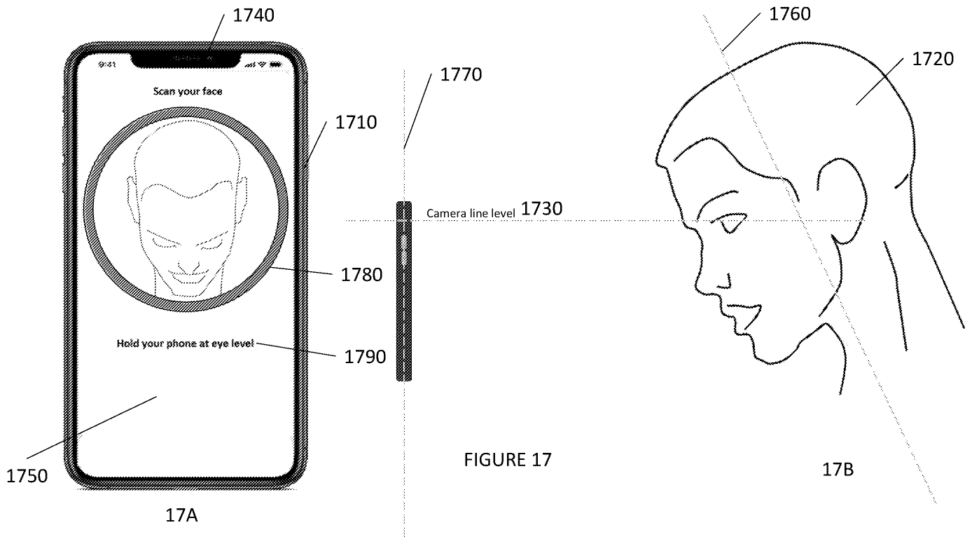

FIG. 17 shows a further example of the orientation of a patient's head 1720 with respect to the mobile communications device 1710 during image capture. Again, the system requires front facial image but in the example of FIG. 17 the pitch requirements are not met in the image. FIG. 17B is a side view to illustrate the pitch angle of a patient's head with respect to the camera. Camera line level is represented as 1730. The plane of the camera, and so the plane of the image, is represented in FIG. 17B as 1770. The angle of the head of the patient is shown as 1760. In the example of FIG. 17, the head of the patient is tilted forwards with respect to the camera plane 1770. This tilt of the head with respect to the camera produces a negative non-zero pitch angle. The head of the patient is not directly facing the camera and an elevated view of the face of the patient appears in the image. In the example of FIG. 17 the pitch angle does not meet the predefined threshold criteria since the pitch angle is outside the threshold values. The application provides feedback to the patient confirming that the captured image does not meet the criteria. This feedback is provided to the patient by presenting a red outline indicator 1780 on the display of mobile communication device 1710. In the example of FIG. 17, further feedback is provided to the patient to help them capture a suitable image in the form of text on the screen of the device. A text feedback instruction 1790 instructs the patient “Hold your phone at eye level”.

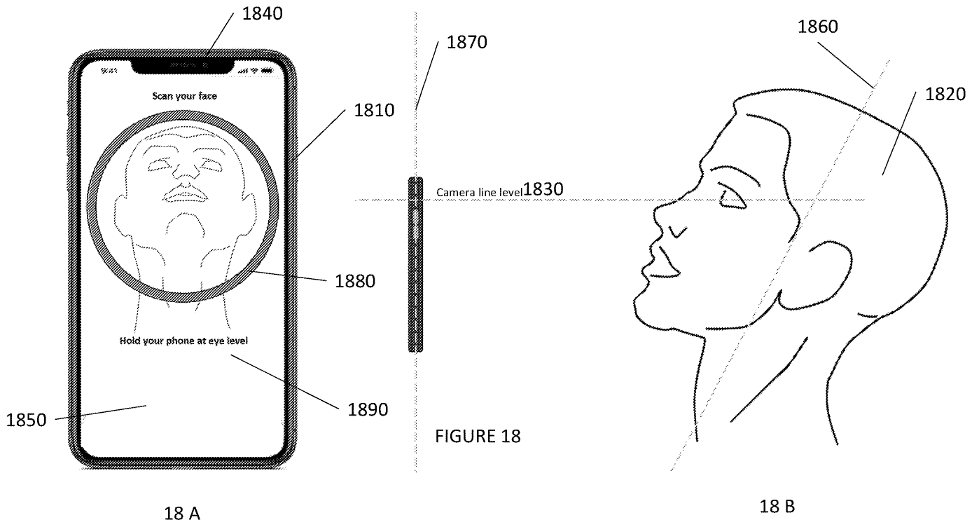

FIG. 18 shows a further example of the orientation of a patient's head 1820 with respect to the mobile communications device 1810 during image capture. Again, the system requires a front facial image but in the example of FIG. 18 the pitch requirements are not met in the image. FIG. 18B is a side view to illustrate the pitch angle of a patient's head with respect to the camera. Camera line level is represented as 1830. The plane of the camera, and so the plane of the image, is represented in FIG. 18B as 1870. The angle of the head of the patient is shown as 1860. In the example of FIG. 18, the head of the patient is tilted backwards with respect to the camera plane 1870. This tilt of the head with respect to the camera produces a positive non-zero pitch angle. The head of the patient is not directly facing the camera and an underside view of the face of the patient appears in the image. In the example of FIG. 18 the pitch angle does not meet the predefined threshold criteria since the pitch angle is outside the threshold values. The application provides feedback to the patient confirming that the captured image does not meet the criteria. This feedback is provided to the patient by presenting a red outline indicator 1880 on the display of mobile communication device 1810. In the example of FIG. 18, further feedback is provided to the patient to help them capture a suitable image in the form of text on the screen of the device. A text feedback instruction 1890 instructs the patient “Hold your phone at eye level”.

FIGS. 16, 17 and 18 provide illustrations of various pitch angles of the head of the patient in the image. Similar calculations may be performed for yaw and roll angles and the application may provide similar patient feedback for those angles to reposition the relative positions of the phone and the face if required.

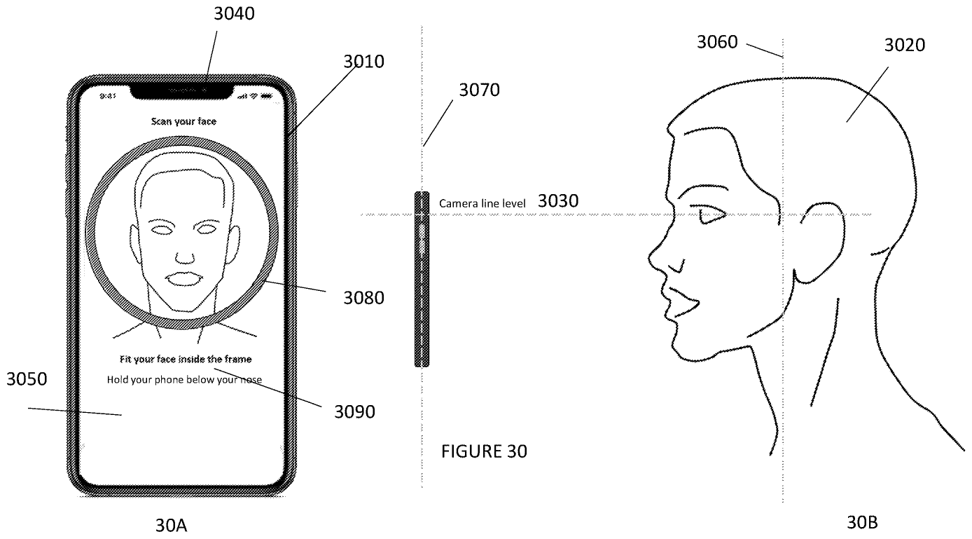

FIGS. 30, 31 and 32 illustrate a situation when a patient is required to acquire an underside facial image. This is a typical example of a patient requiring an under nasal mask and the system requiring underside facial images to obtain measurements for the patients nasal depth and nostril size. In this exemplary embodiment, the system defines criteria of an angle of between 35 degrees to 45 degrees between the camera and the face of the patient.

In FIG. 30, the patient has orientated his head in a front on angle with respect to the camera. The camera 3040 of the user device or mobile device is on the front face 3050 of the user device which includes the display for displaying the image captured by the camera. As discussed above, this arrangement allows the patient to view the image of their face during the image capture process. Camera line level is represented as 3030. The plane of the camera, and so the plane of the image, is represented in FIG. 30b as 3070. The relevant angle of the head of the patient is shown as 3060. In the example of FIG. 30, the head of the patient is directly facing the camera and the angle of the head of the patient relative to the plane 3070 of the camera is approximately zero. This produces a pitch angle of or close to zero. In the example of FIG. 30, the image captured by camera is outside the predefined threshold criteria since the pitch angle is outside the threshold values. The application provides feedback to the patient confirming that the captured image does not meet the criteria. This feedback is provided to the patient by presenting a red outline indicator 3080 on the display of mobile communication device 3010. The coloured indicator provides an indication to the user that the user has incorrectly positioned the device and that the user must reposition his face with respect to the camera. A text feedback instruction 3190 instructs the patient “Hold your phone below your nose”.

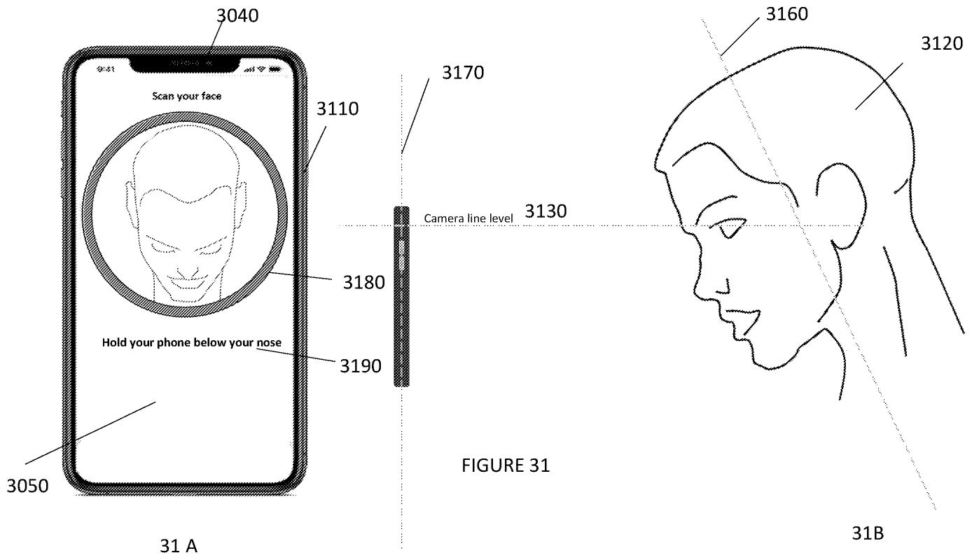

FIG. 31 shows a further example of the orientation of a patient's head 3120 with respect to the mobile communications device 3110 during image capture. Again, the system requires an underside image of the face with an angle of between 35 degrees to 45 degrees between the camera and the face of the patient. In the example of FIG. 31 the pitch requirements are not met in the image. The pitch angle does not meet the predefined threshold criteria since the pitch angle is outside the threshold values. The application provides feedback to the patient confirming that the captured image does not meet the criteria. This feedback is provided to the patient by presenting a red outline indicator 3180 on the display of mobile communication device 3110. In the example of FIG. 29, further feedback is provided to the patient to help them capture a suitable image in the form of text on the screen of the device. A text feedback instruction 3190 instructs the patient “Hold your phone below your nose”.

FIG. 32 shows a further example of the orientation of a patient's head 3020 with respect to the mobile communications device 3010 during image capture. Again, the system requires an underside image of the face with an angle of between 35 degrees to 45 degrees between the camera and the face of the patient. In the example of FIG. 32 the pitch requirements are met in the image. The pitch angle is within the required 35 degrees to 45 degrees range and meets the predefined threshold criteria. The application provides feedback to the patient confirming that the captured image meets the criteria. This feedback is provided to the patient by presenting a green outline indicator on the display of mobile communication device 3210. In the example of FIG. 32, further feedback is provided to the patient to confirm that the camera is in the correct orientation in the form of text on the screen of the device.

FIGS. 16, 17 and 18 and 30 31 and 32 provide illustrations of various pitch angles of the head of the patient in the image. Similar calculations may be performed for yaw and roll angles and the application may provide similar patient feedback for those angles to reposition the relative positions of the phone and the face if required.

Images are processed in real time during use of the camera by the patient and patient feedback is provided in real time. Thus, the system provides the patient with guidance on using the application to help the patient capture usable images for determining the dimensions of the face. This patient feedback supports non-expert users to capture images which can be used to obtain accurate measurements which can calculate accurate dimensions to be used for patient interface sizing.

In further embodiments one of the attributes of an image frame is the distance between the face of the patient and the camera. This attribute is used as a filtering criteria to determine whether an image frame is used to calculate a dimension of a facial feature. Preferably the phone is to be held at a predefined distance from the user's face. In one example the set distance is the focal distance or length of the camera. In another example the set distance is based on the reference feature (i.e. eye width). The reference feature, being eye width is allocated a reference dimension such as 28 mm. The distance of a user's face to the camera, and therefore phone, can be calculated using the reference feature dimension and other retrievable measurements such as the focal length of the camera. Such information may be stored in the metadata of a device or an image captured by the device. Further, the measurement of the reference feature as it appears in an image captured by the device can be calculated by the application. This measurement may be in pixels. The following formula may then be used to find the distance of the face from the camera by taking the ratios of the above-mentioned measurements.

$$\frac{Eye width in mm}{Distance of face to phone in mm} = \frac{Eye width in pixels}{Focal length ( pixel equivalent )}$$

In one example the predefined distance may be a set distance with a tolerance, for example 30 cm+−5 cm. Alternatively the predefined distance may be defined as a range, for example between 15 cm to 45 cm. Visual feedback is provided to the patient to indicate whether the relative position of the camera and the face of the user are within the predefined distance or range.

As shown in FIGS. 16, 17, 18, 30, 31, 32 visual feedback is provided in the form of an indicator which is displayed on the screen as a circle around the image of the face of the patient. The indicator (circle around the face) is a first colour (e.g. red) when the phone is not held at the predefined distance or does not meet other required attributes. If the phone is held at the set distance, then the indicator (circle) is green to indicate that the predefined attributes are met. This is advantageous because it provides a user an easy to understand and visual indicator in order to correctly position the mobile communications device. Other visual feedback, including text, may be presented on the screen to instruct the patient to correctly position the mobile communication device. Further the visual indicator is advantageous because it provides real time feedback to correctly position their head and mobile communications device. Optionally real time audio feedback and/or real time haptic feedback can also be provided. Audio feedback and haptic feedback can be optionally provided in combination with the visual feedback presented on the screen of the mobile communications device.

Other indicators may be used e.g. a tick or some other suitable indicator that visually provides information to a user.

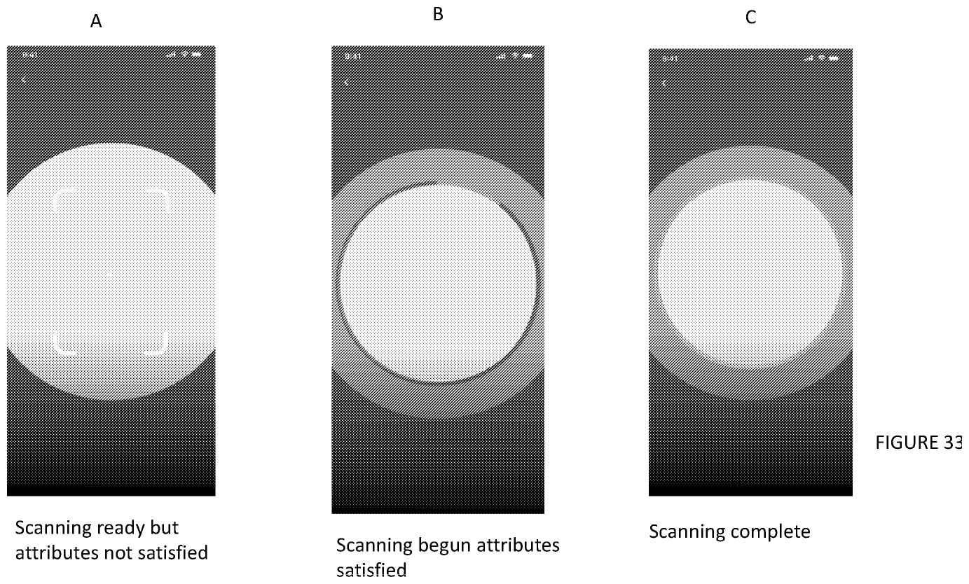

FIG. 33 provides a further illustration of a visual indicator presented to a patient during an image capture process. In the example of FIG. 33 the system runs changing animation on the screen at different stages of the image capture process.

In FIG. 33A the screen includes a highlighted area including a positional indicator within which the patient should position their face. This screen animation is displayed initially and until the patient has correctly positioned their face within the positional indicator.

When the face is correctly positioned within the positional indicator the animation on the screen is updated as shown in FIG. 33B. In 33B a shaded outline appears in the highlighted area and an animated circle appears to indicate that image capture is in progress. The circle may change colour gradually to indicate progress of scanning.

When scanning is complete, the animation is updated again as shown in FIG. 33C.

These visual indicators provide patients with an interactive and guided image capture experience.

Different patient interface categories contact the face at different points of the face, as shown in FIGS. 2 and 3 and described above. Consequently, different facial dimensions are relevant when fitting patient interfaces of different categories. The patient responses are used to identify which patient interface categories will be included in patient interface sizing. The following paragraphs provide examples of facial dimensions that may be relevant for different patient interface categories. After determining the most suitable patient interface category for a patient, example embodiments of the application calculate dimensions of facial features relevant for the determined patient interface category and use these dimensions to select the size of patient interface within the determined category.

FIG. 24 illustrates the seal 2420 between the mask and the face for a full face mask. For a full face mask, example relevant feature dimensions for sizing are shown in FIG. 24. A first relevant dimension is the dimension 2430 from the nasal bridge to the lower lip. Referring to FIG. 10, this is the dimension from landmark (d) nasion to landmark (m) sublabial. A second relevant dimension is the width of the mouth 2450. Referring to FIG. 10, this is the dimension between landmark (k) left labial commissure and landmark (l) right labial commissure. A third relevant dimension is the width of the nose 2440. Referring to FIG. 10, this is the dimension between landmark (h) left alare and landmark (i) right alare.

Referring now to FIG. 19, if the application determines that a patient requires a full face mask at 1935, based on patient responses to the patient questionnaire at 1920, during image analysis at 1945, the application retrieves the coordinates of the six example landmarks relevant to sizing a full face mask, namely: (d) nasion; (m) sublabial; (k) left labial commissure; (I) right labial commissure; (h) left alare and (i) right alare. The dimensions of the features defined by the landmarks, namely: nasal bridge to lower lip; width of the mouth; and, width of the nose, are calculated. The dimensions are then compared with the patient interface sizing data including dimensions or thresholds to determine which size patient interface is suitable for the patient. The patient interface sizing data may be stored in memory 420 of mobile communications device 400. By storing the patient interface sizing data on the mobile communications device the application is able to recommend a patient interface to the patient without requiring a network connection.

In embodiments, the facial detection module determines the coordinates for all facial landmarks in the image. The application identifies the landmarks relevant to the specific patient interface category and retrieves those coordinates to calculate the measurements of the relevant facial features in the image and the dimensions of those relevant facial features.

The sizing process is now described for a nasal face mask with reference to FIG. 25. For a nasal face mask, the relevant facial features are nose height 2530 and nose width 2540. The facial feature of nose height is defined between facial landmark (d) nasion and landmark (j) subnasale. The facial feature of nose width is defined between the left and right alar lobule (landmarks h and i of FIG. 10). Referring again to FIG. 19, when the application determines that a patient requires a nasal face mask at 1935 based on responses to the patient questionnaire at 1920, during image analysis, the application retrieves the coordinates of the four example landmarks relevant to sizing a nasal face mask, namely: (d) nasion; (j) subnasale; left alar lobule (h) and (i) right alar lobule. The dimensions of the features defined by the landmarks, namely: nose height and nose width are then compared with the patient interface sizing data including dimensions or thresholds to determine which size patient interface of nasal face mask is suitable for the patient.

The table below provides example sizing data for nasal face masks. A recommended patient interface size is provided for various nose heights and nose widths. In an exemplary embodiment, the data is stored as a look up table in memory 420 and the application references the sizing data to select a patient interface size for the patient.

| | | DIMENSION OF NOSE HEIGHT | | |
|---|---|---|---|---|
| | | <4.4 cm | 4.4 cm-5.2 cm | >5.2 cm |
| Dimension | <3.7 cm | S | M | L |
| of Nose | 3.7 cm-4.1 cm | M | M | L |
| Width | >4.1 cm | M | L | L |

The patient interface sizing data in the table is for sizing nasal face masks. The look up table provides a known result for the various possible combinations of the dimensions of the relevant features. For example, for nasal masks if the patient's nose height is calculated to be between 4.4-5.2 cm and nose width is calculated to be greater than 4.1 cm, then the most suitable size is a large (L).

Similar look up tables are provided for each patient interface category. For example, to size a full face mask with n relevant dimensions, an n-D lookup table would be used, that is a lookup table or function with n number of input parameters produces known results based on the various possible combinations of the input parameters and their different ranges. Different patient interfaces may have different sizing charts, lookup tables, or sizing functions. The look up tables are stored in memory.

The sizing process is now described for under nose nasal masks, with reference to FIG. 26. For under nose nasal masks, the relevant facial features are nose width 2620 and the nasal length 2630 (i.e. nasal depth). This is because the seal sits under the nose and wraps around under the nose.

Nose width is defined as the dimension between the left alar lobule (feature h in FIG. 10) and the right alar lobule (feature i in FIG. 10). Nasal length is determined for example based on the distance of the pronasal tip (feature g in FIG. 10) to the subnasale (feature j in FIG. 10). Referring again to FIG. 19, when the application determines that a patient requires an under nose nasal mask at 1935 based on responses to the patient questionnaire at 1920, during image analysis, the application retrieves the coordinates of the four example landmarks relevant to sizing an under nose nasal face mask, namely: left alar lobule (h); (i) right alar lobule; pronasal tip (g); and, subnasale (j). The dimensions of the features defined by the landmarks, namely: nasal length and nose height are compared with patient interface sizing data including dimensions or thresholds to determine which size patient interface of under nose nasal mask is suitable for the patient. The dimensions may be calculated using all three (x,y,z) coordinates for the four landmarks, or just using y and z.

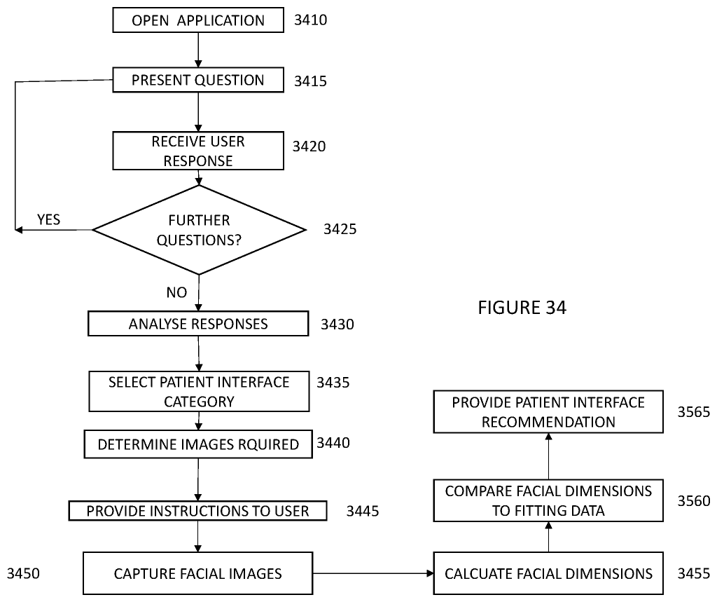

A further description of the steps performed when implemented on a mobile communication device is now described with reference to FIG. 34. At 3410 the software application is run on a mobile communication device. At 3415 predefined questions are displayed to the patient on the screen of the mobile device. User responses are input to the mobile communication device at 3420. The responses are processed and determined whether further questions are required to be presented. The order in which questions are presented or the questions themselves may be predefined or the order may be determined based on answers provided by the patient. At 3430 the system analyses the responses and selects patient interface type at 3435 based on patient answers.

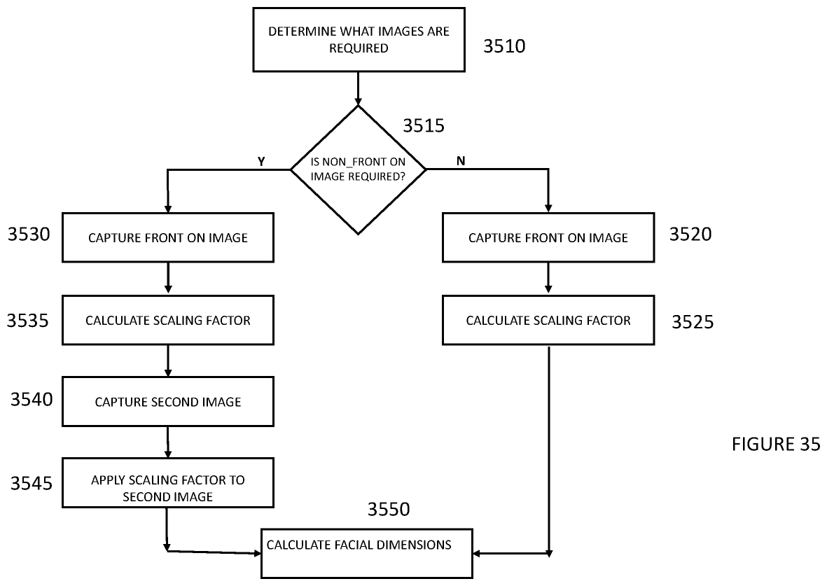

At 3440 the system determines which images are required based on the selected patient interface type. Referring to FIG. 35, the system determines what facial image types are required at 3510 and at 3515 determines whether any images other than a front facial image are required. The process for image capture may vary depending on which images are required.

If a front facial image is required only, for example if all required facial measurements can be obtained from a front facial image, a front facial image is captured at 3520. The system confirms that the image meets any predefined requirements, for example the correct distance, correct angle. A pixel measurement is taken for the eye width using the corner points of the eye and a scaling factor for the image is calculated at 3525. At 3550 and 3455 dimensions for the relevant facial features for the patient interface category are calculated.

If a non-front facial image is required, for example if an underside facial image is required to fit a nasal pillow mask or an under nose mask, a front facial image is captured at 3530 as a scaling image. A pixel measurement is taken for the eye width using the corner points of the eye and a scaling factor for the image is calculated at 3525. The user is then instructed to capture a second facial image type. When the second image type is captured at 3540 the relevant facial features are identified in the image and a pixel measurement is taken for the relevant features. At 3545 the scaling factor is applied to the second image and the relevant facial dimensions are calculated at 3550.

Referring back now to FIG. 34, after the relevant facial dimensions are calculated at 3455, the facial dimensions are compared to the patient interface fitting data for the relevant patient interface category at 3560 and the system identifies a recommended patient interface size for the patient based on the dimensions. At 3565 a patient interface recommendation is presented to the patient.

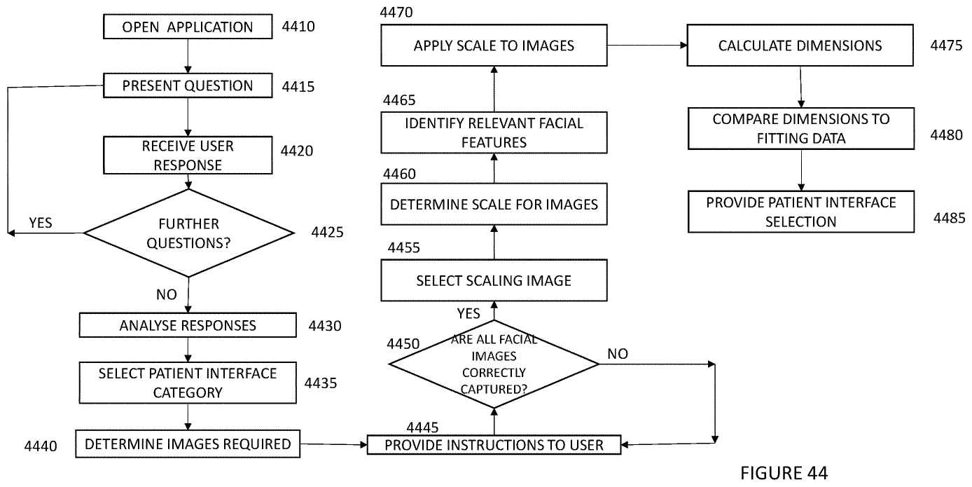

A further description of the steps performed is now provided with reference to the flow diagram of FIG. 44. The process of FIG. 44 is executed on a mobile device of the patient.

At 4410 a patient interface fitting software application is run on the mobile communication device of the patient. The patient interface fitting application may be selected by the user by user input, for example on a touch sensitive display screen by touching the area of the screen displaying an icon for the software application.

The patient interface fitting software application may present a home screen offering options to the patient, including a patient interface fitting option. When the patient interface fitting option is selected by the patient, the application presents a first predefined question to the user on the screen of the mobile communication device at 4415. The question may require subjective or objective response data from the user. At 4420 the system receives a user response in the form of user input into the mobile communication device. At 4425 the application determines if any further questions are required to be presented to the user. The application may present a predefined sequence of questions in a predefined order. Alternatively, the application may process the user responses on receiving a response and determine whether further information is required from the patient based on the responses received.

At 4430 the application analyses the responses received from the user to select a patient interface category suitable for use by the user. The patient interface category is selected by the application at 4435.

At 4440 the application determines what facial image types are required in order to calculate the facial feature dimensions required to fit the patient interface of the selected category. The facial image types may be required to provide measurements of relevant facial features for fitting and/or the image type may be required to provide a scaling factor for facial feature measurements. Embodiments using eye width as the reference feature for scaling require a front on image type to obtain a eye width measurement and a scaling factor for the image. This front on image is used as a scaling image. In some cases, if all required dimensions for fitting can be calculated from the front facial image, for example for a full face patient interface, then the only image required to fit the patient interface a front facial image. In other cases, for example a nasal pillow interface dimensions of nose depth is required and so an underside facial image is required. So for nasal pillow interfaces, the application requires a front facial image to obtain a scaling factor and an underside facial image to provide a measurement of the depth of the nose. In the step of 4440, the application determines which facial image types are required to be captured by the user.

The facial image types may have specific criteria associated with them, for example angle of the face required in the image, distance between the face and the camera, lighting requirements, position of the face within the frame. These specific criteria are identified by the application when determining which image types are required.

At 4445 the application presents instructions to the user for capturing the required facial image types. The instructions are presented to the user on the display of the mobile communication device. The application may activate the camera automatically. The instructions may provide guidance to the user of correct positioning of the face and the camera to assist the user in capturing the required facial images which meet the specific criteria. If multiple facial image types are required, the application will instruct the user to capture a first image at a first orientation of the camera with respect to the face of the patient. When that image has been captured correctly by meeting all required criteria, the application provides further instructions to the user to re-orientate the camera or the face of the patient to capture a further facial image.

At 4450 the application determines whether all required facial images types have been captured correctly. If not, further instructions are provided to the user at 4445.

When all required images have been correctly captured and received by the application the application begins the process of calculating a scaling factor for the images. (Alternatively, the scaling factor may be calculated after those images from which the scaling factor is to be derived have been obtained, but before any other required images have been obtained). When eye width is used as a scaling factor for the images the application selects the front facial image as the scaling image at 4455. The pixel eye width measurement within the image is measured and the predefined eye width dimension is applied to the pixel eye width measurement to calculate a scaling factor for the front facial image at 4460.

At 4465 facial features for which dimensions are required are identified in the images. These facial features may be identified by identification of relevant facial landmarks in the images. A pixel measurement is made for each of the facial features. At 4470 the scaling factor is applied to the measurements. The dimensions of the facial features are calculated at 4475. The scaling factor can be applied to pixel measurements in different images.

At 4480 the calculated facial feature dimensions are compared with the patient interface fitting data and the system identifies a patient interface size for the patient based on the dimensions. At 3565 a patient interface recommendation is presented to the patient on the display.

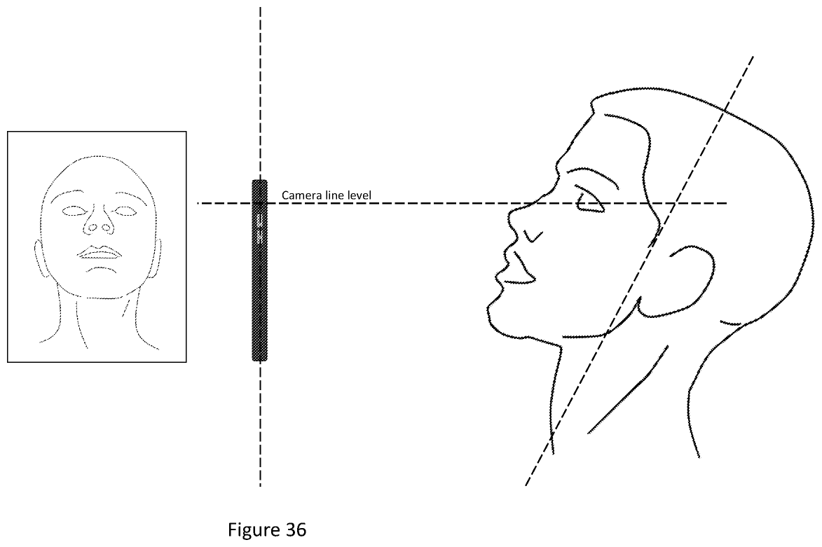

Certain algorithms may be utilised to calculate dimensions in facial images. The following description describes an algorithm for calculating nose depth from an underside facial image, see FIG. 36. For illustrative purposes FIG. 36 shows a selfie camera able to view the nose depth by an underside angle of the face. Preferably the angle is 35-45 degrees as the relative angle between head and phone. The app detects face is at 35-45 degrees relative to phone using same tilt angle detection methods described above. A 35-45 degree angle may be advantageous as it may allow the underside of the nose to be sufficiently visible, but at the same time may not require excessive tilting back of the head on the part of the user/patient.

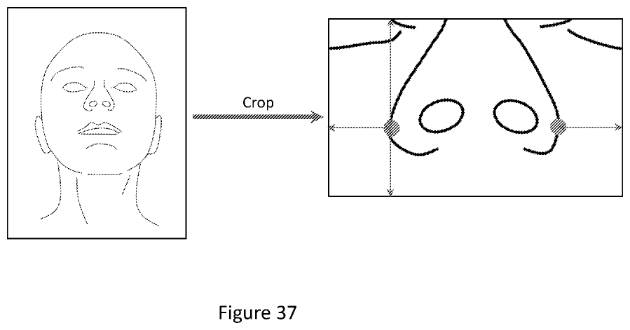

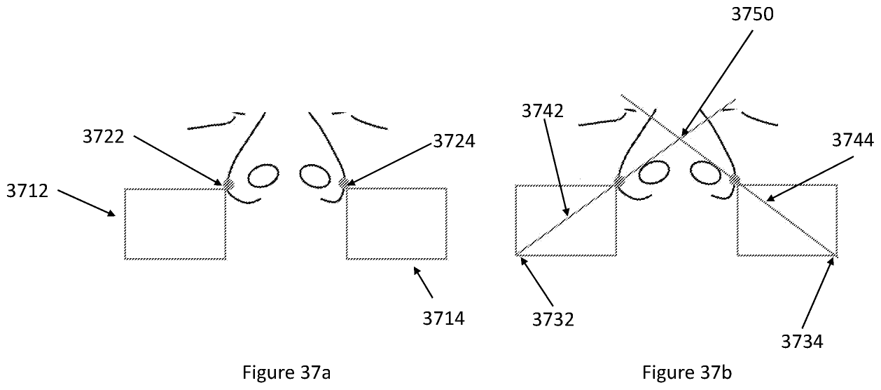

The image is cropped around the area of interest as shown in FIG. 37. This step is technically optional but cropping to a smaller area saves on processing power/time and simplifies the image. The bounds for the cropping box may be found using facial image recognition software. In the example of FIG. 37, landmarks around the edge of the nose (blue dots) are identified and add certain margins (blue arrows) which are drawn from those points to get the bounding box. Alternatively four landmarks could be set for the corners of the bounding box and cropped accordingly.

The size of the margins may be determined by several means. For instance, the system may have a rule that a first number, X1, of pixels are left horizontally to either side of each landmark (such as the nostril edges), and a second number X2 of pixels are left above and below each landmark; and a boundary box is drawn around the area defined by these pixels. This way, the boundary box will extend X1 pixels to the side of each of the landmarks, and 2*X2 pixels from the landmarks in the vertical sense (since X2 extends both above and below).

For example, X1 may be 80 pixels and X2 may be 15 pixels.

Referring now to FIG. 37a, another means may be to specify a ratio of the height vs width of an imaginary “rectangle” 3712 3714 at either side of the image, each having its upper corner co-located with one of the landmarks 3722 3724. So, the width: height ratio of the rectangle may be, for instance, 3:1. The bounding box then extends along the sides of each rectangle that are distal from the landmark. Or alternatively, no boundary is explicitly formed, rather the rectangle itself provides a distal corner through which (along with the landmark) a diagonal line can be drawn to estimate the location of the tip of the nose, as described further below.

Referring now to FIG. 37b, the purpose of defining the size of the margins (in one of the above ways, or in a different way) is to establish a pair of points (as discussed below) on either side of the nose, one of which is the landmark corresponding to the edge of the nose itself 3722 2724, and in the example of FIG. 37b, the other point is the opposite corner of the rectangle 3732 3734, to enable a straight line 3742 3744 to be drawn through each pair of points such that the intersection point 3750 of the two lines gives a good approximation of the location of the tip of the nose. As such, the skilled person will appreciate that the exact spacing of the boundaries (or ratio of the rectangle) will be sized and located so as to result in angled lines 3742 3744 that intersect at the correct point 3750, that is, at a point that reasonably accurately approximates the location of the tip of the patient's nose.

An alternative approach may be, instead of drawing margins or rectangles, to draw a line through each landmark at a prespecified angle, the angle being such that the two lines intersect at a point that reasonably accurately approximates the location of the tip of the patient's nose.

Appropriate dimensions for the margins/rectangles/angled lines may be determined based on statistical dimensions of underside nose profiles. The system may even have several different sets of dimensions, to suit different nose types (e.g. a shallower/flatter nose type versus a deeper nose type). At an earlier step, the nose type may be detected or inputted (such as manually by the patient/camera operator or by automatic detection using the face mapping tools); and the system may then extract the appropriate set of dimensions/algorithm and use this to determine the applicable parameters of the margins/rectangles/angled lines. Another (additional or alternative) means of determining appropriate dimensions for the margins/rectangles/angled lines, and/or for determining nose type, may be the detected (or inputted) dimensions and/or angle/slant of the nostrils.

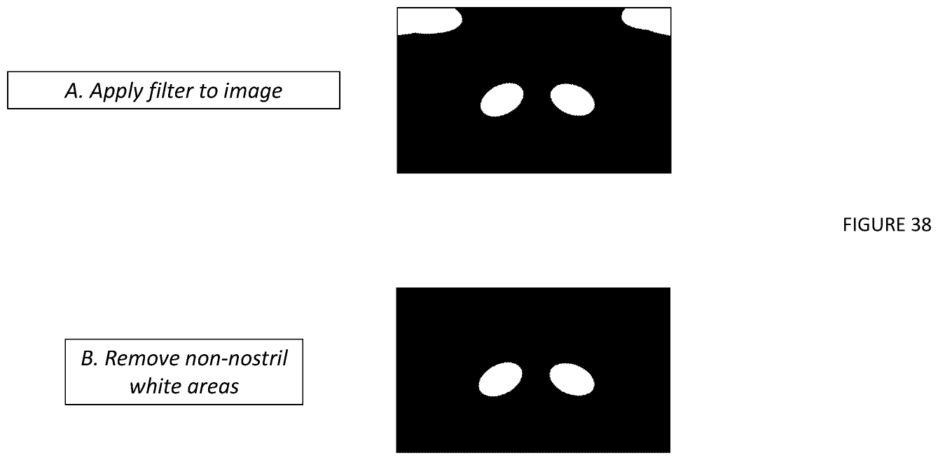

Referring to FIG. 38, filtering is applied to the cropped image to produce a black and white image as shown in FIG. 38A. A black and white image shows clear contrasts and transitions which is easier for computer vision/image recognition software to process given less variation/noise in the image. An absolute filter can be applied to the cropped image as shown in FIG. 38B. For each pixel, if the brightness is above a predefined threshold then the pixel is turned white, if it's below the predefined threshold the pixel is turned black. Alternatively a percentage based filter may be used. This looks at each pixel and asks if the brightness value is greater than or less than a certain percentage of the other pixels in the image. Then the pixel is turned white or black if it is above or below that certain percentage accordingly.

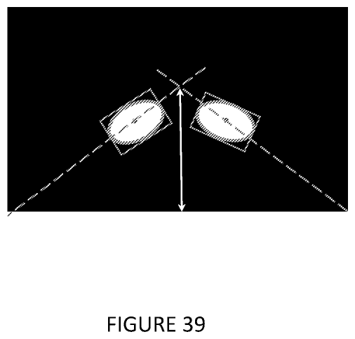

Referring now to FIG. 39, a computer vision algorithm that identifies objects may be used, such as for example from a computer vision library e.g. Open CV. The contours are found using a process defined in the computer vision library. Alternatively, other object recognition methods may be used.

The step is identifying nostrils and then identifying contours of nostril.

A line is drawn from the bottom left corner of the bounding box to the left nostril centre and extended through. The same is done for the right nostril until the two lines intersect. Depth is then calculated as the distance from this intersection point to the bottom bound of the bounding box. As described above, a scaling factor derived from the width of the eye in a front on image of the face can be used to calculate the dimension of the nasal depth. Distance is calculated as pixels and then converted into mm (or another suitable unit) using the scaling factor of eye. Alternatively, a scaling factor of nose width could be used.

Finally, optionally a predetermined further scaling factor (may be applied to the calculated depth dimension to account for distortions related to the angle of the face with respect to the camera or other lens distortions. As described above, this further scaling factor (for example a rectification factor or compensation factor) is applied in addition to the scaling factor.

The output of all the steps above is a depth measurement in pixels.

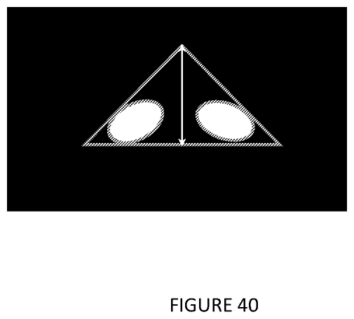

An alternative method is shown in FIG. 40. A “minimum triangle” is drawn around the nostrils—this is done using automatic computer vision library tools. That tool takes any number of points (e.g. the points corresponding to the contour of the nostrils and draws the smallest triangle that encloses all those points). Depth can then be calculated by finding the maximum height of that computer vision drawn bounding triangle. This would be calculated as pixels and then converted to mm (or another suitable unit) using the scaling factor.

Another alternative step is shown in FIG. 41. A minimum bounding box (the smaller box) is drawn around both nostrils once the contours are detected by the computer vision library.

Then applying some margins to that bounding box to draw a slightly larger box. The margins of the larger box are determined by the angles of the nostrils, the narrower the angle/the taller the nose, the larger the larger box for example (or the other margin/rectangle/angled line approximation methods noted above, with appropriate modifications). Then similar to the version above, connect a line between the bottom corners of the larger box to the nostril centroids, find their intersect, and calculate depth from that.

In an alternative method, the system does not use an absolute or static threshold of a greyscale value above which pixels of the image are turned black and below which they are turned white (or vice versa). If a patient is scanning against non-normal or non-regular lighting, that lighting might interfere with the contrast of their face when scanned. E.g. if there is low light on one side of the face, the filtering step might detect that all or a significant portion of the face is below the static threshold, which may result in the nostrils being indistinguishable from the rest of the face in the filtered image. In the alternative example, instead of a static number used as the threshold, a percentile based threshold is used. All pixels in the image are ordered by grey scale value (e.g. smallest to largest) and then the bottom 3rd percentile value is selected as the threshold to be applied during filtering. E.g, for a 100 pixel image this can be done by ordering the pixels from lowest to highest grey scale value and finding the pixel value of the third percentile pixel in terms of grey scale value—then any pixel with the same value and/or lower gets set to white value (255) and any pixel greater gets set to black (0). The changing of values can also happen the other way around, where pixels at or below the threshold are turned black, and the rest turned white. The pixels may also be set to any other grey scale value. Any other percentile may also be used. The threshold may be an inclusive threshold or a non-inclusive threshold.

The method may automatically detect when it may be more suitable to use a dynamic percentile based threshold as opposed to a static threshold, for example by detecting irregular lighting across the face of the user. One way this could be detected is for example by detecting a significant variation in average pixel values between two halves of an image. As an example, if the left half of an image has an average grey scale value 20% greater or less than the average pixel value of the right half of an image, then it may be more suitable to use a dynamic percentile based threshold.

In some images unnatural lighting may create a contrast between the two halves of the face—this is due to the centre of the face and raised nose acting like a shade for one side. As such the dynamic threshold is applied separately to each of the two vertical halves of an image, or each of the four quadrants, or any other suitable divisions of the total image. After applying the dynamic threshold to each of the divisions, for e.g. the two halves, those divisions are then stitched back together. This alternative method, using the dynamic threshold, may also have other applications and uses, such as to account for natural variations in skin tone between users where a single static threshold might not work as effectively given different skin tones will produce varying grey scale values in images of those skin tones.

### Width Sizing

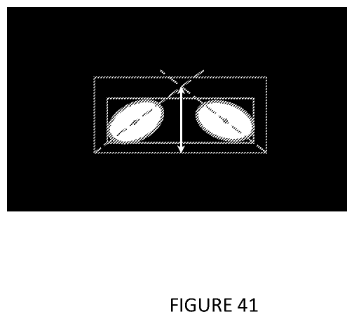

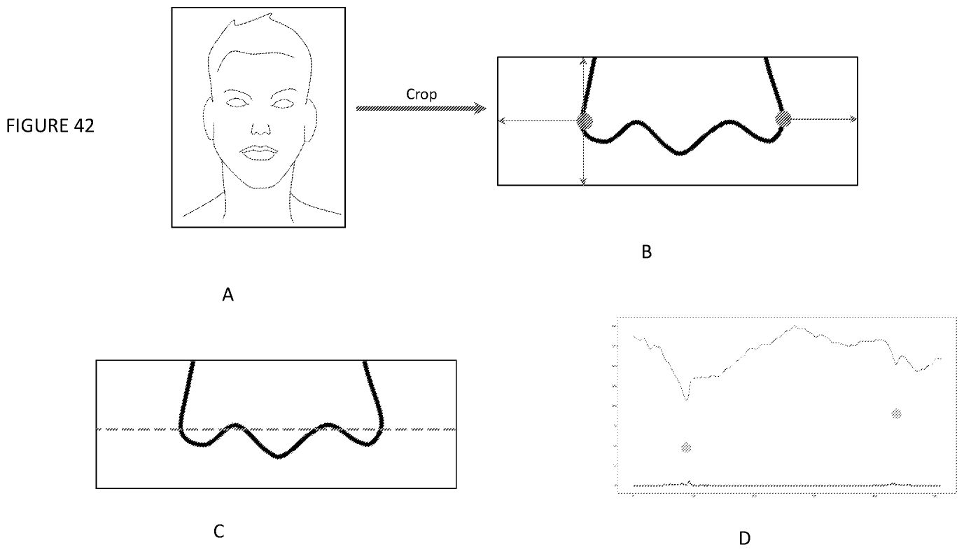

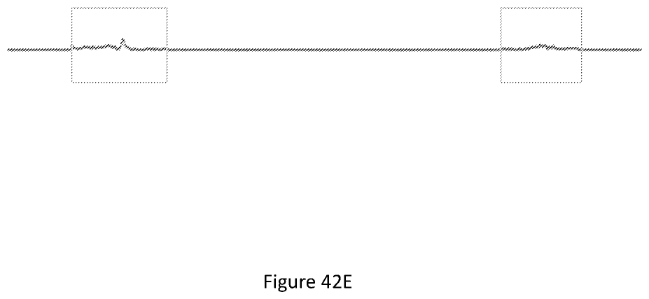

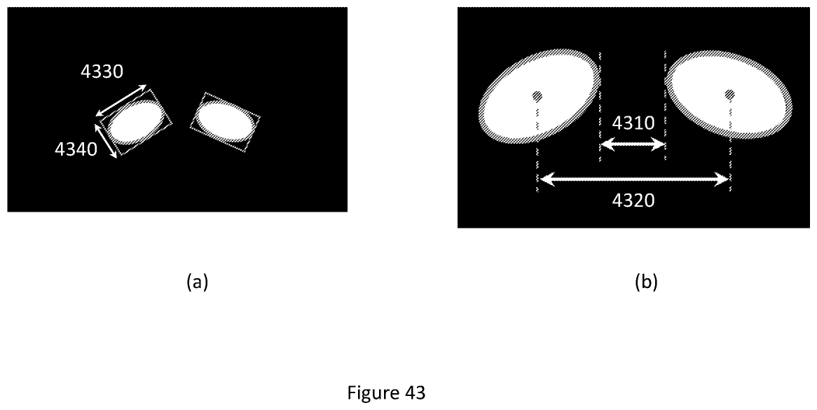

The width of the nose is an important dimension for several mask types. The following description, with reference to FIGS. 42 and 43, provides one technique for calculating nose width.

The first step is to identify the facial landmarks defining the edge of the nose. In the example of FIG. 42 the width is calculated from a front on image of the face FIG. 42A. The landmarks are identified, for example using MediaPipe landmarks, and margins are inserted to crop the relevant section of the image in FIG. 42B.

In the next step freckles, pimples, moles, other imperfections are removed by using image filtering techniques. E.g. image blurring filters, spot fix techniques. Another filtering step is to apply an averaging filter to the middle row of pixels (i.e. the row that coincides with the edges of the nose, shown by the red dashed line in FIG. 42C). Step along the row and for each pixel in the middle row calculate the average of the 7 above and below and itself.

After filtering, the pixel values of the middle row of pixels after all the image processing is complete (middle row shown by the red dashed line in FIG. 42C)—i.e. analyse the grey scale values (0-255) of the middle-row pixels as filtered (averaged) in the step above. These pixel values are shown on graph in FIG. 42D, the red line. We also calculate the absolute gradient of that red line, this absolute gradient line is the blue line on graph below. The greatest gradient values in the left half and the right half are identified, shown by orange dots. The distance between these two points is the nose width.

The gradient is shown in greater detail in FIG. 42E. When we calculate the gradients (blue) of the pixel value line (red) we disregard certain values that don't fall in the ranges depicted by the orange boxes below. We set all gradient values outside the orange boxes to zero. We are assuming that the nose edges (which we need to find the width) can't be outside these boxes and that the nose can't be that wide or narrow. This is to avoid for e.g. accidentally selecting the max gradient to be at the nostril openings-we want to be detecting the edge of the nose, not the opening of the nostril, and in reality these two points (nose edge and nostril opening) see a significant transition between pixel values (light to dark transitions) and we thus need a way to distinguish them.

The bounds of the orange boxes below can be predetermined pixel coordinates e.g. if the cropped image was 500 pixels wide we could for example set to zero the gradient values for the first 80 and last 80 pixels and also the middle 300. Alternatively, we could apply margins to landmarks returned by the software. e.g. set all 80 pixels to the left of the left nose edge landmark to 0 and the same for the pixels to the right of the right nose edge landmark, and also set all points in between the nostril landmarks to zero. Another filter that could be applied is if pixels are within a certain coordinate range (e.g. Pixel 200-300 in the x direction) AND above a certain value then we set the corresponding gradient line value to zero.

A method for calculating nostril sizes is now described with reference to FIGS. 43 (a) and (b). The initial steps are the same as those for calculating the nasal depth. In one example shown in FIG. 43(a) a computer vision library (Open CV) is then used to find the contours of the nostrils and bounding boxes of the nostrils. Take the width and height of the bounding boxes to calculate the major and minor diameters of the nostrils.

The output of all the steps above are nostril height and width measurements in pixels. These measurements are converted to dimensions using the same “eye width reference” method previously disclosed. These measurements/dimensions can be averaged across the two nostrils. The dimensions of the major and minor axes of the nostrils are two parameters needed to properly size a prong interface. Alternatives could be finding the area of the nostril contour, find the perimeter of the nostril contour, using the same computer vision library and well-known geometric relationships/calculations. Preferably we require at least major axis measurement. Identify major axis since nostrils are generally elliptical in shape.

Alternatively, the major and minor axis may be determined based on landmarks on the nostril and object recognition in combination. Objection recognition is used to determine the actual nostrils and then landmarks for determined nostrils could be output. Then the pixel distance of major and minor axis is calculated. Scaling factor is used to determine the actual major and minor axis dimensions.

The nostril opening area may be approximated using the dimensions of the major axis 4330 multiplied by the dimension of the minor axis 4340. Or a more precise area of the ellipse may be calculated using mathematical formulas for ellipse area, being:

$$Area = π \times ( 0.5 \times major axis 4330 ) \times ( 0.5 \times minor axis 4340 )$$

Other approximations may be used to calculate the nostril area, for example assuming the area of the nostril opening to be a circle. This may be particularly relevant if for example the dimensions of major axis and minor axis are similar. Some systems may use a combination of calculating an approximation, an ellipse area and a circle area and then use any one of those or a combination of those areas for sizing.

In some systems a nostril opening area is used by calculating the average of the area of the two nostrils. In other systems the interface sizing may be made based on either the larger opening, or the smaller opening. The interface sizing may be made based on the left nostril or the right nostril.

FIG. 43 (b) shows the septum distance 4310 and the distance between the centre of the nostrils 4320. These can be compared against “patient interface sizing information” to select an appropriately sized cannula interface—this can also be in combination with the nostril opening methods described with respect to FIG. 43 (a). These dimensions can be found using landmarks or image processing techniques.

Some systems deliver gases to a patient through an asymmetrical nasal cannula or nasal interface. An asymmetrical interface or asymmetrical nasal delivery elements, as described herein, refers to an interface where the nasal delivery elements differ in size such as internal and/or external transverse dimensions or diameters, and/or internal and/or external cross-sectional areas. The external cross-sectional area is the cross-sectional area bounded by the outer wall of the nasal delivery element. For non-circular cross-sections, the references herein to a diameter may be interpreted as a transverse dimension. In some configurations, references herein to a diameter include but are not limited to a hydraulic diameter.

The system allows an asymmetrical flow to be delivered through the interface to both nares or to either nare. Asymmetrical flow as described herein refers to a flow that differs within the interface or within the nose or within the interface and the nose. In this way, a different flow may be delivered by each nasal delivery element, or the flow may differ between inspiration and expiration, or the delivered flow may be a combination of the above. An asymmetrical flow may also include partial unidirectional flow.

Delivery of asymmetrical flow may improve clearance of dead space in the upper airways, decrease peak expiratory pressure, increase safety of the therapy particularly for children and infants, and reduce resistance to flow in the interface. An asymmetrical nasal interface and/or nasal delivery elements as described herein includes interfaces or systems configured to produce such asymmetrical flow through asymmetrical nasal delivery elements.

Pressure generated by Nasal High Flow (NHF) depends on flow through the nasal interface, the size of the nasal delivery elements and/or nares of the patient, and the breathing cycle. If flow, leak, or a combination of flow and leak, is asymmetrical through the nasal interface, the flow through the nose may become asymmetrical during breathing. Partial and total unidirectional flow may be types of asymmetrical flow. Partial or total unidirectional flow may provide improved clearance of anatomical dead space as the air is continuously flushed from the upper airways. Partial unidirectional flow may be more comfortable than total unidirectional flow. Total unidirectional flow as described herein includes flow entering one nare by a nasal delivery element and exiting via the other nare via a nasal delivery element, venting to the atmosphere, due to the absence of a nasal delivery element, or the like. Partial unidirectional flow as described herein includes flow that may enter the nose via both nares and leave the nose from one nare, flow that may enter the nose through one nare and leave the nose via both nares, or different proportions of flow that may enter the nose through both nares and different proportions of flow that may leave the nose through both nares, and may be flow that may enter the nose via both nares and leave the nose from one or both nares and optionally via the mouth.

NHF delivered through an asymmetrical nasal interface can involve making an interface in which the nasal delivery elements are of different size, e.g. different length and/or internal diameter or cross-sectional area and/or external diameter or cross-sectional area. Particularly for children or infants, nasal delivery elements will have a small internal diameter and thus higher resistance to gas flow. By using nasal delivery elements that are different lengths, each nasal delivery element may have a different internal diameter (e.g., minimum internal diameter or area). A longer nasal delivery element may have a smaller internal diameter and higher resistance to gas flow; a shorter nasal delivery element may have a larger internal diameter (e.g., larger minimum internal diameter), hence lower resistance to gas flow at the interface. A decreased resistance to flow allows the desired flow to be achieved using lower backpressure, or a lower motor speed of the gas generating device, or a combination of the two.

Asymmetrical nasal delivery elements may cause the peak expiratory pressure to decrease due to the different cross-sectional areas of the nasal delivery elements at the nose which may provide different internal diameters for each nasal delivery element.

The pressure when exhaling against the asymmetric nasal interface may be higher than with a symmetric one, which is beneficial as higher positive-end expiratory pressure (PEEP) is part of the treatment for COPD (pressure here referring to the intrathoracic pressure). Expiratory pressure is dependent on the combined cross-sectional area of the two prongs. Increasing the cross-section of symmetric prongs carries the risk of fully occluding the patient's nares. Using asymmetric prongs allows for an increase in total cross-sectional area without the accompanying occlusion risk. The partially unidirectional flow may reduce turbulence in the patient's nasal cavity, which could improve comfort.

In an example, an asymmetrical nasal interface used with (e.g., coupled via a conduit or breathing tube) a gas generating device, such as an AIRVO™ flow generator from Fisher & Paykel Healthcare Limited, decreases the resistance to flow. This may cause the motor speed of the AIRVO™ to drop from a range of 18,000-22,000 RPM to a range of 14,000-18,000 RPM while continuing to achieve a suitable flow for the desired therapy (e.g., NHF), such as about 8 litres per minute (lpm). The asymmetrical nasal delivery elements may cause a reduction of the backpressure generated in the system if, for example, an incorrectly sized prong forms a seal with a patient's nare.

For a smaller patient, as in an infant or a child, use of asymmetrical nasal delivery elements may reduce over-insertion of both prongs into the nares, when the nares are too small with respect to the prongs, which could result in an undesired semi-seal or seal. Asymmetrical flow may be delivered to the patient even if only one prong is positioned tightly in the nose. The asymmetrical interface improves the performance of the therapy for infants as compressed gas may be used in a system without pressure control.

Automatic sizing systems can be used to fit asymmetric nasal interfaces. Typically, when fitting an asymmetric nasal interface the patient requires the size that produces the greatest occlusion between the larger prong and the nostril it goes into, whilst ensuring that the smaller prong remains unsealed. Even a complete occlusion/seal with the larger prong may still be effective. When fitting an asymmetric nasal interface, the system retrieves the dimensions of the nostril opening and use that dimension to size to the larger prong. As described herein, the nostril opening dimensions are compared with patient interface dimension information to identify the best fitting patient interface.

Some systems may be configured to pick the largest prong size that will fit into a nostril, even if that means occlusion. In the case of symmetrical or regular or non asymmetrical nasal interfaces, the system may be configured to pick prong sizes that fit within the detected nostril such that no seal or occlusion is created between the prongs and the nares.

As described above the application may provide feedback to the patient to confirm whether or not the patient's face is orientated correctly to enable an image of the user's face to be captured at the desired angle. i.e. to provide feedback to the patient to confirm whether the required attributes for the image are met by the current orientation of the face of the user. The desired angle or position for image capture may vary depending on the facial dimension required in order to size the required mask type. The feedback may be provided using visual feedback, for example a visual indicator, the feedback may be provided using haptic feedback, for example a haptic indicator, the feedback may be provided using audio feedback, for example an audio indicator. Other feedback types may be used. Some embodiments may include a combination of feedback types. For example, the feedback may be provided using a combination of visual and audio feedback.

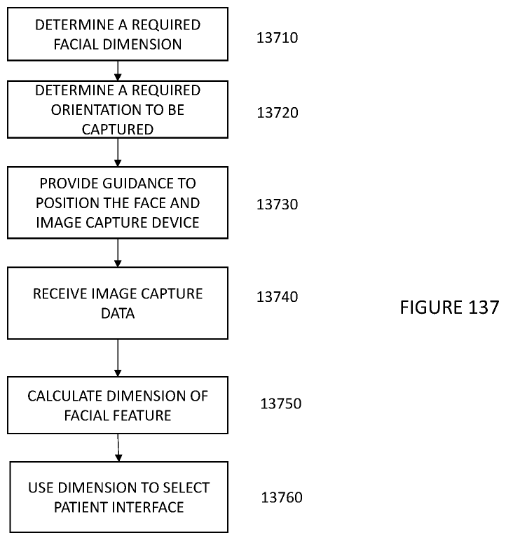

In the following example, and referring to FIG. 137, the application is for selecting a patient interface for a patient for use with a respiratory therapy device, the patient interface suitable to deliver respiratory therapy to the patient. At 13710 the application determines a dimension of a facial feature required in order to select a patient interface for a patient. The facial feature may be the width of the nose, or the depth of the nose etc (the facial feature may vary depending on the type of patient interface in question). Once the required dimension of the facial feature has been determined, at 13720 the application determines a desired orientation of the face of the patient to be captured in a digital image, in order to calculate the dimension of the required facial feature. For example, if the depth of the nose is the required dimension, the application may identify that the required orientation of the face to be captured in the image is an underside orientation. The application may use certain attributes to define the underside image, for example angle of the plane of the camera with respect to the face of the patient. In order to assist the patient to correctly orientate their face, the application may provide guidance to the patient at 13730 to position their face in the desired orientation for image capture. When the application receives image capture data representing at least one digital image of a face of a patient at 13740, the image capture data representing the face of the patient orientated in the desired orientation, the application may calculate a dimension for the facial feature from the image capture data at 13750. The dimension may then be used to select a patient interface for the patient at 13760.

### Position Indicator—Tilt

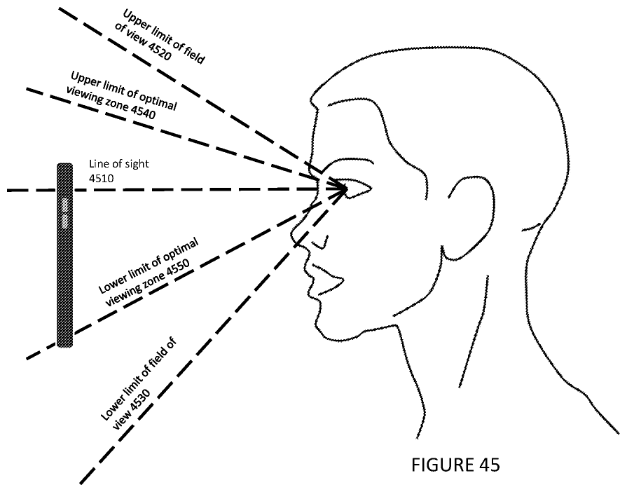

For some required orientations of the face for image capture, for example orientations which require the head to be tilted backwards to obtain an image of the underside of the face, the user's visibility of the screen decreases as the angle of tilt increases. The further the user tilts their head backwards, away from the screen, the less clearly the user is able to see the screen. This situation is now described with respect to FIGS. 45 to 47.

FIG. 45 shows a head orientated with respect to a camera. For the purposes of FIG. 45 the yaw of the head with respect to the camera is zero and so the face of the user is directly facing the camera. The camera may be in a mobile communications device. The orientation of FIG. 45 is a front facial orientation. The face of the patient is directly facing the camera 1640 and the angle of the face of the patient relative to the plane of the camera 1670 is approximately zero. The pitch angle of the face of the user with respect to the camera approximately zero. If the camera captures an image of the user's face in the orientation of FIG. 45, it captures a front facial image. In FIG. 45 the camera is positioned at eye level with respect to the patient's face.

FIG. 45 shows the field of view of the patient in the vertical plane. The user's line of sight projects directly outwards from the user's face. The line of sight projects horizontally from the face of the user when looking directly ahead. The user's field of view extends above and below the line of sight in the vertical plane between an upper limit of the field of view 4520 and a lower limit of the field of view 4530. Objects positioned outside the user's field of view are not visible to the user. The field of view is constant for a fixed head position. For a fixed head position the user may observe objects at different positions within the field of view by moving their eyes. Typically, the field of view extends more below the line of sight than above the line of sight and so typically the field of view is not symmetrical about the line of sight.

Within the field of view is an optimal viewing zone. The optimal viewing zone is positioned inside the field of view with its upper and lower limits being closer to the line of sight compared with the upper and lower limits of the field of view. The optimal viewing zone has an upper limit of optimal viewing zone 4540 and a lower limit of the optimal viewing zone 4550. A user can see objects within the optimal viewing zone more comfortably than objects outside the optimal viewing zone. The optimal viewing zone is sometimes used to configure monitor heights on desks and television positions in rooms. Typically a field of view may be around 120 degrees in the vertical plane and the optimal viewing zone may be around 40 degrees within the field of view. Again, the optimal viewing zone often extends more below the line of sight than above the line of sight and so is typically not symmetrical about the line of sight. The field of view and optimal viewing zone may vary between individuals. For example, some individuals may have a field of view in the vertical plane greater than 120 degrees or less than 120 degrees. Similarly, some individuals may have an optimal viewing zone that is greater or less than 40 degrees. The angle defining the optimal viewing zone may therefore be smaller than the angle defining the total field of view.

Objects positioned within the field of view but outside the optimal viewing zone may be less comfortable to view and also more difficult to see as they approach the limits of the field of view.

One of the challenges of requiring users to capture images at different angles is that as they tilt their head further away from the screen their visibility of the screen is reduced. This can mean that any guidance or feedback presented on the screen becomes more difficult for the user to see. This may result in the user tilting the head back towards the camera in order to better see the instructions and this moves the head out of position for the required image such as when the application identifies that the required orientation of the face to be captured in the image is an underside orientation.

This situation is shown in FIGS. 46 and 47. In FIG. 46 the user has tilted their head backwards away from the camera. The user's field of view has rotated clockwise with respect to the pitch angle in FIG. 46. Although the height of the user's eyes is still level with the camera (i.e. the user has not changed the vertical height of their head) the user's line of sight 4610 now extends above the top of the mobile device 4660. The mobile device 4660 is still positioned within the field of view of the user, within the upper limit of the field of view 4620 and the lower limit of the field of view 4630. However, the whole mobile device is not positioned fully within the optimal viewing zone of the user. The optimal viewing zone extends between the upper limit of the optimal viewing zone 4640 and the lower limit of the optimal viewing zone 4650. A lower portion 4662 of the mobile device 4660 is now positioned outside of the optimal viewing zone of the user. An upper position of 4664 of the mobile device 4660 is positioned within the optimal viewing zone. In the example of FIGS. 45, 46 and 47, the screen of mobile device 4660 extends across the entire face of the mobile device. In the orientation of FIG. 46, the portion of the screen in the lower position 4662 of the mobile device may be unclear to the user or it may be uncomfortable for the user to view this portion.

FIG. 47 shows the user with their head tilted further backwards away from the camera compared with the orientations shown in FIGS. 45 and 46. The user's field of view has rotated further clockwise in FIG. 47. Although the height of the user's eyes is still level with the camera (i.e. the user has not changed the vertical height of their head) the user's line of sight 4710 is now further above the top of the mobile device 4760. The mobile device 4760 is still positioned within the field of view of the user, i.e. within the upper limit of the field of view 4720 and the lower limit of the field of view 4730. The bottom edge of the mobile device 4766 is just within the lower limit of the field of view 4730. The whole mobile device is not positioned fully within the optimal viewing zone of the user. The optimal viewing zone extends between the upper limit of the optimal viewing zone 4740 and the lower limit of the optimal viewing zone 4750. A lower portion 4762 of the mobile device 4760 is positioned outside of the optimal viewing zone of the user. Because the user's head is tilted back further, the amount of the screen of the mobile device outside the optimal viewing zone of the user has increased. An upper portion 4764 of the mobile device 4760 is still positioned within the optimal viewing zone but the portion of the screen within the optimal viewing zone is smaller than in the orientation shown in FIG. 46 as the head has tilted back further. The part of the screen within the optimal viewing zone is becoming smaller as the user tilts their head further back. The portion of the screen within the optimal viewing zone is also moving towards a top edge of the mobile device. In the example of FIGS. 45, 46 and 47, the screen of mobile device 4560, 4660, and 4760 extends across the entire face of the mobile device. In the orientation of FIG. 46, the portion of the screen in the lower position 4762 of the mobile device may be unclear to the user.

In practice if the application requires the user to tilt their head back, to enable an under-nose image. Ordinarily, a user would be ‘penalised’ for doing this in that their ability to see the screen would diminish as they did so. One practical byproduct being that the user is likely to abort the tilted-back position to “check” the instructions on the screen, meaning the whole scan may have to start again.

In some examples, the present application accounts for the change in the line of sight and in particular the fact that parts of the screen may be positioned outside the user's optimal viewing zone as the user changes the orientation of the head with respect to the camera, by positioning visual feedback on the screen at a location towards the user's line of sight. The position of on-screen feedback, for example visual indicators, changes depending on the tilt of the user's head. This accounts/compensates for their changing degree of tilt and thus the change in their ability to see parts of the screen.

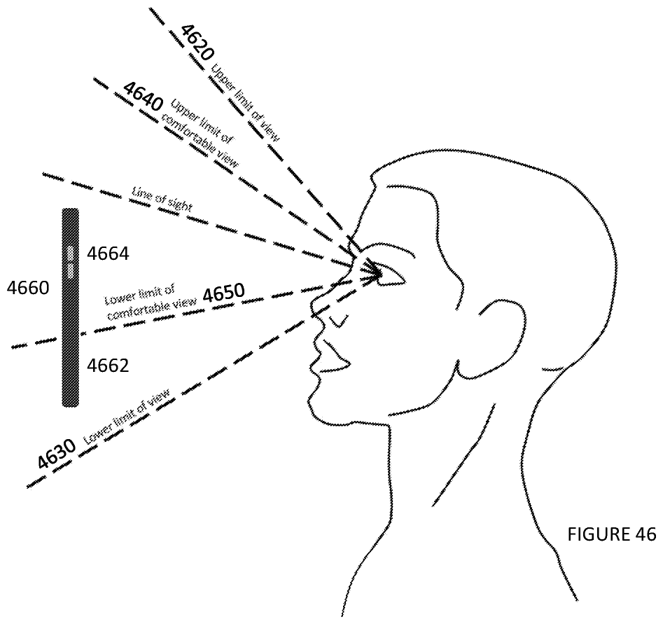

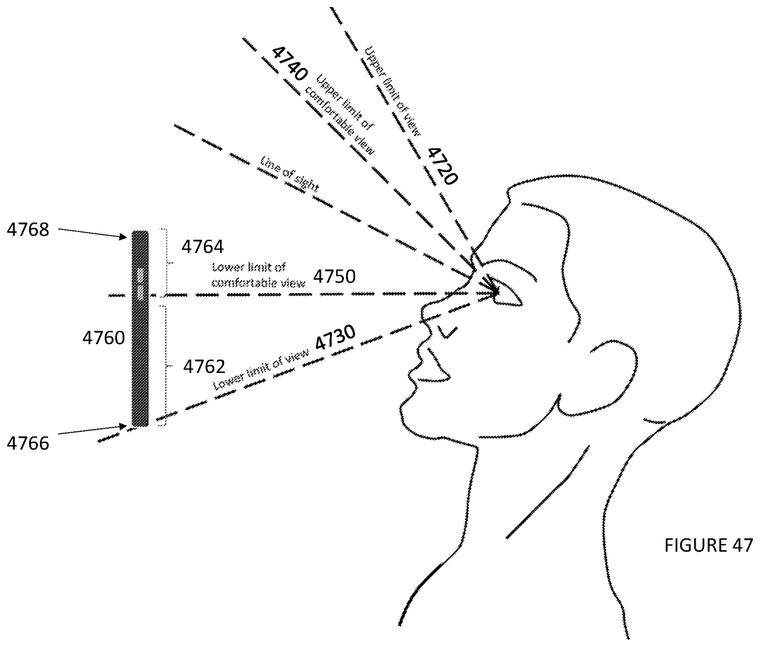

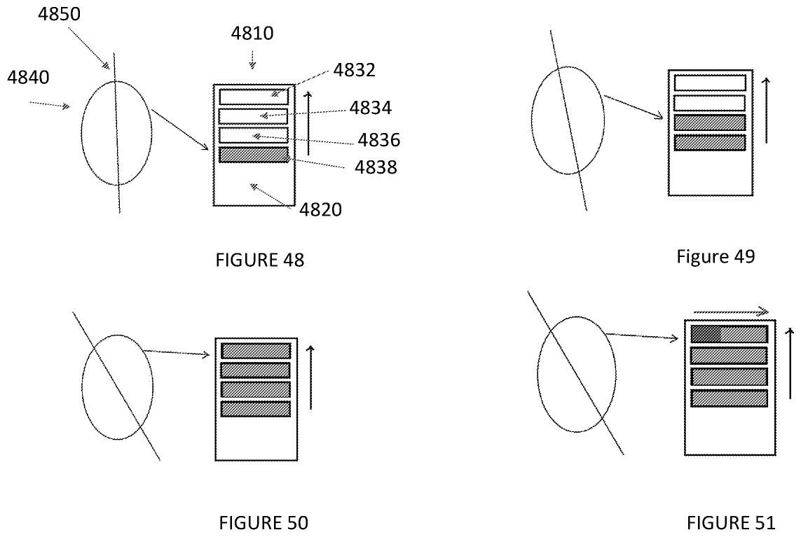

FIGS. 48 to 51 schematically show an example of a visual indicator displayed on a mobile device 4810 to guide a user to orientate their face in the required orientation for an image capture. In the example of FIGS. 48 to 51, the user's desired orientation for the image is an under-nose image, and so the visual indicator is a tilt indicator, configured to prompt the user to tilt their head back by a required amount (degree). (It is possible that previous steps in the process have occurred prior to this point, such as prompts displayed to the user to help them move their face to the correct height and/or distance relative to the screen, and to position the camera in a particular orientation, for example in a vertical orientation). In FIGS. 48 to 51 the screen 4820 is shown in a forward facing orientation, for purposes of illustration, though it will be understood that the user will in fact be facing the screen. The screen 4820 includes four visual indicators 4832 4834 4836 4838. Together the visual indicators form part of a position indicator to assist the user to correctly orientate (and more specifically, tilt) their head in order to capture an image of the head at the required orientation.

In the examples of FIGS. 48 to 51, for the “tilted-back” part of the process, the indicator is provided by a series of horizontal bars on the screen. The horizontal bars are arranged to guide the user to the required orientation by providing an indication of the comparison between the current orientation of the face and the desired orientation of the head. The position indicator is a progressive sequence including a series of indicators displayed at different locations on the screen. As the orientation of the head moves closer to the desired orientation more of the horizontal bars are illuminated or changed in colour. In other embodiments the horizontal bars may not be visible until they are illuminated of changed in colour. Horizontal bars indicating the head of the user being closer to the desired orientation are located closer to the top edge of the screen.

In FIGS. 48 to 51 the orientation of the user's head 4840 is illustrated by axis 4850.

During the guidance and head orientation process, the system continually monitors the orientation of the head of the user. The camera continually receives image data and the system uses the image data to calculate the orientation of the face. The orientation of the face may be calculated using facial detection modules. The facial detection module may comprise a face detection module and a face mesh module. The face detection module allows for real time facial detection and tracking of the face. The face mesh module provides for example a machine learning approach to detect the facial features and landmarks of the user's face as described further above. The face mesh module may calculate the orientation of the face. The orientation may be provided as an angle with respect to the camera (i.e. numerical or empirical orientation). The orientation may be provided as an orientation with respect to the plane of the camera. The orientation may alternatively or additionally be provided by reference to particular facial features which become visible, or cease being visible, or are visible with a predetermined amount of skew or foreshortening, as the head tilts back (i.e. functionally-determined orientation).

The system is configured to generate a position indicator for display on a display screen, the position indicator being configured to assist a user in positioning the user's face at a required non-frontal angle relative to an image-capture device to enable capture by the image-capture device of an image of the user's face at said required angle. The position indicator being configured to dynamically change position and/or appearance on the display screen in response to a detected change in angle of the user's face relative to the image-capture device, such that, for a given angle of the user's face relative to the image-capture device, at least a current-position-indicating portion of the position indicator is visible on the display screen to the user. For a first angle of the user's face, the at least a current-position-indicating portion is positioned in a first position on the display screen; and for a second, different, angle of the user's face, the at least a current-position-indicating portion is positioned in a second, different position on the display screen. The second angle of the user's face being a greater angle relative to the display screen than the first angle, wherein the second position of the at least a current-position-indicating portion of the position indicator compensates for a reduced field of vision, relative to the display screen, of the user at the second angle compared to the first angle.

In FIG. 48, the user starts to tilt their head back from the frontal orientation. At the orientation of FIG. 48, the screen generally falls within the optimal viewing zone. The user still has relatively good visibility of the screen as it falls within the user's field of view. The system receives data representing the face of the user from the camera and calculates the orientation of the face relative to the camera. The orientation is far from the required orientation and so a single bar is presented on the display. In the example of FIG. 48, the lowest bar darkens with colour (or potentially illuminates), indicating say 25% progress (assuming 4 bars).

Although reference has been made to the lowest bar (or the bar corresponding to the user's current orientation) darkening or illuminating it will be understood that the lowest bar may more generally become visible or more visible to the user at the appropriate time.

In FIG. 49 the user tilts their head further back. This moves the head closer towards the desired orientation. The further tilt backwards away from the camera also moves the line of sight of the user further above (i.e. upwards relative to) the camera and the mobile device. This moves the optimal viewing zone upwards, and the filed of view generally upwards. To compensate for the upward movement in the optimal viewing zone the next-highest bar darkens, indicating 50% progress. The position indicator provides confirmation to the user that they are moving closer to the required orientation and so confirms their progress. As the user's field of view is moving upwards, by providing this feedback at a location higher up the screen, and so closer to the user's line of sight, the user has a greater chance of being able to see the visual indicator clearly and/or more comfortably. The feedback is positioned within the user's field of view.

As the user continues to tilt their head back further and move closer to the desired orientation, the horizontal bars continue to be illuminated or changed in colour.

FIG. 50 shows the situation where the user has tilted their head back sufficiently and the head is orientated in the desired position. At this point all bars are illuminated indicating to the user that the head is in the required orientation. At this orientation the user's line of sight is higher and further above (i.e. upwards relative to) the camera and mobile device and so the bar indicating that the head has reached the desired orientation is the uppermost of the bars within the position indicator. In the example of FIGS. 48 to 51, this bar is close to the top edge of the screen. The position indicator provides confirmation to the user that they have reached the required orientation and so confirms their progress. As the user's field of view is moving upwards, by providing this feedback at a location higher up the screen, and so closer to the user's line of sight, the user has a greater chance of being able to see the visual indicator clearly and/or more comfortably. The feedback is positioned within the user's field of view.

In this way, the “position indicator” (or the operative part thereof, which in this embodiment means the illuminated or coloured part thereof) dynamically changes position on the screen depending on the degree of tilt of the user's head, to ensure continued ability to see the progress indicator in spite of tilt; and thus also continued feedback to the user as to whether they are tracking correctly in moving their head towards the desired orientation. The position that the position indicator is displayed on the display screen is changed as the user tilts the head and the angle of the head relative to the camera is changed so the position indicator is maintained within the user's field of view as the user tilts the head back.

Once the user has reached the desired orientation, the top bar 4832 also becomes a “progress indicator” that indicates a progress of a scan or image-capture process. Once the user's head has reached the desired orientation the camera is triggered to capture an image (or potentially more than one image, such as a plurality of images or frames (photo or video)). This image(s) represents the user's face in the desired orientation and so may be used to calculate dimensions of the user's face (and more particularly the relevant facial feature(s)). In some examples, a plurality of images (frames) are captured, and in each frame the relevant facial feature(s) is sized (i.e. its dimension calculated), and then an average dimension is calculated based on the individual dimensions of the feature across the images—this may improve accuracy as compared to just calculating dimensions from a single image or frame. The dimension(s) of the feature(s) may then be used to select a mask for the user. In practice, images may be captured throughout the guidance process (i.e. not only during the “image capture” phase but also during the preceding orientation phase). These may be stored or deleted.

The system may require the camera to be held still for a time period while the image or images are captured. In order to provide guidance to the user, the system may include a progress indicator. The progress indicator may indicate to the user that the head is positioned in the correct orientation and that the head should be held still in that desired orientation for a particular (such as predefined) period of time. The progress indicator may be a visual animation.

In the example of FIG. 51 the progress indicator is a coloured animation which progressively fills up, for example from left to right, the top bar with a colour to indicate progress of the scan. This is the equivalent of the dynamic circular progress bar in FIGS. 33b and 33c, but positioned to enable visibility with the head tilted back.

The top bar of the position indicator is transformed into the progress indicator in the example of FIG. 51. By locating the progress indicator at the highest indicator point on the screen this presents the progress bar towards the line of sight of the user. This increases the probability that the user will be able to see the progress indicator without having to reorientate their head and leaving the desired location.

Throughout the guidance process the system monitors the orientation of the head of the user. If the orientation moves away from the desired location, the indicators within the position indicator may be de-illuminated to advise the user that they are tilting their head away from the desired orientation (this may occur in proportion to the degree by which the user's head tilt has deviated—e.g. the top 1 or 2 bars becoming de-illuminated).

During the guidance process the application may be interrupted or terminated. For example, if while the progress indicator is running and the system detects that the user has re-orientated their face out of the desired orientation, the scan may be interrupted or terminated. The position indicator may de-illuminate some of the indicators to indicate the current orientation of the head, being away from the desired orientation.

In this embodiment, initially, the small tilt means the user can see the screen relatively well. As such, the lower bar colours/lights up to indicate say 25% (or some portion of) progress to the required tilt. As the user tilts back further, they can see a progressively smaller portion of the (top part of the) display. As such, progressively higher bars light up to indicate the user's progress towards the desired orientation—such that, for a given angle of tilt, the user can visually see/confirm their progress. If the indicator remained at say the 1st-bar level, the user may lose sight of it, or at least have compromised sight of it, or find it uncomfortable to view as they moved towards the correct tilt position. Finally, by the same logic, the topmost bar transforms to a “progress bar” during the scan, so the user, with their head tilted back, is still able to clearly see how far through the scanning process they are. This indicates to them to hold still until the progress bar indicates a scan is complete.

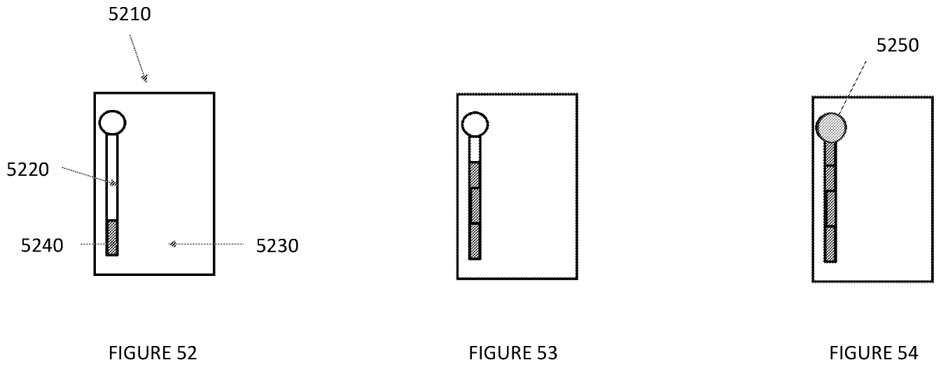

A further example of a position indicator is shown in FIGS. 52 to 54. The position indicator is an example of a visual indicator displayed on a mobile device 4810 to guide a user to orientate their face in the required orientation for an image capture. In the example of FIGS. 52 to 54 the desired orientation for the image is an under-nose image. FIGS. 52 to 54 show examples of the visual indicator displayed on the screen of the mobile device for different orientations of the face of the user.

Position indicator 5220 is displayed on screen 5210. In the example of FIGS. 52 to 54, the position indicator is a visual indicator. The indicator is used to provide the user with guidance to position their face in a particular tilted orientation.

The position indicator of FIGS. 52 to 54 is a bar displayed vertically on the screen 5230 of mobile device 5210. The position indicator fills up with colour (or some other visual effect) from the bottom to the top as the user's face becomes orientated closer and closer to the desired orientation. Colour progressively moving up the bar is an indication to the user that the orientation is moving closer to the desired orientation. If colour moves down the bar then this indicates to the user that the orientation of the face is moving away from the desired orientation.

Again, during the guidance and head orientation process, the system continually monitors the orientation of the head of the user. The camera continually receives image data and the system uses the image data to calculate the orientation of the face. The orientation of the face may be calculated using a facial detection module(s). The facial detection module may comprise a face detection module and a face mesh module. The face detection module allows for real time facial detection and tracking of the face. The face mesh module provides for example a machine learning approach to detect the facial features and landmarks of the user's face. The face mesh module may calculate the orientation of the face. The orientation may be provided as an angle with respect to the camera. The orientation may be provided as an orientation with respect to the plane of the camera. The orientation may also be provided functionally, as described further above.

The system monitors the orientation of the face of the user and indicates the orientation with respect to the desired orientation visually. In the schematics of FIGS. 52 to 54 the orientation of the head is not shown, though it will be understood that the user's face could be shown in real-time on the display screen, such as to the right of the position indicator. In FIG. 52 the orientation of the face of the user is away from the desired orientation. A first indicator is presented on the display to indicate that the orientation is away from the desired orientation. In FIG. 53, as the user re-orientates their face closer to the desired orientation by tilting their head backwards, the system recognises that the new orientation is now closer to the desired orientation and a second indicator is presented on the display. The second indicator includes more colour and moves higher up the bar.

Finally the system detects that the user has orientated his head into the desired orientation. The system recognises that the orientation is now closer than the previous orientation (and, in fact is the desired orientation) and a third indicator is displayed on the screen, shown in FIG. 54. The third indicator confirms to the user that the head is in the correct orientation. In the example of FIG. 54 the bar is now coloured to the top. The example of FIGS. 52-54 show discrete indicators that are displayed. A single continuous indicator may also be used that progressively changes colour or display from its bottom to its top in a similar manner to the position indicator 5220.

The position indicator of FIGS. 52 to 54 is arranged to compensate for the user tilting their head away from the camera and away from the mobile device. As the user tilts their head further back, their line of sight is moved upwards and above (i.e. upwardly relative to) the mobile device. To compensate for the continued movement of the line of sight above and away from (i.e. upwardly relative to) the camera and display of the mobile device, the position indicator displays indicators towards the top of the screen and towards the line of sight of the user to increase the chance of the user seeing the indicator (and their quality of visibility of the indicator as well as comfort of viewing the indicator). The position that the position indicator is displayed on the display screen is changed as the orientation of the head relative to the camera is changed so the position indicator is maintained with the user's field of view as the user tilts the head and changes the angle of the head relative to the camera.

The example of the position indicator of FIGS. 52 to 54 includes a “traffic-light” element 5250 at the top. The traffic light element may be animated as a progress indicator to indicate to the user that the head is in the correct orientation and that the system is now undertaking a scan of the face of the user (such as by collecting one or more images or frames (such as photos or videos) of the face). For example, the traffic light element may be a circle that lights up (or blinks) while the scan is taking place, and then turns to say green once the scan is completed.

The example of FIGS. 52 to 54 operates with the same principle as the embodiment of FIGS. 48 to 51, in that on starting to tilt the head back from a frontal orientation, the user can see most of the column 5220. As the head continues to tilt, their line of sight rotates upwardly relative to the camera and display screen and they can see the screen less and less clearly and then eventually may see less and less of the screen (particularly the bottom portion). This is because the field of view is angled upwards and as the user tilts the head further backwards (increasing the relative angle between the head and the camera) less and less of the display of the device is positioned within the field of view of the user and so the user sees less of the screen. The colour on the bar (indicating their tilt progress) moves higher and higher, to a) indicate they are getting closer to the correct orientation, and also b) remain visible to the user. By moving the bar higher on the display screen the bar is maintained within the field of view of the user. The scan progress indicator 5250 is oriented so that, with the user's head tilted right back, they can see it and know when the scan has finished.

In FIGS. 52 and 53 the position indicator indicates that the tilt/orientation is in progress. In FIG. 54, the position indicator indicates that the head is in the desired orientation at full tilt. At this point of the process the scan can commence, and the traffic light indicator indicates that scanning in progress. The indicator might turn green once scanning is complete.

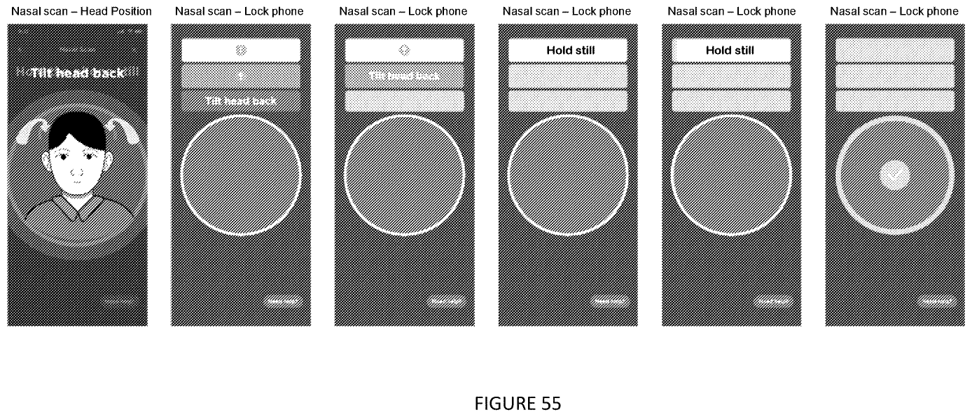

FIG. 55 shows a further example of a position indicator. In the example of FIG. 55, the position indicator includes indicator bars and is animated in a similar way to the example of FIGS. 48 to 51 in which the bars progressively illuminate towards the top of the screen as the user tilts their head back towards the desired orientation. Again, the change in position of the indicator moving towards the top of the screen compensates for the movement of the line of sight upwards and away from the camera and mobile device. In the example of FIG. 55, the position indicator includes text instructions to the user to provide further guidance to assist the user with positioning their head. In the example of FIG. 55 the text instructions also move between different positions on the screen (i.e. upwards on the screen as the user moves towards the desired orientation) along with the illuminated bars.

When the system detects that the user has orientated their head in the correct orientation the position indicator displays a text indicator “hold still”. This is a prompt to the user to remain still while the system captures an image of the user's face. In the example of FIG. 55 an animation (progress indicator) is displayed during the image capture stage (filling up the top bar with colour from left to right) to encourage the user to remain still during this time period. When the image is captured a further indicator is displayed on the screen (in the form of a tick in a green circle) to indicate that the scan is complete.

The example of FIG. 55 includes animated instructions, on-screen text that moves with the “active bar”, arrow indicators. Once the top progress bar is complete the circle also appears green and a green tick appears giving a very clear indication that the “scan/measurement” is complete.

The position indicators of FIGS. 48 to 55 are useful for the under-nose images where the user must tilt their head back and thus progressively gets poorer visibility as they move closer to the correct tilt angle, which the dynamic position indicator compensates for. The techniques described with respect to FIGS. 48 to 55 may also be applied to capturing side view images of the patient's face. For side view images, the position indicator may be arranged across the screen rather than up and down. As the system detects that the camera is being orientated around the head (or that the head is being orientated relative to the camera) from the front of the face towards the side view, the position indicators may be illuminated (or animated in another way) towards the side of the screen. Such systems allow the patient to capture accurate side images.

### Position Indicator—Front Facing—Concentric Circles.

Further examples of position indicators may be used to assist the user in orientating their face in a front facing orientation. Typically for a front facing image the user is provided with guidance to assist in orientating their head with the correct pitch and yaw. The orientation of the user's face is detected during the guided orientating process and feedback is provided to the user to assist in correctly positioning their head at the desired orientation. Note, the front-facing step(s) as described herein may be implemented in conjunction with the above-discussed tilt-orientation step(s). For instance, the front-facing step(s) may first be used to ensure the user's face is at a correct height with respect to the camera, that is to say, at eye level and thus front-facing in terms of pitch (in the sense of the plane of the camera being substantially parallel to the plane of the face). Subsequently, the tilt-orientation step(s), such as the above-discussed illuminated bars, may be employed to prompt the user to tilt their head back to capture a subnasal image. However, in other embodiments the front-facing steps may be used on their own, such as when only a frontal image is required.

In the following example the application is for selecting a patient interface for a patient for use with a respiratory therapy device, the patient interface suitable to deliver respiratory therapy to the patient. The application determines a dimension of a facial feature required in order to select a patient interface for a patient. The facial feature may be the width of the nose, or the depth of the nose etc. Once the dimension of the facial feature has been determined, the application determines a desired orientation of the face of the patient to be captured in a digital image, in order to calculate the dimension of the required facial feature. For example, if the height of the nose is the required dimension, the application may identify that the required orientation of the face to be captured in the image is a front facing orientation. The application may use certain attributes to define the front facing image. In order to assist the patient to correctly orientate their face, the application may provide guidance to the patient to position their face in the desired orientation for image capture. When the application receives image capture data representing at least one digital image of a face of a patient, the image capture data representing the face of the patient orientated in the desired orientation, the application may calculate a dimension for the facial feature from the image capture data. The dimension may then be used to select a patient interface for the patient.

In an example the system provides guidance to the user in the form of a position indicator. The position indicator is displayed on the display interface and comprises a real-time animation to indicate the orientation of the face towards and away from the desired orientation in dependence on the current orientation. The real-time animation comprises a series of indicators to assist the user in orientating the face in the desired orientation.

An example position indicator presents a static indicator representing the desired orientation of the face and a dynamic indicator representing the real-time orientation of the face. The static indicator is displayed at a fixed location on the display interface. The dynamic indicator is displayed at a location on the display interface representative of the current orientation of the face. The static indicator acts as a target for the user to align the dynamic indicator with to achieve the desired orientation of the face. A difference between the display location of the static indicator and the display location of the dynamic indicator on the display interface represents a difference between the current orientation of the user's head and the desired orientation of the user's head.

The dynamic indicator may include the image of the user's face, as seen by the camera.

When the static indicator and the dynamic indicators are aligned, this indicates to the user that the user's face is at the desired orientation.

The desired orientation may include a desired angle with respect to the camera, a desired height of the head with respect to the camera and a desired distance for the head from the camera.

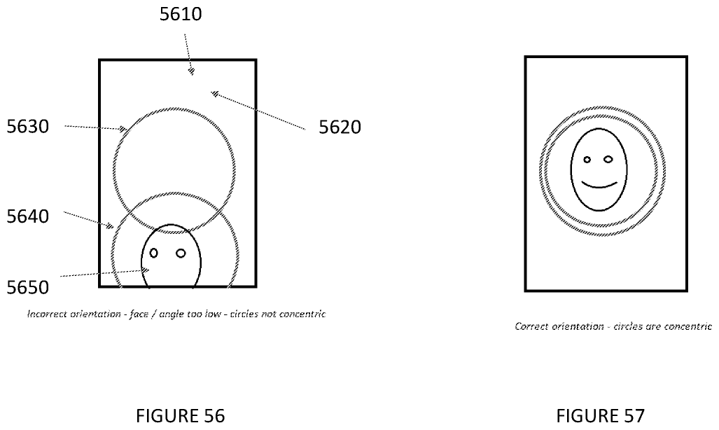

An example of a position indicator is now described with reference to FIGS. 56 and 57. In the example of FIGS. 56 and 57 position indicator includes a static indicator 5630 and a dynamic indicator 5640 displayed on the display screen 5620 of a mobile device 5610. Static indicator 5630 refers to the target/desired orientation of the head of the user in the image. Dynamic indicator 5640 represents the current orientation of the head of the user. In the example of FIGS. 56 and 57, the dynamic indicator includes an image of the user's face 5650 in real time.

The location of the dynamic indicator on the screen indicates the current orientation of the user's head with respect to the desired orientation, which is represented by the static indicator 5630.

In FIG. 56 the current orientation of the user's head is not in the desired orientation. This is displayed on the screen with the dynamic indicator misaligned from the static indicator. In FIG. 57 the current orientation of the head is at the desired orientation. This correct orientation is presented to the user with the static indicator 5630 and the dynamic indicator 5640 being aligned. In the example of FIG. 57 the indicators are circles and the circles are concentric when the head is in the desired orientation.

Correct (frontal) orientation is indicated by the two circles being concentric. But if the user's face is tilted down (or more generally if the user's face is at other than the correct (frontal) orientation), the dynamic circle “follows” them such that the circles are not concentric. This “concentric” display of FIGS. 56 and 57 is useful for aligning and capturing the front-on image. Generally with a front on image there is no concern about line of sight and field of view because the user is looking at the camera. As noted further above, this embodiment of the position indicator may be used when only a frontal image is required. Alternatively, it may also be used as a preliminary step where a sub-nasal image is required-specifically, it may be used to ensure that the camera is at eye level (which may effectively equate to the camera being positioned as for a frontal image), following which the display screen may change to show the position indicator of e.g. FIGS. 48-51, which prompt the user to tilt back their head to get into the required orientation for the sub-nasal image capture.

Note also, the “circle”-type display can be used not only to ensure correct height and front-facing orientation, but also to ensure correct distance from the screen. For instance, the dynamic circle may grow or shrink on the display screen to indicate the user moving towards/away from the screen. When they are at the correct distance, the circle might for example be approximately the same size as the static (background, grey) circle.

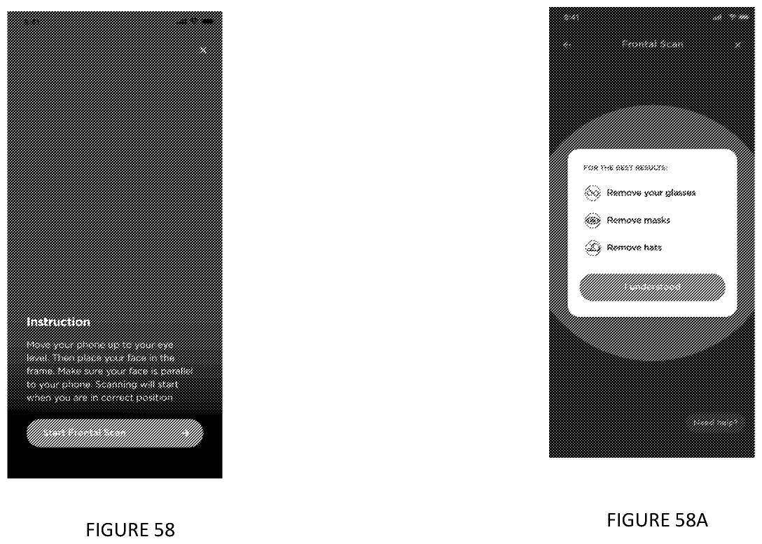

FIGS. 58 and 58A illustrate preliminary instructions that may be provided to a user on initiating a facial scan, in particular a scan requiring a front on image.

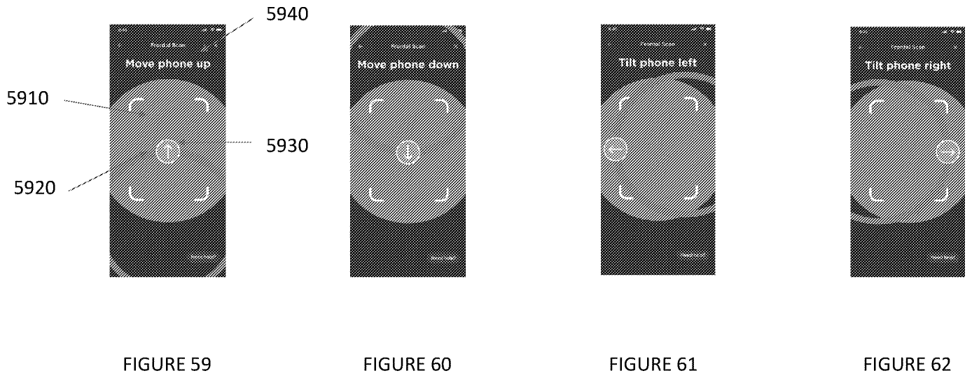

FIGS. 59 to 62 illustrate further examples of presenting a static indicator and a dynamic indicator. In the examples of FIGS. 59 to 62 static indicator represents the target desired orientation for the head of the user. The animation for static indicator 5910 is different from FIGS. 57 and 58 but uses the same principle. Dynamic indicator 5920 is presented by a circle on the screen on the mobile device. Any difference in alignment of the static indicator and the dynamic indicator represents a difference in the current orientation of the face of the user relative to the desired orientation of the face of the user.

In the examples of FIGS. 59 to 62, additional indicators are displayed on the screen of the mobile device to assist the user in correctly orienting their face. The examples include a directional indicator 5930 in the form of an arrow pointing to the direction in which the user should move their head to move towards the desired orientation. The examples include a text indicator 5940 to indicate the direction that the user should move their head to move towards the desired orientation.

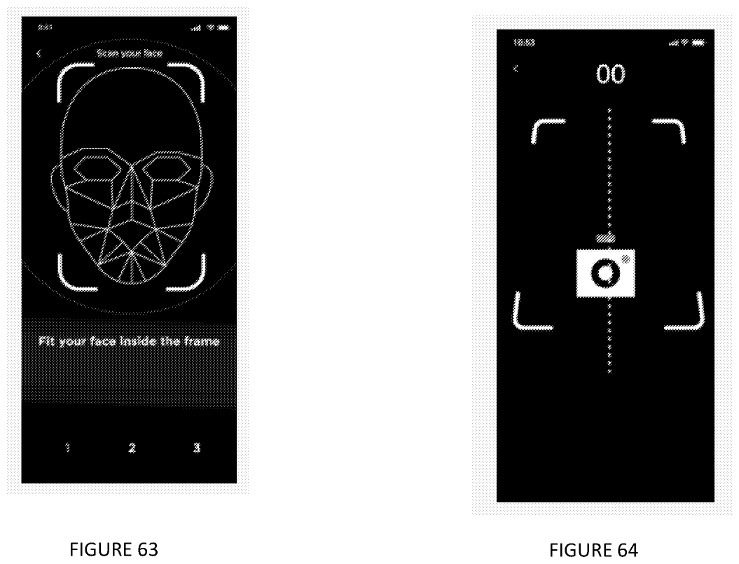

Another example is a “frame”-type box shown in FIGS. 63 and 64 which, when the user is front-on, is visibly oriented parallel to the screen; but acquires “perspective” (see FIG. 64) when the user tilts their head, as a visual representation of undesirable (or, in some embodiments, desired) tilt. In the example of FIG. 64 the frame type box appears wider/broader at its lower end indicating that the bottom portion of the face is closer to the camera/phone than the top portion of the face. This corresponds to the user's head being tilted back. This “foreshortening” effect may increase as the head tilts back further. Similarly, the top end of the frame type box may be wider/broader to indicate the top portion of user's face being closer to the camera/phone than the bottom portion.

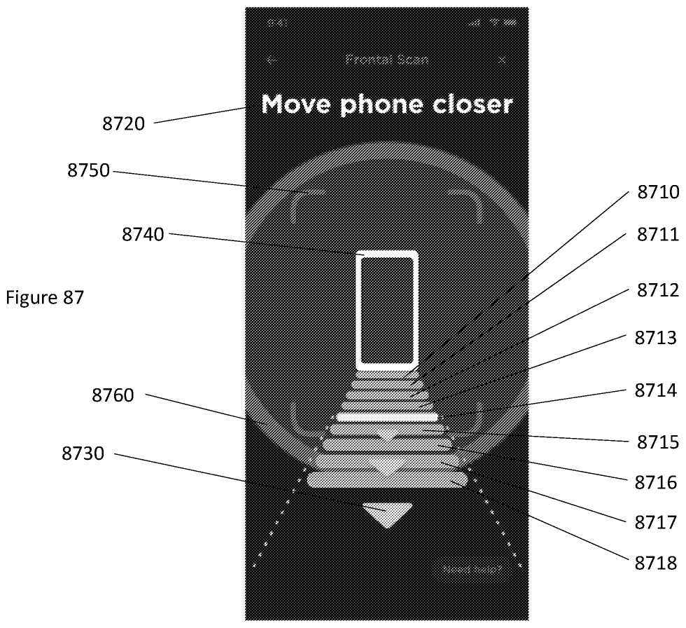

FIGS. 87 to 95 schematically show an example of a visual indicator displayed on a mobile device to guide a user to position their face a desired distance from the camera for an image capture. The system may calculate the distance between the camera and the face of the patient using the techniques described herein. Other techniques for calculating the distance may be used. In the example of FIGS. 87 to 95 the visual indicator is a distance indicator (in some examples previous steps in the process have occurred prior to this point, such as prompts displayed to the user to assist them to move their face to the correct height and/or orientation relative to the screen). In FIGS. 87 to 95 it will be understood that the user will be facing the screen and so will see the indicator displayed on the screen. In other systems, for example in a clinician mode in which a clinician or other third party operates the camera, the camera on the rear of the mobile device may be used, the camera pointing towards the patient and away from the clinician or third party. In such cases the screen is displayed to the clinician or third party and not to the patient.

The screen includes nine visual indicators 8710 8711 8712 8713 8714 8715 8716 8717 8718. Together the visual indicators form part of a position indicator to assist the user to correctly position their face and/or head at the desired distance from the camera in order to capture an image of the head. In example devices where the camera is co-located with the screen the visual indicators also guide the patient to the correct distance from the screen.

In the examples of FIGS. 87 to 95, the indicator is provided by a series of indicator bars on the screen. Different styles of indicator may be used. In the example of FIGS. 87 to 95 the indicator bars are horizontally arranged across the display screen. The horizontal indicator bars are arranged to guide the user towards a desired distance by representing a comparison between the current distance between the face and the camera (as represented by an identified indicator bar) and the desired distance between the face and the camera (as represented by a specific indicator bar aka target distance indicator bar). The indicator is a progressive sequence including a series of indicator bars displayed at different locations on the screen. In the example of FIGS. 87 to 95 the desired distance between the face and the camera is represented by one of the indicator bars 8714. For the purposes of the description of FIGS. 87 to 95 this indicator is referred to as the target distance indicator bar. The target distance indicator bar 8714 is distinguishable from the other indicator bars. In example systems the target distance indicator bar may be represented with a different animation or colour to allow it to be easily distinguishable as a target indicator bar compared with other indicator bars. In the example of FIGS. 87 to 95 the target distance indicator bar is a different colour from the other indicator bars. In further examples, the target indicator bar may include a particular animation, may be a different shape from the other indicator bars, or be presented in some other way to make it recognisable from the other indicator bars.

The current distance between the face of the user and the camera is presented to the patient. As the distance between the face and the camera changes with respect to the target distance the animation of the indicator changes. In the example of FIGS. 87 to 95, indicator bars are animated to identify whether the distance between the camera and the face of the patient is moving closer to the desired distance or further away from the desired distance.

In the example of FIGS. 87 to 95 the current distance between the camera and the face is represented by illuminating one of the indicator bars. The bar representing the current distance between the camera and the face of the patient is referred to as the current distance indicator bar. Other animations or techniques may be used to distinguish the bar representing the current distance of the device compared with the other indicator bars. Indicator bars in closer proximity to the target distance indicator represent separation distances between the face and the camera closer to the desired distance. The closer the current distance indicator bar is to the target distance indicator bar the closer the current distance between the face and the camera is to the desired distance. The indicator bars may not be visible until they are illuminated or changed in colour.

In FIGS. 87 to 95 the indicator is represented as a three dimensional-looking image or animation (i.e. an image or animation which appears visually on the screen like it is three dimensional) in which indicator bars representing the distance between the face and the camera being too great are smaller in the display than indicator bars representing the distance between the face and the camera being too small. The animation of FIGS. 87 to 95 represents a pathway of indicator bars extending away from the patient.

Additional animation or indicators may be provided to assist the patient in reaching the desired orientation between the face and the camera. In the example of FIGS. 87 to 95 text 8720 is displayed to the patient to assist them to reach the correct distance. In the example of FIG. 87 “Move phone closer” is displayed to prompt the patient to bring the camera closer to the face. FIG. 87 also includes arrows 8730 promoting the patient to bring the camera closer to the face. FIG. 87 also includes an animation of a mobile device 8740. The animation of the mobile device is updated as the distance between the face and the camera is changed. As the distance between the face of the patient and the camera is reduced the size of the mobile device animation is increased to represent the mobile device moving towards the face of the user. As the distance between the face of the patient and the camera is increased the size of the mobile device animation is reduced to represent the mobile device moving away from the face of the user.

The animation may be superimposed on a live-image captured by the camera (not shown). If the patient is holding the camera at the correct angle and/or height then the patient's face should be captured within the live-image and displayed on the screen. A frame 8750 may be displayed to assist the patient in maintaining the correct angular orientation and/or height of the camera while the distance between the camera and the face is adjusted. This helps provide a general reference to the patient.

A sequence of animation is now described with respect to FIGS. 87 to 91. In FIG. 87 the camera is too far away from the face. The distance between the face and the camera is greater than the desired distance. This distance is represented in the animation by indicator bar 8710 being illuminated. Indicator bar 8710 is smaller than the target distance indicator bar 8715 and represented as further away in the three dimensional animation. The animation prompts the user to move the camera closer to the face. Additional prompts are presented to the patient to prompt the patient to move the camera closer to the face in the form of text indicator 8720 “Move phone closer” and arrows 8730 pointing downwards on the screen (or towards the patient in a three dimensional animation-sense) directing the patient to move the camera closer to the face.

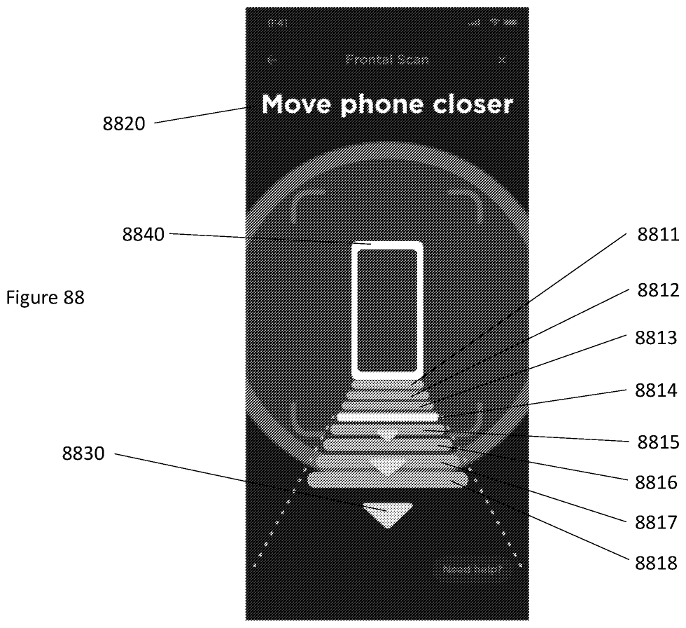

After FIG. 87, the patient moves the camera towards the face. The updated distance between the face and the camera is detected and the animation displayed to the patient is updated. The updated animation is shown in FIG. 88. In FIG. 88 the camera is now closer to the face of the patient than in FIG. 87. But the camera Is still too far away from the face. The distance between the face and the camera is greater than the desired distance. The closer distance is indicated by indicator bar 8811 being illuminated (the previous indicator bar 8710 is obscured by the animation of the mobile device 8840). Indicator bar 8810 is smaller than the target distance indicator 8814 and represented as further away in the three dimensional animation. The animation prompts the user to move the camera closer to the face. Additional prompts are presented to the patient to prompt the patient to move the camera closer to the face in the form of text indicator 8820 “Move phone closer” and arrows 8830 pointing downwards on the screen (or towards the patient in a three dimensional animation-sense) directing the patient to move the camera closer to the face. Animation of the mobile device 8840 is larger than the corresponding animation 8740 in the previous distance represented in FIG. 87 to represent the mobile device being brought closer to the face of the patient.

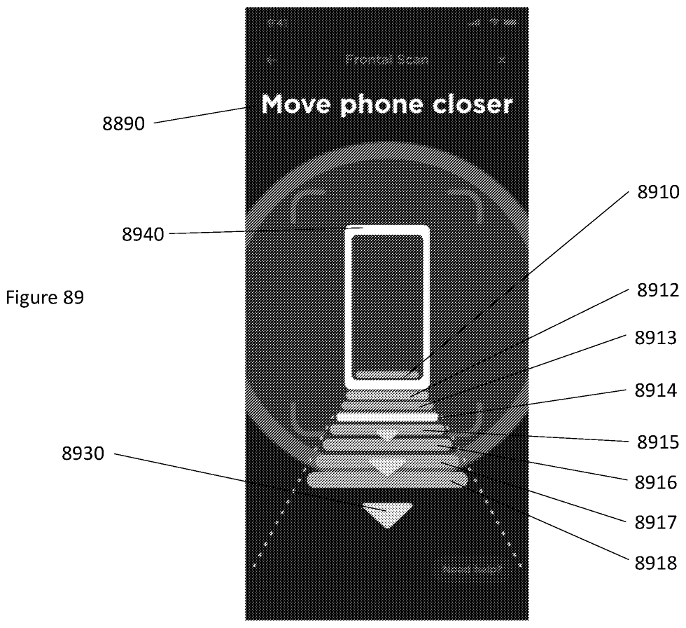

After FIG. 88, the patient moves the camera further towards the face. The updated distance between the face and the camera is detected and the animation displayed to the patient is updated. The updated animation is shown in FIG. 89. In FIG. 89 the camera is now closer to the face of the patient than in FIGS. 88 and 87. But the camera Is still too far away from the face. The distance between the face and the camera is greater than the desired distance. The closer distance is indicated by indicator bar 8912 being illuminated (the previous indicator bar 8811 is obscured by the animation of the mobile device 8940). Indicator 8912 is smaller than the target distance indicator bar 8914 and represented as further away in the three dimensional animation. The animation prompts the user to move the camera closer to the face. Additional prompts are presented to the patient to prompt the patient to move the camera closer to the face in the form of text indicator 8890 “Move phone closer” and arrows 8930 pointing downwards on the screen (or towards the patient in a three dimensional animation-sense) directing the patient to move the camera closer to the face. Animation of the mobile device 8940 is larger than the corresponding animation 8840 in the previous distance represented in FIG. 88 to represent the mobile device having been brought closer to the face of the patient.

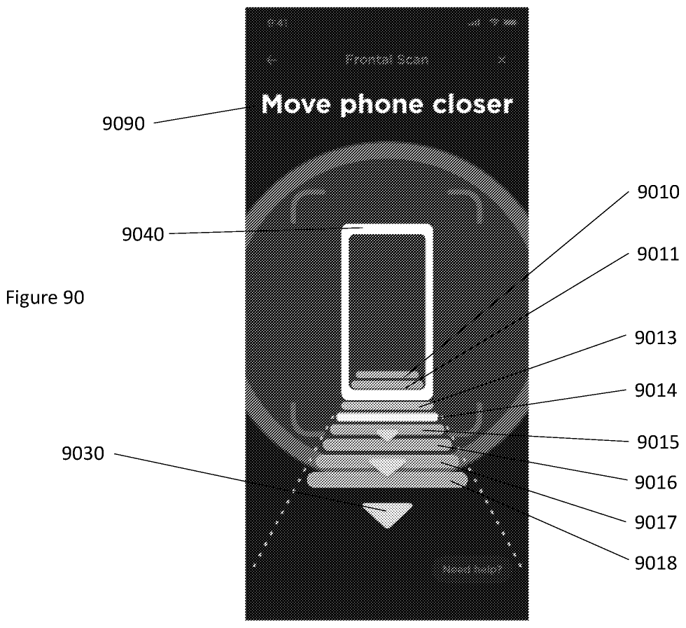

After FIG. 89, the patient moves the camera further towards the face. The updated distance between the face and the camera is detected and the animation displayed to the patient is updated. The updated animation is shown in FIG. 90. In FIG. 90 the camera is now closer to the face of the patient than in FIGS. 89, 88 and 87. But the camera Is still too far away from the face. The distance between the face and the camera is greater than the desired distance. The closer distance is indicated by indicator bar 9013 being illuminated (the previous indicator bar 8912 is obscured by the animation of the mobile device 9040). Indicator bar 9013 is smaller than the target distance indicator 9014 and represented as further away in the three dimensional animation. The animation prompts the user to move the camera closer to the face. Additional prompts are presented to the patient to prompt the patient to move the camera closer to the face in the form of text indicator 9090 “Move phone closer” and arrows 9030 pointing downwards on the screen (or towards the patient in a three dimensional animation-sense) directing the patient to move the camera closer to the face. Animation of the mobile device 9040 is larger than the corresponding animation 8940 in the previous distance represented in FIG. 89 to represent the mobile device having been brought closer to the face of the patient.

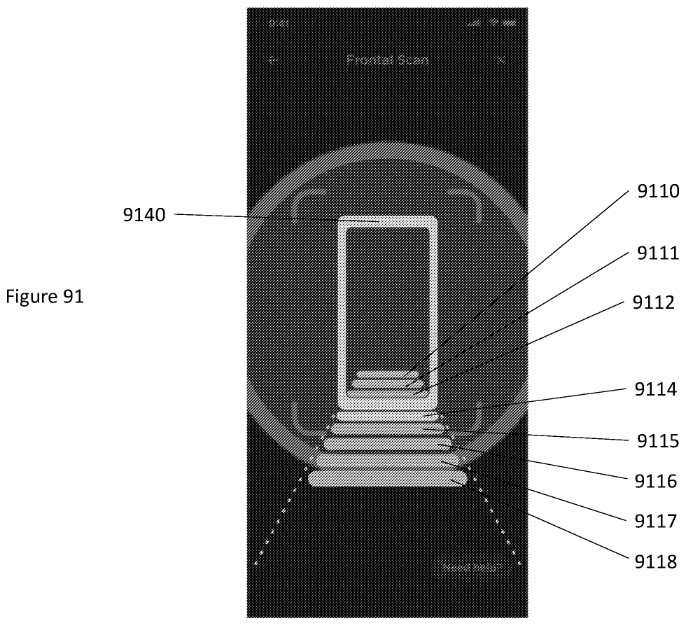

After FIG. 90, the patient moves the camera further towards the face. The updated distance between the face and the camera is detected and the animation displayed to the patient is updated. The updated animation is shown in FIG. 91. In FIG. 91 the camera is now closer to the face of the patient than in FIGS. 90, 89, 88 and 87. The distance between the face and the camera is the desired distance. The desired distance is indicated by target distance indicator bar 9114 being illuminated (the previous indicator bar 9013 is obscured by the animation of the mobile device 9140). The mobile device animation 9140 is also illuminated to indicate to the patient that the distance between the face and the camera meets the desired distance requirements. Animation of the mobile device 9140 is larger than the corresponding animation 9040 in the previous distance represented in FIG. 90 to represent the mobile device having been brought closer to the face of the patient. When the distance between the face and the camera meets the desired requirements further positioning sequences, for example the orientation or tilt sequences described above may be activated. The system may also move into an image capture phase.

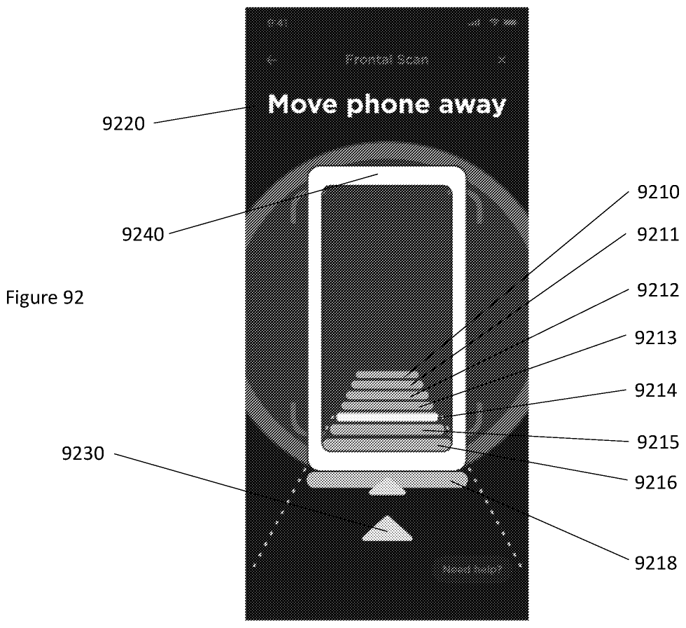

Animation can also be used to indicate to the user that the distance between the face and the camera is too short and that the camera should be moved further away from the face. The sequence of animations shown in FIGS. 92 to 95 show the same animation but to prompt the patient to move the camera away from the face.

In FIG. 92 the camera is too close to the face. The distance between the face and the camera is less than the desired distance. This distance is represented in the animation by indicator bar 9218 being illuminated. Indicator bar 9218 is larger than the target distance indicator bar 9214 and represented as nearer in the three dimensional animation. The animation prompts the user to move the camera further from the face. Additional prompts are presented to the patient to prompt the patient to move the camera further from the face in the form of text indicator 9220 “Move phone away” and arrows 9230 pointing upwards on the screen (or away from the patient in a three dimensional animation-sense) directing the patient to move the camera further from the face.

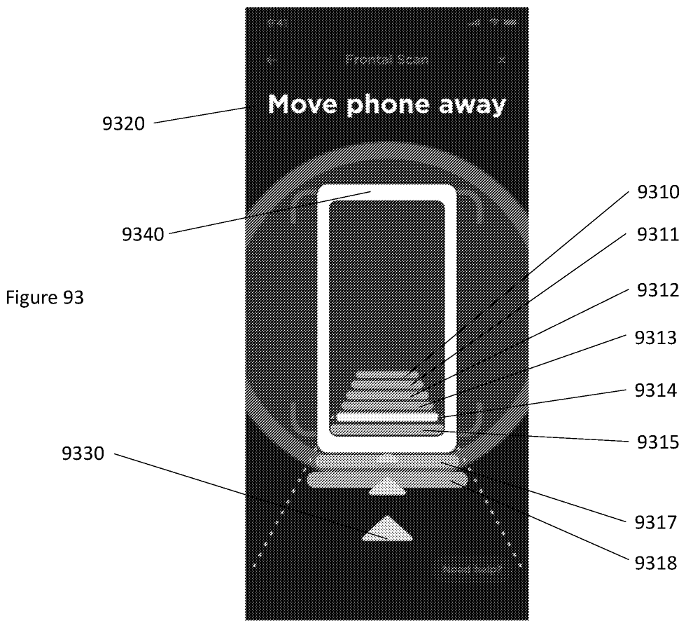

After FIG. 92, the patient moves the camera away from the face. The updated distance between the face and the camera is detected and the animation displayed to the patient is updated. The updated animation is shown in FIG. 93. In FIG. 93 the camera is now further from the face of the patient than in FIG. 92. But the camera is still too close to the face. The distance between the face and the camera is less than the desired distance. The further distance is indicated by indicator bar 9317 being illuminated. Indicator bar 9317 is larger than the target distance indicator 9314 and represented as closer in the three dimensional animation. The animation prompts the user to move the camera further from the face. Additional prompts are presented to the patient to prompt the patient to move the camera further from the face in the form of text indicator 9320 “Move phone away” and arrows 9330 pointing upwards on the screen (or away from the patient in a three dimensional animation-sense) directing the patient to move the camera further away from the face. Animation of the mobile device 9340 is smaller than the corresponding animation 9240 in the previous distance represented in FIG. 92 to represent the mobile device being brought further from the face of the patient.

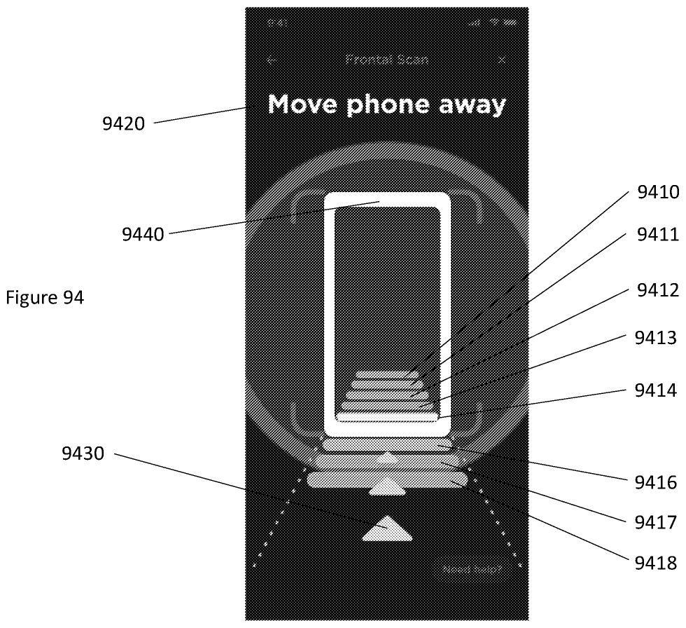

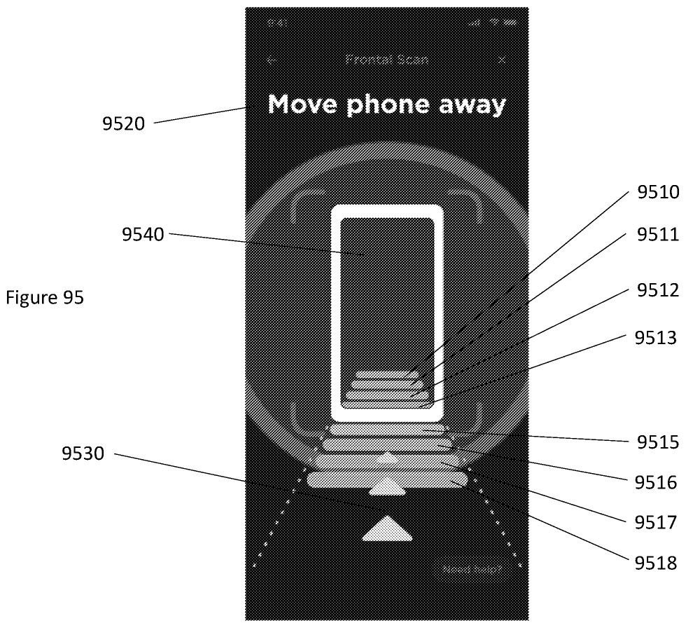

The animation sequences shown in FIGS. 94 and 95 show the changes in animation as the patient moves the camera further from the face and towards the desired distance between the face and the camera. When the system detects that the distance between the face and the camera matches the desired distance, the animation indicates that the distance between the camera and the face is correct, as shown in FIG. 91.

The animation also includes a ring 8760 superimposed on a live-image of the patient. The live-action preview within ring 8760 shows the image that would be captured by the camera at the current distance between the camera and the face. As the distance between the face and the camera is reduced, the face becomes larger within the ring, and as the distance between the face and the camera is increased the face becomes smaller in the ring.

In the animation described with reference to FIGS. 87 to 95, the caricature/animation on the screen is displayed to link to/emulate the variables being measured. For example, when a user moves the phone closer/further, the animation of the mobile device gets larger/smaller. This provides an intuitive indication of mobile device positioning relative to the patient to help the patient to attain the correct distance between the camera and the face for image capture. The “live-time” or “real time” display of the distance provides the patient with real-time feedback in response to changes in position of the camera or face by the patient.

The desired distance between the camera and the patient's face may be calibrated for different camera systems, for example as a function of focal point as discussed further above. In some systems the preferred separation distance between the camera and the face of the patient is between 35 cm and 45 cm.

Once the patient has reached the desired distance between the face and the camera the animation may include a “progress indicator” that indicates a progress of a scan or image-capture process. Once the distance between the camera and the face is correct the camera is triggered to capture an image (or potentially more than one image, such as a plurality of images or frames (photo or video)). For example, the animation of the mobile device may become the progress indicator when the distance between the camera and the face is correct.

Correct alignment/positioning of the face for image capture improves the accuracy of dimensions and measurements calculated from the images. This promotes accuracy of the final interface/mask (size) recommendation as more reliable and accurate measurements of the user's facial dimensions are obtained. The system can be configured to set varying degrees of accuracy for different orientations by varying ranges associated with the desired orientations. For example, the system may set a tolerance of between −6 degree to +6 degrees for tilt of the user's head when capturing an underside facial image to obtain the dimension of the depth of the nose. Other systems may set a smaller range. Other systems may set a larger range. The system may set different ranges for different parameters. Different ranges may also be applicable for different mask types, for example the accuracy of a particular dimension may be very important for some mask types or less important for other mask types.

Potentially there are some angles etc at which the system would still allow an image to be taken, but the angle would be skewed and this would affect measurements and thus the output (mask recommendation.

In example systems there are multiple parameters which may be required to be aligned or positioned in predetermined desired positions/orientations correctly in order to capture an image which may be used to obtain accurate dimensions. Example parameters include the pitch of the camera. In some orientations the camera should be positioned so its lens views horizontally. In embodiments the camera is within a mobile device pointing outwards from the face (i.e. display screen side) of the mobile device. This may be preferable, although it may also be possible for the camera to be oriented pointing outward on the back of the mobile device. In such embodiments the camera can be correctly orientated by monitoring the orientation of the mobile device, for example using a gyroscope or accelerometer. Indicators may be presented to the user on the screen of the device to assist the user in orientating the mobile device and camera correctly. Various versions of the system may include caricatures or various animations on the screen, illustrating how the user should move their phone. These may be used instead of, or in addition to, the text prompts.

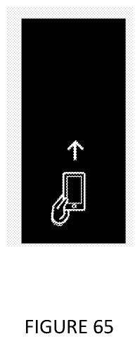

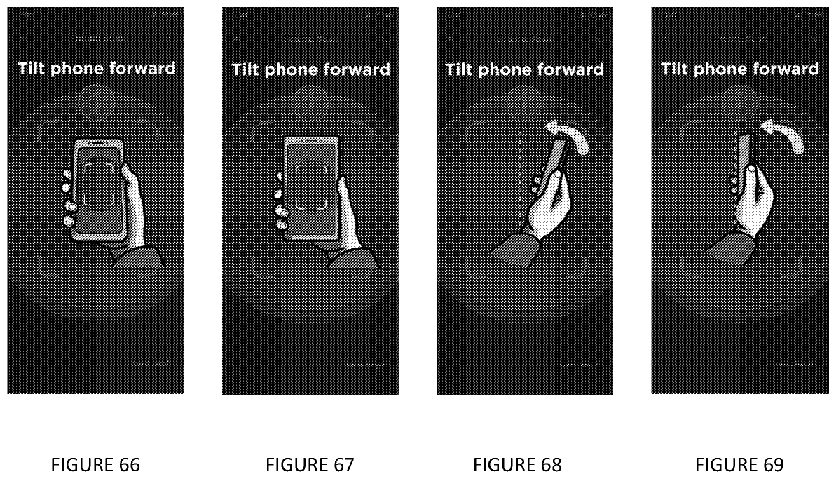

FIGS. 65 to 69 show various exemplary animation indicators presented to the user on the screen to guide the user to position the mobile device in the correct orientation. Other designs of the animation indicators are possible. For instance, a simple instruction to the user to “hold your phone vertically” and/or “hold your phone at eye level” could also be used.

Further parameters include the height of the camera when compared with the face of the user. Preferably the camera should be positioned at eye level. Further parameters include the angle of the user's face. Typically the angle may be defined with respect to the camera. Further parameters include the distance between the camera and the user's face.

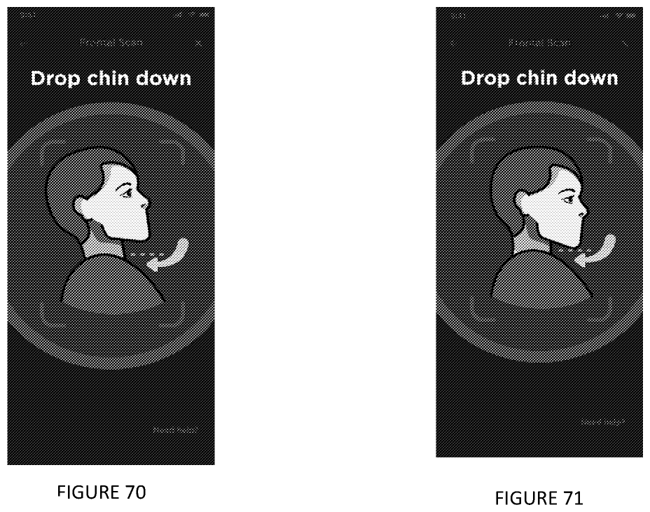

In addition to the visual indicators described above, other animations of the type shown in FIGS. 70 and 71 may also be used to guide the user to tilt their head/face. Similar animations could be used to guide the user to position their head/face in other orientations relative to the camera, such as closer/further or higher/lower.

These parameters may be specific numerical values, or they may be “functional”—e.g. the required distance may just be “such that the entire face is in frame”, or the angle may be such that “the nostrils are sufficiently visible”. Functional may require that a particular feature, typically a facial feature, or group of features is visible in the image. The functional parameters are defined in relation to features appearing in the image rather than specific angles or distances or other measurement-type parameters

During the process of orientating the face for image capture, changing one parameter may affect another. For example, if a user is tilting their head backwards, they may inadvertently change the pitch of the camera, or move their head closer to the camera. During the process of orientating the face multiple parameters may be monitored.

The application may guide the user to the correct parameters in a specific order, for example for accurate placement of the face relative to the camera, a basic sequence may run as follows: first, it is ensured that the user is holding the camera at the right height; second, it is ensured that the phone is at the right distance; third, it is ensured that the angle of the user's face (i.e. front on, or under-nose) is as required.

This sequence may be advantageous in that, for the first 2 steps (height and distance), the above-discussed “frontal” prompts may be used-such as the concentric circles and other types of displayed prompts that are visible to the user when the user is front-on to the camera. Subsequently, once the user is at the right height and distance, the display might switch to the “tilt prompts”, such as the green bars, which guide the user to tilt their head back.

Alternatively, the sequence may be as follows: first, it is ensured that the user is holding the camera at the right height; second, it is ensured that the angle of the user's face (i.e. front on, or under-nose) is as required; third, it is ensured that the phone is at the right distance.

In this alternative sequence, the prompts may accordingly be displayed in an appropriate order—for instance, first the “concentric” display on the screen, to achieve correct height; then the “bars” to achieve correct tilt; and then prompts to achieve correct distance. In this embodiment, the distance prompts may be shown proximate the top of the screen, so that the user can clearly see them even though their head is already tilted back. This may ensure the user maintains the tilted-back orientation when adjusting for distance, and is not tempted to deviate from the tilted-back position in order to better view the prompts on the screen.

During the ongoing orientation process the system may continue to monitor the parameters that have already been met and may interrupt the application if the user moves out of a desired orientation. This process may be “iterative”. So the system may continue to repeat the instructions until the user's facial orientation conforms to all multiple parameter requirements.

In an example, for the “under-nose” photo with the sequence being (“height, distance, angle”), the screen may start off with the concentric circle animation described above to encourage the user to meet the first criterion of positioning the camera at the correct height, which the user will perform in a front on orientation. When the head is at the correct height, the system may also use the circle animation to ensure correct distance (or alternatively, the “horizontal bars in perspective” animation could be used). The system may then initiate the tilt application and the screen may present the horizontal bar indicator described above (at the top portion of the screen) to guide the user to tilt back the head. Alternatively, if the sequence is instead (“height, tilt, distance”), then the screen may again start off by showing the circle animation to get correct height; may then move to the “horizontal bars” animation (at the top of the screen); and may subsequently display instructions as to distance on a portion of the screen (notably the upper portion) that the user, with their head tilted back, is able to see. Otherwise, if they have to “un-tilt” to read the instruction then the process has to start again.

In one example the system provides a method of orienting an image-capture device and a user's face in a required three-dimensional relation relative to one another to enable capture of an image of the user's face at a required position by the image-capture device. The method includes the steps of providing prompts to the user to assist the user to attain a required height of the user's face relative to the image-capture device; providing prompts to the user to assist the user to attain a required angle of the user's face relative to the image-capture device; and providing prompts to the user to assist the user to attain a required distance of the user's face relative to the image-capture device.

The height application, the distance application and the angle application may be executed in a preferred sequence. (For instance, without limitation, the order may be “height-distance-angle”).

When a required value (or value range) has been attained, the attained value is monitored and the application is interrupted if the value subsequently departs from the required value (or value range). When an application is interrupted, the system may provide guidance to the user to re-attain the required value (which has fallen outside the required value). “Interrupted” might not necessarily mean that existing processes are aborted; it might instead (or additionally) mean that additional prompts are displayed, to correct whichever required value has been departed from. The term suspended may also be used herein to have the same meaning as the term interrupted. The terms “interrupted” and “suspended” may indicate that the application is paused until an incorrect parameter is corrected.

When the user has attained at least one of the required height value, required distance value, and required angle value, capturing an image of the user's face with the image capture device may commence.

The prompts may be visual prompts. The prompts may be displayed on the display interface.

In some example systems there is a “compromise point”—for instance the system may determine that the head is in the desired orientation if two of the three parameters are satisfied (or within range); or if two of the three parameters are satisfied (or within range) and the third is within the vicinity (or within a broader range value) of its required range. The same principle holds true if not only the 3 dimensional parameters but also the respective axes (of the user and the phone) and their alignment are considered. For instance, if there are 8 degrees of freedom in total (being the 3 dimensional aspects (height, distance, angle) and the alignment of the respective axes), then say satisfaction of 6 of 8 of these may be sufficient (with the remaining say 2 needing to be within some vicinity of their required ranges).

In some example systems once the head is in the desired orientation, the image-capture device automatically captures the image(s) of the user's face.

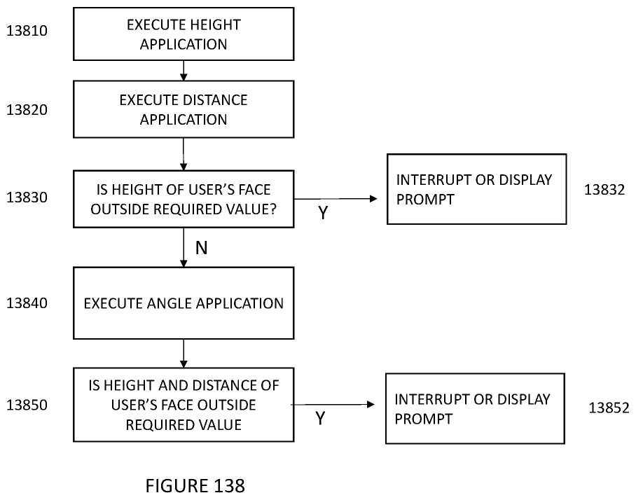

In one example, shown in FIG. 138, the method has a predefined sequence of meeting the desired parameters. The method guides a user to position and orientate their face and an image capture device in a required three-dimensional relation relative to one another for image capture. The method comprises the steps of executing of a height application at 13810 to guide the user to attain a required height value of the user's face with respect to the image capture device. When the user has attained the required height value, triggering a distance application at 13820 to guide the user to attain a required distance value between the user's face and the image capture device. During execution of the distance application, monitoring the height of the user's face with respect to the image capture device at 13830, wherein if the height of the user's face is outside the required height value, interrupting the distance application (or displaying additional prompts relating to height, simultaneously with continuing to run the distance application) at 13832. When the user has attained the required distance value, triggering execution of an angle application at 13840 to guide the user to attain a required angle value of the face of the user with respect to the image capture device. During execution of the angle application, monitoring the height of the user's face with respect to the image capture device, and the distance from the user's face from image capture device at 13850, wherein if the height of the user's face or the distance to the image capture device are outside the predefined height value or distance value, interrupting the angle application (or displaying simultaneous additional prompts as to height and distance) at 13852.

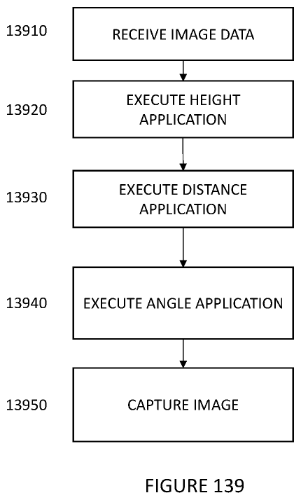

In one example, shown in FIG. 139, the system captures an image when the user has attained at least one of the required height value, required distance value, and required angle value. The method guides a user to position and orientate their face and an image capture device in a required three-dimensional relation relative to one another for image capture. The method comprising the steps of receiving data representing at least one digital image of a face of a patient at 13910. At 13920 the system executes a height application to guide the user to attain a required height value of the user's face with respect to the image capture device. At 13930 the system executes a distance application to guide the user to attain a required distance value between the user's face and the image capture device. At 13940 the system executes an angle application to guide the user to attain a required angle value of the face of the user with respect to the image capture device. At 13950 when the when the user has attained at least one of the required height value, required distance value, and required angle value, capturing an image of the user's face with the image capture device.

### Detecting the Orientation of the User's Face:

The gyroscope and/or accelerometer in the user's mobile device detects the orientation of the mobile device and allows feedback to be provided to make sure that the mobile device itself is held in a vertical orientation. Typically, for most examples the mobile device is held vertical, and the head is orientated (in the sense of angled or tilted) with respect to the vertical mobile device. If the phone detects it's non-vertical, it interrupts (or supplements with additional prompts) the application and instructs the user to re-orientate the mobile device.

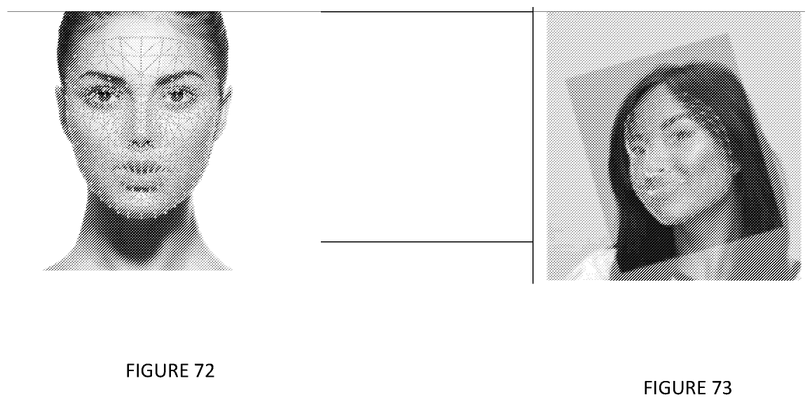

The facial processing technology (including superposing a face mesh on the obtained image) detects facial features/points/landmarks and also the angle of the face relative to the phone. For instance, one way of achieving this may be that the face mesh behaves in a 3D or perspective-like manner in that those “squares/grids” of the mesh which fall away from the viewer at a steeper gradient appear “foreshortened”, such that the mesh appears “denser” there. FIGS. 72 and 73 show the face mesh superimposed onto the image of a face at different angles. Angles may be detected by detecting features of the face that are/appear “foreshortened” in the face mesh, where they shouldn't be if the face were front-on. For instance, in FIG. 73 the squares/grids proximate the left cheek area in the image appear “denser” due to being foreshortened, compared to those proximate the right cheek; and this is one way of detecting that the face is not oriented front-on.

The face mesh (or other facial processing technology) may likewise detect distance (such as by considering focal length of the phone and/or apparent size of facial features) and angle.

Example systems take two 3D objects (the mobile device and the face) and orient them correctly with respect to one another in 3 dimensions—for correct (relative) height, angle, and distance.

The position indicators described above can be used in conjunction with the mask sizing and selection processes also described herein, including to identify a reference feature and derive a reference measurement and further to calculate dimensions of facial features.

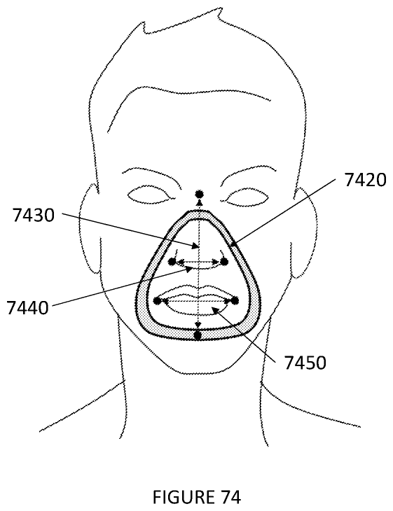

This description provides examples of systems for determining an appropriate patient interface size and/or patient interface type for a patient. Examples of methods for selecting a patient interface size and/or patient interface type for a patient are now described with reference to FIGS. 74 to 86.

As described above, different patient interface types i.e. interface categories are available to patients including full face masks, nasal face masks and sub nasal masks i.e. under nose masks and nasal pillows. In each category of patient interfaces, there are different sizes available to fit faces of different shapes and sizes.

Due to the different shapes of the different mask categories and the different points at which the masks seal to the face, different facial dimensions are important for different mask categories in order to find a good fit and a good seal. Table 1 (provided above) provides an example of the dimensions required in order to fit different patient interface categories accurately. For example, for a full face mask, dimensions are required for the facial features of: nose bridge to the lower lip; mouth width; and nose width. For a nasal face mask, dimensions are required for nose height and nose width. For an under nose nasal mask, dimensions are required for nose width and nose depth. For a nasal pillow, nostril size in the major axis and minor axis is required.

Referring back to FIG. 24, FIG. 24 illustrates the relevant facial feature dimensions required for sizing a full face mask. The first relevant dimension is the dimension 2430 from the nasal bridge to the lower lip. Referring to FIG. 10, this is the dimension from landmark (d) nasion to landmark (m) sublabial. The second relevant dimension is the width of the mouth 2450. Referring to FIG. 10, this is the dimension between landmark (k) left labial commissure and landmark (l) right labial commissure. A third relevant dimension is the width of the nose 2440. Referring to FIG. 10, this is the dimension between landmark (h) left alare and landmark (i) right alare.

To accommodate faces and heads of different sizes, masks are produced in different sizes. As described above, within each patient interface category, patient interfaces may be provided in different sizes, for example XS, S, M, L. For sealing interfaces, the size of the patient interface is generally defined by the seal size, i.e. the size of the patient interface seal that contacts the face. Generally, patients with larger heads require a larger seal size in order to provide an optimal or working seal. (For non-sealing interfaces, criteria other than seal are used to determine the appropriate size of the patient interface, as discussed elsewhere herein). The size of the headgear is also a consideration for effectiveness and comfort and the headgear may also be provided in different sizes depending on the size of the head of the patient. Some patient interface categories may also include an XL patient interface size.

FIG. 74 shows an example of a full face mask for the purpose of explaining the relationship between the dimensions of the various facial features of the patient and the dimensions of the mask.

As shown in FIG. 74 the width of the mouth 7450 is less than the width of the mask in order that the mouth does not contact the seal of the mask. The width of the nose is less than the width of the mask around the nose in order that the nose does not contact or interfere with the seal.

Different mask sizes within the same category have different mask dimensions. These mask dimensions may be proportional between the different mask sizes.

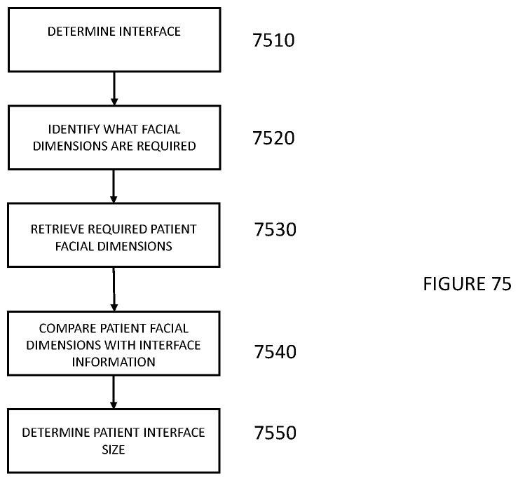

FIG. 75 shows the steps taken to select a patient interface for a patient. In particular FIG. 75 shows the steps taken to select a required patient interface size for a patient. At 7510, the system identifies which patient interface category is required for the patient. This determination may be made by assessing patient responses to a questionnaire, or based on a previous patient interface prescribed to the patient, patient selection, clinician selection, or another means. At 7520 the system identifies which patient facial dimensions are required in order to select a size of patient interface for the patient of the determined interface from 7510. Typically, the system retrieves this information from a lookup table including information of the type shown in Table 1 (above).

At 7530 the system retrieves the required facial dimensions of the patient. The facial dimensions may be retrieved using the image techniques described herein. In other systems the facial dimensions may be retrieved from a database containing patient facial dimension information. In other systems the patient facial dimensions may be provided by the patient or by a clinician. If patient facial images are required to be obtained to determine patient facial dimensions these may be obtained using a camera, for example a camera on a mobile communication device. The camera may be used by the patient, for example in ‘selfie’ mode where the patient captures an image of their face by pointing the camera and the display screen towards their face. Or the camera may be used by a clinician or other third party typically using the rear camera directed at the patient. This mode is sometimes referred to as a ‘clinician mode’.

At 7540 the system compares patient interface sizing information with the retrieved patient facial dimensions. Patient interface sizing information for different sized patient interfaces is compared with the retrieved patient facial dimensions to determine which patient interface size provides the best fit for the patient at 7550. In some embodiments, patient facial dimensions are input into or compared against a set of rules to determine which patient interface size provides the best fit for the patient.

As described before, the patient interface sizing information may include the facial feature dimensions suitable for each size of patient interface. The patient interface sizing information may be a range of facial feature dimensions suitable for each size of patient interface. Typically, the patient interface sizing information is provided for each of the relevant facial feature dimensions required for fitting the patient interface type.

In some embodiments, the system captures the patient facial dimensions for all facial dimensions of the patient relevant for any patient interface. These patient facial dimensions are recorded for the patient. By capturing all patient facial dimensions the system is able to make a sizing assessment for all patient interface categories. Meaning that if the patient wishes to change patient interface category to a different patient interface category in the future the patient dimensions and sizing have already been performed and so the patient does not need to run the sizing process again. Storage or recordal of the patient's facial dimensions may be done in an anonymized fashion whereby the record of the patient's facial dimensions is decoupled from the patient's identity, and a key, password, access code or other security measure(s) is required to retrieve the information and/or match the patient's facial dimensions with the patient's identity. Optionally, a further security measure may be to limit which type(s) of data a given party has authority to access; and/or to put each individual type of data behind a password as well.

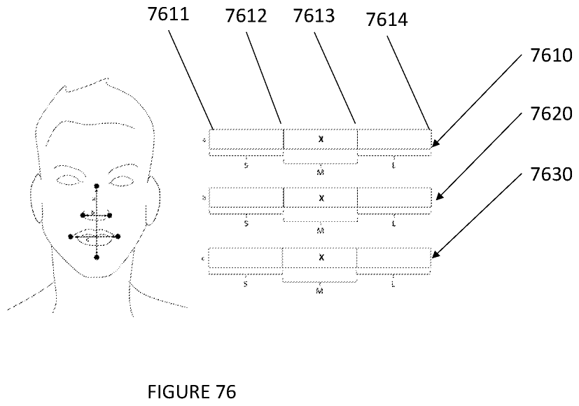

An example of the patient interface selection is now described with respect to FIG. 76. FIG. 76 shows a patient interface size selection for a full face mask. When fitting a full face mask, some examples of required patient facial dimensions are: dimension (a) from the nasal bridge to the lower lip; dimension (b) the width of the nose; and, dimension (c) the width of the mouth.

In FIG. 76, patient interface sizing information is provided for each of the required facial dimensions for patient interface sizes Small(S), Medium (M), Large (L). For some mask categories, additional sizes may be available, for example Extra Small (XS) or Extra Large (XL). For each facial dimension the patient interface sizing information provides a range of dimensions suitable for each patient interface size. For example, for dimension (a) size Small has a dimension between 7611 and 7612, for dimension (b) size Medium has a dimension between 7612 and 7613, for dimension (c) size Large has a dimension between 7613 and 7614. Labels 7611 7612 7613 7614 represent facial dimensions on the charts. The charts may be provided in millimeters (mm) or another unit of length.

The facial feature dimensions of the patient are compared with the patient interface sizing information for each of the required facial dimensions. In FIG. 76 the patient facial dimensions are displayed as an X against the relevant sizing information for the relevant facial dimension. In the example shown in FIG. 76, for dimension (a) the patient facial dimension falls within the dimension range for Medium. For dimension (b) the patient facial dimension falls within the dimension range for Medium. For dimension (c) the patient facial dimension falls within the dimension range for Medium. All patient dimensions are consistent with a single mask size. The patient requires a Medium sized patient interface.

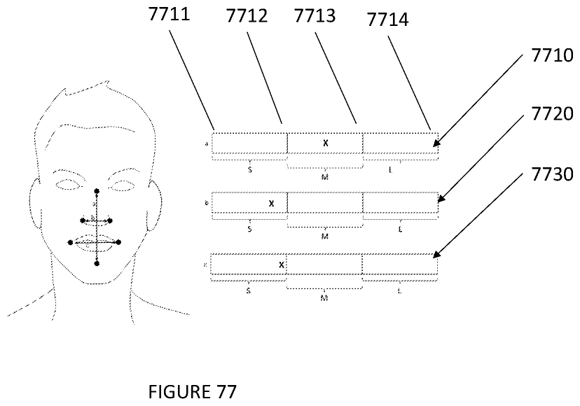

In other examples, all patient facial feature dimensions may not be consistent with a single patient interface size. In FIG. 77, the patient facial feature dimensions are not consistent with a single patient interface size. FIG. 77 again shows example sizing information for a full face patient interface and illustrates mask sizing information for dimension (a) from the nasal bridge to the lower lip; dimension (b) the width of the nose; and, dimension (c) the width of the mouth. In the example shown in FIG. 77, for dimension (a) the patient facial dimension falls within the dimension range for Medium. For dimension (b) the patient facial dimension falls within the dimension range for Small. For dimension (c) the patient facial dimension falls within the dimension range for Small. All patient dimensions are not consistent with a single mask size.

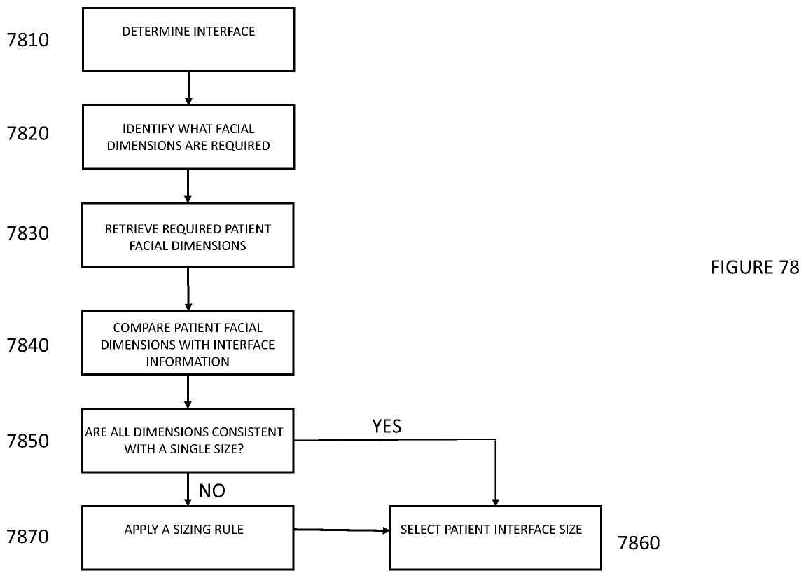

FIG. 78 is a flow diagram showing the steps to select a patient interface for a patient. At 7810, the system identifies which patient interface category is required for the patient. This determination may be made by assessing patient responses to a questionnaire, or based on a previous patient interface prescribed to the patient, patient selection, clinician selection, or another means. At 7820 the system identifies which patient facial dimensions are required in order to select a size of patient interface for the patient of the determined interface from 7810. Typically, the system retrieves this information from a lookup table including information of the type shown in Table 1 (above).

At 7830 the system retrieves the required facial dimensions of the patient. The facial dimensions may be retrieved using the image techniques described herein. In other systems the facial dimensions may be retrieved from a database containing patient facial dimension information. In other systems the patient facial dimensions may be provided by the patient or by a clinician.

At 7840 the system compares patient interface sizing information with the retrieved patient facial dimensions. Patient interface sizing information for different sized patient interfaces is compared with the retrieved patient facial dimensions to determine which patient interface size provides the best fit for the patient. At 7850, the system determines whether all patient facial feature dimensions are consistent with a single patient interface size. If all patient facial feature dimensions are consistent with a single patient interface size at 7850, as in the example of FIG. 76, then the system determines that the patient requires a patient interface of that size at 7860. If all patient facial feature dimensions are not consistent with a single patient interface size at 7850, as in the example of FIG. 77, the system applies a sizing rule at 7870 to determine which patient interface is suitable for the patient.

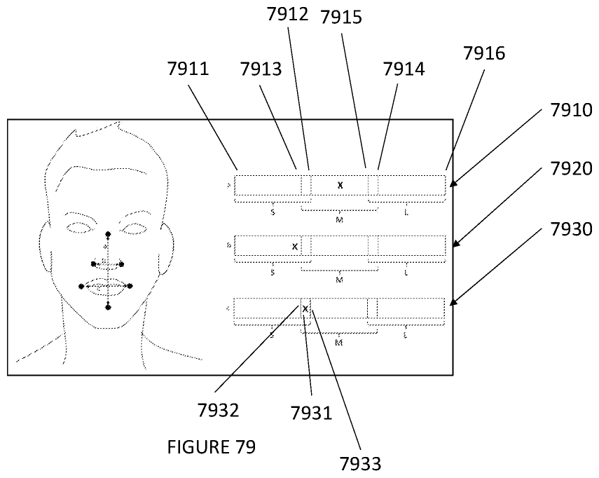

In some embodiments there may be overlap in the patient interface sizing information between sizes. For example, referring now to FIG. 79, for dimension (a) size Small has a dimension between 7911 and 7912, for dimension (b) size Medium has a dimension between 7913 and 7914, for dimension (c) size Large has a dimension between 7915 and 7916. Again, labels 7911 7912 7913 7914 7915 7916 represent facial dimensions, for example provided in millimeters (mm) or another unit of length.

In the example of FIG. 79 a patient having a dimension for feature (a) of between 7913 and 7912 would meet the sizing requirements for both a Small and a Medium patient interface for feature (a). In the example of FIG. 79, the patient facial feature dimension for feature (c) meets the sizing requirements for both a Small and a Medium patient interface. This is illustrated in FIG. 79 by the patient dimension for feature (c) 7931 falling in the overlapping dimension range between 7932 and 7933. In situations where patient dimensions for a feature fall in an overlapping dimension region which meets the requirements for more than one size, the system may apply a sizing rule to determine which patient interface is suitable for the patient.

In some examples the sizing rule at 7870 may be based on a dominant facial dimension related to the determined interface from 7810. For example, if dimensions are not all consistent with one single size, the size of the dominant feature may override the size of the other features leading to a selected patient interface size corresponding to the dominant feature size. The dominant feature may be based on a feature most important for comfort or most important to ensure the interface is effective for delivery the intended therapy, or the dominant feature may be based on any other consideration such as personal preference.

In situations where the dominant feature falls within an overlap of patient interface sizing information such as for feature (c) of FIG. 79, the final patient interface size selected may be left open to the user or be presented as either size of the overlap that the dominant feature falls within. In other examples where this overlap occurs, the final size selected may be based on the non-dominant features. For example, the final size may be based on the most common size between the non-dominant features. In other examples, the final size selected may be a majority across all the determined sizes of the facial dimensions. In the example of FIG. 77 this would result in a size S being selected as two out of the three facial dimensions fall within the S sizing information ranges.

In a further example, the final patient interface size selected may be based on the largest size amongst the identified facial dimensions for the determined interface. In the example shown in FIG. 79 this would be size M as the largest size amongst the features a), b), and c) is a M.

In an example where the system determines that (following the selection/sizing process) multiple sizes may be suitable for a patient multiple choices could be shown to a clinician and/or the patient. And the patient and/or the clinician is prompted to select a patient interface from the multiple choices. In borderline cases a clinician for example may be provided with further insight into what facial measurements have been detected, possibly their dimensions, and what sizes they map to. This may be sent/shown behind the scenes to a clinician for feedback on sizing and provides clinicians with data to have a discussion with patients about their sizing and to make an informed decision. In the example of FIG. 79, the extra information about the dimensions of the individual features might be quite useful to a clinician and/or a patient—i.e. where one measurements e.g. mouth width (c) is borderline and could be fit to two different sizes.

In situations where all patient facial feature dimensions are not consistent with a single patient interface size the rules for size selection may identify an overriding dimension that determines the overall “appropriate size”. For instance, this may be the “largest” dimension—for example if the width of the mouth is a Large but the dimensions of the other facial features are within the dimension range of a Medium or Small, the system may recommend a Large mask size.

In other examples, certain dimensions may be considered to be dominant. So, for example in the case of a full face mask, if the dominant dimensions is the width of the nose, if for a patient the dimension of the width of the nose is within the dimension range for a Medium, the patient is recommended a Medium sized patient interface, regardless of the dimensions of the other features. Different rules may be applied for different mask categories. Combinations of rules may be applied during mask selection.

In some examples the rule for selecting a patient interface size may be based on a combination of the determined facial dimensions. In the example shown in FIG. 79 this could be taking the summation of the dimensions (a), (b), and (c) and comparing this summation with sizing information to determine a suitable patient interface size.

There may be an overlap of suitability between different patient interface sizes for a patient. For example, for a given patient a S and M might both provide an effective seal or comfort level or suitable size for the intended therapy. To illustrate where there is an overlap of suitability of multiple patient interface sizes for a patient the sizing chart may include overlap zones. For example overlap zone 8040 is used to illustrate when both a Small and Medium patient interface are suitable for a patient. Overlap zone 8050 is used to illustrate when a Medium and Large patient interface are both suitable for a patient. By providing the patient with information that multiple patient interface sizes are suitable, the patient may select a patient interface size by personal preference, personal experience, for example the patient may be more comfortable one size or the other or know from experience that one size provides for better therapy, or just prefer a particular size patient interface for some other reason.

Other reasons for presenting an overlap may be related to tolerances in manufacturing and/or sizing/measuring.

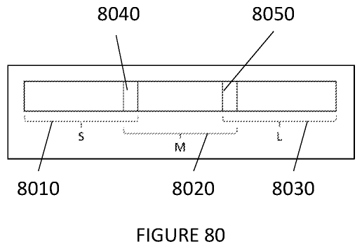

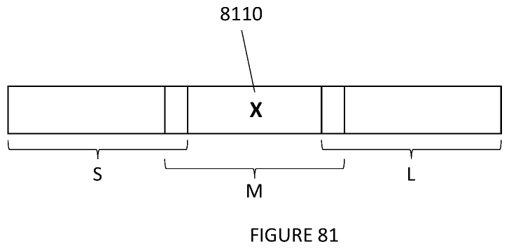

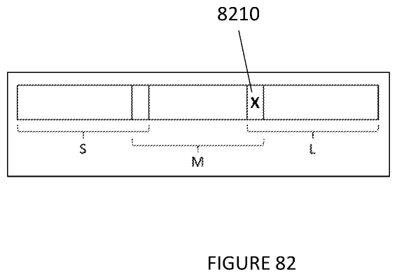

FIGS. 81 and 82 provide examples of patient interface sizing recommendations for presentation to a patient. In FIG. 81, the sizing chart of FIG. 80 is used, showing ranges for Small, Medium, Large patient interfaces and including overlapping size ranges for Small and Medium and also for Medium and Large. In the example of FIG. 81, indicator 8110 is presented in the sizing range for Medium. This indicates to the patient that the recommended patient interface size is Medium. In FIG. 82, the sizing chart of FIG. 80 is used, showing ranges for Small, Medium, Large patient interfaces and including overlapping size ranges for Small and Medium and also for Medium and Large. In the example of FIG. 82, indicator 8210 is presented in the sizing range which overlaps between Medium and Large. This indicates that both a Medium sized patient interface and a Large sized patient interface are both suitable for the patient. The patient may use personal preference to select a patient interface size. And/or the clinician may present a recommendation to the patient.

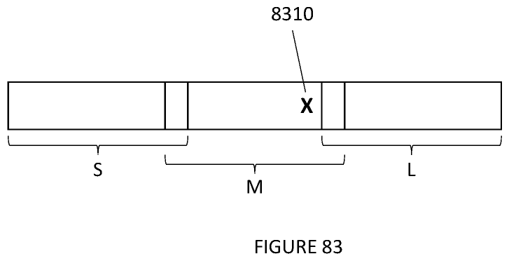

The position of the indicator on the sizing chart may provide an indication of the suitability of that patient interface to the patient. For example, an indicator appearing in the centre of a sizing zone may indicate a high suitability of that patient interface size for the patient compared with other sizes. For example the indicator 8110 in FIG. 81 appears in the centre of the Medium sizing range. This may indicate a high suitability of a Medium for the patient. In the example of FIG. 83 indicator 8310 is clearly within the Medium range, indicating that the recommended patient interface size for the patient is a Medium. However, indicator 8310 is positioned between the centre of the Medium range and the Medium/Large overlap region. This indicates that the patient is a Medium but is towards the Large end of the sizing scale. In FIG. 82, indicator 8210 is presented in the sizing range which overlaps between Medium and Large. This indicates that both a Medium sized patient interface and a Large sized patient interface are both suitable for the patient.

In some examples the suitability level may be presented as a percentage figure or in another way.

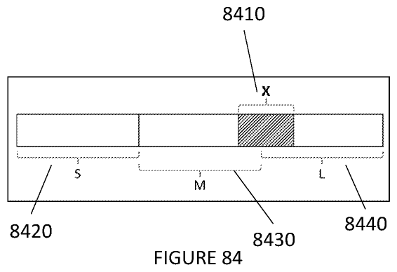

In some examples, the indicator of the recommended size for the patient may be presented as a range on the sizing chart to demonstrate some tolerance levels or uncertainty or flexibility in the recommendation. In FIG. 84 the indicator is shaded region 8410 illustrating a recommended size range on the sizing chart. In FIG. 84, the sizing charts for the Small 8420 Medium 8430 and Large 8440 patient interfaces do not overlap. Indicator 8410 overlaps the Medium range 8430 and the Large range 8440 indicating that the patient is suitable for a Medium or Large size patient interface. Indicator 8410 overlaps more with the Large range than the Medium range indicating the suitability score may lean more towards the Large patient interface.

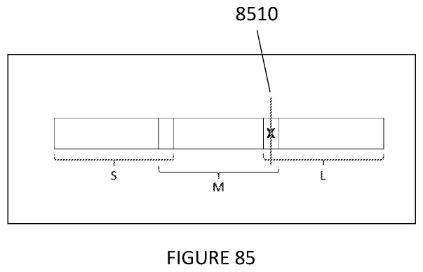

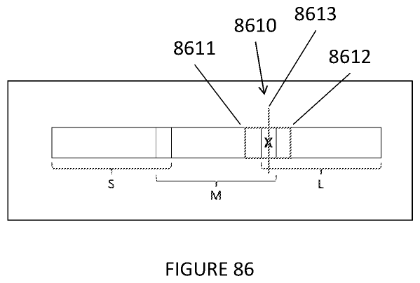

In the example of FIG. 85, indicator 8510 is a “line” showing the exact point where the user is positioned on the sizing scale. In the example of FIG. 86, indicator 8610 is represented as a slider on the sizing chart with the patient's actual size in the middle 8613, and then a “tolerance range” to either side of this, defined between 8611 8612 to indicate a flexibility in what size is suitable for the patient.

In situations when the system determines that a user is suitable for multiple patient interface sizes, for example a Small and a Medium, additional information may be provided to assist the patient or clinician with selecting the patient interface size for the patient. For example, the system may display all of the relevant dimensions of the facial features. These may be displayed with respect to individual sizing charts, for example as shown in FIG. 79. The system may specify the overall size (of the mask), and then the individual facial feature dimensions (and corresponding mask sizes), The additional information may be presented in the form of a click, drop down display which is presented after the initial sizing indication. For example, in the case of FIG. 86, after selection and sizing, the clinician and/or patient is shown the sizing chart of FIG. 86 including slide 8610 which overlaps the Medium and Large fitting recommendations. Upon selecting the indicator 8610, for example by clicking on indicator 8610 on the display, sizing information of the type shown in FIG. 79 showing the patient facial feature dimensions relative to the recommended sizing dimensions for each facial feature is displayed. This additional sizing information may assist the patient or clinician to make a more informed decision of patient interface size in situations when more than one patient interface size would provide the patient with an adequate seal or comfort level or suitability for the intended therapy. The sequence of sizing information may provide a primary display and then a secondary “breakdown” display.

In some implementations, at the initial stage of patient interface selection, for example after initial questionnaire responses have been received, the system may display two patient interface categories to the patient. These patient interface categories are suitable for the patient based on the responses to the questionnaire. For example, the options may include a nasal mask (by default; usually the top-ranking nasal mask), AND either a pillows mask or a full-face mask, depending on questionnaire answers. The presented mask categories are the most suitable patient interface categories for the patient based on the patient responses. Typically, these are presented without a ranking or indication of which one is better, just to provide multiple suitable options to patients. The system may retrieve relevant facial dimensions to fit one or both of the patient interfaces and provide sizing recommendations for one of the patient interfaces or for both of the patient interfaces.

### Re-Scan

After a patient has been initially sized for a patient interface and the patient has used the patient interface for a period of time, systems may include subsequent re-scanning processes to check that the patient remains in the most appropriate mask size. Such re-scanning processes may form part of the ongoing patient journey and aftercare service of a dealer or patient interface manufacturer. The re-scanning process may be provided within a patient interface management application, for example an app for operation on an electronic device, for example a mobile phone or tablet.

The re-scan may be prompted by the dealer or patient interface manufacturer periodically, for example once or twice a year or after a predefined number of therapy sessions or usage sessions or uses, or may be prompted at a predefined time period after the initial mask sizing or wear. Alternatively, the re-scan may be initiated by the patient, for example if the patient is experiencing discomfort during use of the patient interface, or if the patient requires a replacement patient interface or simply if the patient wishes to re-evaluate their selected patient interface.

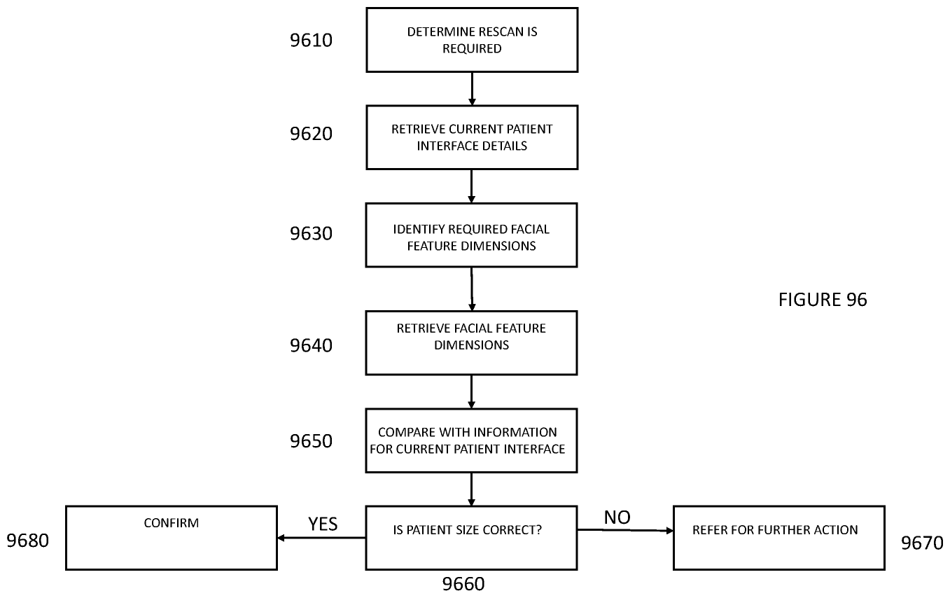

The steps taken during an example re-sizing process are now described with reference to FIG. 96. At 9610 the system determines that a re-scan is required. As described above, the prompt for a re-scan may be initiated by the dealer or mask manufacturer or by the patient or automatically by an application. The re-sizing process may be an option within the patient interface app.

At 9620, the current patient interface details are retrieved. The current patient interface details include at least one of patient interface category, patient interface size. Details may also include the individual dimensions of the patient's facial features as previously calculated. Further information, for example patient interface ID, may also be retrieved. This patient interface information is stored in a database and associated with the patient. (As previously noted, storage may occur in a decoupled or anonymized fashion, with a security key or similar. In some examples, even the retrieval and use of the data at the presently-describe step may occur in a partly or fully anonymized fashion, without reference to the identity of the patient). The database may be stored locally on the patient device or externally on a dealer server or retrieved via a communications network. The system also retrieves the patient interface information for the patient interface category and size, for example from a product database. The patient interface information includes the ranges of facial dimensions suitable for the patient interface category and size. At 9630 the required facial dimensions associated with the current patient interface are identified. For example, if it is identified that the patient is currently prescribed a full face mask then the system identifies that the required facial dimensions are for example: the dimension from the nasal bridge to the lower lip; the width of the mouth; and, the width of the nose.

At 9640 the system retrieves the facial dimensions. The system may use any of the processes described herein to retrieve the required facial dimensions of the patient. This may involve capturing a single front facial view of the patient or facial views from multiple angles, depending on the required facial dimensions. During the facial dimension retrieval step, the system may run a full procedure or may run a reduced facial dimension retrieval process in which only the relevant facial dimensions are retrieved. Such a reduced process may reduce the time and the processing required.

At 9650 the retrieved patient facial dimensions are compared with the patient interface information for the current patient interface size and at 9660 the system determines whether the retrieved patient facial dimensions are within the range of the patient's current patient interface category and size. If the dimensions are within the range of the patient's current patient interface then the system determines that the patient is using the correct patient interface at 9680. If the patient's facial dimensions are not within the range of the patient's current patient interface the system refers back to the dealer or manufacturer for further action (or, in some embodiments, proceeds to recommend or suggest a different size of patient interface to the patient (or to the clinician or other relevant party)).

In another example, the re-scan process may comprise a similar process for selecting a patient interface as described elsewhere in the disclosure as used by a patient for their initial scan. In this example, step 9660 may compare the patient interface size determined by the re-scan process with the patient interface size retrieved at 9620 and confirm that the patient interface size is correct if these two sizes match, and refer for further action if a mismatch is determined.

In another example, the re-scan process may comprise a similar process for selecting a patient interface as described elsewhere in the disclosure as used by a patient for their initial scan such as the methods described with reference to FIGS. 74 to 86. In this example, step 9660 may compare the patient interface size determined by the re-scan process with the patient interface size retrieved at 9620 and/or compare the dimensions and respective size on the size charts of facial features between the re-scan and the initial scan. Patient interface size may be confirmed as correct if these all sizes match. If any of the patient interface size or individual sizes of facial features do not match between the re-scan and the patient's initial scan, the system may take the refer for further action step at 9670.

The advantage of the re-scan procedure is that the patient can maintain confidence that the patient interface they are prescribed remains the most suitable for their therapy. Re-scans can be compared between initial scan and second scan, or between third scan and initial scan, or between second scan and third, between subsequent scans, across all scans. The patient may be scanned at any time based on the triggers described herein. Dimensions and sizing can be tracked, and can provide history of scan data, compare between any set of that data, and use that info to confirm patient interface sizes.

The patient interface sizing process during re-scan may be a reduced processing version of the initial sizing process, providing a quicker process requiring less computer processing. In some examples, the re-scan process may be run entirely locally on the device without requiring a connection to a communications network, for example if the patient device stores the current patient interface product details and may only connect to the dealer or manufacturer servers if there is an inconsistency with the measured patient facial dimensions or determined patient interface size and those of the patient's current patient interface or any of the mismatches described above.

### Animation of the Face to Replace the Video Image.

In some examples described herein the video image captured by the camera is displayed on the display screen to assist the patient during patient interface sizing. In some example systems the video image of the patient is replaced with an animation. The animation may be a caricature. The animation may be an animated face. The orientation of the animation may replicate the orientation of the face and so the patient is provided with the same orientation information as with the video image recorded by the camera. The system uses the same processes as described herein to calculate orientations, angles, and dimensions using the image data captured by the camera. But the image data is replaced with animation for display on the screen.

### Headgear Sizing

As described above, patient interfaces may be held to the face of the patient using headgear. For example, as shown in FIG. 2 and described above, headgear includes a strap 220A extending around the jaw and/or cheek and neck of the patient and a second strap 230A extending around the top of the head of the patient. The size of the headgear is also a consideration for effectiveness and comfort and the headgear may also be provided in different sizes depending on the size of the head of the patient.

In order to fit the headgear correctly for a patient, systems calculate head dimensions relevant to fitting the headgear. Such dimensions may include, for example, dimensions across the forehead, such as the width or height of the forehead, or dimensions related to the circumference around different parts of the head, for example above the ears or below the ears around the neck. Other dimensions may include the height of the head or other vertical measurements associated with the back of the head. Such dimensions may be calculated using landmarks on the face or head in the same way as the dimensions such as the width of the mouth are calculated. The dimensions may be calculated using two dimensional images or facial simulations/reconstructions using face mesh or other techniques to determine dimensions around the side of the head.

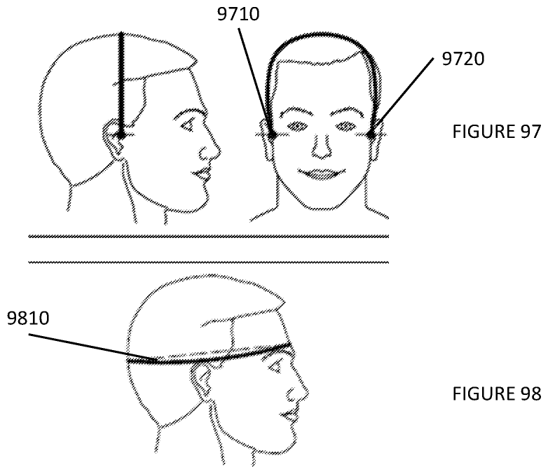

Particular landmarks on the head or face may be relevant for sizing headgear. FIG. 97 shows the bitragion coronal arc. This is described by the surface distance between the right tragion 9710 and the left tragion landmarks across the top of the head in the coronal plane. FIG. 98 shows the head circumference just above the ridges of the eyebrows (supraorbital ridges).

Identification of the landmarks and the angles of the images required in order to obtain the dimensions for these features are determined and the system prompts the user to capture the relevant images using the processes described herein.

It may also be possible for the system to obtain a partial indication of the relevant dimension, and apply algorithms to infer or calculate or estimate the full dimension. For instance, for head circumference, a frontal image may yield the right tragion 9710 and the left tragion 9720 landmarks (and the system may obtain the distance/dimension between same using the protocols described herein). And the system may use an algorithm or formula (such as based on statistical head shapes) to infer, from this frontal distance, an approximate circumference of the head. In other embodiments, the actual circumference of the head (or other relevant dimension) may be directly obtained by requiring facial/head images from a variety of angles.

The head dimensions are compared with dimensions or sizing information of headgear to determine the correct size for the patient. Head dimensions may also be compared with different headgear types to select the correct type and size of headgear for the patient.

The appropriate headgear size may be outputted to the user separately from, or in conjunction with, the appropriate interface size.

### Clinician Mode

An example system is now described in which a clinician initiates and/or manages the patient interface sizing process. The following description is described with a clinician managing the patient interface sizing process. In other examples the patient interface sizing process may be initiated or managed by other third parties, for example the patient or other parties. The use of the patient interface sizing system by a clinician provides clinicians with a digital sizing tool that can be used in place of manual sizing tools.

The patient interface sizing process may be executed in the form of a software application and may be stored and/or accessed/and or executed via a user device of the clinician. The user device may be, for example a mobile phone, tablet, desktop computer or other computing device.

In an example embodiment a system for sizing a patient interface for a patient for use with a respiratory therapy device is provided, the patient interface suitable to deliver respiratory therapy to the patient, comprising the steps of: Initiating a patient interface sizing application; Identifying at least one patient interface type required for sizing for a patient; determining at least one facial image type required to size the at least one patient interface type required for sizing; executing an image capture sequence to capture the at least one facial image type required for sizing and calculating a dimension of at least one facial feature required for sizing for the patient; based on the calculated dimension, determining a suitable size, for the patient, of each of a plurality of patient interfaces within said patient interface type.

A further example embodiment provides a system for sizing a plurality of patient interfaces for a patient for use with a respiratory therapy device, the patient interfaces suitable to deliver respiratory therapy to the patient, comprising the steps of: Initiating a patient interface sizing application; Identifying a plurality of patient interfaces required for sizing for a patient; For each of the plurality of patient interfaces, determining at least one facial feature whose dimension is required in order to size the respective interface; determining at least one facial image type required in respect of each of the at least one facial feature; executing an image capture sequence to capture the at least one facial image type; using the captured at least one facial image type, calculating the dimension of each of the at least one facial feature; based on the calculated dimension of each of the at least one facial feature, determining a suitable size, for the patient, of each of the plurality of patient interfaces.

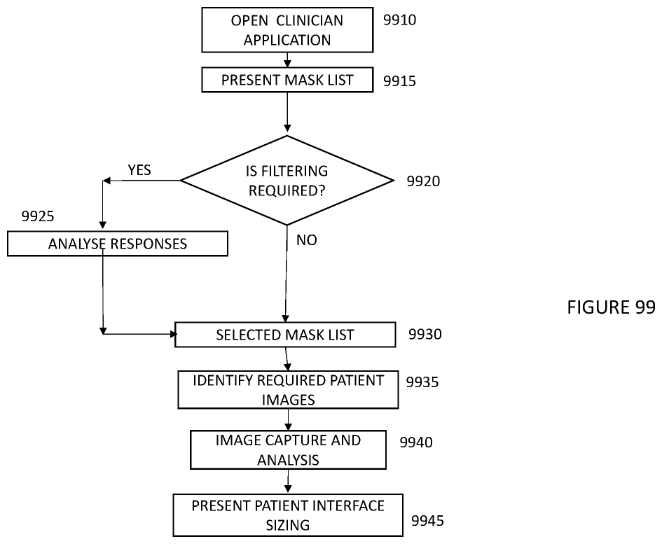

The steps executed in a patient interface sizing system operated by the clinician is now described with reference to the flow diagram of FIG. 99. At 9910 patient interface sizing application is accessed by a clinician on an electronic device, for example a mobile communications device. The patient interface sizing application can exist as a standalone software application or as a module within a more general application. The patient interface sizing application may include access restrictions, for example password requirements, biometric ID requirements, or other security features, to restrict access to authorised users, for example clinicians.

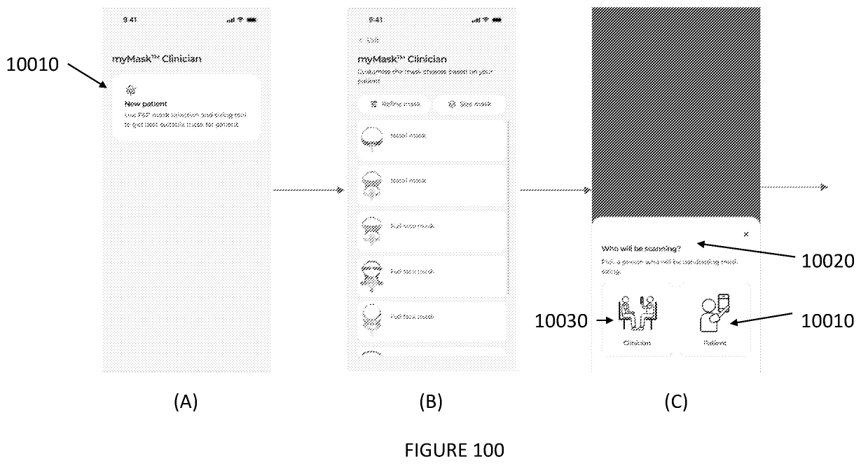

FIG. 100 is an example of a user interface presented to the clinician after accessing the patient interface sizing application. Access to the patient interface sizing application may be restricted by password protection or by biometric identification. After granting access to the application, the application may present a welcome screen as shown in FIG. 100(a). In the example of FIG. 100(A) the system presents an option for a clinician to use the patient interface sizing application to size a patient interface for a patient. Further options may be presented to the clinician within the application in other examples. In the example of FIG. 100(a) the option is presented as a New Patient icon 10010. The clinician can select the New Patient icon 10010 via a user input device, for example a keypad or touch screen. Selection of the New Patient icon by the clinician initiates the patient interface sizing process. The application may also include options to initiate the patient interface sizing application for existing patients, for example for a re-scan or a for a first scan for an existing patient.

At 9915, the application may display a mask list to the user. The mask list may display a full catalogue of masks, including, for example nasal masks, full face masks, under nose masks etc. The masks may be identified as a mask category, for example a nasal mask or full face mask. Alternatively, or additionally, specific mask models may be identified within each mask category, for example within the nasal mask category, various mask models may be identified.

At 9920 the application may provide an option to filter masks within the mask list to a sub-set of masks. If mask filtering is provided, at 9925 the system may display one or more questions to filter the masks and to produce a sub-set of the mask list. The system may receive input from the clinician via an input device in response to the questions. In one example, the clinician provides input to complete a questionnaire. The system filters the masks for selection based on the input responses. The questionnaire may include questions identifying patient mask preferences, and/or questions to do with patients' sleep tendencies and other matters which may affect which masks are and are not suitable for them.

In some examples, the application may include an option to allow the clinician to filter masks within the mask list manually by selecting a sub-set of the masks, for example based on patient preferences or clinician preferences. User (i.e. clinician) responses may be received via a user interface, for example a user input device (for example a keyboard, touchscreen, or other input device).

In some systems, filtering of the mask list may not be provided at 1920.

If filtering is not required or if filtering of the mask list is not provided the system proceeds with the full mask list.

At 9930 the mask list is finalised (either filtered or unfiltered, depending on the application and user inputs). The mask list represents the catalogue of masks for which the patient will be fitted. This may include all mask categories and mask models or a subset of available mask categories and/or mask models, depending on whether the mask list has been filtered or not.

At 9935 the system identifies which measurements, and hence which patient images, are required in order to size the masks. The system may identify the required patient images in order to fit all masks in the mask list. One of the benefits that clinician mode provides is a “one scan for all masks” workflow. The system completes a single scan (i.e. image capture) sequence (see 9940) of the patient to get their facial dimensions which are then used to size all the masks in the mask list. As described in detail above, different mask categories and different mask models require different measurements in order to determine a correct fit.

For example, as described above with reference to FIG. 24, for a full face mask a front facial image is required and the relevant dimensions are: the dimension 2430 from the nasal bridge to the lower lip; the width of the mouth 2450; and, the width of the nose 2440. When a patient requires a full face mask, these three dimensions should be obtained and compared to patient interface fitting data for full face masks to select a full face mask which fits the patient.

For under nose nasal masks, as described above with reference to FIG. 26, the relevant facial features are nose width 2620 and the nasal length 2630 (i.e. nasal depth). This is because the seal sits under the nose and wraps around under the nose. In order to fit an under nose mask, these two dimensions are required and compared to patient interface fitting data for under nose nasal masks to select an under nose nasal mask which fits the patient. The dimensions for nose width and nose depth are obtained from a front facing image and/or an underside image.

The system identifies which mask categories and mask models appear in the mask list. The system identifies which measurements, and thus patient images, are required to size each mask and compiles a full patient image list, this list may or may not be displayed to the user. The system creates or retrieves a scanning (i.e. image capture) sequence for capturing all patient images appearing in the patient image list.

In some example systems the system retrieves a standard scanning sequence to capture the patient images required to size all mask categories and types (regardless of whether the mask list has been filtered). The benefit of capturing images to size all mask categories and types is that if the patient wishes to change mask category after trying a mask, a further scan to size masks may not be required.

A benefit of examples of the system is that clinician mode automatically sizes for all the masks and displays this information to the user allowing them to compare between different mask types and sizes. If subsequently the patient finds their initial choice of mask type was not optimal, they can change to another type and not have to repeat the sizing process.

After identifying which images are required at 9935, at 9940, the application executes a scanning (image capture) sequence. The scanning sequence presents instructions to the user on the display to guide the user to position the camera (and/or position the head/face relative to the camera) to correctly capture the required images of the patient.

The application may provide an option for the user to select which camera to use, when the electronic device includes more than one camera. For example, on a mobile phone having a rear facing camera (for traditional image capture) and a front facing camera (for selfie mode). The application may receive input from the user which allows the user to select whether to use the rear facing camera, typically in the case of a clinician or other third party capturing the images, or the front facing camera (selfie mode), typically in the case of the patient self capturing the images. The user instructions and calculations for angles and distances etc within the scanning sequence may vary depending on whether the user is using the front camera or the rear camera. The scanning sequence may include a specific order for image capture, specific instructions, specific animations, and/or specific measurement criteria designated to each camera. On receiving selection of a camera, the system identifies the required scanning sequence and executes the scanning sequence allocated to the selected camera.

In the example of FIG. 100 (c), the application displays a question to the user “Who will be scanning?” 10020 and provides the user with two options, “Clinician” or “Patient”. Selection of “Clinician” is an instruction to the application to use the rear facing camera of the device (traditional image capture mode), selection of “Patient” is an instruction to the application to use the front facing camera of the device (selfie mode). Other examples provide different input options to allow the user to identify which camera they wish to use for image capture.

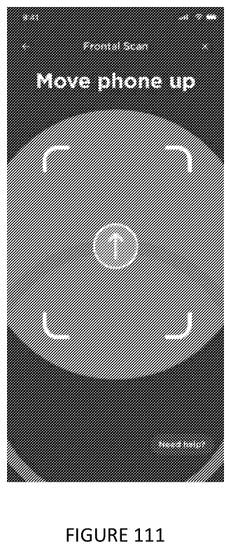

The image capture process typically requires the same image angles (as between the relevant camera and the face/head) to calculate facial dimensions, whether the front camera or rear camera is being used, but the order of the steps, the instructions presented to the user, and/or the animation to guide the user may differ depending on whether the front camera or the rear camera is being used. Example instructions and processes used during use of the front camera (selfie mode) are described above with respect to a patient capturing the images (for example with respect to FIGS. 16 to 18, 59 to 62. Some example instructions and processes used during use of the rear camera (traditional mode) are described below with respect to FIGS. 111-113.

Image capture using the rear camera may involve different usability features to instruct the user to position the device (and/or position the patient's head relative to the device) in the correct position for image capture. But in general, the same image capture criteria needs to be satisfied—i.e. correct (relative) height, relative angle, and distance to face. As discussed below, these image capture criteria may be satisfied via a somewhat different process in clinician mode than in self-scan mode. For instance, the requirement that the phone be vertical may no longer apply (and thus “correct relative height” may refer to the height of the phone relative to a general plane of the front of the face, or relative to a normal to that plane; rather than being strictly a height in a “vertical” direction). Also, in clinician mode the process may, firstly, require the patient to make a “coarse” movement to bring their face into the vicinity of the correct orientation; followed by the clinician making “refining” movements with their phone, to improve or perfect the relative alignment/orientation of the phone relative to the face.

A benefit of the clinician mode is that the system captures images and calculates facial dimensions to allow the system to size the patient for multiple masks. The system may capture multiple required images in a single image capture session by a sequence of positioning instructions to the user. The system may size multiple mask categories and/or multiple mask models. The sizes of all masks may be displayed to the user allowing the user to compare between different masks and sizes.

The patient's sizing/measurement data may be stored in a database. If subsequently the patient finds their initial choice of mask type was not optimal, they can change to another type and not have to repeat the sizing process.

After the image capture process is complete at 9940, the system compares the measured dimensions of the patient with the mask dimensions to determine the correct size masks to fit the patient. This comparison may be performed for the entire supported mask list, or the selected subset of the mask list.

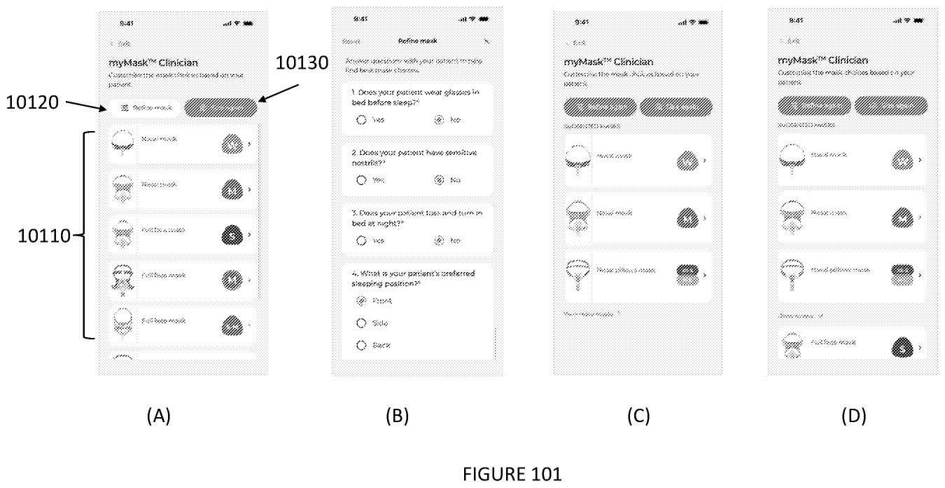

As shown in FIG. 101 (A), the entire mask list 10110 is displayed with recommended sizing for the patient, unless the clinician has prior to scanning “refined” the list by creating a subset of masks, for example by answering a questionnaire or by manually selecting or de-selecting masks.

Recommended patient interface sizes are presented to the user at 9945 (mask list 10110 of FIG. 101(A)). The system may provide an option to filter the list of masks after the scanning process is complete and the images have been captured. In FIG. 101(A) the mask list display includes an icon “Refine mask” 10120. When the system receives a user input for Refine mask it may present a user questionnaire to filter the mask list. An example questionnaire is presented in FIG. 101(B). Such systems have the advantage that a patient may be fitted for all masks in the catalogue but the user can filter the list after image capture and sizing by responding to a questionnaire. The subset of masks that are filtered after completing the questionnaire are displayed. FIG. 101 (C) and FIG. 101 (D) illustrate subsets of masks after completion of the questionnaire. In FIG. 101(D), the top portion of the screen shows sizing for the filtered masks; and the bottom portion of the screen shows sizing for other masks that did not meet the filter criteria, but which the clinician might nonetheless wish to see.

The step of filtering may be performed before or after image capture (And thus before or after sizing). Different user options may be implemented, for example the refinement can be cleared (not shown but a button to clear refinements is accessible). Some systems may provide a user option to view more masks and display a full list of supported masks with corresponding sizes.

The system presents a “Size again” icon. On receiving input for “Size again” the system re-initiates the scanning (image capture) sequence.

### Sizing Charts

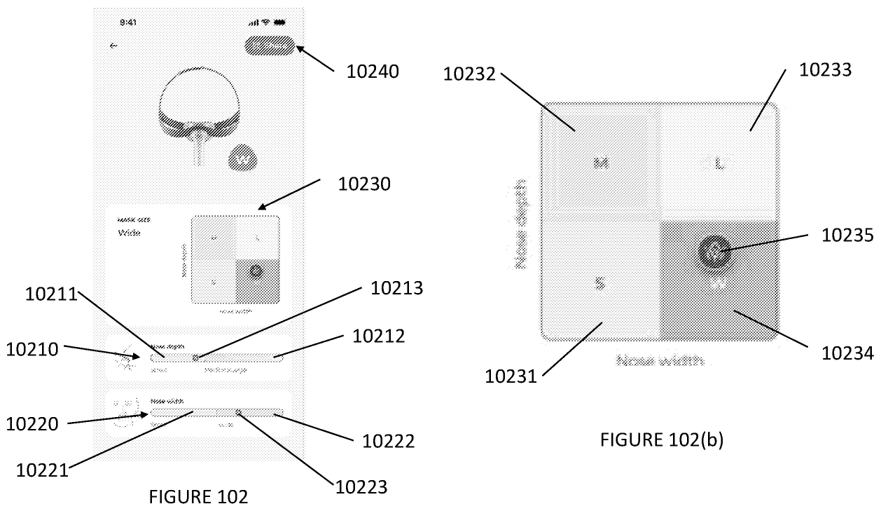

FIGS. 102 to 109 provide examples of sizing charts used to present patient interface sizing information. The sizing charts include an indicator (icon) to identify the most appropriate size patient interface for the patient based on the patient's facial dimensions. These sizing charts provide a clinician, patient or other interested party with a visual indication of how the patient's facial dimensions compare with the different mask sizes and enable the clinician, patient or other interested party to make an informed decision in selecting a patient interface size.

In the example of FIGS. 102 to 109 multi-dimensional sizing charts indicate the expected fit of a mask for a patient. The sizing charts may provide clinicians with greater understanding of fit of a patient interface for a patient. This enables clinicians to make more informed decisions on size selection for patient interfaces and can be helpful to assess size for a remote fitting or any fitting assessment when a patient or patient interface are not available for a manual fitting.

An example embodiment provides a patient interface sizing system for selecting a patient interface size for a patient for use with a respiratory therapy device, the patient interface suitable to deliver respiratory therapy to the patient, comprising: identifying dimensions for multiple facial features required to size a selected patient interface; receiving the dimensions of the multiple facial features; comparing the multiple facial dimensions with patient interface sizing data for the selected patient interface to determine a size of the selected patient interface for the patient; wherein one or more sizing rules related to the multiple facial dimensions are dependent on the selected patient interface; and displaying, on a display, an icon representative of the determined size of the patient interface, said icon being superposed on a chart comprising segments representing one or more sizes of the patient interface; said chart comprising at least a first and second axis representing at least a first and second of the identified dimensions.

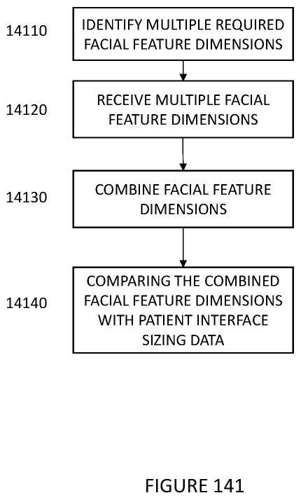

A further example embodiment provides a patient interface fitting system comprising the steps shown in FIG. 141: identifying multiple facial dimension measurements required to fit a patient interface at 14110; receiving the multiple facial dimension measurements at 14120; combining the multiple facial dimension measurements using a combination operation at 14130; and comparing the combined multiple facial measurements with patient interface sizing data for at least one patient interface type to identify a patient interface size for the patient associated with patient interface type at 14140; wherein combination operation is dependent on the patient interface type.

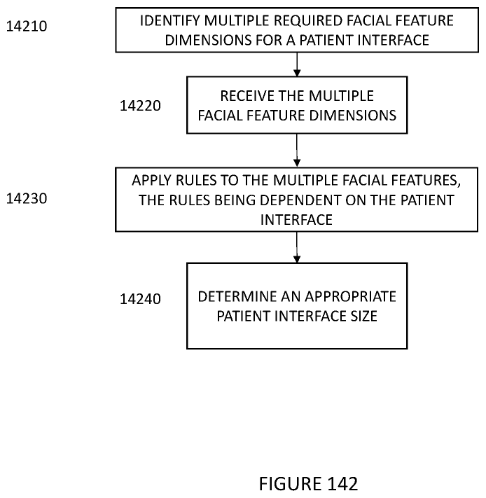

In a further example, now described with reference to FIG. 142, a patient interface sizing method comprising the steps of: identifying multiple facial dimension measurements required to fit a patient interface at 14210; receiving the multiple facial dimension measurements at 14220; comparing the multiple facial measurements with facial interface sizing data using one or more sizing rules related to the multiple facial dimensions at 14230, said one or more rules being dependent on the particular patient interface, to identify a patient interface size for the patient associated with the particular patient interface at 14240.

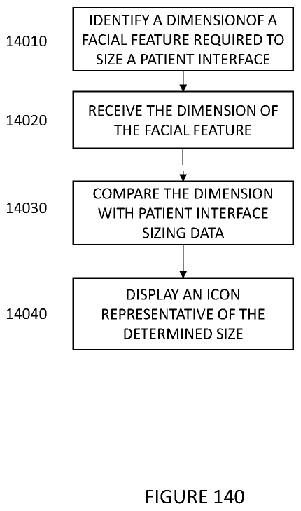

A further example embodiment is now described with reference to FIG. 140 which provides a patient interface sizing system for selecting a patient interface size for a patient for use with a respiratory therapy device, the patient interface suitable to deliver respiratory therapy to the patient. The method comprises identifying a dimension of a facial feature required to size a selected patient interface at 14010; receiving the dimension of the facial feature at 14020; comparing the facial dimension with patient interface sizing data for the selected patient interface at 14030 to determine a size of the selected patient interface for the patient; wherein one or more sizing rules related to the facial dimension are dependent on the selected patient interface; and displaying at 14040, on a display, an icon representative of the determined size of the patient interface, said icon being superposed on a chart comprising segments representing all sizes of the patient interface; said chart comprising an axis representing the identified dimension.

The sizing charts of FIGS. 102 to 109 may be presented to the clinician in response to a request for further information about a patient interface recommendation. In the example screen display of FIG. 101, after the system has obtained a series of facial dimensions from the patient, size recommendations are provided for multiple patient interfaces. As shown in FIG. 101, the patient is fitted as a WIDE for a first Nasal nasal mask, a MEDIUM for a second nasal mask, an XS-S for a nasal pillow mask, a SMALL for a full face mask. The clinician is presented with an option to view further information about the fit of the patient interface. This further information may be presented in the form of a sizing chart. This further information may be accessed via selection of a display icon.

If the clinician is interested to receive further fitting information for the WIDE recommendation for the Nasal mask, the clinician selects the mask within the catalogue screen of FIG. 101. The system receives the clinician input for further information and presents the further information, shown in FIG. 102. FIG. 102 displays further information about the patient fit by displaying sizing charts for the nasal mask. In the case of the nasal mask, two relevant facial dimensions for sizing are nose depth and nose width. The nose width and nose depth of the patient are displayed separately with respect to the mask sizes in separate sizing charts 10210 10220. Nose depth sizing chart 10210 and nose width sizing chart 10220 are one dimensional sizing charts, accounting for the single dimension only.

Each sizing chart 10210 10220 is presented as a one dimensional chart including different mask sizes (for the respective facial dimension in question) in different regions of the chart. In the example of the nose depth sizing chart 10210 the size of the patient interfaces increases from left to right. The region of sizing chart 10211 corresponding to SMALL patient interface size being presented on the left side and the region of the sizing chart corresponding to the MEDIUM/LARGE patient interface size 10212 is presented on the right side. Similarly, the nose width sizing chart 10220 also increases in size from left to right, with the region of sizing chart corresponding to SMALL patient interface size 10221 is presented on the left side and the region of the sizing chart corresponding to the WIDE patient interface size 10222 is presented on the right side. The regions correspond to sizing ranges. The scale for the nose depth sizing chart and nose width sizing chart is not provided in examples 10210 and 10220. The scale of the sizing chart may be a linear scale or may be scaled differently.

An indicator (icon) 10213 10223 is presented on the sizing chart to indicate the patient fit for different patient interface sizes (for the particular dimension in question, e,g, nose depth, nose width). The position of the indicator on the sizing chart identifies where the patient's dimension falls within the particular size. For example, when the indicator is positioned around the middle of a sizing range this indicates a high suitability for that size (for the particular facial dimension), compared with the indicator being positioned towards the end of a size range or in a borderline region between size ranges.

As described above, the relationship between patient interface size and facial dimensions may be more complicated than the comparison of a single facial dimension with dimensions of a patient interface. For some patient interfaces, multiple dimensions are relevant for sizing a patient interface and the combination of these dimensions will determine which patient interface size best fits the patient. Fitting information for patient interfaces for which multiple dimensions are relevant can be represented in a multi-dimensional sizing chart. The sizing chart may be one, two, or three dimensions and may take the form of a graph or other chart type to provide sizing information.

10230 (shown in enlarged view in FIG. 102(b)) illustrates a two dimensional sizing chart for a nasal mask. The sizing chart of 10230 is a combination of the nose width and nose depth sizing charts shown in 10210 and 10220. Within the sizing chart, different regions are associated with different patient interface sizes. The regions indicate a combination of dimensions appropriate to each patient interface size. Sizing charts for different mask types are specific to the fit of those different mask types. By “combination” is meant that the respective dimensions (here nose width and nose depth) are both considered against a set of sizing rules for the particular mask. Those sizing rules will specify how the respective dimensions affect overall size of the mask. For example, one of the dimensions may take precedence over the other, to govern mask size irrespective of what the other dimension is. In another example, one of the dimensions is more important than the other, but if the disparity between the first and second dimensions is over a certain amount, the overall size of the mask will move to the next size up (or down). Accordingly, by “combining” is meant that the respective facial dimensions are considered, in concert, against a set of mask-specific sizing rules that incorporate the respective dimensions.

Sizing chart 10230 is split into four regions 10231 10232 10233 10234, each region representing a different patient interface size. In the example of 10230, the sizing chart includes regions associated with patient interface sizes: Small(S) 10231; Medium (M) 10232; Large (L) 10233; Wide (W) 10234. Sizing chart 10330 is split quite evenly between the sizes with the Small size occupying the bottom left region of the sizing chart; Medium occupying the top left region of the sizing chart; Wide occupying the bottom right region of the sizing chart; Large occupying the top right region of the sizing chart. Size chart 10230 includes nose width on a first (x) axis and nose depth on the second (y) axis.

A patient size indicator 10235 is positioned on sizing chart 10230 to illustrate how the patient's face fits with respect to the mask sizes. The position of indicator 10235 on the sizing chart provides a recommendation of a patient interface size for the patient. The indicator appears in the region of the size recommended for the patient. In the example of FIG. 102, the indicator is positioned within the region corresponding to size ‘Wide’ nasal mask.

Multi-dimensional sizing charts provide the clinician, patient or other interested party with information about the expected fit of a patient interface size for a patient. The position of the patient size indicator may present information about how well the patient interface will fit the patient. For example, an indicator positioned in the middle of a size region may indicate a high suitability of that patient interface size for the patient. An indicator positioned within a size region but which is off-centre from the region is still a recommendation for the patient interface size within which the patient size indicator is positioned, but may give the clinician or patient or other interested party additional information about the likely fit. For example, in the example of FIG. 102, indicator 10335 is within the region for Wide size patient interface but positioned off-centre in the Nose Depth direction and towards the Large size patient interface region. In another example, where an indicator is positioned close to the boundary between two size regions, this may indicate to the clinician that the patient may suit either of those sizes. The clinician may then consult the one-dimensional charts to establish why the patient is borderline between the sizes. The clinician may then discuss options with the patient to determine the most suitable mask; for instance, a given patient may have a preference for a smaller rather than larger size. The fact that the sizing information is presented in both two-dimensional and one-dimensional format gives the clinician a comprehensive understanding of a patient's sizing situation (and the reasons for it, in terms of individual facial dimensions), and enables them to discuss sizing with the patient and consider any sizing alternatives that might also be suitable for the patient.

The axes of the sizing chart may be scaled linearly. Or they may not be scaled linearly. The purpose of the sizing chart may be to convey a visual representation of the recommended fit to the clinician and so may not be scaled, instead configured to represent a visual indication of size.

In other patient interface types, the combination of dimensions may be more complicated, leading to different region shapes within a sizing chart.

The system combines patient facial dimensions to determine the recommended patient interface size. When combining multiple facial dimensions, for example nose width and nose depth to determine a sizing for a nasal mask, the dimensions may be weighted differently in the combination. For example, the nose width may be more significant than the nose depth when establishing patient interface size for a patient. In this case the nose width is the dominant dimension in determining patient interface size. The combination may include other dimension thresholds, for example if nose depth is above x mm then a Small and a Medium are unsuitable, regardless of nose width or other facial dimensions. These dimension weightings are included in the combination. Different mask categories may have different weightings (even if the same dimensions are relevant), for example a nasal mask may weight the nose width and nose depth differently from a full face mask. Also, different mask types within the same category may combine the dimensions differently, for example by applying different weightings. The combinations may be stored in the system in the form of algorithms.

The patient facial dimension combination may be specific to patient interface. This may result in a patient being recommended different patient interface sizes in different patient interfaces.

For example, a first nasal mask may have a fairly generic depth dimension across different sizes meaning that it fits most nose depths, but may be more sensitive on nose width because patient interface sizes have different widths. For this nasal mask the nose width may be weighted more heavily in the combination compared with nose depth. A second, different, nasal mask may have greater variation in nose depth dimensions between sizes and so patient nose depth dimension may be weighted more heavily in the combination.

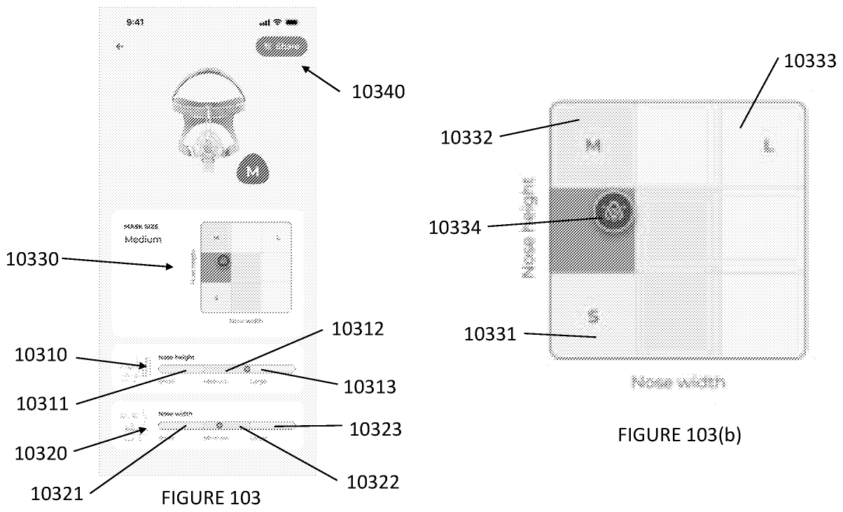

FIG. 103 shows an example of sizing charts for a different type of nasal mask. Although this mask is in the same mask category as the sizing chart in 10230 (i.e. nasal mask) the fit of this mask is different. The sizing charts of FIG. 103 are different from those of 10230.

The relevant dimensions for the mask of FIG. 103 are nose height and nose width. The nose height dimension and nose width dimension are presented individually in one-dimensional sizing charts 10310 (nose height) and 10320 (nose width). The sizing chart for nose height is split into three regions for sizes Small (10311); Medium (10312); Large (10313). In the example of FIG. 103 the patient dimension for nose height falls on the borderline between Medium and Large. The sizing chart for nose width 10320 is split into three regions for sizes Small (10321); Medium (10322); Large (10323). In the example of FIG. 103 the patient dimension for nose height is towards the middle of the Medium region.

Sizing chart 10330 (shown in enlarged view in FIG. 103(b)) is the two dimensional sizing chart for the nasal mask. In the example of FIG. 103, sizing chart 10330 is divided into three regions indicating patient interface sizes: Small 10331; Medium 10332; Large 10333. The regions of sizing chart 10330 are not evenly distributed within the sizing chart and do not cover the same proportional area within the sizing chart. The Large sizing covers around 4/9 of the sizing chart, extending across the large nose width dimension for all nose height dimensions. This is an indication that a large nose width dimension is heavily weighted in the combination for the mask. The Medium patient interface covers 4/9 of the sizing chart. The Medium region covers various section of the sizing chart associated with medium dimension of nose width and nose height. The Small size region 10331 covers only 1/9 of the sizing chart. This covers the bottom left region of the sizing chart associated with small dimensions for nose height and nose width. Indicator 10334 appears in the Medium region of sizing chart 10330 to recommend a Medium patient interface. The position of indicator 10334 is determined by the combination of the facial dimensions for patient for the mask.

Multi-dimension sizing provides a multi-dimensional approach to patient interface sizing. It accounts for the combination of facial dimensions when recommending a patient interface size. The combination of patient facial dimensions may be specific to a mask type or mask category. The combination may be an algorithm to determine the recommended mask size for a patient based on facial dimensions. The combination may be dependent on the particular shape of a mask, the proportions of the mask, the required fit of a mask. Different combinations allow different dimensions to be included in fitting and for different weightings to be applied to different facial dimensions. This provides flexibility in the relationship between facial dimensions and sizes to increase the reliability of remote fitting.

Multi-dimensional sizing charts provide a visual indication of the facial dimensions vs the patient interface size. This can provide clinicians, patients and other interested parties with greater information about the expected fit of different patient interface sizes. Sizing charts may be presented with different axes and different scales. The scale of the axes may be linear, or may not be linear.

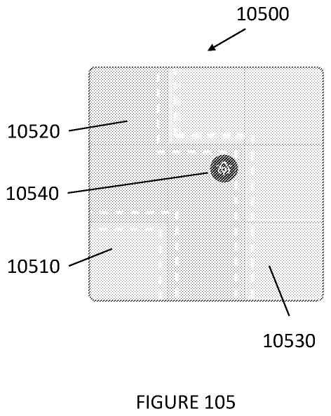

FIG. 105 shows a further example of a sizing chart for a nasal mask. Sizing chart 10500 of FIG. 105 is the same as that shown in FIG. 103. Sizing chart 10500 is divided into three regions representing different patient interface sizes: Small 10510; Medium 10520; Large 10530. As described above, the regions are not evenly distributed within the sizing chart. In the example of FIG. 105 indicator 10540 provides an indication of how the patient's facial dimensions fit against the patient interface sizes. The dashed lines along the edges of each size region indicate that a patient falling in this area may be on the “borderline” between two or more sizes. Such dashed lines (or similar visual representations) may be shown on the sizing chart as a visual cue to the clinician that the patient may be on the border between different sizes. In this situation, as discussed above, the clinician may for instance discuss the matter with the patient. The one-dimensional charts that are also provided may be helpful in this regard, in helping the clinician to understand and explain to the patient why (in terms of respective facial features) they fall on the borderline, and/or why more than one size may be suitable for them.

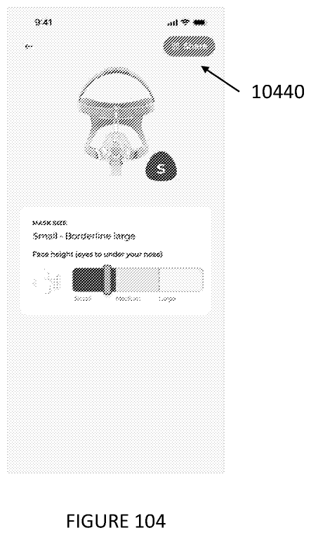

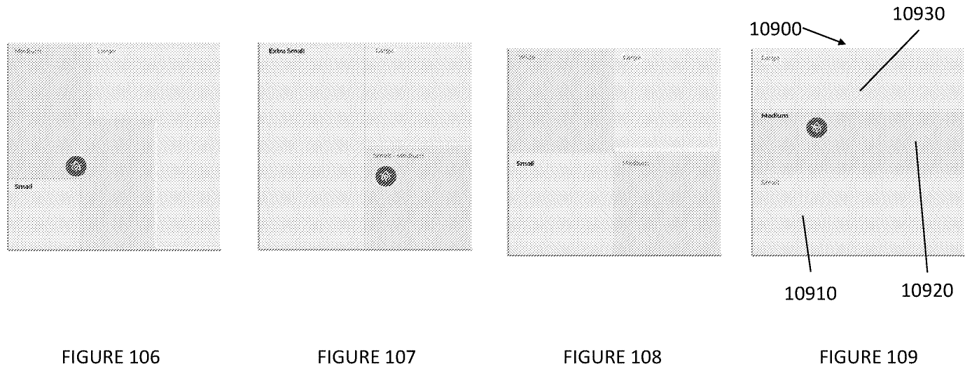

FIG. 106 to FIG. 109 illustrate sizing charts for different patient interfaces. These sizing charts include different shaped regions associated with different patient interface sizes. For example, in FIG. 109, sizing chart 10900 includes three regions 10910 (Small); 10920 (Medium); 10930 (Large). In FIG. 109, for each size, the patient interface size is dependent on the dimension of the Y axis, regardless of the facial dimension of the X axis. This corresponds to FIG. 104, where it can be seen that sizing of the mask is dependent on only one facial dimension, namely face height. Accordingly, in FIG. 104 only the one-dimensional sizing chart is shown (but the chart 10900 of FIG. 109 could additionally, or alternatively, be shown; the chart 10900 effectively varying in only one-dimension.).

QR Code: Sharing with Patient

Typically the clinician mode scanning and mask recommendation process is executed on a clinician system. The system may include a share feature to allow the mask fitting recommendation to be shared from the clinician system to the patient or other interested party. The share feature may appear as an icon 10240 10340 10440 on a mask size information display screen. Selection of the share icon 10240 10340 10440 triggers an information transfer process. Typically, the mask recommendation information is to be made available to the patient. In other cases, the mask recommendation information may be made available to other parties.

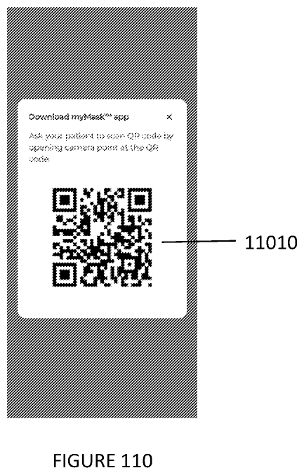

In a first example shown in FIG. 110 the system generates a QR code. The QR code may be scanned directly by the patient, for example if the patient is in the clinician's office during the mask sizing process, or may be transferred to the patient, for example via SMS, email or other communication channel.

The QR code may encode the size information, mask type, and/or clinician details. The data which is encoded in the QR code may be subsequently accessed by a patient device for local configuration of their patient mask application or for display on the patient device for example on a web page for their reference.

The results from the mask recommendation process may automatically be made available to the patient within a patient mask application (for example a dedicated patient application to support the patient with treatment and mask equipment). In such cases the QR code may provide the patient with a link to a patient mask data, for example via a patient mask application or via an internet address. The QR code includes the internet address (URL) and/or a link to the patient mask application (for example Apple Store) and/or a trigger to initiate the software application if it is pre-installed on the patient device. Within the patient mask application, the patient may access the results from the mask recommendation process (i.e. access either the final choice/recommendation of mask, and/or access other masks that were considered during the consult with the clinician). The patient may also be provided with a link to purchase the recommended masks within the patient mask application.

In other examples, QR code 11010 may include a weblink to purchase the recommended mask. For example, via a mask dealer's online website.

In one example process, the QR code is displayed on the clinician's phone (or other device) for the patient to scan or to be transferred to the patient via a communication channel. Scanning of the QR code will trigger the the following invitation process for the transfer of said information. In a first step the patient scans the QR code on their device, this may be performed directly by scanning the QR code when displayed on the clinician system or by scanning or accessing the QR code after it has been transferred to the patient. The QR code triggers the patient device to identify if the patient mask application is installed on the patient device. If it is not installed, the device is directed to a link to install the patient mask application (for example an App Store). When the patient mask application is installed (or if the application is pre-installed on the patient device), the patient device may run a local configuration of the installed app to populate it with the mask type, size information. The clinician details may also be included.

Scanning (i.e. Image Capture) Process for Clinician Mode:

During the clinician mask fitting sequence, when the system determines that image capture will be executed using a front facing camera of the device (traditional image capture), it runs the designated image capture process associated with the front facing camera.

FIG. 111 illustrates an example of an instruction interface displayed during clinician mode in which a clinician is controlling the camera to capture an image of a patient. If the clinician is using a mobile phone, the clinician holds the mobile phone using the front facing camera to capture the image of the patient. Instructions are presented on the screen towards the clinician. The difference in the clinician mode is that the system is capturing images of a third party and not the user of the device.

FIG. 111 shows animation to instruct the clinician to achieve the correct orientation of the camera with respect to the patient's head. The animation including a static indicator and a dynamic indicator. In the example of FIG. 111 the static indicator and dynamic indicator are out of alignment indicating that the mobile phone is not positioned in the correct orientation. Static indicator represents the target desired orientation for the head of the patient. Dynamic indicator represents the current orientation of the head of the patient with respect to the desired orientation. When the dynamic indicator is aligned with the static indicator the camera is in the correct orientation with respect to the head. Any difference in alignment of the static indicator and the dynamic indicator represents a difference in the current orientation of the face of the user relative to the desired orientation of the face of the user, as shown in FIG. 111. The animation of FIG. 111 is similar to that described above with respect to FIGS. 59 to 62 but is configured to instruct a user who may not be the patient.

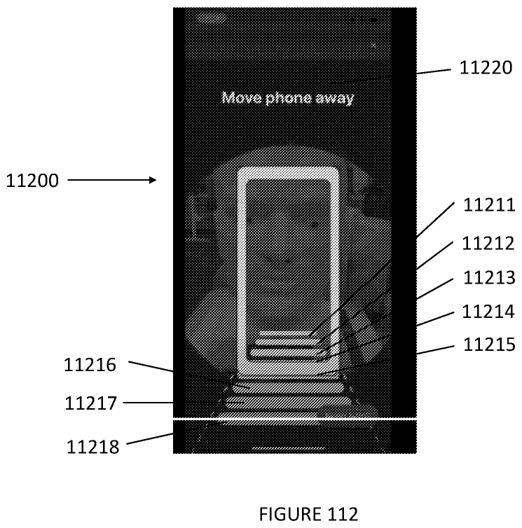

FIG. 112 shows animation to instruct the clinician to achieve the correct distance between the camera and the patient's head. The animation of FIG. 112 is similar to that described above with respect to FIGS. 87 to 95 but is configured to instruct a user who may not be the patient.

The screen 11200 includes multiple visual indicators 11211 11212 11213 11214 11215 11216 11217 11218. Together the visual indicators form part of a position indicator to assist the clinician to correctly position the camera/face or head of the patient at the desired distance from the camera in order to capture an image of the head.

In the example of FIG. 112, the indicator is provided by a series of indicator bars on the screen. Different styles of indicator may be used. In the example of FIG. 112 the indicator bars are horizontally arranged across the display screen. The horizontal indicator bars are arranged to guide the clinician towards a desired distance by representing a comparison between the current distance between the patient face and the camera (as represented by an identified indicator bar) and the desired distance between the face and the camera (as represented by a specific indicator bar). The indicator is a progressive sequence including a series of indicator bars displayed at different locations on the screen. In the example of FIG. 112 the desired distance between the face and the camera is represented by one of the indicator bars 11215. This indicator is referred to as the target distance indicator bar. The target distance indicator bar 11215 is distinguishable from the other indicator bars. In example systems the target distance indicator bar may be represented with a different animation or colour to allow it to be easily distinguishable as a target indicator bar compared with other indicator bars. In the example of FIG. 112 the target distance indicator bar is a different colour from the other indicator bars. In further examples, the target indicator bar may include a particular animation, may be a different shape from the other indicator bars, or be presented in some other way to make it distinguishable from the other indicator bars.

The animation of FIG. 112 is similar to that described above with respect to FIGS. 87 to 95. The current distance between the face of the patient and the camera is presented to the clinician on the screen. As the distance between the face and the camera changes with respect to the target distance the animation of the indicator changes. The current distance between the camera and the face is represented by illuminating one of the indicator bars. The bar representing the current distance between the camera and the face of the patient is referred to as the current distance indicator bar. Other animations or techniques may be used to distinguish the bar representing the current distance of the device compared with the other indicator bars. Indicator bars in closer proximity to the target distance indicator represent separation distances between the face and the camera closer to the desired distance. The closer the current distance indicator bar is to the target distance indicator bar the closer the current distance between the face and the camera is to the desired distance. The indicator bars may not be visible until they are illuminated or changed in colour.

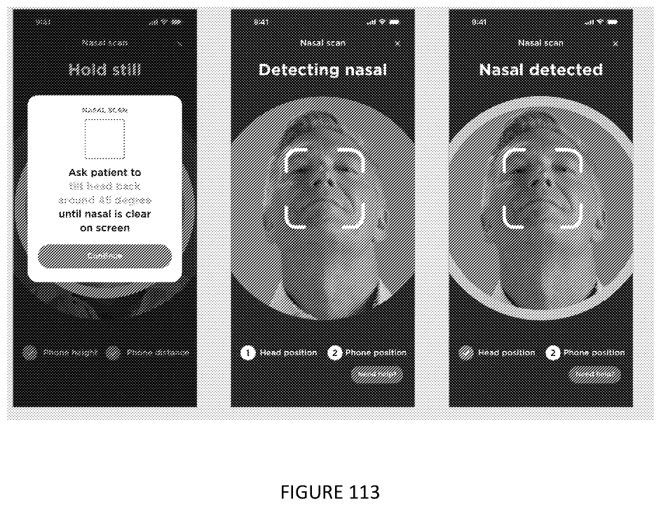

Additional animation or indicators may be provided to assist the patient and/or clinician in reaching the desired orientation between the face and the camera. In the example of FIG. 111 text is displayed to the clinician to assist them to reach the correct height. In the example of FIG. 112, a text instruction “Move phone away” 11220 is displayed to prompt the clinician to move the camera further from the face of the patient. See also FIG. 113 which shows something similar.

The animation of FIG. 112 is superimposed on a live-image captured by the camera and displayed on the screen, so that as the clinician moves the phone (and/or instructs the patient to move their face) in the relevant direction/manner, the dynamic indicator tracks the patient's face (both displayed on the screen) and the clinician is able to see the dynamic indicator relative to the static/target indicator (also displayed on the screen), and thus see if they are getting closer to the required relative orientation between the face and the camera.

In an alternative animation, rings are presented to indicate distance between the camera and the head of the patient. The ring increases and decreases in size proportionate to how close and far the phone is from the patient's face. In other examples, other shapes or graphical representations may be used, such as a box, frame, boundary, arrow, and/or other guiding element that increases and decreases in size proportionate to the distance of the phone from the patient's face. Alternatively, or in addition to the increasing and decreasing ring, or other guiding element a part of the display may change colour when the distance between the camera and the patient's head is within the target range. This may be the ring, or other guiding element itself or a separate indicator that changes colour. In some examples, only a colour changing indicator is used to indicate distance between the camera and the phone, without having any of the displayed elements change in size. The colour changing indicator may change colour continuously through a gradient of colours between a first colour indicating the phone is far from the target distance to a second colour indicating the phone is within the target distance. In other examples, the colour may change discontinuously between a defined number of colours indicating whether or not the phone is within the target distance.

In other examples, clinician mode (the clinician using traditional image capture) and patient mode (the patient using selfie mode for self capture) may incorporate different tolerances. For example wider tolerances may be programmed in clinician mode. Device position requirements may also be different. For example, the requirement for the device to be held vertically may be removed, so that a clinician can place the camera to capture the underside of a patient's nose (and capture the front-on image too) in positions where the patient is lying down. The same “relative angle” between phone and face may be required but different absolute angles of the phone will be necessary as the patient's head position is different.

In some example systems a two-step process may be implemented. A first coarse adjustment may be configured (i.e. Performed). The coarse adjustment may position the patient's head in a particular orientation (for example vertically). A second fine adjustment sequence may follow in which the clinician is directed to position the phone with more precision. The two-step process may be particularly advantageous when the clinician is scanning the patient (as opposed to selfie mode). In selfie mode, the patient can see in real-time the positional instructions and feedback (e.g. the indicators) being displayed on the screen. In contrast, in clinician mode the clinician sees the positional instructions and must relay them (such as verbally) to the patient. If the clinician were to try to instruct the patient to precisely match the instructions being displayed on the screen, this would likely be very difficult, frustrating and time-consuming, as it would involve repeated adjustments of (say) an angle or two by the patient. With the two-step process, the clinician only needs to get the patient to position their head in approximately the correct orientation/position, after which the clinician can move the phone to refine the relative orientation based on feedback from the on-screen indicators.

The two-step process may be discretely divided into two separate steps, such as a first step wherein the system (for instance) instructs the clinician to “tell the patient to tilt their head back”; and a second step in which the system (for instance) instructs the clinician to “now move the phone to achieve the correct relative orientation” (this would be with the aid of the on-screen indicators). Alternatively, the two-step process may be iterative, with the patient moving their head to a first, approximate, orientation, the clinician making a correction by repositioning the phone, then the patient making a further correction by moving their head again, et cetera.

The two-step process may apply to one or more of the scanning/orientation criteria. That is to say, it may apply to all of the height, distance, angle criteria, or it may apply to just one or two of them. For instance, it may apply only to the angle criterion. In such a case, the clinician may firstly ask the patient to tilt their head back (such as “look up at the ceiling”); after which the clinician may look at the indicators on the screen and make fine-tuning adjustments to the relative angle of the phone and the face (by tilting the phone). The other two criteria may be achieved by the clinician on their own, i.e. by moving the phone back to the required distance and the required relative height. The advantage of this is that only one action (or in any case fewer actions) is required of the patient, with the clinician compensating and taking care of the other alignment criteria.

Furthermore, in clinician mode the order/sequence of achieving the respective alignment criteria (height, angle, distance) may optionally be different than when using the system in selfie mode. In selfie mode the “angle” criterion may in some embodiments be last in the sequence (after the height and distance criteria), on the assumption that, once the patient has their head tilted back, it may be difficult for them to read further positioning instructions on the screen. In contrast, the clinician mode is more flexible, as the clinician can see the instructions on their screen regardless of the patient's facial orientation. Thus, in clinician mode the first instruction may (by way of example) relate to getting the patient to tilt their head back; and subsequently the “distance” and “height” instructions may be displayed, for the clinician to fulfill (optionally with help from the patient). Or any other sequence of the positioning/alignment criteria.

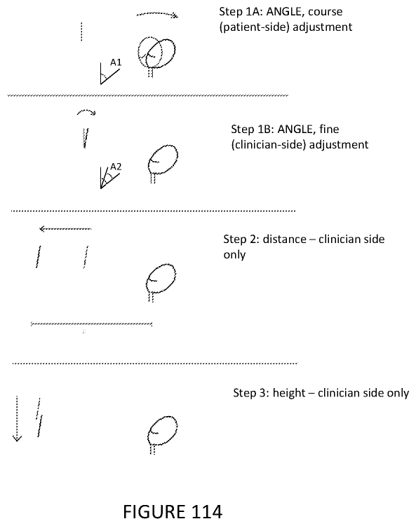

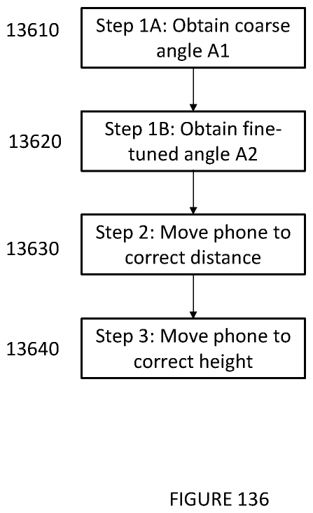

FIG. 114 illustrates an exemplary process for achieving the correct relative orientation between the camera and the patient's face, when in clinician mode (i.e. with the clinician doing the scanning). This is by way of example only, and various other combinations of phases/stages and steps are also possible. The steps taken in FIG. 114 are shown in the flow diagram in FIG. 136.

Steps 1A and 1B relate to achieving the correct relative angle (for example for a subnasal image capture). As noted above, in clinician mode this may optionally be the first step. In this example, the “relative angle” step is achieved, firstly, via a crude/coarse adjustment at Step 1A 13610, followed by a fine-tuning adjustment at Step 1B 13620. In Step 1A 13610, the patient (at the clinician's request) tilts their head back. The clinician may be prompted (via instructions on the display screen such as via animation or other prompt as disclosed herein) to tell the patient to tilt their head back, and may in turn ask the patient to do so. The patient is likely to tilt their head back by a non-precise amount, hence why step 1A is a “coarse” adjustment. The resulting angle between the phone (or otherwise moving the phone) and the patient's face may therefore be A1, being in the vicinity of, but not precisely, the required angle. This is followed by Step 1B 13620, in which the clinician fine-tunes the relative angle (to get it to the required angle) by tilting the phone (or otherwise moving the phone) to compensate for the patient's head tilt. The display screen may specifically prompt the clinician to do so, or the display may continue to display the same thing as in Step 1A, with the clinician inferring that they are to compensate by tilting the phone (or otherwise moving the phone). The result of the clinician fine-tuning the angle by tilting the phone is that the relative angle becomes A2, the required angle. The display may communicate to the clinician when the required angle is achieved, in any of the ways discussed further above.

In Step 2 13630, the clinician moves the phone away from (or in other cases closer to) the patient to achieve the required distance D between the phone and the patient's face. In this example, this is shown as being a clinician-only step, i.e. no further movement is required from the patient; however, this is not intended to be limiting.

In Step 3 13640, the clinician moves the phone down (or in other cases up) (relative to the patient) to achieve the required relative height between the patient's face and the phone. Again, in this example this is a clinician-only step but this is not intended to be limiting. The advantage of steps 2 and 3 being clinician-only is that only one movement (tilting the head back at step 1A) is required of the patient; with the clinician doing the rest. Thus, the process is straightforward and not taxing or frustrating for the patient, and moreover the correct relative orientation is likely quicker and easier to achieve by relying (at least in part) on the clinician moving the phone than on the patient to move their head.

The concept of moving the phone relative to the face has broader application than just “clinician mode”. It can equally apply in the “selfie mode” and any other example or embodiment (as appropriate, and with any required modifications) discussed herein. Furthermore, it may apply (where appropriate, and with any required modifications) in conjunction with other requirements, for instance in conjunction with a requirement that the phone be held vertical. For instance, assuming a requirement that the phone be held vertical, the step of achieving the required angle may entail the patient tilting their head back (while keeping the phone vertical); but the steps of achieving the required height and distance may entail one or more of moving the face and/or moving the phone (while keeping the phone vertical). In such an example, the prompts to the user may include (simultaneously or in sequence) instructing the phone operator to both keep the phone vertical, and to move the phone as required (e.g. closer to/further from the face, or up or down relative to the face).

In some embodiments the camera (image capture device) is moved into a specific orientation for image capture. In some examples provided above the camera is orientated into a vertical orientation before the image capture process is commenced. In other embodiments other non-vertical orientations of the camera may be used. In other examples, the application may require the camera and the face or head of the user to be in a predefined relative angle, to capture either a front-on facial image or a particular angle of the face, for example a sub-nasal image. The orientation of the camera may be recorded when the camera and the face of the user are in the predefined relative angle using orientation sensors associated with the camera (for example in the device having the camera). This may be useful, for example, in the situation when a user is unable to move their head, for example if they are lying down, reclining, bedbound, or have limited mobility through their neck. The camera may be configured to automatically detect when the predefined relative angle between the camera and face is achieved (for example using the sensors). Alternatively, the system may include a button or input means, such as on the display. As the user moves (in a relative sense) the face and the camera, they visually determine when the face and camera are in the required relative relation, and at that point they press the button or actuate the input means, and the system logs that angle as being the “:base” or “start” angle.

This initial camera orientation may be referred to as a base angle or starting angle of the camera and if further angles are required for images these can be captured relative to this base or starting angle. This allows for images to be captured from different angles and guidance to be provided to the user in the scenarios in which the head is held still and the position of the camera is adjusted to capture images from different angles or positions (or in any case when the head/face and the camera are moving relative to each other). (In some embodiments the step of obtaining the base or starting angle may be performed more than once during the image capture process, for instance between each set of image capture types [frontal, sub-nasal]).

In embodiments where the phone (i.e. the image capture device) can be non-vertical during the image-capture process—for example but without limitation the embodiment of FIG. 114, or when the patient is for instance lying in bed or reclining—the process may involve the additional step of indicating or obtaining a base angle, or starting angle, of the phone (which may be thought of as a reference angle).

The base angle or starting angle may, for example, be the angle at which the phone and the face are parallel (or substantially parallel) to each other. Alternatively, the base angle or starting angle may be the angle at which the phone and the face are at a required orientation or angle relative to each other. This may be a numerical angle or a functional angle (e.g. where the nostrils are visible, or another facial feature is visible).

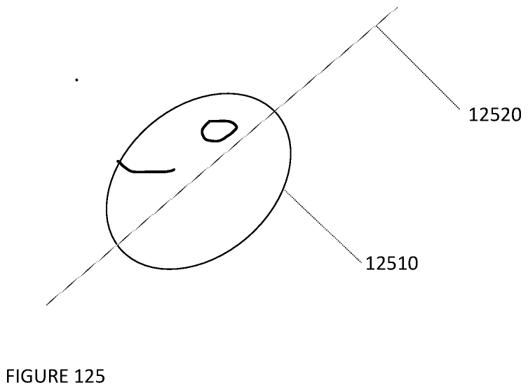

FIG. 125 shows an example in which the camera is orientated into a non-vertical base angle or starting angle. In the example of FIG. 125, the head of the patient 12510 is in a non-vertical orientation and head is supported, for example by a pillow, and so is in a fixed position. The absolute angle of the head to the vertical (or to put it differently, the general “plane” or “axis” of the face or head”), is illustrated by reference line 12520.

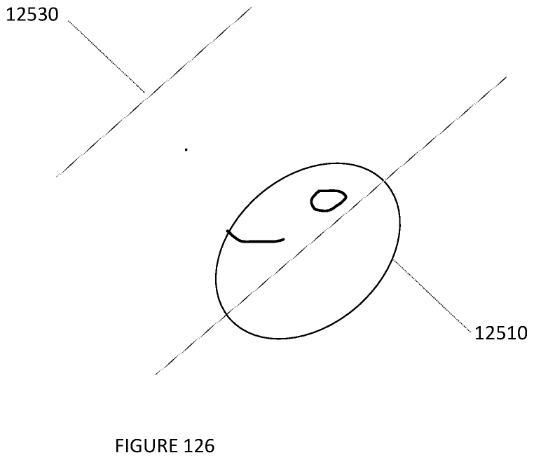

FIG. 126 shows the head 12510 and includes mobile communication device 12530. Mobile communications device 12530 executes the image capture application and includes a camera for capturing images of the patient's face and orientation sensors monitoring the orientation of the mobile communications device (and camera). In the example of FIGS. 125 and 126 the mobile communications device may be manually controlled by the patient (in selfie-mode) with the display screen pointed towards the patient to allow the patient to see various instructions, prompts and images, or may be manually controlled by a clinician or other third party (in regular mode) with the display screen pointed away from the patient to allow the clinician to view the display screen.

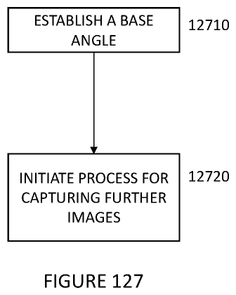

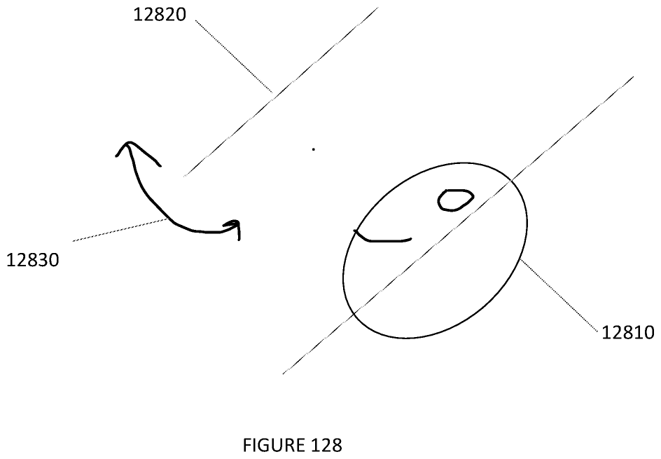

FIG. 127 shows the sequence of steps performed in an example embodiment. FIGS. 128 to 130 illustrate the relative angles between the face of the patient and a mobile phone containing a camera at the different steps in the sequence. At 12710 the system establishes a base angle.

The base angle or starting angle may be obtained by the phone operator (such as the clinician) manually moving the phone 12820 into approximately the correct orientation, such as by sight; and subsequently entering input into the phone (such as via a button displayed on the interface) to indicate that this is the base angle or starting angle. The button may be of any suitable configuration, such as a “START” button, or a “CORRECT ANGLE” button; and the interface may also display instructions prompting the user as to what to do, i.e. to manoeuvre the phone into the correct orientation. For instance, if a front-on starting position is required, in which the face is parallel to the plane of the camera, the instructions may be to the effect of “hold phone parallel to face”. If a sub-nasal starting position of the patient is required, the instructions may be to the effect of “tilt phone to approximately X degrees”, or “tilt phone until the patient's nostrils become visible”. FIG. 128 shows the face of the patient 12810 and the mobile phone 12820. The arrow 12830 indicates the tilt of the phone being changed relative to the angle of the face.

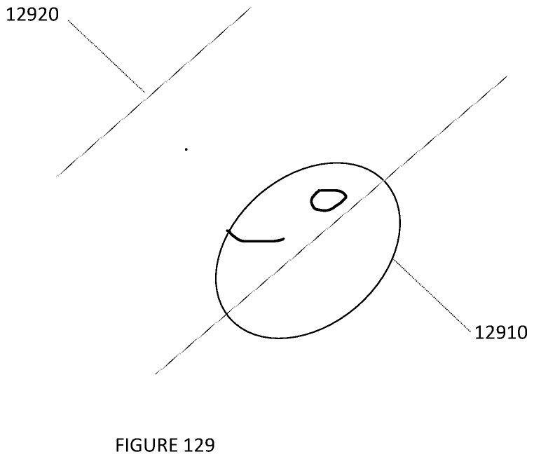

When the mobile phone 12820 is at the correct angle with respect to the patient's face 12810 (as represented in FIG. 129) the user may press the button or otherwise indicates that the phone is at the base angle or the starting angle, the angle at that point in time of the phone may be recorded as the base or starting angle. The orientation of the phone, such as its tilt away from vertical, may be determined for instance using the gyroscope or accelerometer of the phone, or via another suitable means. In the example of FIG. 129, the mobile phone 12920 is aligned parallel with the face of the patient 12910

In an alternative embodiment, instead of the user manually indicating (such as via a button) that the phone is oriented at the base angle or starting angle, the system may be configured to automatically detect this. For instance, the system may be configured to, with reference to the live-action preview of the user's face, calculate or determine in real-time a relative angle between the phone and the face, and automatically detect when the correct or required relative starting angle is achieved. For instance, the system may do this by considering apparent distances between (or sizes of) different features on the face, and inferring angles from this; and/or the system may detect whether particular facial features (such as nostrils) are visible, or sufficiently visible. This may be determined by a facial mesh function which automatically detects various facial features and calculates the orientation between the camera and the face based on the facial features. The application may issue an initial prompt to the user, for example, “hold phone parallel to face”. As the application monitors the relative angle between the camera and the face of the patient it may issue further prompts to the user-such as to move or pivot the phone slowly relative to the face; tilt the phone towards the face; tilt the phone away from the face—but the actual detection of the phone being at the correct angle may be automated in such an embodiment. Once the system recognizes that the base angle or starting angle has been achieved, by moving the camera into the required angle with respect to the face, a message may be displayed on the phone, to notify the clinician (or phone operator) that the phone is at the correct starting angle. The orientation of the camera when it is in the correct angle with respect to the face may be recorded.

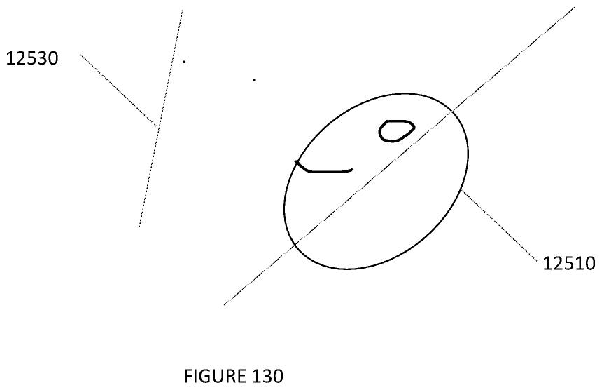

When the base angle or starting angle has been established at 12710 the application runs the process for capturing any further images at 12720. These may include front-on images to the patient's face having the camera parallel to the patient's face, sub nasal images, or images from different relative angles between the face of the patient and the camera. FIG. 130 shows an example of the mobile phone positioned to capture a sub-nasal image.

Subsequently, the system calculates a relative angle between the phone and the patient's face as the two are moved relative to each other, as opposed to simply an angle of the patient's face relative to the vertical. In other words, the phone is still the (or a) reference plane, but the angle of the phone itself is taken into account. The relative angle may be calculated based on measurements taken from orientation sensors on the phone to calculate adjustment of the orientation of the camera. Alternatively, or in addition, computer software may monitor the image of the face of the user and detect the relative angle, for example by selecting and monitoring the facial features, for example by using a facial mesh image recognition model.

It will be understood that specifying a starting angle or base angle, relative to which subsequent orientation determinations are made, may be particularly useful in cases where the patient is, for example, reclining or lying in bed or has restricted movement in their neck, and the clinician (or even the patient themselves) must do the scan without the benefit of holding the phone vertical. In such a scenario, being able to indicate (or detect) when the phone is (for instance) parallel to the face, and taking this as the phone's base angle or starting angle relative to which subsequent steps are performed, means that the scanning and sizing process can in effect take place much as described above, but with the phone's reference axis being tilted by a given amount (which is able to be quantified by the system).

In such an embodiment, subsequent positioning (or movement) of the phone relative to the face, such as to obtain images from different angles, can be achieved by moving the face and/or the phone. The displayed instructions may be neutral, such as “increase a relative angle between the phone and the face”, or they may specify that either the phone or the face (or both) is to move.

In some embodiments, instead of a live-action preview of the user's face per se, the live-action view can instead be in the form of an animation, such as a caricature of the face, that mimics the user's movement in real time but presents this in the form of a caricature or animation instead of an actual image of the user's face or in the form of a combination of a caricature or animation and an actual image of the user's face.

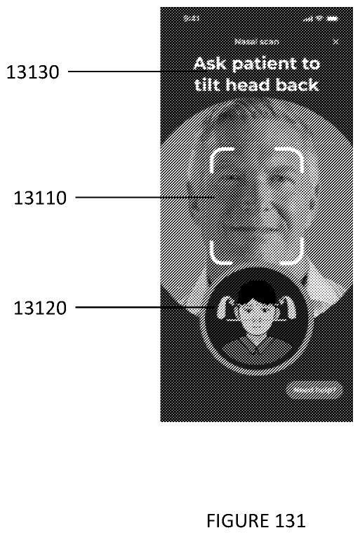

The FIGS. 131 to 135 are an example of this. A caricature of a face moves on the screen in a manner that mirrors the user's or patient's actual head/face movements (in real time). Indicators on the screen provide guidance to the user as to how to move the face into the desired orientation.

FIG. 131 shows an example of a user interface which displays a caricature of the patient's face. The user interface displays “position indicators” to provide guidance to the user as to how to adjust the position of the face of the patient into the desired orientation. The example of FIG. 131 could be implemented in the clinician mode or the “selfie” mode. FIG. 131 includes a real time image of the patient as captured by the camera 13110; a caricature representing the face of the patient 13120, the caricature is animated to move as the patient's face moves; and prompts presented in the form of text 13130 to provide guidance to the user to help position the patient's face. The example of FIG. 131 is operating in clinician mode, in which a clinician or other third party is presented with the images and guidance to capture an image of the subject patient. In other examples, the system works in patient mode in which the patient holds and adjusts the camera operating in ‘selfie’ mode and views the display screen. Although in FIG. 131 the image of the patient's face as captured by the camera, the caricature and the text prompt are all included on the display, in other examples one, two or all three of these components may be included, for example the display may only include the caricature.

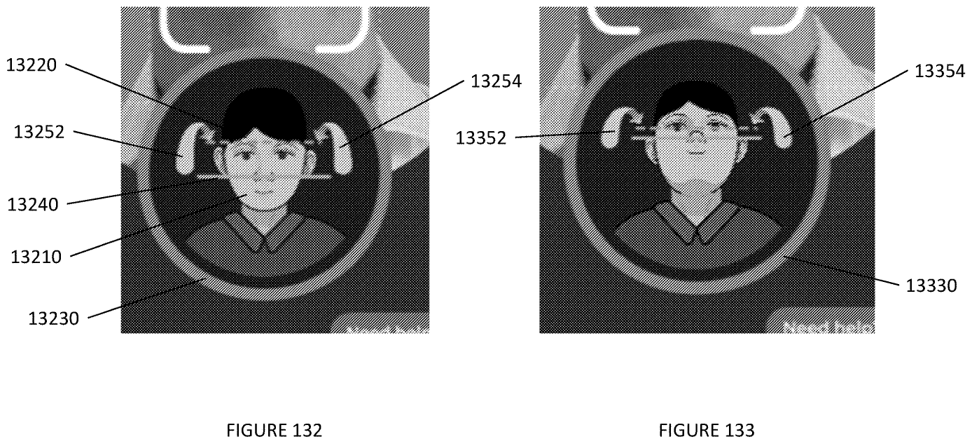

FIGS. 132 to 135 illustrate the caricature in more detail. Referring to FIG. 132, the caricature 13210 represents the face of the patient in the orientation currently captured by the camera. The display includes a static indicator provided by the dashed line, which is static on the screen; in other words its position within the circle 13230 does not change. A dynamic indicator 13240 is provided by a solid line. The solid line 13240 is drawn across the caricature's nose and which follows the nose as the caricature (mirroring the patient) tilts their head. When the two indicators overlap/align, the face of the patient is in the correct position for image capture. Further indicators 13252 13254 are also present on the screen. These indicators 13252 13254 are arrows indicating the direction the patient should tilt their head. Peripheral circle 13230 provides further guidance to the user on whether the current orientation of the patient's head matches the required orientation. In the examples of FIGS. 131 to 135 the peripheral circle 13230 is presented in a first state (i.e. colour red) when the orientation does not match the required orientation, and changes state, (i.e. turns from red to green) when the correct orientation is achieved.

In FIG. 132, the patient's face needs to be tilted backwards to meet the required orientation. The peripheral circle 13230 is red to indicate the face orientation does not match the required orientation and arrows 13252 13254 are arched backwards to indicate that the patient's face needs to be tilted backwards to meet the required orientation.

In FIG. 133, the patient's face has been tilted backwards from the orientation of FIG. 132. The patient's face needs to be tilted backwards further to meet the required orientation. The peripheral circle 13330 is red to indicate the face orientation does not match the required orientation and arrows 13352 13354 are arched backwards to indicate that the patient's face needs to be tilted backwards to meet the required orientation.

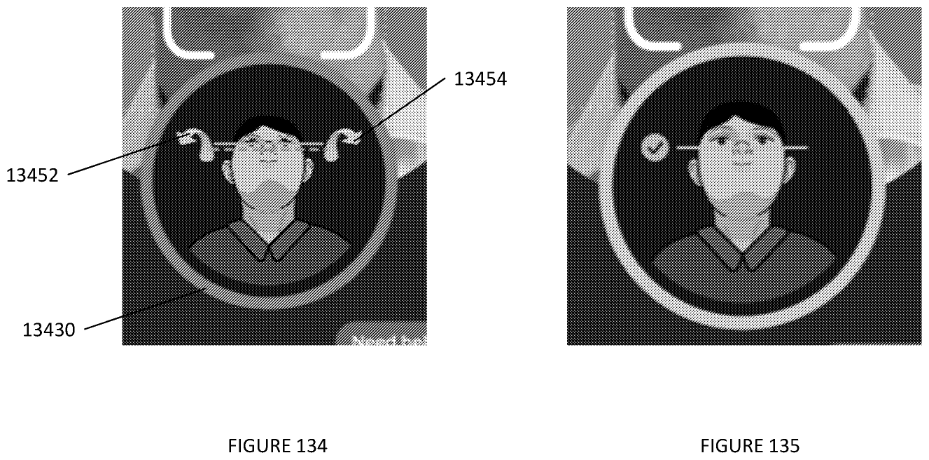

In FIG. 134, the patient's face has been tilted further backwards from the orientation of FIG. 133. The patient's face now needs to be tilted forwards to meet the required orientation. The peripheral circle 13430 is red to indicate the face orientation does not match the required orientation and arrows 13452 13454 are arched forwards to indicate that the patient's face needs to be tilted forwards to meet the required orientation.

In FIG. 135 the face of the patient is in the correct orientation. The static indicator and dynamic indicator are aligned. The peripheral circle 13530 has changed state to green to indicate that the face of the patient is in the correct orientation.

Additional text prompts may be presented to the user during the face positioning process, for example “Ask Patient to Tilt Head Back” or “Ask Patient to Tilt Head Forward” to provide additional guidance to the user.

In the clinician mode, further or additional display elements may be present in some embodiments. Though discussed here in conjunction with the clinician mode, these display elements may also be utilized in the self-scan mode. In either mode, these additional elements may be used either alone or in any appropriate combination with any of the other positioning/orientation display elements discussed further above.

Firstly, the display may comprise guidelines, in the form of a grid or similar pattern superposed on the screen (and present simultaneously with the real-time display of the patient's face), to help the clinician visually assess how far from the target position/orientation the patient's face is (relative to the camera), and/or, during movement, to help the clinician visually assess the magnitude of the relative movement between the camera and the face (relative to the target position/orientation).

Secondly, the display may comprise a feature bounding element which surrounds the facial feature the capturing of which is desired in a particular orientation. The feature bounding element may surround the entire face, or some portion of the face; and may accordingly vary in size. The feature bounding element “locks on to” and dynamically follows the patient's face as it changes position/orientation in the real-time display due to relative movement between the face and the camera (including, optionally, changing size as the patient moves forward or back). The feature-bounding element may serve, on its own, to help the clinician visually assess the position of the face and/or the magnitude of any relative movement. Additionally, or alternatively, the feature-bounding element may also need to align with another (static) element to indicate correct orientation. The static element may be the grid/guidelines, or it may be some other element.

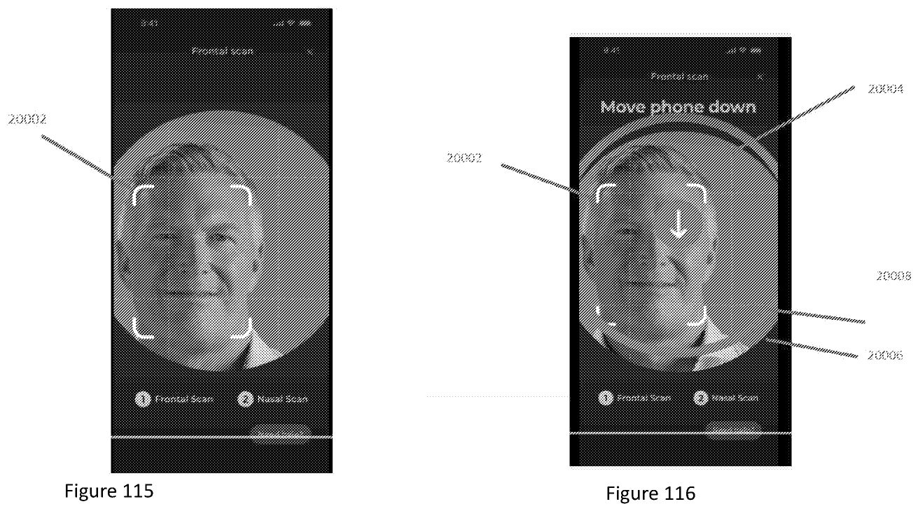

In the example of FIGS. 115-119, the feature-bounding element surrounds the entire face. In FIG. 115 a front-on image capture is desired. As such, the goal is for the feature-bounding element (and thus the face) to be substantially centrally located on the display, as this will indicate that the user is facing forward. In various embodiments, the clinician might judge this by sight; they might do it by aligning the feature bounding element with the centrally-located gridlines; they might seek to align the feature bounding element with another static element; or there might be another (separate) set of indicators that promote alignment, with the feature bounding element serving as a further but separate visual indicator.

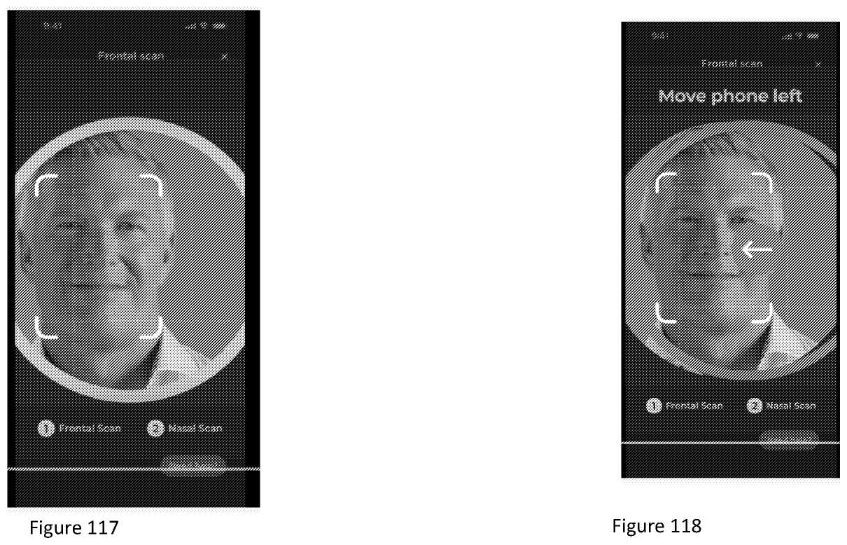

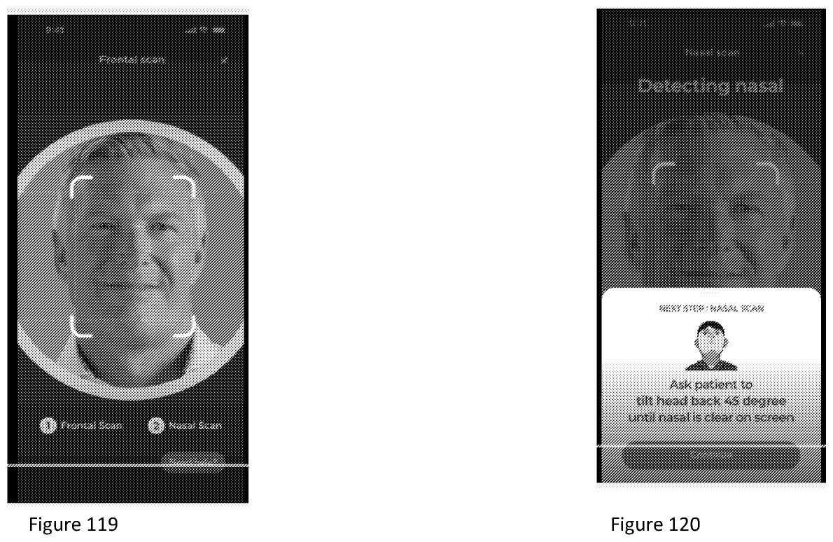

In FIG. 115, the feature-bounding element 20002 is shown locking on to the user's face. In FIG. 116, as the patient's face moves (in a relative sense) upwardly and thus closer to the centre of the frame, the feature-bounding element 20002 follows the face and thus moves relative to the gridlines 20004, compared to where it was in FIG. 115, and this may in its own right help the clinician to orientate the face centrally on the display. In addition, in FIG. 115 there are also additional positioning indicators, namely a static 20006 and dynamic 20008 indicator, similar to those described above. In FIG. 117 an interim green shade of the dynamic indicator (overlaid with the static indicator may indicate that the user's face is at the correct (relative) height. In FIGS. 118, 119 the same process is repeated to ensure the user's face moves sideways (relatively) so as to be in the centre of the frame. It will be seen that, as the user gets closer to the correct (central) position, the feature-bounding element 20002 comes closer to alignment with the centremost gridlines. Thus, as seen in FIG. 119, in this embodiment correct alignment is indicated both by coincidence of the static and dynamic indicators and by alignment of the feature-bounding element with the centremost gridlines. Once the face is in the correct position, the scanning process may take place substantially as described above.

Turning to FIGS. 120-124, these show the “under-nose” phase of the image capture process. In FIG. 120, the clinician is prompted to ask the patient to tilt their head back. This corresponds to the first, “coarse” stage discussed above, i.e. it can be expected that the patient will tilt their head back by an approximate amount, but that this will not perfectly align with the required angle.

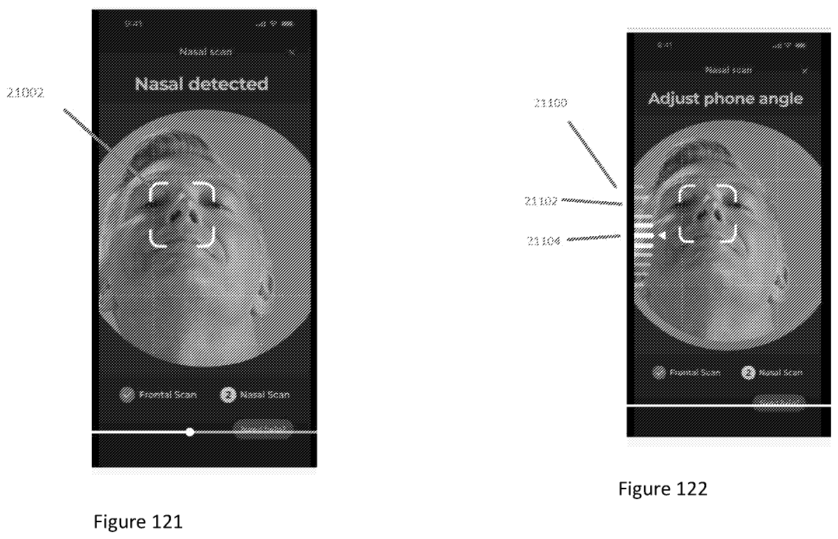

In FIG. 121, the feature-bounding element 21002 locks onto the nasal region, i.e. surrounds the nasal area (since the sub-nasal scan is for the purpose of capturing nasal dimensions), making this a visual “point of focus” for the clinician as they subsequently move the phone into the correct orientation.

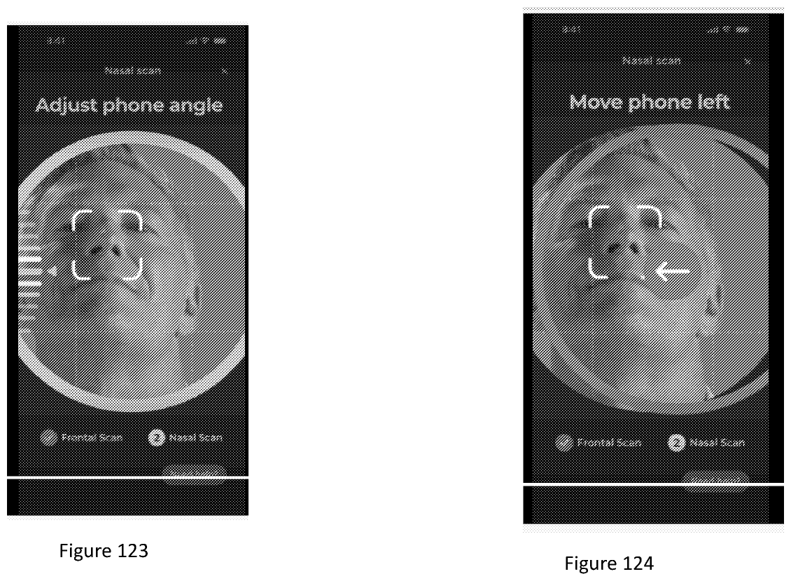

In FIG. 122, the second, “fine-tuning” phase is shown, namely the phase where the clinician moves the phone once the user has initially tilted their head back, to fine-tune the relative angle. To this end, in FIG. 122 there is a relative angle indicator 21100, in the form of a series of horizontal bars, with the current bar (21102) (corresponding to the current relative inclination) highlighted (in red), and the “target” bar (21104) (corresponding the correct relative inclination) being more prominent than the others. As seen in FIG. 123, when the clinician has fine-tuned the angle of the phone to achieve the correct orientation, the “target” bar is highlighted (in green). Optionally, the feature-bounding element may also visibly move relative to the gridlines as the relative orientation changes, to provide a further visual guide for the clinician.

In FIGS. 122 and 123, it can be seen that a further, supplementary, indicator is provided by the coloured peripheral ring. In this embodiment the peripheral ring does not move (and is not a paired static-dynamic couplet), rather its colour alone serves as a further indicator of whether the orientation is correct (green) or incorrect (red). Such a colour-only indicator, whether in the form of a ring/frame or some other form, and whether alone or in conjunction with other indicators, could be employed in any of the other embodiments discussed herein.

As shown in FIG. 124, once the correct angle is achieved the clinician may then further move the phone sideways to achieve the correct (generally central) position of the face (again optionally with the aid of a static and dynamic indicator), and other requirements such as the correct relative height and distance. As discussed above, these steps may be done by “clinician fine-tuning” alone, i.e. without requiring further movement on the part of the patient. The scanning step can then take place, substantially as discussed above.

Also of note in FIGS. 115-124 is that the display includes, at the bottom, an indicator of whether the current image capture phase relates to frontal scanning or nasal scanning—i.e. front-on or under-nose; with the “frontal” indicator becoming ticked once that phase has been completed. This may further help the clinician to understand the scanning process and the alignment that is being aimed for.

As described above, in exemplary embodiments, the selection of the patient interface category for a patient from the responses to the questionnaire is used to determine which dimensions may be required for patient interface sizing. The questionnaire is presented and the patient responses are used to determine the category of patient interface. Once the category is identified, the specific landmarks that are required for that patient interface category are identified in the application. All landmarks may be gathered, but the calculation of distance between specific landmarks are done by the application based on the patient interface category identified. Accurate measurements for different landmarks may be obtained by viewing the face from different angles. Embodiments, determine which angles are required. Instructions are provided to the user to position the camera appropriately to capture the required images. A reference feature, for example eye width, may be used to produce a scaling factor for images. The scaling factor may be used in images from different angles.

In the embodiments described above, the application and various databases have been stored locally on the mobile communications device. Additionally, all processing during patient interface selection is performed on the mobile communications device. This arrangement avoids the need for any network connections during a patient interface selection process. Local processing and data retrieval may also reduce the time taken to run the mask selection process. One advantage is that questions and images can be processed locally and only the calculated mask size needs to be transmitted, for example when ordering a product. This reduces the data sent and reduces data costs.

However, further embodiments execute the patient interface sizing application using a distributed data storage and processing architecture. In such embodiments, databases, for example the patient interface sizing database, or questionnaire database, may be located remotely from the mobile communications device and accessed via a communication network during execution of the patient interface selection application. Processing, for example facial landmark identification may be performed in remote servers and the mobile communications device may send captured images across the communications network for processing. In other examples, processing of questionnaire responses may be done remotely. Such embodiments leverage external processing capabilities and data storage facilities.

In the embodiments described above the application has been executed on a mobile communications device. In further embodiments the application, or parts of the application, may be executed on a respiratory therapy device.

The examples described provide an automated manner of recommending a patient interface category and a patient interface size in the specific category of patient interface that is selected for the patient. Embodiments are configured to enable a non-professional user using non-professional equipment to capture data to enable the selection of a suitable patient interface for use with a respiratory therapy device. Sizing determination can take place using a single camera which allows the application to be executed on smartphones or other mobile communication devices. Embodiments do not require use of any other phone functions/sensors e.g. accelerometers.

Embodiments provide an application which allows for remote patient interface selection and sizing. This allows for remote patient set up and reduces the need for the patient to come into a specialist office for patient interface fitting and set up. The application can also provide general patient interface information and provide instructions regarding user instructions, cleaning instructions and troubleshooting as additional information.

The application uses the palpebral fissure width as a reference measurement within the image of the face of the patient. The palpebral fissure is detectable in a facial image using facial feature detection software and is less likely to be obscured by the eye lid of the patient compared with features of the eye, for example the iris or pupil. The greater width of the eye, compared with smaller facial features or eye features like the iris, enables the application to capture accurate measurements even when the patient does not hold their head still or the device being used is not able to capture higher resolution images. Use of the palpebral fissure as a reference measurement also allows the application to measure a single eye width or measurement of two eye widths to be measured and averaged. The corners of the eye can also be detected from the contrast between the whites of the eye and the skin.

Embodiments account for tilt of the patient's head and filters out measurement that may cause errors due to excessive tilt (i.e. Pitch). Similar filtering can be used for roll and yaw. The described embodiments are also advantageous because the tilt does not use the inertial measurement unit (e.g. an accelerometer or gyroscope) of the mobile communications device which can reduce the processing load and time on the processor of the mobile communications device. This also means that less sophisticated devices which might not have inertial measurement units can still be used to implement the described examples.

The sizing measurements can be performed even when the phone distance from the face varies. There is a preferred distance to ensure that the facial features of interest are captured at a high enough resolution to obtain accurate dimensions. There is a visual guide that helps the user navigate and use the sizing app. Sizing can be performed in many different environments e.g. outdoor light, indoor light. Sizing can be performed regardless of user orientation i.e. user can be lying down or sitting or standing. This provides a more robust sizing app to size patient interfaces.

Example embodiments are configured to capture images from a single image only and the patient is not required to take profile images or multiple images from different angles.

Example embodiments provide real time processing of images/video frames. This reduces processing loads and doesn't require large caching/memory requirements. Exemplary embodiments do not require large memory or caching, frames/images are not stored but processed and discarded as received.

The examples above describe ‘selecting’. In example embodiments the selection involves identifying a patient interface.

It is to be understood that, if any prior art publication is referred to herein, such reference does not constitute an admission that the publication forms a part of the common general knowledge in the art, in Australia or any other country.

In the claims which follow and in the preceding description, except where the context requires otherwise due to express language or necessary implication, the word “comprise” or variations such as “comprises” or “comprising” is used in an inclusive sense, namely, to specify the presence of the stated features but not to preclude the presence or addition of further features in various embodiments of the invention.

It is to be understood that the aforegoing description refers merely to exemplary embodiments of the invention, and that variations and modifications will be possible thereto without departing from the spirit and scope of the invention, the ambit of which is to be determined from the following claims.

1-41. (canceled)

42. A method for selecting a patient interface for a patient for use with a respiratory therapy device, the patient interface suitable to deliver respiratory therapy to the patient, comprising the steps of:
determining a dimension of a facial feature required in order to select a patient interface for a patient;
determining a desired orientation of a face of the patient, relative to an image-capture device, to be captured in a digital image, in order to determine the dimension of the required facial feature;
providing guidance to position the face of the patient and the image-capture device in the desired orientation relative to each other for image capture;
receiving image capture data representing at least one digital image of a face of the patient, the image capture data representing the face of the patient orientated in the desired orientation relative to the image-capture device;
calculating the dimension for the facial feature from the image capture data; and, using the dimension to select the patient interface for the patient.

43. The method according to claim 42 comprising the step of determining an orientation of the image capture device, comparing the orientation of the image capture device to a predefined orientation, and providing guidance to position the image capture device into the predefined orientation in dependence on the comparison.

44. The method according to claim 42 comprising the step of receiving selection of an orientation of the image capture device, the selected orientation being a reference orientation, wherein the reference orientation is selected by the patient or selected automatically, and wherein subsequent measurements are taken with respect to the reference orientation.

45. The method according to claim 44 wherein the reference orientation for the image capture device is the orientation of the image capture device when the face of the patient is at a predefined orientation relative to the image capture device.

46. The method for selecting a patient interface for a patient for use with a respiratory therapy device according to claim 42,
wherein the step of providing guidance to position the face of the patient and the image-capture device in the desired orientation relative to each other for image comprises:
receiving data representing at least one digital image of the face of the patient;
determining, from the received image capture data representing at least one digital image of the face of the patient, a first orientation of the face of the patient relative to the image capture device, and presenting using a position indicator a first indicator associated with the first orientation;
receiving further data representing at least one digital image of the face of the patient, and determining, from the received further data representing at least one digital image of the face of the patient, a second orientation of the face of the patient, said second orientation being different from the first orientation, and presenting using the position indicator a second indicator associated with the second orientation;
wherein the first indicator indicates a comparison between the first orientation and the desired orientation, and the second indicator indicates a comparison between the second orientation and the desired orientation.

47. The method for selecting a patient interface for a patient for use with a respiratory therapy device according to claim 42,
wherein the step of providing guidance to position the face of the patient and the image-capture device in the desired orientation relative to each other for image capture comprises:
on a display interface displaying a static indicator representing the desired orientation of the face of the patient relative to the image capture device, the static indicator being displayed at a fixed location on the display interface;
receiving data representing at least one digital image of the face of the patient;
determining, from the received data representing at least one digital image of the face of the patient, an orientation of the face of the patient relative to the image capture device;
on the display interface, displaying a dynamic indicator representing a current orientation of the face of the patient relative to the image capture device, wherein a difference between the location of the static indicator and the dynamic indicator on the display interface is representative of a difference between the current orientation of the face of the patient relative to the image capture device and the desired orientation of the face of the patient relative to the image capture device.

48. The method for selecting a patient interface for a patient for use with a respiratory therapy device according to claim 42, wherein the step of providing guidance to position the face of the patient and the image capture device in the desired orientation relative to each other for image capture comprises:
providing prompts to assist the patient to attain a required height of the face of the patient relative to the image-capture device;
providing prompts to assist the patient to attain a required angle of the face of the patient relative to the image-capture device; and
providing prompts to assist the patient to attain a required distance between the face of the patient and the image-capture device.

49. (canceled)

50. The method according to claim 46 wherein the first indicator and the second indicator are visual indicators; the visual indicators are displayed on a display interface of an electronic device; and/or the visual indicators are displayed on a display interface of the image capture device.

51. The method according to claim 46 wherein the position indicator comprises a progressive animation sequence to indicate the orientation of the face towards and away from the desired orientation in dependence on a current orientation of the face of the patient relative to the image capture device, the progressive animation sequence comprising a series of indicators displayed at different locations on a display interface, the first and second indicators being indicators of the sequence of indicators.

52. The method according to claim 46 wherein the position indicator is configured to dynamically change position and/or appearance in response to a detected change in angle of the face of the patient relative to the image capture device.

53. The method according to claim 46 comprising the step of determining a field of view of the patient and wherein the position indicator is displayed on a display interface at a location on the display interface within the field of view of the patient.

54. The method according to claim 53 wherein the field of view of the patient is determined using an angular range with respect to a front of the face of the patient or with respect to an eye of the patient at said determined orientation.

55. The method according to claim 53 comprising the step of determining a position of the image capture device and wherein the field of view of the patient is determined using the position of the image capture device.

56. The method according to claim 46 wherein the position indicator is located on a first portion of a display interface, said first portion at least partly defined by a first edge of the display interface.

57. The method according to claim 42 wherein, on determining that the orientation of the face of the patient is in the desired orientation, presenting an image capture indicator to indicate that the image capture device is capturing an image of the face of the patient, the image capture indicator being displayed on a display interface at a location within a field of view of the patient.

58. The method according to claim 42 wherein on determining that the orientation of the face of the patient is in the desired orientation, triggering an image capture process, the image capture process comprising the steps of:
displaying an image capture indicator on a display interface at a location on the display interface;
capturing an image of the face of the patient with the image capture device for an image capture time period; and
during said image capture time period, monitoring the orientation of the face of the patient;
wherein during the image capture time period if the orientation of the face of the patient changes from the desired orientation, at least one of the following takes place: terminating the image capture process, suspending the image-capture process, or providing further guidance to position the face of the patient and the image-capture device in the desired orientation relative to each other.

59. The method according to claim 58 wherein upon terminating or suspending the image capture process, presenting a different indicator, and/or an additional indicator, from the image capture indicator.

60. The method according to claim 46 wherein the position indicator presents an animation sequence to indicate the orientation of the face of the patient in relation to the desired orientation, wherein the animation sequence increases or decreases in size proportionate to how close or far the face of the patient is from the desired orientation.

61. (canceled)

62. The method according to claim 46 wherein, in addition to the first and second indicators, the position indicator further comprises non-current-position-indicating portions corresponding to other potential, non-current, orientations of the face of the patient relative to the image capture device.

63-95. (canceled)

96. A system for selecting a patient interface for a patient for use with a respiratory therapy device, the patient interface suitable to deliver respiratory therapy to the patient, the system being configured to perform the steps of:
determining a dimension of a facial feature required in order to select a patient interface for a patient;
determining a desired orientation of a face of the patient, relative to an image-capture device, to be captured in a digital image, in order to determine the dimension of the required facial feature;
providing guidance to position the face of the patient and the image-capture device in the desired orientation relative to each other for image capture;
receiving image capture data representing at least one digital image of the face of the patient, the image capture data representing the face of the patient orientated in the desired orientation relative to the image-capture device;
calculating the dimension for the facial feature from the image capture data; and, using the dimension to select the patient interface for the patient.
